(19) United States
(12) Patent Application Publication
(10) Pub. No.: US 2015/0140640 A1
(43) Pub. Date: May 21, 2015

(54) PROCESS FOR GROWING NATURAL OR ENGINEERED HIGH LIPID ACCUMULATING STRAIN ON CRUDE GLYCEROL AND/OR OTHER SOURCES OF WASTE CARBON FOR THE PRODUCTION OF OILS, FUELS, OLEOCHEMICALS, AND OTHER VALUABLE ORGANIC COMPOUNDS

(71) Applicant: Kiverdi, Inc., Berkeley, CA (US)

(72) Inventors: John S. Reed, Emeryville, CA (US); Peter Dalla-Betta, Hayward, CA (US); Itzhak Kurek, San Francisco, CA (US); Christer Jansson, Berkeley, CA (US); Michael Siani-Rose, San Francisco, CA (US); Lisa Dyson, Berkeley, CA (US)

(73) Assignee: Kiverdi, Inc., Berkeley, CA (US)

(21) Appl. No.: 14/361,603

(22) PCT Filed: Nov. 29, 2012
(86) PCT No.: PCT/US2012/067112

§ 371 Date: May 29, 2014

Related U.S. Application Data
(63) Continuation-in-part of application No. 13/623,089, filed on Sep. 19, 2012.
Application No. 13/623,089 is a continuation-in-part of application No. PCT/US2011/034218, filed on Apr. 27, 2011.
Application No. PCT/US2011/034218 is a continuation-in-part of application No. PCT/US2010/001402, filed on May 12, 2010.
Application No. PCT/US2010/001402 is a continuation-in-part of application No. 12/613,550, filed on Nov. 6, 2009, now abandoned.
(60) Provisional application No. 61/536,056, filed on Sep. 19, 2011.
(60) Provisional application No. 61/328,184, filed on Apr. 27, 2010.
(60) Provisional application No. 61/111,794, filed on Nov. 6, 2008.
(60) Provisional application No. 61/564,812, filed on Nov. 29, 2011.
(60) Provisional application No. 61/542,823, filed on Oct. 4, 2011.
(60) Provisional application No. 61/616,560, filed on Mar. 28, 2012.

(51) Int. Cl. C12N 1/20

(52) U.S. Cl. CPC C12N 1/20
USPC 435/252.34; 435/252.3

(57) ABSTRACT

Disclosed herein are microorganisms capable of growing on crude glycerol and/or glycerol and/or methanol, or combinations thereof. In some embodiments the microorganisms are knallgas bacteria that produce or secrete at least 10% of lipid by weight. Also disclosed are methods of converting crude glycerol and/or glycerol and/or methanol produced as byproduct of processes including but not limited to biodiesel production, into organic carbon molecules such as triacylglycerol useful for industrial processes including but not limited to the production of additional biodiesel. Also disclosed are methods of manufacturing chemicals or producing precursors to chemicals useful in oleochemicals, jet fuel, diesel fuel, and biodiesel fuel. Exemplary chemicals or precursors to chemicals useful in fuel and/or oleochemical production are alkanes, alkenes, alkynes, fatty acid alcohols, fatty acid aldehydes, methyl esters, ethyl esters, alkyl esters, with carbon chains between five and twenty four carbon atoms long.

### RELATED APPLICATIONS

This application claims priority under 35 U.S.C. §119(e) to U.S. Provisional Patent Application No. 61/564,812, filed Nov. 29, 2011 and entitled “PROCESS FOR GROWING NATURAL OR ENGINEERED HIGH LIPID ACCUMULATING STRAIN ON CRUDE GLYCEROL AND/OR OTHER SOURCES OF WASTE CARBON FOR THE PRODUCTION OF OILS, FUELS, OLEOCHEMICALS, AND OTHER VALUABLE ORGANIC COMPOUNDS”. This application is also a continuation-in-part of U.S. patent application Ser. No. 13/623,089, filed Sep. 19, 2012, and entitled “INDUSTRIAL FATTY ACID ENGINEERING GENERAL SYSTEM FOR MODIFYING FATTY ACIDS,” which is a continuation-in-part of International Patent Application No. PCT/US2011/34218, filed Apr. 27, 2011, and entitled “USE OF OXYHYDROGEN MICROORGANISMS FOR NON-PHOTOSYNTHETIC CARBON CAPTURE AND CONVERSION OF INORGANIC AND/OR C1 CARBON SOURCES INTO USEFUL ORGANIC COMPOUNDS,” which is a continuation-in-part of International Patent Application No. PCT/US2010/001402, filed May 12, 2010, and entitled “BIOLOGICAL AND CHEMICAL PROCESS UTILIZING CHEMOAUTOTROPHIC MICROORGANISMS FOR THE CHEMOSYNTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS,” which is a continuation-in-part of U.S. patent application Ser. No. 12/613,550, filed Nov. 6, 2009, and entitled “BIOLOGICAL AND CHEMICAL PROCESS UTILIZING CHEMOAUTOTROPHIC MICROORGANISMS FOR THE CHEMOSYNTHETIC FIXATION OF CARBON DIOXIDE AND/OR OTHER INORGANIC CARBON SOURCES INTO ORGANIC COMPOUNDS, AND THE GENERATION OF ADDITIONAL USEFUL PRODUCTS,” which claims the benefit of U.S. Provisional Patent Application No. 61/111,794, filed Nov. 6, 2008, and entitled, “BIOLOGICAL AND CHEMICAL PROCESS UTILIZING CHEMOAUTOTROPHIC MICROORGANISMS FOR THE RECYCLING OF CARBON FROM CARBON DIOXIDE AND OTHER INORGANIC CARBON SOURCES THROUGH CHEMOSYNTHESIS INTO BIOFUEL AND ADDITIONAL USEFUL PRODUCTS.” Each of these applications is incorporated herein by reference in its entirety for all purposes.

### FIELD OF THE INVENTION

This disclosure relates to compositions capable of producing and methods of the producing oils, fuels, and oleochemicals through cultivating bacteria that grow on crude glycerol, also called crude glycerine or bio-crude, produced through processes such as bio-diesel production, and/or that grow on other sources of waste or low value carbon such as methanol. This disclosure further relates to methods of converting low value or waste sources of carbon into useful organic molecules such as fatty acid alcohols, fatty acid aldehydes, fatty acid esters, lipids, alkanes, alkenes, and alkynes. The bacteria of the invention can be genetically engineered for use in the methods or other aspects of the invention described herein.

### BACKGROUND OF THE INVENTION

Sustainable and renewable sources of oleochemicals, such as are used in lubricants, surfactants, monomers, soaps, personal care products, as well as liquid fuels to operate machinery, aircraft, and vehicles, are necessary to reduce the amount of carbon dioxide emissions in the atmosphere, as well as to reduce global energy consumption based upon petrochemicals.

Increased demand for energy by the global economy has placed increasing pressure on the cost of hydrocarbons and petrochemicals. Aside from energy, many industries, including plastics and chemical manufacturers, rely heavily on the availability of oils and hydrocarbons as a feedstock of their manufacturing processes. Cost-effective alternatives to current sources of supply could help mitigate the upward pressure on energy demand and raw material costs.

Plant-based productions of oils or oleochemicals such as from palm oil are known but are associated with heavy deforestation of sensitive rainforest habitat and environmental damage.

Microbial systems for the production of lipids or oils are known. Algal systems have been developed to create oil through photosynthesis. However insufficient yields limit the effectiveness, economic feasibility, practicality and commercial adoption. Algal, bacterial, and yeast systems have been developed for the production of oil or oleochemicals from a sugar feedstock. However high feedstock costs and problems with food versus oleochemical production conflicts make this a doubtful approach.

Crude glycerol byproduct from bio-diesel production, as well as other processes including but not limited to those involving the transesterification triacylglycerols with methanol, ethanol, and/or other alcohols, represents a low cost source of carbon and energy that at times has been considered a waste product. In addition to containing glycerol, crude glycerol usually contains a methanol contaminant of around 20%, but which can be greater or less than this amount. The price of crude glycerol has fallen as low as less than 1 cent/lb during 2008-2009-when the material was either burned or sprayed into coal mines to control dust [OUTLOOK '11: Bumpy ride likely for US oleochemicals http://www.icis.com/Articles/2010/12/28/9421467/outlook-11-bumpy-ride-likely-for-us-oleochemicals.html]—up to current prices (2011) of about 12-16 cents/lb [ICIS Pricing Glycerine (US Gulf) http://www.icispricing.com/il_shared/Samples/SubPage170.asp]. Even at the price of $0.16/lb crude glycerol is far below the world price of sugar in terms of cost per unit energy content and cost per unit carbon content. Hence crude glycerol can serve as a much cheaper energy and carbon source than sugar for the growth of microorganisms to produce higher value chemicals including but not limited to oils, oleochemicals, and fuels. However in order to utilize this low cost feedstock to produce oleochemicals through microbial production a microorganism is needed that can not only use glycerol as an energy and carbon source for synthesis and growth, but can also tolerate and/or grow on the impurities in crude glycerol including but not limited to methanol. If higher purity glycerol (i.e. lower levels of methanol and/or other impurities) than what is characteristic of crude glycerol is required for microbial growth, then the cost advantage of using glycerol relative to sugar largely or entirely disappears. Hence for the embodiment of the present invention targeting crude glycerol as the energy and carbon source for microbial growth, a tolerance of contaminants in crude glycerol such as methanol, ethanol, matter organic non-glycerol (MONG), and salts is essential. Additionally a microorganism that is suitable for economically converting crude glycerol into oils and/or oleochemicals should be able to synthesize high quantities of lipids. In summary the type of microorganism that is desirable for the present invention must be able to grow on glycerol, and tolerate and/or grow on methanol as well as other impurities present in crude glycerol, and be able to direct a high proportion of the carbon and energy provided by the glycerol, and/or the methanol in crude glycerol, and/or other waste or low value feedstocks, into lipid products.

There is a need to identify a set of microorganisms that can grow on crude glycerol with methanol contamination and/or other alcohol contaminants, as well as other waste or low cost energy and carbon sources, that can synthesize commercially viable sets of organic carbon chains of at least five carbon atoms long, and particularly lipids, in a commercially feasible method. There is a need to identify microorganisms not limited metabolically by typical carbon and energy inputs, and a microorganism that can additionally utilize crude glycerol, glycerol, methanol, other alcohols, and other non-sugar organic compounds, enabling a capability of using lower cost feedstocks than sugar for the microbial production of oils and/or oleochemicals.

### SUMMARY OF THE INVENTION

The present invention characterizes and enables microorganisms to be used for the production of organic compounds including but not limited to lipids, oils, or oleochemicals from low cost and/or waste energy and carbon sources including but not limited to the crude glycerol byproduct of biodiesel production. The present invention allows the crude glycerol byproduct of biodiesel production to be converted into additional triacylglycerols and/or other neutral lipids, which can be in turn converted into additional biodiesel, thereby increasing the yield of biodiesel produced from a given initial input of triacylglycerol and/or other neutral lipid into the biodiesel production process. The present technology allows the development of new genetically enhanced strains of microorganisms that can be used to produce and/or secrete targeted organic compounds including but not limited to oleochemicals and/or drop-in liquid fuels, such as are currently only produced economically in bulk from petroleum or higher plants, directly from low cost and/or waste energy and carbon sources including but not limited to the crude glycerol byproduct of biodiesel production.

The microorganisms and methods of the present invention enable low cost synthesis of chemicals and fuels, which can compete on price with petrochemicals and higher-plant derived oleochemicals, and which will generally have a substantially lower price than oleochemicals produced through heterotrophic growth on sugar or microbial phototrophic synthesis.

The invention relates to a composition comprising a microorganism that converts a waste or low cost energy and carbon source, including but not limited to the crude glycerol byproduct of processes such as biodiesel production, into one or more lipids. In some embodiments, the composition comprises a microorganism, wherein the microorganism is a knallgas microorganism (also known as an oxyhydrogen microorganism). In some embodiments, the composition comprises a microorganism, wherein the microorganism is chosen from the genera Rhodococcus or Gordonia. In some embodiments, the composition comprises a microorganism, wherein the microorganism is Rhodococcus opacus. In some embodiments, the composition comprises a microorganism, wherein the microorganism is Rhodococcus opacus (DSM 43205) or Rhodococcus opacus (DSM 43206). In some embodiments, the composition comprises a microorganism, wherein the microorganism is Cupriavidus necator (DSM531). In some embodiments, the composition comprises a crude glycerol feedstock as a carbon and energy source for microbial growth wherein glycerol comprises 70 to 90 percent by weight of the crude glycerol. In some embodiments glycerol comprises 50 to 70 percent by weight of the crude glycerol. In some embodiments glycerol comprises less than 50 percent by weight of the crude glycerol. In some embodiments, the composition comprises a crude glycerol feedstock as a carbon and energy source for microbial growth wherein methanol comprises 10 to 20 percent by weight of the crude glycerol. In some embodiments methanol comprises less than 1 percent by weight of the crude glycerol. In some embodiments methanol comprises over 20 percent by weight of the crude glycerol. In some embodiments, the composition comprises a crude glycerol feedstock as a carbon and energy source for microbial growth wherein free fatty acids comprise 1 to 10 percent by weight of the crude glycerol. In some embodiments free fatty acids comprise less than 1 percent by weight of the crude glycerol. In some embodiments free fatty acids comprise over 10 percent by weight of the crude glycerol. In some embodiments, the composition comprises a crude glycerol feedstock as a carbon and energy source for microbial growth wherein MONG comprise 1 to 10 percent by weight of the crude glycerol. In some embodiments MONG comprises less than 1 percent by weight of the crude glycerol. In some embodiments MONG comprises over 10 percent by weight of the crude glycerol. In some embodiments, the composition comprises a crude glycerol feedstock as a carbon and energy source for microbial growth wherein salts comprise 5 to 10 percent by weight of the crude glycerol. In some embodiments salts comprises less than 5 percent by weight of the crude glycerol. In some embodiments salts comprise 10 to 15 percent by weight of the crude glycerol. In some embodiments salts comprise over 15 percent by weight of the crude glycerol.

In some embodiments, the composition comprises a microorganism wherein the microorganism can naturally grow on crude glycerol and/or glycerol and/or methanol and/or ethanol, and wherein the microorganism can naturally accumulate lipid to 50% or more of the cell biomass by weight. In some embodiments the microorganisms have a native ability to send a high flux of carbon down the fatty acid biosynthesis pathway. In some embodiments the microorganism exhibiting these traits is Rhodococcus opacus (DSM 43205 or DSM 43206).

In some embodiments, the composition comprises a microorganism and a process wherein the microorganism can grow on the crude glycerol byproduct of biodiesel production, including any methanol or other alcohol contaminants in said crude glycerol, and convert the crude glycerol into additional triacylglycerols (TAGs) and/or other neutral lipids, which are in turn extracted from the cell mass using methods known in the art of microbial oil production. The extracted lipids are then converted into additional biodiesel through transesterification in some embodiments, or sold as a raw oil feedstock for the production of additional biodiesel in other embodiments. In some embodiments the composition comprises a microorganism and a process that increase the yield of biodiesel from an initial input of triacylglycerol and/or other neutral lipid into the biodiesel production process by converting the crude glycerol byproduct of the process into additional TAGs and/or other neutral lipids that can be fed back into the biodiesel production process for the production of additional biodiesel. In some embodiments the crude glycerol byproduct resulting from the transesterification of TAGs and/or other neutral lipids produced by the strains of the present invention grown on crude glycerol, is used to further grow the strains of the present invention and produce additional TAGs and/or other neutral lipids. In some embodiments the microorganism in the composition is the strain Rhodococcus opacus (DSM 43205) and/or Rhodococcus opacus (DSM 43206).

In some embodiments, the invention relates to a naturally occurring or non-naturally occurring microorganism capable of converting crude glycerol and/or glycerol and/or methanol into targeted oleochemical products. In some embodiments, the invention relates to a non-naturally occurring microorganism capable of converting crude glycerol and/or glycerol and/or methanol into targeted oleochemical products where the wild-type microorganism is capable of growing on crude glycerol and/or glycerol and/or methanol, but is either not capable of synthesizing said targeted oleochemical products, or is capable of synthesizing the targeted oleochemicals, but is not capable of synthesizing the targeted biochemical products at the concentration and/or efficiency of the non-natural microorganism. In such microorganisms, one or more proteins or enzymes are expressed in the microorganism, thereby modifying, extending, diverting, enhancing, promoting, or otherwise altering the lipid biosynthesis pathway or its regulation for the synthesis and/or enhanced synthesis of a targeted lipid-based product, oleochemical, or hydrocarbon.

In some embodiments, the invention relates to a non-naturally occurring microorganism capable of converting crude glycerol and/or glycerol and/or methanol into targeted oleochemical products, where the wild-type microorganism is capable of growing on crude glycerol and/or glycerol and/or methanol and/or other waste energy and carbon sources and is capable of synthesizing said targeted oleochemical products, but the non-naturally occurring microorganism is capable of synthesizing the targeted biochemical products at a higher concentration and/or efficiency than the wild-type microorganism due to the overexpression and/or underexpression of one or more proteins or enzymes.

In some embodiments, the invention relates to compositions comprising one or more bacterial cells that consist of zero, one, two, or three exogenous nucleic acid sequences where said bacteria can grow on crude glycerol and/or glycerol and/or methanol and/or other waste energy and carbon sources as a source of carbon and/or energy.

In some embodiments, the invention relates to compositions comprising one or more bacterial cells of Rhodococcus opacus (DSM 43205) that consist of zero, one, two, or three exogenous nucleic acid sequences.

In some embodiments one, two, or three exogenous nucleic acid sequences encode one or more thioesterase proteins.

In some embodiments, the invention relates to compositions comprising one or more bacterial cells that consist of two exogenous nucleic acid sequences that encode the following proteins: fatty acid acyl-ACP reductase, a fatty acid aldehyde decarbonylase, where said bacteria can grow using crude glycerol and/or glycerol and/or methanol and/or other waste energy and carbon sources as a source of carbon and/or energy.

In some embodiments, the invention relates to compositions comprising one or more bacterial cells that consist of three exogenous nucleic acid sequences that encode the following proteins: fatty acid acyl-ACP reductase, a fatty acid aldehyde decarbonylase, and a thioesterase, where said bacteria can grow using crude glycerol and/or glycerol and/or methanol and/or other waste energy and carbon sources as a source of carbon and/or energy.

In some embodiments, the non-natural bacterial cell produces and/or secretes one or more lipids in an amount that is greater than the amount of lipids produced and/or secreted by the same cell not comprising the exogenous nucleic acid sequence.

In some embodiments, the non-natural bacterial cell produces and/or secretes one or more lipids having a given carbon chain length, where the amount of said lipid produced and/or secreted is greater than the amount produced and/or secreted by the same cell not comprising the exogenous nucleic acid sequence.

In some embodiments, the non-natural bacterial cell produces and/or secretes one or more lipid molecules in an amount that is less than the amount of lipids produced and/or secreted by the same cell not comprising the exogenous nucleic acid sequence.

In some embodiments, the non-natural bacterial cell produces and/or secretes one or more hydrocarbons in an amount that is greater than the amount of hydrocarbons produced and/or secreted by the same cell not comprising the exogenous nucleic acid sequence.

In some embodiments, the non-natural bacterial cell produces and/or secretes one or more lipids or hydrocarbons in a ratio greater than the ratio of lipids or hydrocarbons produced and/or secreted by the same cell not comprising the one or more exogenous nucleic acid sequences.

In some embodiments, the bacterial cell produces and/or secretes one or more lipids or hydrocarbons, wherein at least 50% of the one or more lipids or hydrocarbons have 5 to 24 carbon atoms. In some embodiments, the bacterial cell produces and/or secretes one or more lipids or hydrocarbons, wherein at least 60% of the one or more lipids or hydrocarbons have 5 to 24 carbon atoms. In some embodiments, the bacterial cell produces and/or secretes one or more lipids or hydrocarbons, wherein at least 70% of the one or more lipids or hydrocarbons have 5 to 24 carbon atoms. In some embodiments, the bacterial cell produces and/or secretes one or more lipids or hydrocarbons, wherein at least 75% of the one or more lipids or hydrocarbons have 5 to 24 carbon atoms. In some embodiments, the bacterial cell produces and/or secretes one or more lipids or hydrocarbons, wherein at least 80% of the one or more lipids or hydrocarbons have 5 to 24 carbon atoms.

In some embodiments, the bacterial cell or compositions comprising the bacterial cell comprise at least one exogenous nucleic acid sequence that is integrated into the genome of the cell.

In some embodiments, the invention relates to a composition comprising a microorganism that converts crude glycerol and/or glycerol and/or methanol into one or more lipids or hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase. In some embodiments the microorganism is Rhodococcus opacus. In some embodiments the microorganism is Cupriavidus necator.

In some embodiments, the invention relates to a composition comprising a microorganism that converts crude glycerol and/or glycerol and/or methanol into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase, wherein the one or more hydrocarbons have a carbon chain length of at least 8 carbon atoms. In some embodiments, the invention relates to a composition comprising a microorganism that converts crude glycerol and/or glycerol and/or methanol into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the one or more hydrocarbons comprise a mixture of hydrocarbon molecules having a carbon chain length from 5 carbon atoms to 24 carbon atoms. In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the one or more lipids comprise a quantity of at least one alkane, alkene, alkyne, fatty alcohol, fatty ester, and/or fatty aldehyde at a level higher than the quantity of the alkane, alkene, alkyne, fatty alcohol, fatty ester, and/or fatty aldehyde in the same microorganism not comprising the heterologous nucleic acid sequences. In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 60% of one or more lipids by weight.

In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 65% of one or more lipids by weight. In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 70% of one or more hydrocarbons by weight.

In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 75% of one or more lipids by weight. In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 80% of one or more lipids by weight.

In some embodiments, the invention relates to a composition comprising a microorganism that converts crude glycerol and/or glycerol and/or methanol into one or more lipids, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the microorganism produces and/or secretes at least 85% of one or more lipids by weight. In some embodiments, the invention relates to a composition comprising a microorganism that converts crude glycerol and/or glycerol and/or methanol into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein less than 10% by weight of the hydrocarbons produced is methane.

In some embodiments, the invention relates to a composition comprising a microorganism that converts crude glycerol and/or glycerol and/or methanol into one or more organic compounds, wherein less than 10% by weight of the organic compounds produced are organic acids with carbon chain length of four carbons or less.

In some embodiments, the invention relates to a composition comprising a microorganism that converts crude glycerol and/or glycerol and/or methanol into one or more organic compounds, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein less than 10% by weight of the organic compounds produced are organic acids with carbon chain length of four carbons or less.

In some embodiments, the invention relates to a composition comprising a microorganism that converts crude glycerol and/or glycerol and/or methanol into one or more lipids, wherein at least one lipid produced is a component or a precursor of a component of biodiesel fuel.

In some embodiments, the invention relates to a composition comprising a microorganism that converts crude glycerol and/or glycerol and/or methanol into one or more lipids or hydrocarbons, wherein at least one lipid produced is a component or a precursor of a component of jet fuel, diesel fuel, or biodiesel fuel.

In some embodiments, the invention relates to a composition comprising a microorganism that converts crude glycerol and/or glycerol and/or methanol into one or more lipids or hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein at least one lipid produced is a component or a precursor of a component of jet fuel, diesel fuel, or biodiesel fuel.

In some embodiments, the invention relates to a composition comprising a microorganism that converts crude glycerol and/or glycerol and/or methanol into one or more hydrocarbons, wherein the microorganism comprises at least a first and a second exogenous nucleic acid sequence, wherein the first exogenous nucleic acid sequence encodes fatty acid acyl-ACP reductase and the second exogenous nucleic acid sequence encodes a fatty acid aldehyde decarbonylase; wherein the hydrocarbons produced comprise a mixture of at least two hydrocarbons having a carbon backbone from 5 to 24 carbon atoms.

The present invention also relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts crude glycerol and/or glycerol and/or methanol into lipids. In some embodiments, the invention relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts crude glycerol and/or glycerol and/or methanol; wherein the cell produces and/or secretes at least 75% of one or more hydrocarbons by weight. In some embodiments, the invention elates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts crude glycerol and/or glycerol and/or methanol into lipid; wherein the cell produces and/or secretes at least 75% of one or more hydrocarbons by weight when cultured at least 42 degrees Celsius for at least 1 hour. In some embodiments, the bacterial cell is cultured without exposure to light.

In some embodiments, the invention relates to a bacterial cell wherein the cell converts crude glycerol and/or glycerol and/or methanol into a triacylglycerol or mixture of triacylglycerols; wherein the cell is a strain of Rhodococcus opacus. In some embodiments the strain is Rhodococcus opacus (DSM 43205). In some embodiments the strain is Rhodococcus opacus (DSM 43206).

In some embodiments, the invention relates to a bacterial cell comprising at least two exogenous nucleic acid sequences, wherein the at least two exogenous nucleic acid sequences encode fatty acid acyl-ACP reductase and fatty acid aldehyde decarbonylase, and wherein the cell converts crude glycerol and/or glycerol and/or methanol into a hydrocarbon or mixture of hydrocarbons, and/or other lipids; wherein the cell is a strain of Rhodococcus opacus.

In some embodiments, the invention relates to a bacterial cell comprising a first, a second, and a third exogenous nucleic acid sequence, wherein the first exogenous nucleic acid sequence encodes fatty acid acyl-ACP reductase, the second exogenous nucleic acid sequence encodes a fatty acid aldehyde decarbonylase, and the third exogenous nucleic acid sequence encodes a thioesterase; and wherein the cell converts crude glycerol and/or glycerol and/or methanol into a lipid or mixture of lipids. In some embodiments, the bacterial cell comprises no more than eight exogenous nucleic acids that encode a lipid pathway enzyme. In some embodiments, the bacterial cell comprises no more than seven exogenous nucleic acids that encode a lipid pathway enzyme. In some embodiments, the bacterial cell comprises no more than six exogenous nucleic acids that encode a lipid pathway enzyme. In some embodiments, the bacterial cell comprises no more than five exogenous nucleic acids that encode a lipid pathway enzyme. In some embodiments, the bacterial cell comprises no more than four exogenous nucleic acids that encode a lipid pathway enzyme. In some embodiments, the bacterial cell comprises no more than three exogenous nucleic acids that encode a lipid pathway enzyme. In some embodiments, the bacterial cell comprises no more than two exogenous nucleic acids that encode a lipid pathway enzyme. In some embodiments, the bacterial cell comprises no more than one exogenous nucleic acid that encodes a lipid pathway enzyme. In some embodiments, the bacterial cell comprises no more than eight exogenous nucleic acids that encode a protein. In some embodiments, the bacterial cell comprises no more than seven exogenous nucleic acids that encode a protein. In some embodiments, the bacterial cell comprises no more than six exogenous nucleic acids that encode a protein. In some embodiments, the bacterial cell comprises no more than five exogenous nucleic acids that encode a protein. In some embodiments, the bacterial cell comprises no more than four exogenous nucleic acids that encode a protein. In some embodiments, the bacterial cell comprises no more than three exogenous nucleic acids that encode a protein. In some embodiments, the bacterial cell comprises no more than two exogenous nucleic acids that encode a protein. In some embodiments, the bacterial cell comprises no more than one exogenous nucleic acid that encodes a protein.

In some embodiments the invention relates to a method of producing a lipid or mixture of lipids in a microorganism population comprising the cell or the composition described herein, wherein the method comprises: culturing a population of microorganisms comprising the cell or the composition described herein in a feedstock comprising crude glycerol and/or glycerol and/or methanol.

In some embodiments, the invention relates to a method of producing a lipid or mixture of lipids, wherein the method comprises: culturing a population of bacterial cells comprising the cell or the composition described herein in a feedstock comprising crude glycerol and/or glycerol and/or methanol. In some embodiments, the microorganism population comprises a bacterial strain of Rhodococcus opacus. In some embodiments, the bacterial strain is Rhodococcus opacus (DSM 43205). In some embodiments, the bacterial strain is Rhodococcus opacus (DSM 43206).

In some embodiments, the invention relates to a method of producing a lipid or mixture of lipids, wherein the method comprises: culturing a population of bacterial cells comprising the cell or the composition described herein in a feedstock comprising methanol, a common impurity of crude glycerol, with or without the addition of glycerol. In some embodiments, the microorganism population comprises a bacterial strain of Rhodococcus opacus. In some embodiments, the bacterial strain is Rhodococcus opacus (DSM 43205). In some embodiments, the bacterial strain is Rhodococcus opacus (DSM 43206).

In some embodiments, the method comprises a population of microorganisms or bacterial cell described herein that produces and/or secretes lipids of a weight equal to or greater than 10% of the total percentage of cellular dry matter. In some embodiment, the method comprises a population of microorganisms or bacterial cell described herein that produces and/or secretes lipids of a weight equal to or greater than 20% of the total percentage of cellular dry matter. In some embodiment, the method comprises a population of microorganisms or bacterial cell described herein that produces and/or secretes lipids of a weight equal to or greater than 30% of the total percentage of cellular dry matter. In some embodiments, the method comprises a population of microorganisms or bacterial cell described herein that produces and/or secretes lipids of a weight equal to or greater than 40% of the total percentage of cellular dry matter. In some embodiment, the method comprises a population of microorganisms or bacterial cell described herein that produces and/or secretes lipids of a weight equal to or greater than 50% of the total percentage of cellular dry matter. In some embodiments, the method comprises a population of microorganisms or bacterial cells described herein that produces and/or secretes lipids of a weight equal to or greater than 60% of the total percentage of cellular dry matter. In some embodiments, the method comprises a population of microorganisms or bacterial cells described herein that produces and/or secretes lipids of a weight equal to or greater than 70% of the total percentage of cellular dry matter. In some embodiments, the method comprises a population of microorganisms or bacterial cell described herein that produces of secretes lipids of a weight equal to or greater than 75% of the total percentage of cellular dry matter. In some embodiment, the method comprises a population of microorganisms or bacterial cell described herein that produces of secretes lipids of a weight equal to or greater than 80% of the total percentage of cellular dry matter. In some embodiments, the method comprises a population of microorganisms or bacterial cell described herein that produces of secretes lipids of a weight equal to or greater than 85% of the total percentage of cellular dry matter. In some embodiments, the bacterial cell or composition comprising the bacterial cell produces and/or secretes at least 10% of the total percentage of the cellular dry matter or 10% by weight. In some embodiment, the method comprises a population of microorganisms comprising a bacterial cell described herein that produces or secretes lipids, wherein at least 5% of the lipids have carbon backbones from 5 to 24 carbon atoms in length. In some embodiments, the method comprises a population of microorganisms comprising a bacterial cell described herein that produces or secretes lipids, wherein at least 10% of the lipids have carbon backbones from 5 to 24 carbon atoms in length. In some embodiments, the method comprises a population of microorganisms comprising a bacterial cell described herein that produces or secretes lipids, wherein at least 15% of the lipids have carbon backbones from 5 to 24 carbon atoms in length. In some embodiments, the method comprises a population of microorganisms comprising a bacterial cell described herein that produces or secretes lipids, wherein at least 20% of the lipids have carbon backbones from 5 to 24 carbon atoms in length.

In some embodiments, the method comprises a population of microorganisms comprising a bacterial cell described herein that produces or secretes lipids, wherein at least 5% or 10% or 15% or 20% of the lipids have carbon backbones that are suitable for conversion to biodiesel through methods known in the art such as transesterification.

In some embodiments of the invention, the invention relates to a method of producing and/or secreting a lipid or mixture of lipids by culturing a population of microorganisms comprising a bacterial cell described herein, wherein the exogenous nucleic acid sequences are operably linked to a promoter that is inducible in response to a first stimulus, and wherein the method further comprises: culturing the population of bacterial cells for a first period of time in the presence of a first stimulus to produce one or more lipids chosen from an alkane, alkene, alkyne, fatty acid alcohol, fatty acid ester, fatty acid aldehyde, and/or TAG.

In some embodiments, the bacterial cell is Rhodococcus opacus or the population of microorganisms comprises a Rhodococcus cell.

In some embodiments, the bacterial cell comprises no more than five exogenous nucleic acid sequences that encode a lipid pathway enzyme. In some embodiments the bacterial cell comprises at least a first and a second exogenous nucleic acid sequence but no more than five exogenous nucleic acid sequences, wherein the first exogenous nucleic acid sequence encodes fatty acid acyl-ACP reductase and the second exogenous nucleic acid sequence encodes a fatty acid aldehyde decarbonylase.

In some embodiments, the invention relates to a method of producing one or more fatty acid alcohols, fatty acid aldehydes, fatty acid esters, alkanes, alkenes, alkynes, TAGs, other neutral lipids, or any combination thereof comprising exposing a bacterial cell to crude glycerol and/or glycerol and/or methanol and/or any mixture thereof; wherein the bacterial cell is capable of converting crude glycerol and/or glycerol and/or methanol into one or more fatty acid alcohols, fatty acid aldehydes, fatty acid esters, alkanes, alkenes, alkynes, TAGs, neutral lipids. In some embodiments the microorganism comprises at least a first exogenous nucleic acid and a second exogenous nucleic acid, wherein the first exogenous nucleic acid encodes fatty acid acyl-ACP reductase and the second exogenous nucleic acid encodes fatty acid aldehyde decarbonylase. In some embodiments, the first and second exogenous nucleic acids are heterologous nucleic acid sequences. In some embodiments, the bacterial cell comprises at least a first, a second, and a third exogenous nucleic acid sequence, wherein the first exogenous nucleic acid sequence encodes a fatty acid acyl-ACP reductase, the second exogenous nucleic acid sequence encodes a fatty acid aldehyde decarbonylase, and the third exogenous nucleic acid sequence encodes a thioesterase. In some embodiments, the bacterial cell comprises at least a first exogenous nucleic acid sequence, wherein the first exogenous nucleic acid sequence encodes a thioesterase. In some embodiments, the bacterial cell comprises no more than five exogenous nucleic acid sequences that encode a lipid pathway enzyme.

In some embodiments, the invention relates to a method of manufacturing one or more lipids, comprising (a) culturing a cell described herein in a reaction vessel or bioreactor in the presence of crude glycerol and/or glycerol and/or methanol, wherein the cell produces and/or secretes one or more lipids in an quantity equal to or greater than at least 10% of the cell's total dry cellular mass; and (b) separating the one or more lipids from reaction vessel. In some embodiments, the method further comprises purifying the one or more lipids after separation from the reaction vessel or bioreactor. In some embodiments, the one or more lipids is a component of or a precursor to a component of jet fuel, diesel fuel, or biodiesel fuel.

In some embodiments the nucleic acid sequence is given by SEQ ID NO:5 and/or SEQ ID NO: 6. In some embodiments the nucleic acid sequence has at least 50, 60, 70, 75, 80, 85, 90, 91, 92, 93, 94, 95, 96, 97, 98 or 99% nucleotide homology to one or more of SEQ ID NOs: 5 or 6.

In some embodiments, the invention relates to a bioreactor comprising the composition or bacterial cells described herein.

In some embodiments, the invention relates to a system for the production of one or more lipids or mixture of lipids, comprising a bioreactor, which comprises: (a) a microorganism population comprising a cell described herein; and (b) an inlet connected to a feedstock source allowing delivery of a feedstock comprising crude glycerol and/or glycerol and/or methanol. In some embodiments, the lipid or mixture of lipids comprise at least one component of or one precursor to a component of jet fuel, diesel fuel, or biodiesel fuel.

### BRIEF DESCRIPTION OF THE DRAWINGS

FIG. 1 describes the taxonomic names afforded to the chemoautotrophic, knallgas, and oleaginous microorganisms used in selected embodiments of the invention;

FIG. 2 shows the 16S rRNA gene based-rooted phylogenetic tree of gordoniaceae, mycobacteriaceae, nocardiaceae and burkholderiaceae; Bar, 0.01% estimated sequence divergence;

FIG. 3 shows the sequence similarity of Rhodococcus opacus (DSM 43205) 16S rRNA gene (NR_{—}026186.1) to members of the family gordoniaceae, mycobacteriaceae, nocardiaceae and burkholderiaceae. The Genbank accession numbers, DNA length and % identity of analyzed genes are indicated;

FIG. 4 describes the nucleotide sequence alignment of the 16S rRNA genes (SEQ ID NOs: 15-43, respectively).

FIG. 5 demonstrates the growth of chemotrophic, knallgas, and oleaginous microorganisms in flasks under heterotrophic and chemotrophic conditions and on methanol. Bacterial growth was measured using optical density (OD) detection at 650 nm. Media and growth conditions are described in the Examples section below;

FIG. 6 describes the measured lipid content of microorganisms on heterotrophic and chemotrophic growth conditions as a percentage of total cellular dry matter (CDM). Cells were grown under conditions described in FIG. 5, harvested after 72 hr (unless otherwise indicated) and analyzed by gas chromatography. For CDM, total dry weight was determined gravimetrically. Cellular lipid content was determined using method AOAC 996.06; (AOAC stands for Association of Analytical Communities);

FIG. 7 describes the fatty acid profile of R. opacus (DSM 44193) under heterotrophic growth conditions. Cells were harvested after 72 hr and analyzed by gas chromatography;

FIG. 8 describes the fatty acid profile R. opacus (DSM43205) under heterotrophic (A) and chemoautotrophic (B) growth conditions. Cells were harvested after 72 hours of growth and analyzed by gas chromatography;

FIG. 9 describes the fatty acid profile Rhodococcus sp. (DSM 3346) under heterotrophic (A) chemoautotrophic (B) growth conditions. Cells were harvested after 72 hr and analyzed by gas chromatography;

FIG. 10 describes shuttle vectors (A) and genetic elements (B) for transformation and gene expression of in chemoautotrophic and oleaginous microorganisms. MCS: multiple cloning site;

FIG. 11 describes the map of the plasmids pSeqCO1(A; SEQ ID: 01), pSeqCO2 (B; SEQ ID: 02), pVer1(C; SEQ ID: 03) and pVer2 (D; SEQ ID: 04) described in FIG. 10. The genetic elements are indicated;

FIG. 12 describes the transformation of chemoautotrophic, knallgas, and oleaginous microorganisms with shuttle vectors described in FIG. 10;

FIG. 13 describes the growth of knallgas microbe Cupriavidus necator (DSM531) transformed with the plasmid (Y) pSeqCO2 (SEQ ID:2) and untransformed (N) on different kanamycin concentrations. Single colony of transformants and control were grown LB medium (per 1 L: 10 g Bacto-tryptone, 5 g yeast extract, 10 g NaCl pH=7.0) at 30° C. in the indicated kanamycin concentrations. The growth was measured using O.D after the indicated number of days;

FIG. 14 describes the formation of fatty alcohols in oleaginous bacteria. The role of the fatty acyl-CoA reductases (FAR) gene in the biosynthesis pathway is shown. The Arabidopsis genes FAR1 (SEQ ID: 05), FAR2 (SEQ ID: 06) and FAR3 (SEQ ID: 07) were cloned into pSeqCO2 plasmid using the indicated restriction sites to give pSeqCO2::FAR1, pSeqCO2::FAR2, pSeqCO2::FAR3;

FIG. 15 describes the pathway for formation of fatty alcohols in burkholderiaceae using of the fatty acyl-CoA reductases (FAR) gene;

FIG. 16 describes the cloning strategy of FAR gene into pSeqCO2 plasmids. The Arabidopsis genes FAR1 (SEQ ID: 05), FAR2 (SEQ ID: 06) and FAR3 (SEQ ID: 07) were cloned into pSeqCO2 plasmid using the indicated restriction sites to give pSeqCO2::FAR1, pSeqCO2::FAR2, pSeqCO2::FAR3;

FIG. 17 describes the effect of FAR genes expression on fatty acid synthesis in the knallgas microbe Cupriavidus necator. C. necator cells were transformed with pSeqCO2::FAR1 (Cn-F1), pSeqCO2::FAR2 (Cn-F2) and control pSEqCO2 (Cn-P). Cells were harvested (3,000×g for 20 min at 4° C.) and fatty acids were analyzed by gas chromatography;

FIG. 18 describes the pathway for formation of hydrocarbons in oleaginous bacteria using the enzymes fatty acid acyl-ACP reductase (FadDR) and fatty acid aldehyde decarbonylase by (FAD) genes. Genes from the cyanobacterium (Synechocystis sp. PCC 6803) used in the experiment were FadR (SEQ ID: 08) and FAD (SEQ ID: 09) driven by the Synechocystis sp. Rubisco large subunit promoter (SEQ ID: 09) were cloned into pSeqCO2 plasmid using the indicated restriction sites to give pSeqCO2::FUEL;

FIG. 19 describes the pathway for formation of hydrocarbons in burkholderiaceae using the enzymes fatty acid acyl-ACP reductase (FadDR) and fatty acid aldehyde decarbonylase by (FAD) genes;

FIG. 20 describes the restriction map related to the cloning strategy of FadDR and FAD genes into pSeqCO2 plasmid transformed for the experiment. Genes from the cyanobacterium (Synechocystis sp. PCC 6803) used in the experiment were FadR (SEQ ID: 08) and FAD (SEQ ID: 09) driven by the Synechocystis sp. Rubisco large subunit promoter (SEQ ID: 10) were cloned into pSeqCO2 plasmid using the indicated restriction sites to give pSeqCO2::FUEL;

FIGS. 21A and 21B describe the production of hydrocarbons in the knallgas microbe Cupriavidus necator transformed with pSeqCO2::FUEL (Cn_FUEL2.1) and empty vector (Cn-P). GC chromatogram of hydrocarbon (indicated in red) extracted from transformants grown in 50 ml LB media under previously identified conditions;

FIG. 22 describes the hydrocarbons specific products and distribution (percentage in parentheses) from the knallgas microbe Cupriavidus necator transformed with pSeqCO2::FUEL (Cn_FUEL2.1 and Cn_FUEL2.2) and empty vector (Cn-P);

FIG. 23 describes the effect of pSeqCO2::FUEL (Cn_FUEL2.1 and 2.2) and empty vector (Cn-P) on the fatty acids distribution under the experimental conditions described previously;

FIG. 24 describes the modification of the fatty acid chain length by the enzymatic action of thioesterase (TE) in oleaginous bacteria;

FIG. 25 describes the modification of the fatty acid chain length by the enzymatic action of fatty acyl-ACP thioesterase (TE) in burkholderiaceae;

FIG. 26 describes the similarity of Rhodococcus opacus (B4) thioesterases protein sequence (YP_{—}002784058.1) to other organisms. The Genbank accession numbers, amino acid length and % identity of analyzed proteins are indicated;

FIGS. 27A-27G describe the fluorescence intensity of Rhodococcus Sp exposed to 0, 5, 10, and 20 seconds of (FIGS. 27B, 27C, 27D and 27E respectively) of UV light and stained with Nile Red. FACS analysis of untreated cells (negative control; no Nile Red staining and no UV exposure) (FIG. 27F) and mutated population with increased lipid content (G; P3) are shown;

FIG. 28 describes the chemoautotrophic growth of Cupriavidus necator transformed with pSeqCO2::FUEL (Cn-FUEL2.1), empty vector (Cn-P) and untransformed (Cn). Bacterial growth was measured at O.D650 after 12 days;

FIG. 29 describes the affect of FAR genes expression on biosynthesis of cyclotetradecane in the knallgas microbe Cupriavidus necator. C. necator cells were transformed with pSeqCO2::FAR1 (Cn-F1), pSeqCO2::FAR2 (Cn-F2) and control pSEqCO2 (Cn-P). Cells were harvested (3,000×g for 10 min at 4° C.) and alkanes were analyzed by gas chromatography;

FIG. 30 shows a schematic block flow diagram of a process for utilizing a low cost feedstock such as crude glycerol and/or glycerol and/or methanol to produce oleochemicals using the microorganisms of the present invention;

FIG. 31 shows a schematic block flow diagram of a process for utilizing a low cost feedstock such as crude glycerol and/or glycerol and/or methanol to produce lipids using the microorganisms of the present invention with additional post-processing steps converting the lipids to drop-in fuels such as jet fuel and/or diesel;

FIG. 32 shows a schematic block flow diagram of a process for utilizing a low cost feedstock such as crude glycerol from biodiesel production to produce lipids such as TAGs using the microorganisms of the present invention, that are converted into additional biodiesel through transesterification. The biomass coproducts can be sold as a protein or nutrient source, or can be denatured and reused as a nutrient source in the bioreactor step of the process;

FIG. 33 shows the growth curve for Rhodococcus opacus (DSM 43205) grown on glycerol. Optical density at 650 nm is given versus time. Highest dry cell mass density reached was equal to 25 g/liter;

FIG. 34 shows the growth curve for Rhodococcus opacus (DSM 43205) grown on methanol;

FIG. 35 cost per energy of crude glycerol compared to sugar in 2011;

FIG. 36 shows the cost per carbon of crude glycerol compare to sugar in 2011;

FIG. 37 shows the dicarboxylic acid compound 6 hexane-dioic or adipic acid made by fermentation of unmodified knallgas microbe Cupriavidus necator (DSM 531) strain, extracted from pellet. Other dicarboxylic acids (number of carbons 12, 14, 16, 19, 20, 22) can be made by methods described in patent text;

FIG. 38 shows the different fatty acids made naturally by cultivation of unmodified knallgas microbes Rhodococcus opacus (DSM 43205) and Cupriavidus necator (DSM 531) (number of carbons 13, 14, 15, 16, 17, 18, 19, 20, 22, 23, 24). Introduction of thioesterase yielded production of C12:0 fatty acid in modified knallgas microbe Cupriavidus necator (DSM 531) strain, which was not seen in the natural strain;

FIG. 39 shows the hydroxylation sites for fatty acids. Cultivation of unmodified knallgas microbes Rhodococcus opacus (DSM 43205) and Cupriavidus necator (DSM 531) strains yielded 2-hydroxy and 3-hydroxy C12 fatty acids, 2-hydroxy and 3-hydroxy C14 fatty acids, 2-hydroxy C16 fatty acid, and 3-hydroxy C18 fatty acid. Introduction of hydroxylases will permit omega-hydroxylation at various sites for fatty acids (number of carbons 10, 12, 14, and 18);

FIG. 40 shows unsaturated fatty acids, naturally produced by cultivation of unmodified knallgas microbes Rhodococcus opacus (DSM 43205) and Cupriavidus necator (DSM 531) strains. Introduction of desaturases will permit desaturation at various sites on different length fatty acids;

FIG. 41 shows fatty alcohols, straight chain alkanes hydroxylated on the end. These appear in our cultivation of genetically-modified knallgas microbes Cupriavidus necator (DSM 531) cells. Introduction of FAR genes, enables making n-hydroxylated alkanes of any length;

FIG. 42 shows straight chain alkanes made by genetically-modified version of knallgas microbe Cupriavidus necator (DSM 531) cells (number of carbons 18, 20, 21, 24, 25, 26, 27, 28);

FIG. 43 show eicosanes (n=20 alkanes) produced by genetically-modified knallgas microbe Cupriavidus necator (DSM 531) cells, including straight chain C20, 1,19 diene eicosane, 20-bicyclo[10.8.0]eicosane. These alkanes are not produced by the native strain;

FIG. 44 shows cyclic alkanes of varying lengths produced by genetically-modified knallgas microbe Cupriavidus necator (DSM 531) cells. These alkanes are not produced by the native strain;

FIG. 45 shows unsaturated alkanes with double and triple bonds, derived from genetically modified knallgas microbe Cupriavidus necator (DSM 531) cells. These alkanes are not produced by the native strain, with the exception of squalene, which is produced by the native strain, but then produced at 4-8× in Cupriavidus necator (DSM 531) strain modified with the FAR gene;

FIG. 46 shows the increase in the production C12:0 fatty acid in modified knallgas microbe Cupriavidus necator (DSM 531) strain caused by the introduction of an exogenous thioesterase enzyme which was not seen in same strain without the exogenous thioesterase enzyme (i.e. the Control); and

FIG. 47 plots a sample of the hydrocarbons produced by the knallgas microbe Cupriavidus necator transformed with pSeqCO2::FUEL (Cn_FUEL2.1 and Cn_FUEL2.2) and empty vector (Cn-P).

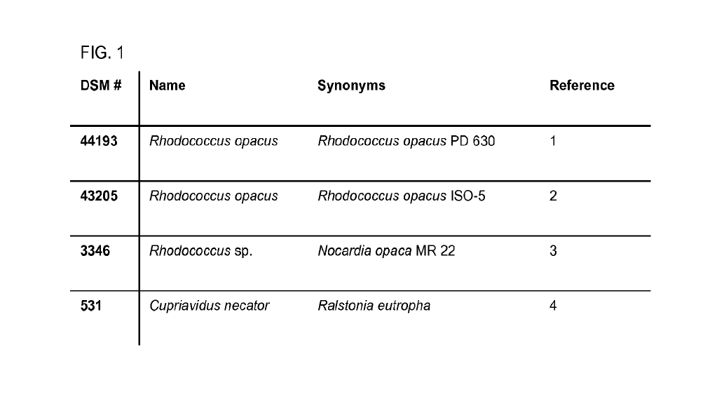

### DETAILED DESCRIPTION OF THE INVENTION

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The terms “amino acid” refer to a molecule containing both an amine group and a carboxyl group that are bound to a carbon, which is, designated the α-carbon. Suitable amino acids include, without limitation, both the D- and L-isomers of the naturally occurring amino acids, as well as non-naturally occurring amino acids prepared by organic synthesis or other metabolic routes. In some embodiments, a single “amino acid” might have multiple sidechain moieties, as available per an extended aliphatic or aromatic backbone scaffold. Unless the context specifically indicates otherwise, the term amino acid, as used herein, is intended to include amino acid analogs.

The term “biodiesel” refers to a biologically produced fatty acid alkyl ester suitable for use as a fuel in a diesel engine.

The term “biomass” refers to a material produced by growth and/or propagation of cells. Biomass may contain cells and/or intracellular contents as well as extracellular material, includes, but is not limited to, compounds secreted by a cell.

The term “bioreactor” or “fermentor” refers to a closed or partially closed vessel in which cells are grown and maintained. The cells may be, but are not necessarily held in liquid suspension. In some embodiments rather than being held in liquid suspension, cells may alternatively be growing and/or maintained in contact with, on, or within another non-liquid substrate including but not limited to a solid growth support material.

The term “catalyst” refers to a chemical actor, such as a molecule or macromolecular structure, which accelerates the speed at which a chemical reaction occurs where a reactant or reactants is converted into a product or products, while the catalyst is not turned into a product itself, or otherwise changed or consumed at the completion of the chemical reaction. After a catalyst participates in one chemical reaction, because it is unchanged, it may participate in further chemical reactions, acting on additional reactants to create additional products. To accelerate a chemical reaction a catalyst decreases the activation energy barrier across the reaction path allowing it to occur at a colder temperature, or faster at a given temperature. In this way a more rapid approach of the system to chemical equilibrium may be achieved. Catalysts subsume enzymes, which are protein catalysts.

The term “CoA” or “coenzyme A” refers to an organic cofactor for condensing enzymes involved in fatty acid synthesis and oxidation, pyruvate oxidation, acetyl or other acyl group transfer, and in other acetylation.

The term “cofactor” subsumes all molecules needed by an enzyme to perform its catalytic activity. In some embodiments, the cofactor is any molecule apart from the substrate.

A “conservative amino acid substitution” is one in which the amino acid residue is replaced with an amino acid residue having a similar side chain. Families of amino acid residues having similar side chains have been defined in the art. These families include amino acids with basic side chains (e.g., K, R, H), acidic side chains (e.g., D, E), uncharged polar side chains (e.g., G, N, Q, S, T, Y, C, H), nonpolar side chains (e.g., G, A, V, L, I, P, F, M, W), beta-branched side chains (e.g., T, V, I) and aromatic side chains (e.g., Y, F, W, H). Thus, a predicted nonessential amino acid residue in an amino acid sequence encoded by an exogenous nucleic acid sequence, for example, is replaced with another amino acid residue from the same side chain family. Other examples of acceptable substitutions are substitutions based on isosteric considerations (e.g. norleucine for methionine) or other biochemical properties (e.g. 2-thienylalanine for phenylalanine).

As used herein, “enzyme fragment” is meant to refer to a fragment of an enzyme that includes the sequences sufficient to function substantially similar to the function of the wild-type enzyme upon which the fragment sequence is based. Fragments are generally 10 or more amino acids in length.

The terms “exogenous gene” means a nucleic acid that has been recombinantly introduced into a cell, which encodes the synthesis of RNA and/or protein. In some embodiments, the exogenous gene is introduced by transformation. In some embodiments, the exogenous gene is introduced into the cell by electroporation. A transformed cell may be referred to as a recombinant cell, into which additional exogenous gene(s) may be introduced. The exogenous gene put into the host species may be taken from a different species (this is called heterologous), or it may naturally occur within the same species (this is homologous as defined below). Therefore, exogenous genes subsume homologous genes that are integrated within or introduced to regions of the genome, episome, or plasmid that differ from the locations where the gene naturally occurs. Multiple copies of the exogenous gene may be introduced into the cell. An exogenous gene may be present in more than one copy within the host cell or transformed cell.

As used herein, the term “expressible form” refers to gene constructs that contain the necessary regulatory elements operably linked to a coding sequence that encodes an enzyme or fragment thereof capable of conferring enzymatic activity to a cell, such that when present in the cell, the coding sequence will be expressed.

SEQ ID NO:1 refers to Sequesco plasmid sequence 1.

SEQ ID NO:2 refers to Sequesco plasmid sequence 2.

SEQ ID NO: 3 refers to Sequesco plasmid Ver1 plasmid sequence.

SEQ ID NO:4 refers to Sequesco plasmid Ver2 plasmid sequence.

SEQ ID NO:5 refers to Arabidopsis gene FAR1.

SEQ ID NO: 6 refers to Arabidopsis gene FAR2.

SEQ ID NO: 7 refers to Arabidopsis gene FAR3.

SEQ ID NO:8 refers to cyanobacterium FadR.

SEQ ID NO:9 refers to cyanobacterium FAD.

SEQ ID NO:10 refers to cyanobacterium Rubisco large subunit promoter

SEQ ID NO:11, refers to the 16S rRNA sequence from the genus Rhodococcus opacus DSM43205

SEQ ID NO:12 refers to the 16S rRNA sequence from the genus Rhodococcus opacus B4.

SEQ ID NO:13 refers to the 16S rRNA sequence from the genus Ralstonia.

SEQ ID NO:14 refers to Rhodococcus opacus TE

The terms “fatty acyl-ACP thioesterase” (TE) mean an enzyme that catalyzes the cleavage of a fatty acid from an acyl carrier protein (ACP) during lipid synthesis.

The terms “fatty acyl-CoA reductase” (FAR) refers to an enzyme catalyzing the reaction that produces a fatty alcohol from an acyl-CoA molecule by reduction.

The terms “fatty acyl-ACP/acyl-CoA reductase” (FadR) refers to an enzyme catalyzing the reaction that produces a fatty aldehyde from an acyl-ACP or acyl-CoA molecule by reduction.

The terms “fatty aldehyde decarbonylase” (FAD) refers to an enzyme catalyzing the reaction that produces an alkane from a fatty aldehyde molecule by decarbonylization.

The terms “fatty aldehyde reductase” refers to an enzyme catalyzing the reaction that produces a fatty alcohol from a fatty aldehyde molecule by reduction.

As used herein, the term “functional fragment” is meant to refer to a fragment of any polypeptide or amino acid sequence that is encoded by an exogenous nucleic acid sequence of the present invention that retains its ability to function like the amino acid sequence to which the fragment is homologous. Functional fragments of enzymes are at least about 5 amino acids in length derived from enzyme and may comprise non-wild-type amino acid sequences. One having ordinary skill in the art can readily determine whether a protein or peptide is a functional fragment of a particular amino acid sequence by examining its sequence and testing its ability to function in a fashion similar to that function of the amino acid sequence upon which the fragment is based. Truncated versions of exogenous proteins may be prepared and tested using routine methods and readily available starting material. As used herein, the term “functional fragment” is also meant to refer to peptides, polypeptides, amino acid sequence linked by non-peptidal bonds, or proteins which comprise an amino acid sequence that is identical or substantially homologous to at least a portion of the exogenous amino acid sequence and which are capable of functioning in a similar function to the exogenous amino acid sequence to which the fragment is homologous. The term “substantially homologous” refers to an amino acid sequence that has conservative substitutions. One having ordinary skill in the art can produce functional fragments of the FAR, FadD, FAD, and thioesterase amino acid sequences following the disclosure provided herein and well known techniques. The functional fragments thus identified may be used and formulated in place of full length FAR, FadD, FAD, and thioesterase without undue experimentation.

As used herein, “homologous” refers to the sequences homology between two nucleic acid sequences or two amino acid sequences. Two nucleic acid sequences or two amino acid sequences that are sufficiently homologous to retain immunogenic function are “homologues.” Sequence homology for nucleotides and amino acids may be determined using FASTA, BLAST and Gapped BLAST (Altschul et al., Nuc. Acids Res., 1997, 25, 3389, which is incorporated herein by reference in its entirety) and PAUP* 4.0b10 software (D. L. Swofford, Sinauer Associates, Massachusetts). “Percentage of similarity” is calculated using PAUP* 4.0b10 software (D. L. Swofford, Sinauer Associates, Massachusetts). The average similarity of the enzymatic sequence or 16S rRNA sequence is calculated compared to all sequences in the phylogenic tree. Briefly, the BLAST algorithm, which stands for Basic Local Alignment Search Tool is suitable for determining sequence similarity (Altschul et al., J. Mol. Biol., 1990, 215, 403410, which is incorporated herein by reference in its entirety). Software for performing BLAST analyses is publicly available though the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, preferably less than about 0.1, more preferably less than about 0.01, and most preferably less than about 0.001.

The term “hydrocarbon” refers to a molecule composed exclusively of carbon and hydrogen atoms with the carbons bonded covalently in a branched, cyclic, linear, or partially cyclic chain and with hydrogen atoms covalently bonded to the carbons such that the chemical octet rule for the carbons is generally satisfied. In some hydrocarbons there may occur some number of double or triple bonds between adjacent carbon atoms in the chain. Thus, the label hydrocarbon subsumes branched, cyclic, linear, branched, or partially cyclic alkanes (also called paraffins), alkenes (also called olefins), and alkynes. The structure of hydrocarbon molecules range from the smallest, methane (CH₄), a primary component of natural gas, to high molecular weight complex molecules including asphaltenes present in bitumens crude oil, and petroleum. Other examples include dodecane (C12), hexadecane (C16), or octadecane (C18) etc. Hydrocarbons of the present invention may be in gaseous, liquid, or solid phases, either as singly or in multiply coexisting phases. In some embodiments, the hydrocarbons are selected from one or more of the following: linear, branched, cyclic, or partially cyclic alkanes, alkenes, lipids, and paraffin.

The term “hydrophobic fraction” gives the fraction of matter that has low solubility in water and greater solubility in a hydrophobic phase than in an aqueous phase. In some embodiments, the hydrophobic fraction is non-polar. In some embodiments, the genetically modified bacterial cells described herein increase the hydrophobic fraction of hydrocarbons in a cell as compared to the same cell that is not genetically modified.

The term “improve lipid yield” refers to an increase in the lipid production of an organism through any means. In some embodiments, the increase is caused by raising the cell dry weight density of a microbial culture and/or raising the fraction of cell mass that is composed of lipid and/or reducing the cell doubling time and/or the biomass doubling time, resulting in an overall increase in the lipid production rate per unit volume.

The terms “jet fuel” means a fuel useful for igniting in the engine of an aircraft comprising a mixture of kerosene (mixture of C9-C16 alkanes of a certain percentage) combined with typical additives. In some embodiments the jet fuel may comprise a mixture of ingredients specified by the Jet A-1, Jet A, Jet B, JP1, JP-2, JP-3, JP-4, JP-5, JP-6, JP-7, JP-8, or other similar compositions. In some embodiments, the jet fuels comprise at least one or more typical additive chosen form antioxidants (including phenolic antioxidants), static inhibitors, corrosion inhibitors, fuel system icing inhibitors, lubrication improvers, biocides, and thermal stability improvers (DOD 1992; IARC 1989; Pearson 1988). These additives are used only in specified amounts, as governed by military specifications (DOD 1992; IARC 1989). Straight-run kerosene, the basic component of the kerosene used for jet fuels, consists of hydrocarbons with carbon numbers mostly in the C9-C16 range. Like all jet fuels, straight-run kerosene consists of a complex mixture of aliphatic and aromatic hydrocarbons (LARC 1989). Aliphatic alkanes (paraffins) and cycloalkanes (naphthenes) are hydrogen saturated, clean burning, and chemically stable and together constitute the major part of kerosene (IARC 1989). In some embodiments, the jet fuel comprises from between about 10%-20% aromatics and less than 1% of olefins. In some embodiments, the boiling range of the jet fuels is well above the boiling point of benzene. In some embodiments, the jet fuel comprises less than or equal to 0.02% of benzene and less than or equal to 0.01% of PAHs.

The term “knallgas” refers to the mixture of molecular hydrogen and oxygen gas. A “knallgas microorganism” is a microbe that can use hydrogen as an electron donor and oxygen as an electron acceptor in the generation of intracellular energy carriers such as Adenosine-5′-triphosphate (ATP). The terms “oxyhydrogen” and “oxyhydrogen microorganism” can be used synonymously with “knallgas” and “knallgas microorganism” respectively.

The terms “lipids” refers to category of molecules that can be dissolved in nonpolar solvents (such as chloroform and/or ether) and which also have low or no solubility in water. The hydrophobic character of lipids molecules typically results from the presence of long chain hydrocarbon sections within the molecule. Lipids subsume the following molecule types: hydrocarbons, fatty acids (saturated and unsaturated), fatty alcohols, fatty aldehydes, monoglycerides, diglycerides, triglycerides, phospholipids, sphingolipids, sterols such as cholesterol and steroid hormones, fat-soluble vitamins (such as vitamins A, D, E and K), polyketides, terpenoids, and waxes.

The term “lipid modification enzyme” corresponds to an enzyme that catalyzes a reaction changing a lipid's covalent bonds such as TE, FAR, FadR, FAD, fatty aldehyde reductase, or lipase. Any enzyme that catalyzes a reaction step or steps in lipid synthesis, catabolism, or modification, including carrier proteins, is called a “lipid pathway enzyme”.

The term “lysate” refers to the liquid containing a mixture and/or a solution of cell contents that result from cell lysis.

The term “lysis” refers to the rupture of the plasma membrane and if present the cell wall of a cell such that a significant amount of intracellular material escapes to the extracellular space. Lysis can be performed using electrochemical, mechanical, osmotic, thermal, or viral means. In some embodiments, the methods of the present invention comprise performing a lysis of cells or microorganisms described herein in order to separate a lipid or mixture of lipids from the contents of a bioreactor. In some embodiments, the methods of the present invention comprise performing a lysis of cells or microorganisms described herein in order to separate a lipid or mixture of lipids from the contents of a bioreactor.

The terms “microorganism” and “microbe” mean microscopic single celled life forms.

The term “molecule” means any distinct or distinguishable structural unit of matter comprising one or more atoms, and includes for example hydrocarbons, lipids, polypeptides and polynucleotides.

The term “oleaginous” refers to something that is rich in oil or produces oil in high quantities.

The term “organic compound” refers to any gaseous, liquid, or solid chemical compounds which contain carbon atoms with the following exceptions that are consider inorganic: carbides, carbonates, simple oxides of carbon, cyanides, and allotropes of pure carbon such as diamond and graphite.

The term “precursor to” or “precursor of” jet fuel, diesel fuel, or biodiesel fuel means a lipid intermediate of one or more of the components of jet, diesel fuel, or biodiesel fuel. For instance, jet fuel is jet fuel is a complex mixture of hydrocarbons that varies depending on crude source and manufacturing process. Consequently, it is impossible to define the exact composition of jet fuel. Specification of jet fuel has therefore evolved primarily as a performance specification rather than a compositional specification and the hydrocarbons typically range between 8 and 17 carbon atoms in hydrocarbon chain length. In some embodiments, a precursor to jet fuel may be composition comprising at least one hydrocarbon having a carbon chain length of 8, 9, 10, 11, 12, 13, 14, 15, 16, 17, 18, or more carbon atoms and having the commonly known specifications for Jet A-1, Jet A, Jet B, JP1, JP-2, JP-3, JP-4, JP-5, JP-6, JP-7, JP-8 fuel when in isolation or mixture with other hydrocarbons. In some embodiments, the precursor to jet fuel is a mixture of different carbon backbone lengths of 8, 9, 10, 11, 12, 13, 14, 15, 16, 17, 18, or more carbon atoms with the commonly known specifications for Jet A-1, Jet A, Jet B, JP1, JP-2, JP-3, JP-4, JP-5, JP-6, JP-7, JP-8 fuel, or other jet fuels. In some embodiments, the precursor to jet fuel may be one or more hydrocarbons that, when exposed to cracking and/or deoxygention and/or isomerization, may be used as a component of Jet A-1, Jet A, Jet B, JP1, JP-2, JP-3, JP-4, JP-5, JP-6, JP-7, JP-8 fuel or other jet fuels.

“Promoter” is a control DNA sequence that regulates transcription. For purposes of the invention, a promoter may includes nucleic acid sequences near the start site of transcription that are required for proper function of the promoter, as for example, a TATA element for a promoter of polymerase II type. Promoters of the present invention can include distal enhancer or repressor elements that may lie in positions up to many thousands of base pairs away from the start site of transcription. The term “inducible promoter” refers to an operable linkage between a promoter and a nucleic acid where the promoter's mediation of nucleic acid transcription is sensitive to a specific stimulus. In some embodiments, the inducible promoter requires a cofactor, which can be added to the environment of the composition comprising the nucleic acid sequence that contains the inducible promoter. An “operable linkage” refers to an operative connection between nucleic acid sequences, such as for example between a control sequence (e.g. a promoter) and another sequence that codes for a protein i.e. a coding sequence. If a promoter can regulate transcription of an exogenous gene then it is in operable linkage with the gene.

### Bacterial Species

The invention relates to bacterial strains that comprise zero or more exogenous nucleic acid sequences. The present invention results from the discovery that certain strains of knallgas bacteria and particular related microorganisms provide unforeseen advantages in the economic and large scale production of chemicals, oils, fuels, and other hydrocarbon or lipid substances from waste carbon feedstocks such as crude glycerol and/or glycerol and/or methanol, and also from the discovery of genetic techniques and systems for modifying these microorganisms for improved performance in these applications. The lipids and other biochemicals synthesized by the microorganisms of the present invention can be applied to uses including but not limited to transportation fuel, petrochemical substitutes, as ingredients in animal feed, food, personal care, and cosmetic products. In some embodiments triglycerides produced in the present invention can be converted by transesterification to long-chain fatty acid esters useful as biodiesel fuel. In some embodiments of the present invention enzymatic and chemical processes can be utilized to produce alkanes, alkenes, alkynes, fatty aldehydes, fatty alcohols, fatty esters, and fatty acids. Some embodiments enable the production of renewable jet fuel, diesel, or other hydrocarbons. Some embodiments enable the production of renewable biodiesel. In addition, the present invention gives methods for culturing and/or modifying bacteria for improved lipid yield and/or lower production costs when grown on crude glycerol and/or glycerol and/or methanol. In some embodiments the genetically modified bacteria produce more of a certain type or types of lipid molecules as compared to the same bacteria that is not genetically modified.

The present invention relates to compositions comprising and methods of using microorganisms to produce and/or secrete carbon-based products from conversion of waste or low cost carbon feedstocks including but not limited to crude glycerol and/or glycerol and/or methanol. The present invention relates to methods and mechanisms to confer production and/or secretion of carbon-based products of interest including but not limited to ethylene, chemicals, polymers, n-alkanes, branched alkanes, cycloalkanes, alkenes, alkynes, fatty alcohols, fatty acids, fatty aldehydes, hydrocarbons, isoprenoids, methyl esters, ethyl esters, alkyl esters, lipids, TAGs, neutral lipids, proteins, polysaccharides, nutraceutical, or pharmaceutical products or intermediates thereof in obligate or facultative knallgas organisms such that these organisms convert crude glycerol and/or glycerol and/or methanol into the aforementioned products.

The production of hydrocarbons or other lipids with carbon chain lengths longer than C₄ is most commonly and efficiently accomplished biologically through fatty acid biosynthesis [Fischer, Klein-Marcuschamer, Stephanolpoulos, Metabolic Engineering (2008) 10, 295-304]. The initial molecule entering into the fatty acid biosynthesis pathway is acetyl-coenzyme A (acetyl-CoA), a central metabolite from which many high value biochemicals can be derived. In some embodiments, the invention utilizes microorganisms with a naturally occurring pathway for the conversion of crude glycerol and/or glycerol and/or methanol to acetyl-CoA. In some embodiments, the invention utilizes microorganisms that can fix C1 compounds including methanol through the reductive tricarboxylic acid cycle, the Calvin-Benson-Bassham cycle, and/or the Wood-Ljungdahl pathway. In some embodiments the microorganisms naturally produce enzymes that catalyze the conversion of crude glycerol and/or glycerol and/or methanol to produce acetyl-CoA, utilizing crude glycerol and/or glycerol and/or methanol as an energy and/or carbon source.

The following gives the net reaction for synthesis of Palmitic acid (C16) starting from Acetyl-CoA:

8Acetyl-CoA+7ATP+H₂O+14NADPH+14H⁺->Palmitic acid+8CoA+14NADP⁺+7ADP+7Pᵢ

The invention relates to a cell and compositions comprising a cell of the class Actinobacteria comprising zero or more exogenous genes. The invention also relates to cells and compositions comprising cells of the family of Nocardiaceae comprising zero or more exogenous genes. The invention also relates to a cell and compositions comprising a cell of a type characterized as a knallgas bacteria. The invention relates to cells and compositions comprising cells of Corynebacterium, Gordonia, Rhodococcus, Mycobacterium and Tsukamurella comprising zero or more exogenous gene. In some embodiments, the invention relates to cells of the family of Nocardiaceae, wherein the cell is not a cell of the genus Mycobacterium. In some embodiments, the invention provides a cell and compositions comprising a cell of the genus Rhodococcus, and in some embodiments the cell is a strain of the species Rhodococcus sp., Rhodococcus opacus. In some embodiments the cell is strain Rhodococcus opacus DSM number 43205 or 43206. In some embodiments, the invention provides cells and compositions comprising a cell of the genus Rhodococcus, wherein the cell or composition comprising a cell of Rhodococcus is non-infectious to animals and/or plants. In some embodiments, the invention provides cells and compositions comprising a cell of the genus Rhodococcus, wherein the Rhodococcus cell or composition comprising a Rhodococcus cell is non-infectious to humans. In some embodiments, the invention provides cells and compositions comprising a cell of the genus Rhodococcus, wherein the Rhodococcus cell or composition comprising a Rhodococcus cell is non-infectious to plants. In some embodiments, the invention relates to a Rhodococcus cell or composition comprising a Rhodococcus cell, wherein the cell is not a species selected from Rhodococcus equi or Rhodococcus fascians.

In some embodiments, the invention relates to a Rhodococcus cell or composition comprising a Rhodococcus cell, wherein the cell is incapable of producing any acrylic acid or acrylamide. In some embodiments, the invention relates to a Rhodococcus cell or composition comprising a Rhodococcus cell, wherein the cell produces less than 10, 9, 8, 7, 6, 5, 4, 3, 2, or 1% of its weight of total dry cellular matter in acrylamide or acrylic/methylacrylic acid. In some embodiments, the invention relates to a Rhodococcus cell or composition comprising a Rhodococcus cell, wherein the cell is not from the species Rhodococcus rhodochrous. In some embodiments, the invention relates to Rhodococcus cell or composition comprising a Rhodococcus cell, wherein the cell is incapable of producing 10-hydroxy-12-octadecenoic acid. In some embodiments, the invention relates to a Rhodococcus cell or composition comprising a Rhodococcus cell, wherein the cell is unable to produce more than 10, 9, 8, 7, 6, 5, 4, 3, 2, or 1% of its weight of total dry cellular matter in 10-hydroxy-12-octadecenoic acid. In some embodiments, the invention relates to Rhodococcus cell or composition comprising a Rhodococcus cell, wherein the cell is incapable of producing optically-active 4-amino-3-hydroxybutyric acid. In some embodiments, the invention relates to a Rhodococcus cell or composition comprising a Rhodococcus cell, wherein the cell is unable to produce more than 10, 9, 8, 7, 6, 5, 4, 3, 2, or 1% of its weight of total dry cellular matter in optically-active 4-amino-3-hydroxybutyric acid.

In some embodiments, the cell or compositions comprising one of more cells is not E. coli. In some embodiments, the cell or compositions comprising one of more cells is from the genus Rhodococcus but is not for the species equi. In some embodiments, the cell of the present invention is not pathogenic to animals or plants. In some embodiments, the cell of the present invention is not pathogenic to humans. In some embodiments, the cell or compositions comprising one of more cells is from the genus Ralstonia.

In some embodiments, the cell or compositions comprising the one or more cells have a 16S rRNA sequence with at least 50, 60, 70, 75, 80, 85, 90, 91, 92, 93, 94, 95, 96, 97, 98 or 99% nucleotide homology to one or more of SEQ ID NOs: 11 or 12. In some embodiments, the cell or compositions comprising the one or more cells have a 16S rRNA sequence with at least 70, 75, 80, 85, 90, 91, 92, 93, 94, 95, 96, 97, 98 or 99% nucleotide homology to one or more of SEQ ID NOs: 11. In some embodiments, the cell or compositions comprising the one or more cells have a 16S rRNA sequence with at least 70, 75, 80, 85, 90, 91, 92, 93, 94, 95, 96, 97, 98 or 99% nucleotide homology to one or more of SEQ ID NOs: 12. In some embodiments, the cell or compositions comprising the one or more cells have a 16S rRNA sequence with at least 70, 75, 80, 85, 90, 91, 92, 93, 94, 95, 96, 97, 98 or 99% nucleotide homology to one or more of SEQ ID NOs: 13.

In some embodiments, the microorganism of the claimed invention does not require any type of sugar to grow and/or metabolize and/or synthesize lipid molecules. In some embodiments, the microorganism can grow and/or metabolize lipids in a slightly anaerobic or extremely anaerobic environment. In some embodiments, the microorganism of the claimed invention is a facultative microorganism

Microbial culturing in the present invention is performed both for the sake of implementing genetic modifications, and for production of organic compounds, and specifically lipids and/or hydrocarbons (e.g., alkanes, fatty acids, fatty alcohols, fatty aldehydes, fatty esters, methyl esters, ethyl esters, alkyl esters, triacylglycerols, other neutral lipids). Microbial culturing with the aim of genetic manipulation is generally performed at a small benchtop scale and often under conditions that select for genetically modified traits. Microbial culturing aimed at the commercial production of organic compounds and specifically lipids and/or hydrocarbons is typically performed in bioreactors at much greater scale (e.g., 500 L to 1,000,000 L bioreactor volumes and higher). In certain embodiments the microorganisms of the present invention are grown in a liquid media inside a bioreactor using the methods of the invention. In some embodiments, the bioreactor containing the microorganisms is constructed of opaque materials that keep the culture in darkness. Bioreactors constructed out of opaque materials such as steel or reinforced concrete can be designed to have extremely big working volumes. In some embodiments of the present invention steel fermenters 50,000 liter and greater in volume are utilized. In some embodiments of the present invention egg-shape or cylindrical digesters 3,000,000 liters and greater in volume are utilized.

The bioreactor or fermentor is used to culture cells through the various phases of their physiological cycle. A bioreactor is utilized for the cultivation of cells, which may be maintained at particular phases in their growth curve. The use of bioreactors is advantageous in many ways for cultivating microbial growth. For certain embodiments, oleaginous cell mass, which is used to produce oleochemicals or fuel, is grown to high densities in liquid suspension. Generally the control of growth conditions including control of dissolved oxygen, and other gases, as well as other dissolved nutrients, trace elements, temperature and pH, is facilitated in a bioreactor.

Nutrient media as well as gases can be added to the bioreactor as either a batch addition, or periodically, or in response to a detected depletion or programmed set point, or continuously over the period the culture is grown and/or maintained. For certain embodiments, the bioreactor at inoculation is filled with a starting batch of nutrient media and/or gases at the beginning of growth, and no additional nutrient media and/or gases are added after inoculation. For certain embodiments, nutrient media and/or gases are added periodically after inoculation. For certain embodiments, nutrient media and/or gas is added after inoculation in response to a detected depletion of nutrient and/or gas. For certain embodiments, nutrient media and/or gas is added continuously after inoculation.

In some embodiments, a crude glycerol feedstock serves as a carbon and energy source for microbial growth wherein glycerol comprises 70 to 90 percent by weight of the crude glycerol. In some embodiments glycerol comprises 50 to 70 percent by weight of the crude glycerol. In some embodiments glycerol comprises less than 50 percent by weight of the crude glycerol. In some embodiments, the composition comprises a crude glycerol feedstock as a carbon and energy source for microbial growth wherein methanol comprises 10 to 20 percent by weight of the crude glycerol. In some embodiments methanol comprises less than 1 percent by weight of the crude glycerol. In some embodiments methanol comprises over 20 percent by weight of the crude glycerol. In some embodiments, the composition comprises a crude glycerol feedstock as a carbon and energy source for microbial growth wherein free fatty acids comprise 1 to 10 percent by weight of the crude glycerol. In some embodiments free fatty acids comprise less than 1 percent by weight of the crude glycerol. In some embodiments free fatty acids comprise over 10 percent by weight of the crude glycerol. In some embodiments, the composition comprises a crude glycerol feedstock as a carbon and energy source for microbial growth wherein MONG comprise 1 to 10 percent by weight of the crude glycerol. In some embodiments MONG comprises less than 1 percent by weight of the crude glycerol. In some embodiments MONG comprises over 10 percent by weight of the crude glycerol. In some embodiments, the composition comprises a crude glycerol feedstock as a carbon and energy source for microbial growth wherein salts comprise 5 to 10 percent by weight of the crude glycerol. In some embodiments salts comprises less than 5 percent by weight of the crude glycerol. In some embodiments salts comprise 10 to 15 percent by weight of the crude glycerol. In some embodiments salts comprise over 15 percent by weight of the crude glycerol.

For certain embodiments the bioreactors have mechanisms to enable mixing of the nutrient media that include but are not limited to spinning stir bars, blades, impellers, or turbines, spinning, rocking, or turning vessels, gas lifts and sparging. The culture media may be mixed continuously or intermittently.

The ports that are standard in bioreactors may be utilized to deliver, or withdraw, gases, liquids, solids, and/or slurries, into the bioreactor vessel enclosing the microbes of the present invention. Many bioreactors have multiple ports for different purposes (e.g. ports for media addition, gas addition, probes for pH and DO, sampling), and a given port may be used for various purposes during the course of a microbial cultivation run. As an example, a port might be used to add nutrient media to the bioreactor at one point in time and at another time might be used for sampling. Preferably, the multiple use of a sampling port can be performed without introducing contamination or invasive species into the growth environment. A valve or other actuator enabling control of the sample flow or continuous sampling can be provided to a sampling port. For certain embodiments the bioreactors are equipped with at least one port suitable for culture inoculation that can additionally serve other uses including the addition of media or gas. Bioreactors ports enable control of the gas composition and flow rate into the culture environment. For example the ports can be used as gas inlets into the bioreactor through which gases are pumped. For some embodiments gases that may be pumped into a bioreactor include oxygen, syngas, producer gas, hydrogen gas, CO2, air, air/CO₂ mixtures, ammonia, nitrogen, noble gases, such as argon, as well as other gases. Raising the gas flow rate into a bioreactor can enhance mixing of the culture and produce turbulence if the gas inlet is positioned under the surface of the liquid media such that gas bubbles or sparges up through the media. In some embodiments, a bioreactor comprises gas outlet ports for gas escape and pressure release. In some embodiments, gas inlets and outlets are preferably equipped with check valves to prevent gas backflow.

The present invention relates to bioreactors that comprise a cell, which comprises zero or more exogenous nucleic acid sequences that encodes a lipid pathway enzyme. The present invention relates to a system of at least one bioreactor that comprise a cell, which comprises zero or more exogenous nucleic acid sequences that encodes a lipid pathway enzyme. In some embodiments, the system comprises two or more, three or more, or four or more bioreactors, at least one of which comprise a cell, which comprises zero or more nucleic acid sequences that encodes a lipid pathway enzyme. In some embodiments, the system of bioreactors comprises at least a first and second bioreactor, wherein the first bioreactor comprises a cell; and wherein the second bioreactor comprises a microorganism derived from a different species. In some embodiments, the system of bioreactors comprises a first bioreactor that comprises the cell of the present invention and a second bioreactor comprising a microalgal or bacterial cell.

In some embodiments, the cells of the present invention are capable of producing desaturated lipids between 5 and 24 carbon atoms long at greater than 18 grams per liter volume of culture per three day period. In some embodiments, the cells of the present invention are capable of producing desaturated alkanes between 8 and 18 carbon atoms long at greater than or equal to 18 grams per liter volume of culture per three day period, wherein the desaturated alkanes are desaturated at a carbon position other than carbon-9.

### Genetic Modifications

The present invention relates to methods of modifying a bacterial cell to express one or more exogenous nucleic acid sequences that encodes one or more enzymes to enable conversion of crude glycerol and/or glycerol and/or methanol into useful carbon-based products of interest in an amount greater than an amount of carbon-based products produced by the same bacterial cell that does not express the exogenous nucleic acid sequences. Methods of selecting and manufacturing nucleic acid sequences for modification of bacterial cells are known and can be performed by transformation, electroporation, phage infection of bacteria, or other techniques for nucleic acid transfer generally known in the art. Standard recombinant DNA and molecular cloning techniques useful for the invention are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987), all of which are incorporated by reference in their entireties.

The invention relates to genetic constructs comprising one or more exogenous genes that encode one or more amino acid sequences to enable conversion of crude glycerol and/or glycerol and/or methanol, into useful carbon-based products of interest in an amount greater than an amount of carbon-based products produced by the same bacterial cell that does not express the exogenous nucleic acid sequence or sequences. Another aspect of the present invention relates to compositions that comprise at least one bacterial cell, which comprises at least one nucleic acid sequence that encodes at least one exogenous amino acid sequence that functions as a fatty acid acyl-ACP reductase, a fatty acid aldehyde decarbonylase and/or a thioesterase. In some embodiments, the bacterial cell is transformed with one or more, two or more, three or more, four or more, or five or more exogenous nucleic acid sequences that encode one or more amino acid sequences to enable conversion of crude glycerol and/or glycerol and/or methanol, into useful carbon-based products of interest in an amount greater than an amount of carbon-based products produced by the same bacterial cell that does not express the exogenous nucleic acid sequence or sequences. According to the present invention, genetic material that encodes the enzyme is delivered to a bacterial cell in an expressible form. The genetic material, DNA or RNA, is taken up by the cells of the invention and expressed. The enzyme or enzymes that are thereby produced can biochemically modify lipid molecules to remove or add hydroxyl groups, remove or add carbonyl groups, remove or add carbon-carbon double bonds, remove or add carbon-carbon triple bonds, remove or add aldehyde groups, or remove or add ester groups to lipid molecules in lipid.

In some embodiments, the genetic constructs of the present invention comprise DNA, RNA, or combinations of both DNA and RNA. In some embodiments, the genetic construct of the present invention is a plasmid. It will be appreciated that, in some embodiments, the plasmid contains a variety of open reading frames (ORFs) encoding proteins of many diverse functions, including those enzymes that enable hydrocarbon or lipid modification, glutathione-S transferase (GST) activity, origins of replication, multiple cloning sites, promoters, and/or termination sequences. It is contemplated therefore that a host cell transformed with the plasmid will demonstrate the ability to modify a variety of hydrocarbons as well as maintain its copy number in the cytoplasm of the cell. The glutathione-S transferases (GSTs) represent a large group of detoxification enzymes. GSTs catalyze the conjugation of glutathione, homoglutathione and other glutathione-like analog via sulfhydryl group, to a large range of hydrophobic, electrophilic compounds. The conjugation can result in detoxification of these compounds. GST genes are found in both prokaryotic (e.g., E. coli) and eukaryotic organisms (e.g., yeast, plant and human). Although the homologies between the GSTs from prokaryotes and eukaryotes were low, many of the residues assigned to be important for the enzymatic function or structure in the eukaryotes were found to be conserved in prokaryotic GSTs (Nishida et al., J. Biol Chem 269:32536-32541 (1994)). It has been suggested that bacterial GST may represent a defense against the effects of antibiotics (Piccolomini et al., J Gen Microbiol 135:3119-3125 (1989)). Accordingly it is contemplated that a host strain transformed with the plasmid will have the ability detoxify harmful compounds via conjugation of those compounds to glutathione.

In some embodiments, the instant plasmid additionally encodes a variety of maintenance proteins, useful for maintaining, stabilizing and replicating the plasmid. It is contemplated that these genes may be used in conjunction with other bacterial plasmids deficient in these functions for the increased stabilization or robust maintenance of the plasmid. In some embodiments, the plasmid comprises maintenance proteins of particular interest including the REP origin of replication (encoded by ORF 38) the TRA proteins (TRAI, TRAJ and TRAK, encoded by ORF's 23, 24 and 25 respectively) and the VAG proteins (VAGD and VAGC, encoded by ORF's 33 and 34 respectively). The tra gene family is known to be involved in plasmid conjugation, a process that promotes DNA transfer from a donor to a recipient cell mediated by physical contact (Firth et al, Escherichia coli and Salmonella: Cellular and Molecular Biology, ASM press (1996)). Among tra gene products, TraI and TraK proteins are reported to be required for efficient plasmid site-specific recombination (Paterson et al. J. Bacteriol 181:2572-2583 (1999)). Furthermore, TraI is required for conjugal DNA transfer. Fukuda and Ohtsubo (Genes Cells 2:735-751 (1997)) reported that TraI has the activity of site- and strand-specific nicking of the supercoiled plasmid DNA. TraJ, traJ gene product, regulates transcription originating at the tra operon promoter P.sub.traY. (Firth et al., Escherichia coli and Salmonella: Cellular and Molecular Biology, ASM press (1996)). The stabilization proteins VAGC and VAGD encoded by vagC and vagD are involved in the maintaining the plasmid as an autonomous replicating unit. Bacterial maintenance proteins of particular interest on the pSeq and pVer plasmids include.

SEQ ID NO: 1
TCGCGCGTTT CGGTGATGAC GGTGAAAACC TCTGACACAT GCAGCTCCCG GAGACGGTCA CAGCTTGTCT
GTAAGCGGAT GCCGGGAGCA GACAAGCCCG AGCGCGCAAA GCCACTACTG CCACTTTTGG AGACTGTGTA CGTCGAGGGC
CTCTGCCAGT GTCGAACAGA CATTCGCCTA CGGCCCTCGT CTGTTCGGGC TCAGGGCGCG TCAGCGGGTG TTGGCGGGTG
TCGGGGCTGG CTTAACTATG CGGCATCAGA GCAGATTGTA CTGAGAGTGC ACCATATGCG GTGTGAAATA AGTCCCGCGC
AGTCGCCCAC AACCGCCCAC AGCCCCGACC GAATTGATAC GCCGTAGTCT CGTCTAACAT GACTCTCACG TGGTATACGC
CACACTTTAT CCGCACAGAT GCGTAAGGAG AAAATACCGC ATCAGGCGCC ATTCGCCATT CAGGCTGCGC AACTGTTGGG
AAGGGCGATC GGTGCGGGCC TCTTCGCTAT GGCGTGTCTA CGCATTCCTC TTTTATGGCG TAGTCCGCGG TAAGCGGTAA
GTCCGACGCG TTGACAACCC TTCCCGCTAG CCACGCCCGG AGAAGCGATA TACGCCAGCT GGCGAAAGGG GGATGTGCTG
CAAGGCGATT AAGTTGGGTA ACGCCAGGGT TTTCCCAGTC ACGACGTTGT AAAACGACGG CCAGTGCCAA ATGCGGTCGA
CCGCTTTCCC CCTACACGAC GTTCCGCTAA TTCAACCCAT TGCGGTCCCA AAAGGGTCAG TGCTGCAACA TTTTGCTGCC
GGTCACGGTT GCTTGCATGC CTGCAGGTCG ACGGGCCCGG GATCCGATGC TCTTCCGCTA AGATCTGCCG CGGCCGCGTC
CTCAGAAGAA CTCGTCAAGA AGGCGATAGA CGAACGTACG GACGTCCAGC TGCCCGGGCC CTAGGCTACG AGAAGGCGAT
TCTAGACGGC GCCGGCGCAG GAGTCTTCTT GAGCAGTTCT TCCGCTATCT AGGCGATGCG CTGCGAATCG GGAGCGGCGA
TACCGTAAAG CACGAGGAAG CGGTCAGCCC ATTCGCCGCC AAGCTCTTCA GCAATATCAC GGGTAGCCAA TCCGCTACGC
GACGCTTAGC CCTCGCCGCT ATGGCATTTC GTGCTCCTTC GCCAGTCGGG TAAGCGGCGG TTCGAGAAGT CGTTATAGTG
CCCATCGGTT CGCTATGTCC TGATAGCGGT CCGCCACACC CAGCCGGCCA CAGTCGATGA ATCCAGAAAA GCGGCCATTT
TCCACCATGA TATTCGGCAA GCAGGCATCG GCGATACAGG ACTATCGCCA GGCGGTGTGG GTCGGCCGGT GTCAGCTACT
TAGGTCTTTT CGCCGGTAAA AGGTGGTACT ATAAGCCGTT CGTCCGTAGC CCATGGGTCA CGACGAGATC CTCGCCGTCG
GGCATGCGCG CCTTGAGCCT GGCGAACAGT TCGGCTGGCG CGAGCCCCTG ATGCTCTTCG TCCAGATCAT GGTACCCAGT
GCTGCTCTAG GAGCGGCAGC CCGTACGCGC GGAACTCGGA CCGCTTGTCA AGCCGACCGC GCTCGGGGAC TACGAGAAGC
AGGTCTAGTA CCTGATCGAC AAGACCGGCT TCCATCCGAG TACGTGCTCG CTCGATGCGA TGTTTCGCTT GGTGGTCGAA
TGGGCAGGTA GCCGGATCAA GCGTATGCAG GGACTAGCTG TTCTGGCCGA AGGTAGGCTC ATGCACGAGC GAGCTACGCT
ACAAAGCGAA CCACCAGCTT ACCCGTCCAT CGGCCTAGTT CGCATACGTC CCGCCGCATT GCATCAGCCA TGATGGATAC
TTTCTCGGCA GGAGCAAGGT GGGATGACAG GAGATCCTGC CCCGGCACTT CGCCCAATAG CAGCCAGTCC GGCGGCGTAA
CGTAGTCGGT ACTACCTATG AAAGAGCCGT CCTCGTTCCA CCCTACTGTC CTCTAGGACG GGGCCGTGAA GCGGGTTATC
GTCGGTCAGG CTTCCCGCTT CAGTGACAAC GTCGAGCACA GCTGCGCAAG GAACGCCCGT CGTGGCCAGC CACGATAGCC
GCGCTGCCTC GTCCTGCAGT TCATTCAGGG GAAGGGCGAA GTCACTGTTG CAGCTCGTGT CGACGCGTTC CTTGCGGGCA
GCACCGGTCG GTGCTATCGG CGCGACGGAG CAGGACGTCA AGTAAGTCCC CACCGGACAG GTCGGTCTTG ACAAAAAGAA
CCGGGCGCCC CTGCGCTGAC AGCCGGAACA CGGCGGCATC AGAGCAGCCG ATTGTCTGTT GTGCCCAGTC GTGGCCTGTC
CAGCCAGAAC TGTTTTTCTT GGCCCGCGGG GACGCGACTG TCGGCCTTGT GCCGCCGTAG TCTCGTCGGC TAACAGACAA
CACGGGTCAG ATAGCCGAAT AGCCTCTCCA CCCAAGCGGC CGGAGAACCT GCGTGCAATC CATCTTGTTC AATCATGATA
TCCCTTAATT AACCGTTAAC ACTAGTTCAG TATCGGCTTA TCGGAGAGGT GGGTTCGCCG GCCTCTTGGA CGCACGTTAG
GTAGAACAAG TTAGTACTAT AGGGAATTAA TTGGCAATTG TGATCAAGTC TCCATCTCGC CGTGTATGCG GGCCTGACGG
ATCAACGTTC CCACCGAGCC AGTCGAGATG TTCATCTGGT CGGCGATCTG CCGGTACTTC AAACCTTGTT AGGTAGAGCG
GCACATACGC CCGGACTGCC TAGTTGCAAG GGTGGCTCGG TCAGCTCTAC AAGTAGACCA GCCGCTAGAC GGCCATGAAG
TTTGGAACAA TGCGCAGTTC CACAGCCTTC TTGCGGCGTT CCTGCGCACG AGCGATGTAG TCGCCTCGGT CTTCGGCGAC
GAGCCGTTTG ATGGTGCTTT TCGAGACGCC ACGCGTCAAG GTGTCGGAAG AACGCCGCAA GGACGCGTGC TCGCTACATC
AGCGGAGCCA GAAGCCGCTG CTCGGCAAAC TACCACGAAA AGCTCTGCGG GAACTTGTCA GCCAACTCCT GCGCGGTCTG
CGTGCGACGC ATCACGCGTT CTGCAGCACC CATCAGTCCG TCCCCTCTGC TGCTGCGAAC AGTGCCGATC CTTGAACAGT
CGGTTGAGGA CGCGCCAGAC GCACGCTGCG TAGTGCGCAA GACGTCGTGG GTAGTCAGGC AGGGGAGACG ACGACGCTTG
TCACGGCTAG GATCGACCTT CTTGAGCTTC GGCCGCGGCG CGGTGGCGTT CTTCCGTACC GCTTCCGTTT TTGCGCTGCT
GCTCACTTTG CCGCGGCGTG CCTGGATTTT CTAGCTGGAA GAACTCGAAG CCGGCGCCGC GCCACCGCAA GAAGGCATGG
CGAAGGCAAA AACGCGACGA CGAGTGAAAC GGCGCCGCAC GGACCTAAAA CGAGAACTCG GCGGCGGTGA AGGTGCGGTG
GGTCCAGTGG GCGACTGATT TGCCGATCTG CTCGGCCTCG GCCCGACTCA TGGGGCCGAT CCCGTCGTTG GCTCTTGAGC
CGCCGCCACT TCCACGCCAC CCAGGTCACC CGCTGACTAA ACGGCTAGAC GAGCCGGAGC CGGGCTGAGT ACCCCGGCTA
GGGCAGCAAC GCGTCGAGGG TGAAGTTGGT CAGGGCGGTG AAGTCGGTGA CCATCTGCCG CCACACAGTG ATCGACGGGT
AGTTCTGTTT CCGGATCTCG CGGTAGGCCC CGCAGCTCCC ACTTCAACCA GTCCCGCCAC TTCAGCCACT GGTAGACGGC
GGTGTGTCAC TAGCTGCCCA TCAAGACAAA GGCCTAGAGC GCCATCCGGG ATTCCCGGGT GCGGTCGAAC AGTTCGACGT
TCCGGCCCGT TTCGGTCCTG ACCTGTGTCT TGCGGCCGTA GTCCGGTGGG GCGGGGAAAC GGTCACCGAG TAAGGGCCCA
CGCCAGCTTG TCAAGCTGCA AGGCCGGGCA AAGCCAGGAC TGGACACAGA ACGCCGGCAT CAGGCCACCC CGCCCCTTTG
CCAGTGGCTC CGCTTTTGCG AGGCCTTTGA GCGAGTACGG ATCCGAGGGA CCCCAGACCG TCGTCCAGTG CGGGTGGATC
GGGTTCTGGG TGAGCTGCTG CGCGTAGCCC GCGAAAACGC TCCGGAAACT CGCTCATGCC TAGGCTCCCT GGGGTCTGGC
AGCAGGTCAC GCCCACCTAG CCCAAGACCC ACTCGACGAC GCGCATCGGG TGATCGGCGC CGACCACCGA GGCGATCAGC
CCCTGGTTCA CCCGGTCGTA GAGCCGCAGC GGGCCCTGTC GGGCTGCCTG GAGGGTGTAG ACCGGGCTTT ACTAGCCGCG
GCTGGTGGCT CCGCTAGTCG GGGACCAAGT GGGCCAGCAT CTCGGCGTCG CCCGGGACAG CCCGACGGAC CTCCCACATC
TGGCCCGAAA CGAGCAGCCA CCACAGGTGC GCGTGCTCGG TCGCGGGATT GATCGTCATC ACGGTCGGAT CGGGCAGATC
CGCGTTACGT GCGGCCCACT GCGCCTGGTC GCTCGTCGGT GGTGTCCACG CGCACGAGCC AGCGCCCTAA CTAGCAGTAG
TGCCAGCCTA GCCCGTCTAG GCGCAATGCA CGCCGGGTGA CGCGGACCAG GTCGTCCACG TCGAGCACCA AGCCCAACCT
GATCGACGGG GTGCGGGCCG CAATGTAGCG GCGGGTGAGC GCCTCCGCGC GCGGCTGCGG CCACTGCCCG CAGCAGGTGC
AGCTCGTGGT TCGGGTTGGA CTAGCTGCCC CACGCCCGGC GTTACATCGC CGCCCACTCG CGGAGGCGCG CGCCGACGCC
GGTGACGGGC TCCCGGACGT AGTCATCCGT CGCGTGCGGG TATTTGAACC GCCAGCGGTC CAACCAGGCG TCAACAGCAG
CGGTCATGAC CGCCAAGCTA GGGCCGGATC AGGGCCTGCA TCAGTAGGCA GCGCACGCCC ATAAACTTGG CGGTCGCCAG
GTTGGTCCGC AGTTGTCGTC GCCAGTACTG GCGGTTCGAT CCCGGCCTAG TGTACCGATC GGGGGAGGCG CGCCGCAAAT
TATTTAAGAG TCTCGCTAGC AAACCATGTC AGGTGTTGCG GTGGGTTCCG GGTAAACCTC CACCCGAATT ACATGGCTAG
CCCCCTCCGC GCGGCGTTTA ATAAATTCTC AGAGCGATCG TTTGGTACAG TCCACAACGC CACCCAAGGC CCATTTGGAG
GTGGGCTTAA ATTTAAGAGT CTCGCTAGCT AAGCCCTATC TGATGCTGCG CGGGGGGTCC TTCGCACTGA ATCTCAAAGG
TGGCCGGCTG AATTTCGTCG CGCGAAAACC TAAATTCTCA GAGCGATCGA TTCGGGATAG ACTACGACGC GCCCCCCAGG
AAGCGTGACT TAGAGTTTCC ACCGGCCGAC TTAAAGCAGC GCGCTTTTGG TCCCTGGACA GTTCTGGAAT TCAGCAAGAG
GTGTGTCTGA ACTTCGGTGT TTTTTTGGGG GGTGACTCCA GCGGGGTGGG CACAACGCGA ACAGAGACCT AGGGACCTGT
CAAGACCTTA AGTCGTTCTC CACACAGACT TGAAGCCACA AAAAAACCCC CCACTGAGGT CGCCCCACCC GTGTTGCGCT
TGTCTCTGGA TGTGTGTACG ACGGCGGGAG GTAAGTCGGG TACGGCTCGG ACTGCGGTAG AGCAACCGTC GAATCGATTT
CGAGCAGAGC GAGCAGAGCA AGATATTCCA ACACACATGC TGCCGCCCTC CATTCAGCCC ATGCCGAGCC TGACGCCATC
TCGTTGGCAG CTTAGCTAAA GCTCGTCTCG CTCGTCTCGT TCTATAAGGT AAACTCCGGG GTTCCTCGGC GGCCTCCCCC
GTCTGTTTGC TCAACCGAGG GAGACCTGGC GGTCCCGCGT TTCCGGACGC GCGGGACCGC CTACCGCTCG TTTGAGGCCC
CAAGGAGCCG CCGGAGGGGG CAGACAAACG AGTTGGCTCC CTCTGGACCG CCAGGGCGCA AAGGCCTGCG CGCCCTGGCG
GATGGCGAGC AGAGCGGAAG AGCATCTAGA TGCATTCGCG AGGTACCGAG CTCGAATTCG TAATCATGGT CATAGCTGTT
TCCTGTGTGA AATTGTTATC CGCTCACAAT TCTCGCCTTC TCGTAGATCT ACGTAAGCGC TCCATGGCTC GAGCTTAAGC
ATTAGTACCA GTATCGACAA AGGACACACT TTAACAATAG GCGAGTGTTA TCCACACAAC ATACGAGCCG GAAGCATAAA
GTGTAAAGCC TGGGGTGCCT AATGAGTGAG CTAACTCACA TTAATTGCGT TGCGCTCACT GCCCGCTTTC AGGTGTGTTG
TATGCTCGGC CTTCGTATTT CACATTTCGG ACCCCACGGA TTACTCACTC GATTGAGTGT AATTAACGCA ACGCGAGTGA
CGGGCGAAAG CAGTCGGGAA ACCTGTCGTG CCAGCTGCAT TAATGAATCG GCCAACGCGC GGGGAGAGGC GGTTTGCGTA
TTGGGCGCTC TTCCGCTTCC TCGCTCACTG GTCAGCCCTT TGGACAGCAC GGTCGACGTA ATTACTTAGC CGGTTGCGCG
CCCCTCTCCG CCAAACGCAT AACCCGCGAG AAGGCGAAGG AGCGAGTGAC ACTCGCTGCG CTCGGTCGTT CGGCTGCGGC
GAGCGGTATC AGCTCACTCA AAGGCGGTAA TACGGTTATC CACAGAATCA GGGGATAACG CAGGAAAGAA TGAGCGACGC
GAGCCAGCAA GCCGACGCCG CTCGCCATAG TCGAGTGAGT TTCCGCCATT ATGCCAATAG GTGTCTTAGT CCCCTATTGC
GTCCTTTCTT CATGTGAGCA AAAGGCCAGC AAAAGGCCAG GAACCGTAAA AAGGCCGCGT TGCTGGCGTT TTTCCATAGG
CTCCGCCCCC CTGACGAGCA TCACAAAAAT GTACACTCGT TTTCCGGTCG TTTTCCGGTC CTTGGCATTT TTCCGGCGCA
ACGACCGCAA AAAGGTATCC GAGGCGGGGG GACTGCTCGT AGTGTTTTTA CGACGCTCAA GTCAGAGGTG GCGAAACCCG
ACAGGACTAT AAAGATACCA GGCGTTTCCC CCTGGAAGCT CCCTCGTGCG CTCTCCTGTT CCGACCCTGC GCTGCGAGTT
CAGTCTCCAC CGCTTTGGGC TGTCCTGATA TTTCTATGGT CCGCAAAGGG GGACCTTCGA GGGAGCACGC GAGAGGACAA
GGCTGGGACG CGCTTACCGG ATACCTGTCC GCCTTTCTCC CTTCGGGAAG CGTGGCGCTT TCTCATAGCT CACGCTGTAG
GTATCTCAGT TCGGTGTAGG TCGTTCGCTC GCGAATGGCC TATGGACAGG CGGAAAGAGG GAAGCCCTTC GCACCGCGAA
AGAGTATCGA GTGCGACATC CATAGAGTCA AGCCACATCC AGCAAGCGAG CAAGCTGGGC TGTGTGCACG AACCCCCCGT
TCAGCCCGAC CGCTGCGCCT TATCCGGTAA CTATCGTCTT GAGTCCAACC CGGTAAGACA CGACTTATCG GTTCGACCCG
ACACACGTGC TTGGGGGGCA AGTCGGGCTG GCGACGCGGA ATAGGCCATT GATAGCAGAA CTCAGGTTGG GCCATTCTGT
GCTGAATAGC CCACTGGCAG CAGCCACTGG TAACAGGATT AGCAGAGCGA GGTATGTAGG CGGTGCTACA GAGTTCTTGA
AGTGGTGGCC TAACTACGGC TACACTAGAA GGTGACCGTC GTCGGTGACC ATTGTCCTAA TCGTCTCGCT CCATACATCC
GCCACGATGT CTCAAGAACT TCACCACCGG ATTGATGCCG ATGTGATCTT GGACAGTATT TGGTATCTGC GCTCTGCTGA
AGCCAGTTAC CTTCGGAAAA AGAGTTGGTA GCTCTTGATC CGGCAAACAA ACCACCGCTG GTAGCGGTGG CCTGTCATAA
ACCATAGACG CGAGACGACT TCGGTCAATG GAAGCCTTTT TCTCAACCAT CGAGAACTAG GCCGTTTGTT TGGTGGCGAC
CATCGCCACC TTTTTTTGTT TGCAAGCAGC AGATTACGCG CAGAAAAAAA GGATCTCAAG AAGATCCTTT GATCTTTTCT
ACGGGGTCTG ACGCTCAGTG GAACGAAAAC AAAAAAACAA ACGTTCGTCG TCTAATGCGC GTCTTTTTTT CCTAGAGTTC
TTCTAGGAAA CTAGAAAAGA TGCCCCAGAC TGCGAGTCAC CTTGCTTTTG TCACGTTAAG GGATTTTGGT CATGAGATTA
TCAAAAAGGA TCTTCACCTA GATCCTTTTA AATTAAAAAT GAAGTTTTAA ATCAATCTAA AGTATATATG AGTGCAATTC
CCTAAAACCA GTACTCTAAT AGTTTTTCCT AGAAGTGGAT CTAGGAAAAT TTAATTTTTA CTTCAAAATT TAGTTAGATT
TCATATATAC AGTAAACTTG GTCTGACAGT TACCAATGCT TAATCAGTGA GGCACCTATC TCAGCGATCT GTCTATTTCG
TTCATCCATA GTTGCCTGAC TCCCCGTCGT TCATTTGAAC CAGACTGTCA ATGGTTACGA ATTAGTCACT CCGTGGATAG
AGTCGCTAGA CAGATAAAGC AAGTAGGTAT CAACGGACTG AGGGGCAGCA GTAGATAACT ACGATACGGG AGGGCTTACC
ATCTGGCCCC AGTGCTGCAA TGATACCGCG AGACCCACGC TCACCGGCTC CAGATTTATC AGCAATAAAC CATCTATTGA
TGCTATGCCC TCCCGAATGG TAGACCGGGG TCACGACGTT ACTATGGCGC TCTGGGTGCG AGTGGCCGAG GTCTAAATAG
TCGTTATTTG CAGCCAGCCG GAAGGGCCGA GCGCAGAAGT GGTCCTGCAA CTTTATCCGC CTCCATCCAG TCTATTAATT
GTTGCCGGGA AGCTAGAGTA AGTAGTTCGC GTCGGTCGGC CTTCCCGGCT CGCGTCTTCA CCAGGACGTT GAAATAGGCG
GAGGTAGGTC AGATAATTAA CAACGGCCCT TCGATCTCAT TCATCAAGCG CAGTTAATAG TTTGCGCAAC GTTGTTGCCA
TTGCTACAGG CATCGTGGTG TCACGCTCGT CGTTTGGTAT GGCTTCATTC AGCTCCGGTT CCCAACGATC GTCAATTATC
AAACGCGTTG CAACAACGGT AACGATGTCC GTAGCACCAC AGTGCGAGCA GCAAACCATA CCGAAGTAAG TCGAGGCCAA
GGGTTGCTAG AAGGCGAGTT ACATGATCCC CCATGTTGTG CAAAAAAGCG GTTAGCTCCT TCGGTCCTCC GATCGTTGTC
AGAAGTAAGT TGGCCGCAGT GTTATCACTC TTCCGCTCAA TGTACTAGGG GGTACAACAC GTTTTTTCGC CAATCGAGGA
AGCCAGGAGG CTAGCAACAG TCTTCATTCA ACCGGCGTCA CAATAGTGAG ATGGTTATGG CAGCACTGCA TAATTCTCTT
ACTGTCATGC CATCCGTAAG ATGCTTTTCT GTGACTGGTG AGTACTCAAC CAAGTCATTC TGAGAATAGT TACCAATACC
GTCGTGACGT ATTAAGAGAA TGACAGTACG GTAGGCATTC TACGAAAAGA CACTGACCAC TCATGAGTTG GTTCAGTAAG
ACTCTTATCA GTATGCGGCG ACCGAGTTGC TCTTGCCCGG CGTCAATACG GGATAATACC GCGCCACATA GCAGAACTTT
AAAAGTGCTC ATCATTGGAA AACGTTCTTC CATACGCCGC TGGCTCAACG AGAACGGGCC GCAGTTATGC CCTATTATGG
CGCGGTGTAT CGTCTTGAAA TTTTCACGAG TAGTAACCTT TTGCAAGAAG GGGGCGAAAA CTCTCAAGGA TCTTACCGCT
GTTGAGATCC AGTTCGATGT AACCCACTCG TGCACCCAAC TGATCTTCAG CATCTTTTAC TTTCACCAGC CCCCGCTTTT
GAGAGTTCCT AGAATGGCGA CAACTCTAGG TCAAGCTACA TTGGGTGAGC ACGTGGGTTG ACTAGAAGTC GTAGAAAATG
AAAGTGGTCG GTTTCTGGGT GAGCAAAAAC AGGAAGGCAA AATGCCGCAA AAAAGGGAAT AAGGGCGACA CGGAAATGTT
GAATACTCAT ACTCTTCCTT TTTCAATATT CAAAGACCCA CTCGTTTTTG TCCTTCCGTT TTACGGCGTT TTTTCCCTTA
TTCCCGCTGT GCCTTTACAA CTTATGAGTA TGAGAAGGAA AAAGTTATAA ATTGAAGCAT TTATCAGGGT TATTGTCTCA
TGAGCGGATA CATATTTGAA TGTATTTAGA AAAATAAACA AATAGGGGTT CCGCGCACAT TTCCCCGAAA TAACTTCGTA
AATAGTCCCA ATAACAGAGT ACTCGCCTAT GTATAAACTT ACATAAATCT TTTTATTTGT TTATCCCCAA GGCGCGTGTA
AAGGGGCTTT AGTGCCACCT GACGTCTAAG AAACCATTAT TATCATGACA TTAACCTATA AAAATAGGCG TATCACGAGG
CCCTTTCGTC TCACGGTGGA CTGCAGATTC TTTGGTAATA ATAGTACTGT AATTGGATAT TTTTATCCGC ATAGTGCTCC
GGGAAAGCAG
SEQ ID NO: 2
GGGGAGCCGC GCCGAAGGCG TGGGGGAACC CCGCAGGGGT GCCCTTCTTT GGGCACCAAA GAACTAGATA TAGGGCGAAA
TGCGAAAGAC TTAAAAATCA CCCCTCGGCG CGGCTTCCGC ACCCCCTTGG GGCGTCCCCA CGGGAAGAAA CCCGTGGTTT
CTTGATCTAT ATCCCGCTTT ACGCTTTCTG AATTTTTAGT ACAACTTAAA AAAGGGGGGT ACGCAACAGC TCATTGCGGC
ACCCCCCGCA ATAGCTCATT GCGTAGGTTA AAGAAAATCT GTAATTGACT GCCACTTTTA TGTTGAATTT TTTCCCCCCA
TGCGTTGTCG AGTAACGCCG TGGGGGGCGT TATCGAGTAA CGCATCCAAT TTCTTTTAGA CATTAACTGA CGGTGAAAAT
CGCAACGCAT AATTGTTGTC GCGCTGCCGA AAAGTTGCAG CTGATTGCGC ATGGTGCCGC AACCGTGCGG CACCCTACCG
CATGGAGATA AGCATGGCCA GCGTTGCGTA TTAACAACAG CGCGACGGCT TTTCAACGTC GACTAACGCG TACCACGGCG
TTGGCACGCC GTGGGATGGC GTACCTCTAT TCGTACCGGT CGCAGTCCAG AGAAATCGGC ATTCAAGCCA AGAACAAGCC
CGGTCACTGG GTGCAAACGG AACGCAAAGC GCATGAGGCG TGGGCCGGGC TTATTGCGAG GCGTCAGGTC TCTTTAGCCG
TAAGTTCGGT TCTTGTTCGG GCCAGTGACC CACGTTTGCC TTGCGTTTCG CGTACTCCGC ACCCGGCCCG AATAACGCTC
GAAACCCACG GCGGCAATGC TGCTGCATCA CCTCGTGGCG CAGATGGGCC ACCAGAACGC CGTGGTGGTC AGCCAGAAGA
CACTTTCCAA GCTCATCGGA CTTTGGGTGC CGCCGTTACG ACGACGTAGT GGAGCACCGC GTCTACCCGG TGGTCTTGCG
GCACCACCAG TCGGTCTTCT GTGAAAGGTT CGAGTAGCCT CGTTCTTTGC GGACGGTCCA ATACGCAGTC AAGGACTTGG
TGGCCGAGCG CTGGATCTCC GTCGTGAAGC TCAACGGCCC CGGCACCGTG TCGGCCTACG GCAAGAAACG CCTGCCAGGT
TATGCGTCAG TTCCTGAACC ACCGGCTCGC GACCTAGAGG CAGCACTTCG AGTTGCCGGG GCCGTGGCAC AGCCGGATGC
TGGTCAATGA CCGCGTGGCG TGGGGCCAGC CCCGCGACCA GTTGCGCCTG TCGGTGTTCA GTGCCGCCGT GGTGGTTGAT
CACGACGACC AGGACGAATC ACCAGTTACT GGCGCACCGC ACCCCGGTCG GGGCGCTGGT CAACGCGGAC AGCCACAAGT
CACGGCGGCA CCACCAACTA GTGCTGCTGG TCCTGCTTAG GCTGTTGGGG CATGGCGACC TGCGCCGCAT CCCGACCCTG
TATCCGGGCG AGCAGCAACT ACCGACCGGC CCCGGCGAGG AGCCGCCCAG CCAGCCCGGC CGACAACCCC GTACCGCTGG
ACGCGGCGTA GGGCTGGGAC ATAGGCCCGC TCGTCGTTGA TGGCTGGCCG GGGCCGCTCC TCGGCGGGTC GGTCGGGCCG
ATTCCGGGCA TGGAACCAGA CCTGCCAGCC TTGACCGAAA CGGAGGAATG GGAACGGCGC GGGCAGCAGC GCCTGCCGAT
GCCCGATGAG CCGTGTTTTC TAAGGCCCGT ACCTTGGTCT GGACGGTCGG AACTGGCTTT GCCTCCTTAC CCTTGCCGCG
CCCGTCGTCG CGGACGGCTA CGGGCTACTC GGCACAAAAG TGGACGATGG CGAGCCGTTG GAGCCGCCGA CACGGGTCAC
GCTGCCGCGC CGGTAGCACT TGGGTTGCGC AGCAACCCGT AAGTGCGCTG TTCCAGACTA ACCTGCTACC GCTCGGCAAC
CTCGGCGGCT GTGCCCAGTG CGACGGCGCG GCCATCGTGA ACCCAACGCG TCGTTGGGCA TTCACGCGAC AAGGTCTGAT
TCGGCTGTAG CCGCCTCGCC GCCCTATACC TTGTCTGCCT CCCCGCGTTG CGTCGCGGTG CATGGAGCCG GGCCACCTCG
ACCTGAATGG AAGCCGGCGG AGCCGACATC GGCGGAGCGG CGGGATATGG AACAGACGGA GGGGCGCAAC GCAGCGCCAC
GTACCTCGGC CCGGTGGAGC TGGACTTACC TTCGGCCGCC CACCTCGCTA ACGGATTCAC CGTTTTTATC AGGCTCTGGG
AGGCAGAATA AATGATCATA TCGTCAATTA TTACCTCCAC GGGGAGAGCC TGAGCAAACT GTGGAGCGAT TGCCTAAGTG
GCAAAAATAG TCCGAGACCC TCCGTCTTAT TTACTAGTAT AGCAGTTAAT AATGGAGGTG CCCCTCTCGG ACTCGTTTGA
GGCCTCAGGC ATTTGAGAAG CACACGGTCA CACTGCTTCC GGTAGTCAAT AAACCGGTAA ACCAGCAATA GACATAAGCG
GCTATTTAAC GACCCTGCCC CCGGAGTCCG TAAACTCTTC GTGTGCCAGT GTGACGAAGG CCATCAGTTA TTTGGCCATT
TGGTCGTTAT CTGTATTCGC CGATAAATTG CTGGGACGGG TGAACCGACG ACCGGGTCGA ATTTGCTTTC GAATTTCTGC
CATTCATCCG CTTATTATCA CTTATTCAGG CGTAGCACCA GGCGTTTAAG GGCACCAATA ACTTGGCTGC TGGCCCAGCT
TAAACGAAAG CTTAAAGACG GTAAGTAGGC GAATAATAGT GAATAAGTCC GCATCGTGGT CCGCAAATTC CCGTGGTTAT
ACTGCCTTAA AAAAATTACG CCCCGCCCTG CCACTCATCG CAGTCGGCCT ATTGGTTAAA AAATGAGCTG ATTTAACAAA
AATTTAACGC GAATTTTAAC TGACGGAATT TTTTTAATGC GGGGCGGGAC GGTGAGTAGC GTCAGCCGGA TAACCAATTT
TTTACTCGAC TAAATTGTTT TTAAATTGCG CTTAAAATTG AAAATATTAA CGCTTACAAT TTCCATTCGC CATTCAGGCT
GCGCAACTGT TGGGAAGGGC GATCGGTGCG GGCCTCTTCG CTATTACGCC AGCTGGCGAA TTTTATAATT GCGAATGTTA
AAGGTAAGCG GTAAGTCCGA CGCGTTGACA ACCCTTCCCG CTAGCCACGC CCGGAGAAGC GATAATGCGG TCGACCGCTT
AGGGGGATGT GCTGCAAGGC GATTAAGTTG GGTAACGCCA GGGTTTTCCC AGTCACGACG TTGTAAAACG ACGGCCAGTG
AGCGCGCGTA ATACGACTCA TCCCCCTACA CGACGTTCCG CTAATTCAAC CCATTGCGGT CCCAAAAGGG TCAGTGCTGC
AACATTTTGC TGCCGGTCAC TCGCGCGCAT TATGCTGAGT CTATAGGGCG AATTGGAGCT CCACCGCGGT GGCGGCCGCT
CTAGAACTAG TGGATCCCCC GGGCTGCAGG AATTCGATAT CAAGCTTATC GATACCGTCG GATATCCCGC TTAACCTCGA
GGTGGCGCCA CCGCCGGCGA GATCTTGATC ACCTAGGGGG CCCGACGTCC TTAAGCTATA GTTCGAATAG CTATGGCAGC
ACCTCGAGGG GGGGCCCGGT ACCCAGCTTT TGTTCCCTTT AGTGAGGGTT AATTGCGCGC TTGGCGTAAT CATGGTCATA
GCTGTTTCCT GTGTGAAATT TGGAGCTCCC CCCCGGGCCA TGGGTCGAAA ACAAGGGAAA TCACTCCCAA TTAACGCGCG
AACCGCATTA GTACCAGTAT CGACAAAGGA CACACTTTAA GTTATCCGCT CACAATTCCA CACAACATAC GAGCCGGAAG
CATAAAGTGT AAAGCCTGGG GTGCCTAATG AGTGAGCTAA CTCACATTAA TTGCGTTGCG CAATAGGCGA GTGTTAAGGT
GTGTTGTATG CTCGGCCTTC GTATTTCACA TTTCGGACCC CACGGATTAC TCACTCGATT GAGTGTAATT AACGCAACGC
CTCACTGCCC GCTTTCCAGT CGGGAAACCT GTCGTGCCAG CTGCATTAAT GAATCGGCCA ACGCGCGGGG AGAGGCGGTT
TGCGTATTGG GCGCATGCAT GAGTGACGGG CGAAAGGTCA GCCCTTTGGA CAGCACGGTC GACGTAATTA CTTAGCCGGT
TGCGCGCCCC TCTCCGCCAA ACGCATAACC CGCGTACGTA AAAAACTGTT GTAATTCATT AAGCATTCTG CCGACATGGA
AGCCATCACA AACGGCATGA TGAACCTGAA TCGCCAGCGG CATCAGCACC TTGTCGCCTT TTTTTGACAA CATTAAGTAA
TTCGTAAGAC GGCTGTACCT TCGGTAGTGT TTGCCGTACT ACTTGGACTT AGCGGTCGCC GTAGTCGTGG AACAGCGGAA
GCGTATAATA TTTGCCCATG GGGGTGGGCG AAGAACTCCA GCATGAGATC CCCGCGCTGG AGGATCATCC AGCCGGCGTC
CCGGAAAACG ATTCCGAAGC CGCATATTAT AAACGGGTAC CCCCACCCGC TTCTTGAGGT CGTACTCTAG GGGCGCGACC
TCCTAGTAGG TCGGCCGCAG GGCCTTTTGC TAAGGCTTCG CCAACCTTTC ATAGAAGGCG GCGGTGGAAT CGAAATCTCG
TGATGGCAGG TTGGGCGTCG CTTGGTCGGT CATTTCGAAC CCCAGAGTCC CGCTCAGAAG GGTTGGAAAG TATCTTCCGC
CGCCACCTTA GCTTTAGAGC ACTACCGTCC AACCCGCAGC GAACCAGCCA GTAAAGCTTG GGGTCTCAGG GCGAGTCTTC
AACTCGTCAA GAAGGCGATA GAAGGCGATG CGCTGCGAAT CGGGAGCGGC GATACCGTAA AGCACGAGGA AGCGGTCAGC
CCATTCGCCG CCAAGCTCTT TTGAGCAGTT CTTCCGCTAT CTTCCGCTAC GCGACGCTTA GCCCTCGCCG CTATGGCATT
TCGTGCTCCT TCGCCAGTCG GGTAAGCGGC GGTTCGAGAA CAGCAATATC ACGGGTAGCC AACGCTATGT CCTGATAGCG
GTCCGCCACA CCCAGCCGGC CACAGTCGAT GAATCCAGAA AAGCGGCCAT TTTCCACCAT GTCGTTATAG TGCCCATCGG
TTGCGATACA GGACTATCGC CAGGCGGTGT GGGTCGGCCG GTGTCAGCTA CTTAGGTCTT TTCGCCGGTA AAAGGTGGTA
GATATTCGGC AAGCAGGCAT CGCCATGGGT CACGACGAGA TCCTCGCCGT CGGGCATGCG CGCCTTGAGC CTGGCGAACA
GTTCGGCTGG CGCGAGCCCC CTATAAGCCG TTCGTCCGTA GCGGTACCCA GTGCTGCTCT AGGAGCGGCA GCCCGTACGC
GCGGAACTCG GACCGCTTGT CAAGCCGACC GCGCTCGGGG TGATGCTCTT CGTCCAGATC ATCCTGATCG ACAAGACCGG
CTTCCATCCG AGTACGTGCT CGCTCGATGC GATGTTTCGC TTGGTGGTCG AATGGGCAGG ACTACGAGAA GCAGGTCTAG
TAGGACTAGC TGTTCTGGCC GAAGGTAGGC TCATGCACGA GCGAGCTACG CTACAAAGCG AACCACCAGC TTACCCGTCC
TAGCCGGATC AAGCGTATGC AGCCGCCGCA TTGCATCAGC CATGATGGAT ACTTTCTCGG CAGGAGCAAG GTGAGATGAC
AGGAGATCCT GCCCCGGCAC ATCGGCCTAG TTCGCATACG TCGGCGGCGT AACGTAGTCG GTACTACCTA TGAAAGAGCC
GTCCTCGTTC CACTCTACTG TCCTCTAGGA CGGGGCCGTG TTCGCCCAAT AGCAGCCAGT CCCTTCCCGC TTCAGTGACA
ACGTCGAGCA CAGCTGCGCA AGGAACGCCC GTCGTGGCCA GCCACGATAG CCGCGCTGCC AAGCGGGTTA TCGTCGGTCA
GGGAAGGGCG AAGTCACTGT TGCAGCTCGT GTCGACGCGT TCCTTGCGGG CAGCACCGGT CGGTGCTATC GGCGCGACGG
TCGTCCTGCA GTTCATTCAG GGCACCGGAC AGGTCGGTCT TGACAAAAAG AACCGGGCGC CCCTGCGCTG ACAGCCGGAA
CACGGCGGCA TCAGAGCAGC AGCAGGACGT CAAGTAAGTC CCGTGGCCTG TCCAGCCAGA ACTGTTTTTC TTGGCCCGCG
GGGACGCGAC TGTCGGCCTT GTGCCGCCGT AGTCTCGTCG CGATTGTCTG TTGTGCCCAG TCATAGCCGA ATAGCCTCTC
CACCCAAGCG GCCGGAGAAC CTGCGTGCAA TCCATCTTGT TCAATCATGC GAAACGATCC GCTAACAGAC AACACGGGTC
AGTATCGGCT TATCGGAGAG GTGGGTTCGC CGGCCTCTTG GACGCACGTT AGGTAGAACA AGTTAGTACG CTTTGCTAGG
TCATCCTGTC TCTTGATCAG ATCTTGATCC CCTGCGCCAT CAGATCCTTG GCGGCAAGAA AGCCATCCAG TTTACTTTGC
AGGGCTTCCC AACCTTACCA AGTAGGACAG AGAACTAGTC TAGAACTAGG GGACGCGGTA GTCTAGGAAC CGCCGTTCTT
TCGGTAGGTC AAATGAAACG TCCCGAAGGG TTGGAATGGT GAGGGCGCCC CAGCTGGCAA TTCCGGTTCG CTTGCTGTCC
ATAAAACCGC CCAGTCTAGC TATCGCCATG TAAGCCCACT GCAAGCTACC TGCTTTCTCT CTCCCGCGGG GTCGACCGTT
AAGGCCAAGC GAACGACAGG TATTTTGGCG GGTCAGATCG ATAGCGGTAC ATTCGGGTGA CGTTCGATGG ACGAAAGAGA
TTGCGCTTGC GTTTTCCCTT GTCCAGATAG CCCAGTAGCT GACATTCATC CCAGGTGGCA CTTTTCGGGG AAATGTGCGC
GCCCGCGTTC CTGCTGGCGC AACGCGAACG CAAAAGGGAA CAGGTCTATC GGGTCATCGA CTGTAAGTAG GGTCCACCGT
GAAAAGCCCC TTTACACGCG CGGGCGCAAG GACGACCGCG TGGGCCTGTT TCTGGCGCTG GACTTCCCGC TGTTCCGTCA
GCAGCTTTTC GCCCACGGCC TTGATGATCG CGGCGGCCTT GGCCTGCATA TCCCGATTCA ACCCGGACAA AGACCGCGAC
CTGAAGGGCG ACAAGGCAGT CGTCGAAAAG CGGGTGCCGG AACTACTAGC GCCGCCGGAA CCGGACGTAT AGGGCTAAGT
ACGGCCCCAG GGCGTCCAGA ACGGGCTTCA GGCGCTCCCG AAGGTCTCGG GCCGTCTCTT GGGCTTGATC GGCCTTCTTG
CGCATCTCAC GCGCTCCTGC TGCCGGGGTC CCGCAGGTCT TGCCCGAAGT CCGCGAGGGC TTCCAGAGCC CGGCAGAGAA
CCCGAACTAG CCGGAAGAAC GCGTAGAGTG CGCGAGGACG GGCGGCCTGT AGGGCAGGCT CATACCCCTG CCGAACCGCT
TTTGTCAGCC GGTCGGCCAC GGCTTCCGGC GTCTCAACGC GCTTTGAGAT TCCCAGCTTT CCGCCGGACA TCCCGTCCGA
GTATGGGGAC GGCTTGGCGA AAACAGTCGG CCAGCCGGTG CCGAAGGCCG CAGAGTTGCG CGAAACTCTA AGGGTCGAAA
TCGGCCAATC CCTGCGGTGC ATAGGCGCGT GGCTCGACCG CTTGCGGGCT GATGGTGACG TGGCCCACTG GTGGCCGCTC
CAGGGCCTCG TAGAACGCCT AGCCGGTTAG GGACGCCACG TATCCGCGCA CCGAGCTGGC GAACGCCCGA CTACCACTGC
ACCGGGTGAC CACCGGCGAG GTCCCGGAGC ATCTTGCGGA GAATGCGCGT GTGACGTGCC TTGCTGCCCT CGATGCCCCG
TTGCAGCCCT AGATCGGCCA CAGCGGCCGC AAACGTGGTC TGGTCGCGGG TCATCTGCGC CTTACGCGCA CACTGCACGG
AACGACGGGA GCTACGGGGC AACGTCGGGA TCTAGCCGGT GTCGCCGGCG TTTGCACCAG ACCAGCGCCC AGTAGACGCG
TTTGTTGCCG ATGAACTCCT TGGCCGACAG CCTGCCGTCC TGCGTCAGCG GCACCACGAA CGCGGTCATG TGCGGGCTGG
TTTCGTCACG GTGGATGCTG AAACAACGGC TACTTGAGGA ACCGGCTGTC GGACGGCAGG ACGCAGTCGC CGTGGTGCTT
GCGCCAGTAC ACGCCCGACC AAAGCAGTGC CACCTACGAC GCCGTCACGA TGCGATCCGC CCCGTACTTG TCCGCCAGCC
ACTTGTGCGC CTTCTCGAAG AACGCCGCCT GCTGTTCTTG GCTGGCCGAC TTCCACCATT CGGCAGTGCT ACGCTAGGCG
GGGCATGAAC AGGCGGTCGG TGAACACGCG GAAGAGCTTC TTGCGGCGGA CGACAAGAAC CGACCGGCTG AAGGTGGTAA
CCGGGCTGGC CGTCATGACG TACTCGACCG CCAACACAGC GTCCTTGCGC CGCTTCTCTG GCAGCAACTC GCGCAGTCGG
CCCATCGCTT CATCGGTGCT GGCCCGACCG GCAGTACTGC ATGAGCTGGC GGTTGTGTCG CAGGAACGCG GCGAAGAGAC
CGTCGTTGAG CGCGTCAGCC GGGTAGCGAA GTAGCCACGA GCTGGCCGCC CAGTGCTCGT TCTCTGGCGT CCTGCTGGCG
TCAGCGTTGG GCGTCTCGCG CTCGCGGTAG GCGTGCTTGA GACTGGCCGC CACGTTGCCC CGACCGGCGG GTCACGAGCA
AGAGACCGCA GGACGACCGC AGTCGCAACC CGCAGAGCGC GAGCGCCATC CGCACGAACT CTGACCGGCG GTGCAACGGG
ATTTTCGCCA GCTTCTTGCA TCGCATGATC GCGTATGCCG CCATGCCTGC CCCTCCCTTT TGGTGTCCAA CCGGCTCGAC
GGGGGCAGCG CAAGGCGGTG TAAAAGCGGT CGAAGAACGT AGCGTACTAG CGCATACGGC GGTACGGACG GGGAGGGAAA
ACCACAGGTT GGCCGAGCTG CCCCCGTCGC GTTCCGCCAC CCTCCGGCGG GCCACTCAAT GCTTGAGTAT ACTCACTAGA
CTTTGCTTCG CAAAGTCGTG ACCGCCTACG GCGGCTGCGG CGCCCTACGG GCTTGCTCTC GGAGGCCGCC CGGTGAGTTA
CGAACTCATA TGAGTGATCT GAAACGAAGC GTTTCAGCAC TGGCGGATGC CGCCGACGCC GCGGGATGCC CGAACGAGAG
CGGGCTTCGC CCTGCGCGGT CGCTGCGCTC CCTTGCCAGC CCGTGGATAT GTGGACGATG GCCGCGAGCG GCCACCGGCT
GGCTCGCTTC GCTCGGCCCG GCCCGAAGCG GGACGCGCCA GCGACGCGAG GGAACGGTCG GGCACCTATA CACCTGCTAC
CGGCGCTCGC CGGTGGCCGA CCGAGCGAAG CGAGCCGGGC TGGACAACCC TGCTGGACAA GCTGATGGAC AGGCTGCGCC
TGCCCACGAG CTTGACCACA GGGATTGCCC ACCGGCTACC CAGCCTTCGA CCACATACCC ACCTGTTGGG ACGACCTGTT
CGACTACCTG TCCGACGCGG ACGGGTGCTC GAACTGGTGT CCCTAACGGG TGGCCGATGG GTCGGAAGCT GGTGTATGGG
ACCGGCTCCA ACTGCGCGGC CTGCGGCCTT GCCCCATCAA TTTTTTTAAT TTTCTCTGGG GAAAAGCCTC CGGCCTGCGG
CCTGCGCGCT TCGCTTGCCG TGGCCGAGGT TGACGCGCCG GACGCCGGAA CGGGGTAGTT AAAAAAATTA AAAGAGACCC
CTTTTCGGAG GCCGGACGCC GGACGCGCGA AGCGAACGGC GTTGGACACC AAGTGGAAGG CGGGTCAAGG CTCGCGCAGC
GACCGCGCAG CGGCTTGGCC TTGACGCGCC TGGAACGACC CAAGCCTATG CGAGTGGGGG CAACCTGTGG TTCACCTTCC
GCCCAGTTCC GAGCGCGTCG CTGGCGCGTC GCCGAACCGG AACTGCGCGG ACCTTGCTGG GTTCGGATAC GCTCACCCCC
CAGTCGAAGG CGAAGCCCGC CCGCCTGCCC CCCGAGCCTC ACGGCGGCGA GTGCGGGGGT TCCAAGGGGG CAGCGCCACC
TTGGGCAAGG CCGAAGGCCG GTCAGCTTCC GCTTCGGGCG GGCGGACGGG GGGCTCGGAG TGCCGCCGCT CACGCCCCCA
AGGTTCCCCC GTCGCGGTGG AACCCGTTCC GGCTTCCGGC CGCAGTCGAT CAACAAGCCC CGGAGGGGCC ACTTTTTGCC
GGAGGCGTCA GCTAGTTGTT CGGGGCCTCC CCGGTGAAAA ACGGCCTC
SEQ ID NO: 3
GGGGAGCCGC GCCGAAGGCG TGGGGGAACC CCGCAGGGGT GCCCTTCTTT GGGCACCAAA GAACTAGATA
TAGGGCGAAA TGCGAAAGAC TTAAAAATCA CCCCTCGGCG CGGCTTCCGC ACCCCCTTGG GGCGTCCCCA CGGGAAGAAA
CCCGTGGTTT CTTGATCTAT ATCCCGCTTT ACGCTTTCTG AATTTTTAGT ACAACTTAAA AAAGGGGGGT ACGCAACAGC
TCATTGCGGC ACCCCCCGCA ATAGCTCATT GCGTAGGTTA AAGAAAATCT GTAATTGACT GCCACTTTTA TGTTGAATTT
TTTCCCCCCA TGCGTTGTCG AGTAACGCCG TGGGGGGCGT TATCGAGTAA CGCATCCAAT TTCTTTTAGA CATTAACTGA
CGGTGAAAAT CGCAACGCAT AATTGTTGTC GCGCTGCCGA AAAGTTGCAG CTGATTGCGC ATGGTGCCGC AACCGTGCGG
CACCCTACCG CATGGAGATA AGCATGGCCA GCGTTGCGTA TTAACAACAG CGCGACGGCT TTTCAACGTC GACTAACGCG
TACCACGGCG TTGGCACGCC GTGGGATGGC GTACCTCTAT TCGTACCGGT CGCAGTCCAG AGAAATCGGC ATTCAAGCCA
AGAACAAGCC CGGTCACTGG GTGCAAACGG AACGCAAAGC GCATGAGGCG TGGGCCGGGC TTATTGCGAG GCGTCAGGTC
TCTTTAGCCG TAAGTTCGGT TCTTGTTCGG GCCAGTGACC CACGTTTGCC TTGCGTTTCG CGTACTCCGC ACCCGGCCCG
AATAACGCTC GAAACCCACG GCGGCAATGC TGCTGCATCA CCTCGTGGCG CAGATGGGCC ACCAGAACGC CGTGGTGGTC
AGCCAGAAGA CACTTTCCAA GCTCATCGGA CTTTGGGTGC CGCCGTTACG ACGACGTAGT GGAGCACCGC GTCTACCCGG
TGGTCTTGCG GCACCACCAG TCGGTCTTCT GTGAAAGGTT CGAGTAGCCT CGTTCTTTGC GGACGGTCCA ATACGCAGTC
AAGGACTTGG TGGCCGAGCG CTGGATCTCC GTCGTGAAGC TCAACGGCCC CGGCACCGTG TCGGCCTACG GCAAGAAACG
CCTGCCAGGT TATGCGTCAG TTCCTGAACC ACCGGCTCGC GACCTAGAGG CAGCACTTCG AGTTGCCGGG GCCGTGGCAC
AGCCGGATGC TGGTCAATGA CCGCGTGGCG TGGGGCCAGC CCCGCGACCA GTTGCGCCTG TCGGTGTTCA GTGCCGCCGT
GGTGGTTGAT CACGACGACC AGGACGAATC ACCAGTTACT GGCGCACCGC ACCCCGGTCG GGGCGCTGGT CAACGCGGAC
AGCCACAAGT CACGGCGGCA CCACCAACTA GTGCTGCTGG TCCTGCTTAG GCTGTTGGGG CATGGCGACC TGCGCCGCAT
CCCGACCCTG TATCCGGGCG AGCAGCAACT ACCGACCGGC CCCGGCGAGG AGCCGCCCAG CCAGCCCGGC CGACAACCCC
GTACCGCTGG ACGCGGCGTA GGGCTGGGAC ATAGGCCCGC TCGTCGTTGA TGGCTGGCCG GGGCCGCTCC TCGGCGGGTC
GGTCGGGCCG ATTCCGGGCA TGGAACCAGA CCTGCCAGCC TTGACCGAAA CGGAGGAATG GGAACGGCGC GGGCAGCAGC
GCCTGCCGAT GCCCGATGAG CCGTGTTTTC TAAGGCCCGT ACCTTGGTCT GGACGGTCGG AACTGGCTTT GCCTCCTTAC
CCTTGCCGCG CCCGTCGTCG CGGACGGCTA CGGGCTACTC GGCACAAAAG TGGACGATGG CGAGCCGTTG GAGCCGCCGA
CACGGGTCAC GCTGCCGCGC CGGTAGCACT TGGGTTGCGC AGCAACCCGT AAGTGCGCTG TTCCAGACTA ACCTGCTACC
GCTCGGCAAC CTCGGCGGCT GTGCCCAGTG CGACGGCGCG GCCATCGTGA ACCCAACGCG TCGTTGGGCA TTCACGCGAC
AAGGTCTGAT TCGGCTGTAG CCGCCTCGCC GCCCTATACC TTGTCTGCCT CCCCGCGTTG CGTCGCGGTG CATGGAGCCG
GGCCACCTCG ACCTGAATGG AAGCCGGCGG AGCCGACATC GGCGGAGCGG CGGGATATGG AACAGACGGA GGGGCGCAAC
GCAGCGCCAC GTACCTCGGC CCGGTGGAGC TGGACTTACC TTCGGCCGCC CACCTCGCTA ACGGATTCAC CGTTTTTATC
AGGCTCTGGG AGGCAGAATA AATGATCATA TCGTCAATTA TTACCTCCAC GGGGAGAGCC TGAGCAAACT GTGGAGCGAT
TGCCTAAGTG GCAAAAATAG TCCGAGACCC TCCGTCTTAT TTACTAGTAT AGCAGTTAAT AATGGAGGTG CCCCTCTCGG
ACTCGTTTGA GGCCTCAGGC ATTTGAGAAG CACACGGTCA CACTGCTTCC GGTAGTCAAT AAACCGGTAA ACCAGCAATA
GACATAAGCG GCTATTTAAC GACCCTGCCC CCGGAGTCCG TAAACTCTTC GTGTGCCAGT GTGACGAAGG CCATCAGTTA
TTTGGCCATT TGGTCGTTAT CTGTATTCGC CGATAAATTG CTGGGACGGG TGAACCGACG ACCGGGTCGA ATTTGCTTTC
GAATTTCTGC CATTCATCCG CTTATTATCA CTTATTCAGG CGTAGCACCA GGCGTTTAAG GGCACCAATA ACTTGGCTGC
TGGCCCAGCT TAAACGAAAG CTTAAAGACG GTAAGTAGGC GAATAATAGT GAATAAGTCC GCATCGTGGT CCGCAAATTC
CCGTGGTTAT ACTGCCTTAA AAAAATTACG CCCCGCCCTG CCACTCATCG CAGTCGGCCT ATTGGTTAAA AAATGAGCTG
ATTTAACAAA AATTTAACGC GAATTTTAAC TGACGGAATT TTTTTAATGC GGGGCGGGAC GGTGAGTAGC GTCAGCCGGA
TAACCAATTT TTTACTCGAC TAAATTGTTT TTAAATTGCG CTTAAAATTG AAAATATTAA CGCTTACAAT TTCCATTCGC
CATTCAGGCT GCGCAACTGT TGGGAAGGGC GATCGGTGCG GGCCTCTTCG CTATTACGCC AGCTGGCGAA TTTTATAATT
GCGAATGTTA AAGGTAAGCG GTAAGTCCGA CGCGTTGACA ACCCTTCCCG CTAGCCACGC CCGGAGAAGC GATAATGCGG
TCGACCGCTT AGGGGGATGT GCTGCAAGGC GATTAAGTTG GGTAACGCCA GGGTTTTCCC AGTCACGACG TTGTAAAACG
ACGGCCAGTG AGCGCGCGTA ATACGACTCA TCCCCCTACA CGACGTTCCG CTAATTCAAC CCATTGCGGT CCCAAAAGGG
TCAGTGCTGC AACATTTTGC TGCCGGTCAC TCGCGCGCAT TATGCTGAGT CTATAGGGCG AATTGGAGCT CCACCGCGGT
GGCGGCCGCT CTAGAACTAG TGGATCCCCC GGGCTGCAGG AATTCGATAT CAAGCTTATC GATACCGTCG GATATCCCGC
TTAACCTCGA GGTGGCGCCA CCGCCGGCGA GATCTTGATC ACCTAGGGGG CCCGACGTCC TTAAGCTATA GTTCGAATAG
CTATGGCAGC ACGGGCCCGG GATCCGATGC TCTTCCGCTA AGATCTTTTA CTAGTTCAGT CCATCTCGCC GTGTATGCGG
GCCTGACGGA TCAACGTTCC CACCGAGCCA TGCCCGGGCC CTAGGCTACG AGAAGGCGAT TCTAGAAAAT GATCAAGTCA
GGTAGAGCGG CACATACGCC CGGACTGCCT AGTTGCAAGG GTGGCTCGGT GTCGAGATGT TCATCTGGTC GGCGATCTGC
CGGTACTTCA AACCTTGTTT GCGCAGTTCC ACAGCCTTCT TGCGGCGTTC CTGCGCACGA GCGATGTAGT CAGCTCTACA
AGTAGACCAG CCGCTAGACG GCCATGAAGT TTGGAACAAA CGCGTCAAGG TGTCGGAAGA ACGCCGCAAG GACGCGTGCT
CGCTACATCA CGCCTCGGTC TTCGGCGACG AGCCGTTTGA TGGTGCTTTT CGAGACGCCG AACTTGTCAG CCAACTCCTG
CGCGGTCTGC GTGCGACGCA TCACGCGTTC GCGGAGCCAG AAGCCGCTGC TCGGCAAACT ACCACGAAAA GCTCTGCGGC
TTGAACAGTC GGTTGAGGAC GCGCCAGACG CACGCTGCGT AGTGCGCAAG TGCAGCACCC ATCAGTCCGT CCCCTCTGCT
GCTGCGAACA GTGCCGATCG ATCGACCTTC TTGAGCTTCG GCCGCGGCGC GGTGGCGTTC TTCCGTACCG ACGTCGTGGG
TAGTCAGGCA GGGGAGACGA CGACGCTTGT CACGGCTAGC TAGCTGGAAG AACTCGAAGC CGGCGCCGCG CCACCGCAAG
AAGGCATGGC CTTCCGTTTT TGCGCTGCTG CTCACTTTGC CGCGGCGTGC CTGGATTTTC GAGAACTCGG CGGCGGTGAA
GGTGCGGTGG GTCCAGTGGG CGACTGATTT GAAGGCAAAA ACGCGACGAC GAGTGAAACG GCGCCGCACG GACCTAAAAG
CTCTTGAGCC GCCGCCACTT CCACGCCACC CAGGTCACCC GCTGACTAAA GCCGATCTGC TCGGCCTCGG CCCGACTCAT
GGGGCCGATC CCGTCGTTGG CGTCGAGGGT GAAGTTGGTC AGGGCGGTGA AGTCGGTGAC CATCTGCCGC CGGCTAGACG
AGCCGGAGCC GGGCTGAGTA CCCCGGCTAG GGCAGCAACC GCAGCTCCCA CTTCAACCAG TCCCGCCACT TCAGCCACTG
GTAGACGGCG CACACAGTGA TCGACGGGTA GTTCTGTTTC CGGATCTCGC GGTAGGCCCA TTCCCGGGTG CGGTCGAACA
GTTCGACGTT CCGGCCCGTT TCGGTCCTGA GTGTGTCACT AGCTGCCCAT CAAGACAAAG GCCTAGAGCG CCATCCGGGT
AAGGGCCCAC GCCAGCTTGT CAAGCTGCAA GGCCGGGCAA AGCCAGGACT CCTGTGTCTT GCGGCCGTAG TCCGGTGGGG
CGGGGAAACG GTCACCGAGC GCTTTTGCGA GGCCTTTGAG CGAGTACGGA TCCGAGGGAC CCCAGACCGT GGACACAGAA
CGCCGGCATC AGGCCACCCC GCCCCTTTGC CAGTGGCTCG CGAAAACGCT CCGGAAACTC GCTCATGCCT AGGCTCCCTG
GGGTCTGGCA CGTCCAGTGC GGGTGGATCG GGTTCTGGGT GAGCTGCTGC GCGTAGCCCT GATCGGCGCC GACCACCGAG
GCGATCAGCC CCTGGTTCAC CCGGTCGTAG GCAGGTCACG CCCACCTAGC CCAAGACCCA CTCGACGACG CGCATCGGGA
CTAGCCGCGG CTGGTGGCTC CGCTAGTCGG GGACCAAGTG GGCCAGCATC AGCCGCAGCG GGCCCTGTCG GGCTGCCTGG
AGGGTGTAGA CCGGGCTTTC GAGCAGCCAC CACAGGTGCG CGTGCTCGGT CGCGGGATTG ATCGTCATCA TCGGCGTCGC
CCGGGACAGC CCGACGGACC TCCCACATCT GGCCCGAAAG CTCGTCGGTG GTGTCCACGC GCACGAGCCA GCGCCCTAAC
TAGCAGTAGT CGGTCGGATC GGGCAGATCC GCGTTACGTG CGGCCCACTG CGCCTGGTCG TCGTCCACGT CGAGCACCAA
GCCCAACCTG ATCGACGGGG TGCGGGCCGC GCCAGCCTAG CCCGTCTAGG CGCAATGCAC GCCGGGTGAC GCGGACCAGC
AGCAGGTGCA GCTCGTGGTT CGGGTTGGAC TAGCTGCCCC ACGCCCGGCG AATGTAGCGG CGGGTGAGCG CCTCCGCGCG
CGGCTGCGGC CACTGCCCGT CCCGGACGTA GTCATCCGTC GCGTGCGGGT ATTTGAACCG CCAGCGGTCC TTACATCGCC
GCCCACTCGC GGAGGCGCGC GCCGACGCCG GTGACGGGCA GGGCCTGCAT CAGTAGGCAG CGCACGCCCA TAAACTTGGC
GGTCGCCAGG AACCAGGCGT CAACAGCAGC GGTCATGACC GCCAAGCTAG GGCCGGATCT GTACCGATCG GGGGAGGCGC
GCCGCAAATT ATTTAAGAGT CTCGCTAGCA TTGGTCCGCA GTTGTCGTCG CCAGTACTGG CGGTTCGATC CCGGCCTAGA
CATGGCTAGC CCCCTCCGCG CGGCGTTTAA TAAATTCTCA GAGCGATCGT AACCATGTCA GGTGTTGCGG TGGGTTCCGG
GTAAACCTCC ACCCGAATTA TTTAAGAGTC TCGCTAGCTA AGCCCTATCT GATGCTGCGC GGGGGGTCCT TTGGTACAGT
CCACAACGCC ACCCAAGGCC CATTTGGAGG TGGGCTTAAT AAATTCTCAG AGCGATCGAT TCGGGATAGA CTACGACGCG
CCCCCCAGGA TCGCACTGAA TCTCAAAGGT GGCCGGCTGA ATTTCGTCGC GCGAAAACCT CCCTGGACAG TTCTGGAATT
CAGCAAGAGG TGTGTCTGAA CTTCGGTGTT AGCGTGACTT AGAGTTTCCA CCGGCCGACT TAAAGCAGCG CGCTTTTGGA
GGGACCTGTC AAGACCTTAA GTCGTTCTCC ACACAGACTT GAAGCCACAA TTTTTGGGGG GTGACTCCAG CGGGGTGGGC
ACAACGCGAA CAGAGACCTT GTGTGTACGA CGGCGGGAGG TAAGTCGGGT ACGGCTCGGA CTGCGGTAGA AAAAACCCCC
CACTGAGGTC GCCCCACCCG TGTTGCGCTT GTCTCTGGAA CACACATGCT GCCGCCCTCC ATTCAGCCCA TGCCGAGCCT
GACGCCATCT GCAACCGTCG AATCGATTTC GAGCAGAGCG AGCAGAGCAA GATATTCCAA AACTCCGGGG TTCCTCGGCG
GCCTCCCCCG TCTGTTTGCT CAACCGAGGG CGTTGGCAGC TTAGCTAAAG CTCGTCTCGC TCGTCTCGTT CTATAAGGTT
TTGAGGCCCC AAGGAGCCGC CGGAGGGGGC AGACAAACGA GTTGGCTCCC AGACCTGGCG GTCCCGCGTT TCCGGACGCG
CGGGACCGCC TACCGCTCGA GAGCGGAAGA GCATCTAGAT GCATTCGCGA GGTACCCAGC TTTTGTTCCC TCTGGACCGC
CAGGGCGCAA AGGCCTGCGC GCCCTGGCGG ATGGCGAGCT CTCGCCTTCT CGTAGATCTA CGTAAGCGCT CCATGGGTCG
AAAACAAGGG TTTAGTGAGG GTTAATTGCG CGCTTGGCGT AATCATGGTC ATAGCTGTTT CCTGTGTGAA ATTGTTATCC
GCTCACAATT CCACACAACA TACGAGCCGG AAATCACTCC CAATTAACGC GCGAACCGCA TTAGTACCAG TATCGACAAA
GGACACACTT TAACAATAGG CGAGTGTTAA GGTGTGTTGT ATGCTCGGCC AAGCATAAAG TGTAAAGCCT GGGGTGCCTA
ATGAGTGAGC TAACTCACAT TAATTGCGTT GCGCTCACTG CCCGCTTTCC AGTCGGGAAA CCTGTCGTGC TTCGTATTTC
ACATTTCGGA CCCCACGGAT TACTCACTCG ATTGAGTGTA ATTAACGCAA CGCGAGTGAC GGGCGAAAGG TCAGCCCTTT
GGACAGCACG CAGCTGCATT AATGAATCGG CCAACGCGCG GGGAGAGGCG GTTTGCGTAT TGGGCGCATG CATAAAAACT
GTTGTAATTC ATTAAGCATT CTGCCGACAT GTCGACGTAA TTACTTAGCC GGTTGCGCGC CCCTCTCCGC CAAACGCATA
ACCCGCGTAC
GGAAGCCATC ACAAACGGCA TGATGAACCT GAATCGCCAG CGGCATCAGC ACCTTGTCGC CTTGCGTATA ATATTTGCCC
ATGGGGGTGG GCGAAGAACT CCTTCGGTAG TGTTTGCCGT ACTACTTGGA CTTAGCGGTC GCCGTAGTCG TGGAACAGCG
GAACGCATAT TATAAACGGG TACCCCCACC CGCTTCTTGA CCAGCATGAG ATCCCCGCGC TGGAGGATCA TCCAGCCGGC
GTCCCGGAAA ACGATTCCGA AGCCCAACCT TTCATAGAAG GCGGCGGTGG AATCGAAATC GGTCGTACTC TAGGGGCGCG
ACCTCCTAGT AGGTCGGCCG CAGGGCCTTT TGCTAAGGCT TCGGGTTGGA AAGTATCTTC CGCCGCCACC TTAGCTTTAG
TCGTGATGGC AGGTTGGGCG TCGCTTGGTC GGTCATTTCG AACCCCAGAG TCCCGCTCAG AAGAACTCGT CAAGAAGGCG
ATAGAAGGCG ATGCGCTGCG AGCACTACCG TCCAACCCGC AGCGAACCAG CCAGTAAAGC TTGGGGTCTC AGGGCGAGTC
TTCTTGAGCA GTTCTTCCGC TATCTTCCGC TACGCGACGC AATCGGGAGC GGCGATACCG TAAAGCACGA GGAAGCGGTC
AGCCCATTCG CCGCCAAGCT CTTCAGCAAT ATCACGGGTA GCCAACGCTA TGTCCTGATA TTAGCCCTCG CCGCTATGGC
ATTTCGTGCT CCTTCGCCAG TCGGGTAAGC GGCGGTTCGA GAAGTCGTTA TAGTGCCCAT CGGTTGCGAT ACAGGACTAT
GCGGTCCGCC ACACCCAGCC GGCCACAGTC GATGAATCCA GAAAAGCGGC CATTTTCCAC CATGATATTC GGCAAGCAGG
CATCGCCATG GGTCACGACG CGCCAGGCGG TGTGGGTCGG CCGGTGTCAG CTACTTAGGT CTTTTCGCCG GTAAAAGGTG
GTACTATAAG CCGTTCGTCC GTAGCGGTAC CCAGTGCTGC AGATCCTCGC CGTCGGGCAT GCGCGCCTTG AGCCTGGCGA
ACAGTTCGGC TGGCGCGAGC CCCTGATGCT CTTCGTCCAG ATCATCCTGA TCGACAAGAC TCTAGGAGCG GCAGCCCGTA
CGCGCGGAAC TCGGACCGCT TGTCAAGCCG ACCGCGCTCG GGGACTACGA GAAGCAGGTC TAGTAGGACT AGCTGTTCTG
CGGCTTCCAT CCGAGTACGT GCTCGCTCGA TGCGATGTTT CGCTTGGTGG TCGAATGGGC AGGTAGCCGG ATCAAGCGTA
TGCAGCCGCC GCATTGCATC GCCGAAGGTA GGCTCATGCA CGAGCGAGCT ACGCTACAAA GCGAACCACC AGCTTACCCG
TCCATCGGCC TAGTTCGCAT ACGTCGGCGG CGTAACGTAG AGCCATGATG GATACTTTCT CGGCAGGAGC AAGGTGAGAT
GACAGGAGAT CCTGCCCCGG CACTTCGCCC AATAGCAGCC AGTCCCTTCC CGCTTCAGT TCGGTACTAC CTATGAAAGA
GCCGTCCTCG TTCCACTCTA CTGTCCTCTA GGACGGGGCC GTGAAGCGGG TTATCGTCGG TCAGGGAAGG GCGAAGTCAC
ACAACGTCGA GCACAGCTGC GCAAGGAACG CCCGTCGTGG CCAGCCACGA TAGCCGCGCT GCCTCGTCCT GCAGTTCATT
CAGGGCACCG GACAGGTCGG TGTTGCAGCT CGTGTCGACG CGTTCCTTGC GGGCAGCACC GGTCGGTGCT ATCGGCGCGA
CGGAGCAGGA CGTCAAGTAA GTCCCGTGGC CTGTCCAGCC TCTTGACAAA AAGAACCGGG CGCCCCTGCG CTGACAGCCG
GAACACGGCG GCATCAGAGC AGCCGATTGT CTGTTGTGCC CAGTCATAGC CGAATAGCCT AGAACTGTTT TTCTTGGCCC
GCGGGGACGC GACTGTCGGC CTTGTGCCGC CGTAGTCTCG TCGGCTAACA GACAACACGG GTCAGTATCG GCTTATCGGA
CTCCACCCAA GCGGCCGGAG AACCTGCGTG CAATCCATCT TGTTCAATCA TGCGAAACGA TCCTCATCCT GTCTCTTGAT
CAGATCTTGA TCCCCTGCGC GAGGTGGGTT CGCCGGCCTC TTGGACGCAC GTTAGGTAGA ACAAGTTAGT ACGCTTTGCT
AGGAGTAGGA CAGAGAACTA GTCTAGAACT AGGGGACGCG CATCAGATCC TTGGCGGCAA GAAAGCCATC CAGTTTACTT
TGCAGGGCTT CCCAACCTTA CCAGAGGGCG CCCCAGCTGG CAATTCCGGT TCGCTTGCTG GTAGTCTAGG AACCGCCGTT
CTTTCGGTAG GTCAAATGAA ACGTCCCGAA GGGTTGGAAT GGTCTCCCGC GGGGTCGACC GTTAAGGCCA AGCGAACGAC
TCCATAAAAC CGCCCAGTCT AGCTATCGCC ATGTAAGCCC ACTGCAAGCT ACCTGCTTTC TCTTTGCGCT TGCGTTTTCC
CTTGTCCAGA TAGCCCAGTA AGGTATTTTG GCGGGTCAGA TCGATAGCGG TACATTCGGG TGACGTTCGA TGGACGAAAG
AGAAACGCGA ACGCAAAAGG GAACAGGTCT ATCGGGTCAT GCTGACATTC ATCCCAGGTG GCACTTTTCG GGGAAATGTG
CGCGCCCGCG TTCCTGCTGG CGCTGGGCCT GTTTCTGGCG CTGGACTTCC CGCTGTTCCG CGACTGTAAG TAGGGTCCAC
CGTGAAAAGC CCCTTTACAC GCGCGGGCGC AAGGACGACC GCGACCCGGA CAAAGACCGC GACCTGAAGG GCGACAAGGC
TCAGCAGCTT TTCGCCCACG GCCTTGATGA TCGCGGCGGC CTTGGCCTGC ATATCCCGAT TCAACGGCCC CAGGGCGTCC
AGAACGGGCT TCAGGCGCTC AGTCGTCGAA AAGCGGGTGC CGGAACTACT AGCGCCGCCG GAACCGGACG TATAGGGCTA
AGTTGCCGGG GTCCCGCAGG TCTTGCCCGA AGTCCGCGA CCGAAGGTCT CGGGCCGTCT CTTGGGCTTG ATCGGCCTTC
TTGCGCATCT CACGCGCTCC TGCGGCGGCC TGTAGGGCAG GCTCATACCC CTGCCGAACC GGCTTCCAGA GCCCGGCAGA
GAACCCGAAC TAGCCGGAAG AACGCGTAGA GTGCGCGAGG ACGCCGCCGG ACATCCCGTC CGAGTATGGG GACGGCTTGG
GCTTTTGTCA GCCGGTCGGC CACGGCTTCC GGCGTCTCAA CGCGCTTTGA GATTCCCAGC TTTTCGGCCA ATCCCTGCGG
TGCATAGGCG CGTGGCTCGA CGAAAACAGT CGGCCAGCCG GTGCCGAAGG CCGCAGAGTT GCGCGAAACT CTAAGGGTCG
AAAAGCCGGT TAGGGACGCC ACGTATCCGC GCACCGAGCT CCGCTTGCGG GCTGATGGTG ACGTGGCCCA CTGGTGGCCG
CTCCAGGGCC TCGTAGAACG CCTGAATGCG CGTGTGACGT GCCTTGCTGC CCTCGATGCC GGCGAACGCC CGACTACCAC
TGCACCGGGT GACCACCGGC GAGGTCCCGG AGCATCTTGC GGACTTACGC GCACACTGCA CGGAACGACG GGAGCTACGG
CCGTTGCAGC CCTAGATCGG CCACAGCGGC CGCAAACGTG GTCTGGTCGC GGGTCATCTG CGCTTTGTTG CCGATGAACT
CCTTGGCCGA CAGCCTGCCG GGCAACGTCG GGATCTAGCC GGTGTCGCCG GCGTTTGCAC CAGACCAGCG CCCAGTAGAC
GCGAAACAAC GGCTACTTGA GGAACCGGCT GTCGGACGGC TCCTGCGTCA GCGGCACCAC GAACGCGGTC ATGTGCGGGC
TGGTTTCGTC ACGGTGGATG CTGGCCGTCA CGATGCGATC CGCCCCGTAC TTGTCCGCCA AGGACGCAGT CGCCGTGGTG
CTTGCGCCAG TACACGCCCG ACCAAAGCAG TGCCACCTAC GACCGGCAGT GCTACGCTAG GCGGGGCATG AACAGGCGGT
GCCACTTGTG CGCCTTCTCG AAGAACGCCG CCTGCTGTTC TTGGCTGGCC GACTTCCACC ATTCCGGGCT GGCCGTCATG
ACGTACTCGA CCGCCAACAC CGGTGAACAC GCGGAAGAGC TTCTTGCGGC GGACGACAAG AACCGACCGG CTGAAGGTGG
TAAGGCCCGA CCGGCAGTAC TGCATGAGCT GGCGGTTGTG AGCGTCCTTG CGCCGCTTCT CTGGCAGCAA CTCGCGCAGT
CGGCCCATCG CTTCATCGGT GCTGCTGGCC GCCCAGTGCT CGTTCTCTGG CGTCCTGCTG TCGCAGGAAC GCGGCGAAGA
GACCGTCGTT GAGCGCGTCA GCCGGGTAGC GAAGTAGCCA CGACGACCGG CGGGTCACGA GCAAGAGACC GCAGGACGAC
GCGTCAGCGT TGGGCGTCTC GCGCTCGCGG TAGGCGTGCT TGAGACTGGC CGCCACGTTG CCCATTTTCG CCAGCTTCTT
GCATCGCATG ATCGCGTATG CGCAGTCGCA ACCCGCAGAG CGCGAGCGCC ATCCGCACGA ACTCTGACCG GCGGTGCAAC
GGGTAAAAGC GGTCGAAGAA CGTAGCGTAC TAGCGCATAC CCGCCATGCC TGCCCCTCCC TTTTGGTGTC CAACCGGCTC
GACGGGGGCA GCGCAAGGCG GTGCCTCCGG CGGGCCACTC AATGCTTGAG TATACTCACT GGCGGTACGG ACGGGGAGGG
AAAACCACAG GTTGGCCGAG CTGCCCCCGT CGCGTTCCGC CACGGAGGCC GCCCGGTGAG TTACGAACTC ATATGAGTGA
AGACTTTGCT TCGCAAAGTC GTGACCGCCT ACGGCGGCTG CGGCGCCCTA CGGGCTTGCT CTCCGGGCTT CGCCCTGCGC
GGTCGCTGCG CTCCCTTGCC TCTGAAACGA AGCGTTTCAG CACTGGCGGA TGCCGCCGAC GCCGCGGGAT GCCCGAACGA
GAGGCCCGAA GCGGGACGCG CCAGCGACGC GAGGGAACGG
SEQ ID NO: 4
GGGGAGCCGC GCCGAAGGCG TGGGGGAACC CCGCAGGGGT GCCCTTCTTT GGGCACCAAA GAACTAGATA
TAGGGCGAAA TGCGAAAGAC TTAAAAATCA CCCCTCGGCG CGGCTTCCGC ACCCCCTTGG GGCGTCCCCA CGGGAAGAAA
CCCGTGGTTT CTTGATCTAT ATCCCGCTTT ACGCTTTCTG AATTTTTAGT ACAACTTAAA AAAGGGGGGT ACGCAACAGC
TCATTGCGGC ACCCCCCGCA ATAGCTCATT GCGTAGGTTA AAGAAAATCT GTAATTGACT GCCACTTTTA TGTTGAATTT
TTTCCCCCCA TGCGTTGTCG AGTAACGCCG TGGGGGGCGT TATCGAGTAA CGCATCCAAT TTCTTTTAGA CATTAACTGA
CGGTGAAAAT CGCAACGCAT AATTGTTGTC GCGCTGCCGA AAAGTTGCAG CTGATTGCGC ATGGTGCCGC AACCGTGCGG
CACCCTACCG CATGGAGATA AGCATGGCCA GCGTTGCGTA TTAACAACAG CGCGACGGCT TTTCAACGTC GACTAACGCG
TACCACGGCG TTGGCACGCC GTGGGATGGC GTACCTCTAT TCGTACCGGT CGCAGTCCAG AGAAATCGGC ATTCAAGCCA
AGAACAAGCC CGGTCACTGG GTGCAAACGG AACGCAAAGC GCATGAGGCG TGGGCCGGGC TTATTGCGAG GCGTCAGGTC
TCTTTAGCCG TAAGTTCGGT TCTTGTTCGG GCCAGTGACC CACGTTTGCC TTGCGTTTCG CGTACTCCGC ACCCGGCCCG
AATAACGCTC GAAACCCACG GCGGCAATGC TGCTGCATCA CCTCGTGGCG CAGATGGGCC ACCAGAACGC CGTGGTGGTC
AGCCAGAAGA CACTTTCCAA GCTCATCGGA CTTTGGGTGC CGCCGTTACG ACGACGTAGT GGAGCACCGC GTCTACCCGG
TGGTCTTGCG GCACCACCAG TCGGTCTTCT GTGAAAGGTT CGAGTAGCCT CGTTCTTTGC GGACGGTCCA ATACGCAGTC
AAGGACTTGG TGGCCGAGCG CTGGATCTCC GTCGTGAAGC TCAACGGCCC CGGCACCGTG TCGGCCTACG GCAAGAAACG
CCTGCCAGGT TATGCGTCAG TTCCTGAACC ACCGGCTCGC GACCTAGAGG CAGCACTTCG AGTTGCCGGG GCCGTGGCAC
AGCCGGATGC TGGTCAATGA CCGCGTGGCG TGGGGCCAGC CCCGCGACCA GTTGCGCCTG TCGGTGTTCA GTGCCGCCGT
GGTGGTTGAT CACGACGACC AGGACGAATC ACCAGTTACT GGCGCACCGC ACCCCGGTCG GGGCGCTGGT CAACGCGGAC
AGCCACAAGT CACGGCGGCA CCACCAACTA GTGCTGCTGG TCCTGCTTAG GCTGTTGGGG CATGGCGACC TGCGCCGCAT
CCCGACCCTG TATCCGGGCG AGCAGCAACT ACCGACCGGC CCCGGCGAGG AGCCGCCCAG CCAGCCCGGC CGACAACCCC
GTACCGCTGG ACGCGGCGTA GGGCTGGGAC ATAGGCCCGC TCGTCGTTGA TGGCTGGCCG GGGCCGCTCC TCGGCGGGTC
GGTCGGGCCG ATTCCGGGCA TGGAACCAGA CCTGCCAGCC TTGACCGAAA CGGAGGAATG GGAACGGCGC GGGCAGCAGC
GCCTGCCGAT GCCCGATGAG CCGTGTTTTC TAAGGCCCGT ACCTTGGTCT GGACGGTCGG AACTGGCTTT GCCTCCTTAC
CCTTGCCGCG CCCGTCGTCG CGGACGGCTA CGGGCTACTC GGCACAAAAG TGGACGATGG CGAGCCGTTG GAGCCGCCGA
CACGGGTCAC GCTGCCGCGC CGGTAGCACT TGGGTTGCGC AGCAACCCGT AAGTGCGCTG TTCCAGACTA ACCTGCTACC
GCTCGGCAAC CTCGGCGGCT GTGCCCAGTG CGACGGCGCG GCCATCGTGA ACCCAACGCG TCGTTGGGCA TTCACGCGAC
AAGGTCTGAT TCGGCTGTAG CCGCCTCGCC GCCCTATACC TTGTCTGCCT CCCCGCGTTG CGTCGCGGTG CATGGAGCCG
GGCCACCTCG ACCTGAATGG AAGCCGGCGG AGCCGACATC GGCGGAGCGG CGGGATATGG AACAGACGGA GGGGCGCAAC
GCAGCGCCAC GTACCTCGGC CCGGTGGAGC TGGACTTACC TTCGGCCGCC CACCTCGCTA ACGGATTCAC CGTTTTTATC
AGGCTCTGGG AGGCAGAATA AATGATCATA TCGTCAATTA TTACCTCCAC GGGGAGAGCC TGAGCAAACT GTGGAGCGAT
TGCCTAAGTG GCAAAAATAG TCCGAGACCC TCCGTCTTAT TTACTAGTAT AGCAGTTAAT AATGGAGGTG CCCCTCTCGG
ACTCGTTTGA GGCCTCAGGC ATTTGAGAAG CACACGGTCA CACTGCTTCC GGTAGTCAAT AAACCGGTAA ACCAGCAATA
GACATAAGCG GCTATTTAAC GACCCTGCCC CCGGAGTCCG TAAACTCTTC GTGTGCCAGT GTGACGAAGG CCATCAGTTA
TTTGGCCATT TGGTCGTTAT CTGTATTCGC CGATAAATTG CTGGGACGGG TGAACCGACG ACCGGGTCGA ATTTGCTTTC
GAATTTCTGC CATTCATCCG CTTATTATCA CTTATTCAGG CGTAGCACCA GGCGTTTAAG GGCACCAATA ACTTGGCTGC
TGGCCCAGCT TAAACGAAAG CTTAAAGACG GTAAGTAGGC GAATAATAGT GAATAAGTCC GCATCGTGGT CCGCAAATTC
CCGTGGTTAT ACTGCCTTAA AAAAATTACG CCCCGCCCTG CCACTCATCG CAGTCGGCCT ATTGGTTAAA AAATGAGCTG
ATTTAACAAA AATTTAACGC GAATTTTAAC TGACGGAATT TTTTTAATGC GGGGCGGGAC GGTGAGTAGC GTCAGCCGGA
TAACCAATTT TTTACTCGAC TAAATTGTTT TTAAATTGCG CTTAAAATTG AAAATATTAA CGCTTACAAT TTCCATTCGC
CATTCAGGCT GCGCAACTGT TGGGAAGGGC GATCGGTGCG GGCCTCTTCG CTATTACGCC AGCTGGCGAA TTTTATAATT
GCGAATGTTA AAGGTAAGCG GTAAGTCCGA CGCGTTGACA ACCCTTCCCG CTAGCCACGC CCGGAGAAGC GATAATGCGG
TCGACCGCTT AGGGGGATGT GCTGCAAGGC GATTAAGTTG GGTAACGCCA GGGTTTTCCC AGTCACGACG TTGTAAAACG
ACGGCCAGTG AGCGCGCGTA ATACGACTCA TCCCCCTACA CGACGTTCCG CTAATTCAAC CCATTGCGGT CCCAAAAGGG
TCAGTGCTGC AACATTTTGC TGCCGGTCAC TCGCGCGCAT TATGCTGAGT CTATAGGGCG AATTGGAGCT CCACCGCGGT
GGCGGCCGCT CTAGAACTAG TGGATCCCCC GGGCTGCAGG AATTCGATAT CAAGCTTTTA CGCCCCGCCC GATATCCCGC
TTAACCTCGA GGTGGCGCCA CCGCCGGCGA GATCTTGATC ACCTAGGGGG CCCGACGTCC TTAAGCTATA GTTCGAAAAT
GCGGGGCGGG TGCCACTCAT CGCAGTACTG TTGTAATTCA TTAAGCATTC TGCCGACATG GAAGCCATCA CAAACGGCAT
GATGAACCTG AATCGCCAGC GGCATCAGCA ACGGTGAGTA GCGTCATGAC AACATTAAGT AATTCGTAAG ACGGCTGTAC
CTTCGGTAGT GTTTGCCGTA CTACTTGGAC TTAGCGGTCG CCGTAGTCGT CCTTGTCGCC TTGCGTATAA TATTTGCCCA
TGGTGAAAAC GGGGGCGAAG AAGTTGTCCA TATTGGCCAC GTTTAAATCA AAACTGGTGA AACTCACCCA GGAACAGCGG
AACGCATATT ATAAACGGGT ACCACTTTTG CCCCCGCTTC TTCAACAGGT ATAACCGGTG CAAATTTAGT TTTGACCACT
TTGAGTGGGT GGGATTGGCT GAGACGAAAA ACATATTCTC AATAAACCCT TTAGGGAAAT AGGCCAGGTT TTCACCGTAA
CACGCCACAT CTTGCGAATA TATGTGTAGA CCCTAACCGA CTCTGCTTTT TGTATAAGAG TTATTTGGGA AATCCCTTTA
TCCGGTCCAA AAGTGGCATT GTGCGGTGTA GAACGCTTAT ATACACATCT AACTGCCGGA AATCGTCGTG GTATTCACTC
CAGAGCGATG AAAACGTTTC AGTTTGCTCA TGGAAAACGG TGTAACAAGG GTGAACACTA TCCCATATCA TTGACGGCCT
TTAGCAGCAC CATAAGTGAG GTCTCGCTAC TTTTGCAAAG TCAAACGAGT ACCTTTTGCC ACATTGTTCC CACTTGTGAT
AGGGTATAGT
CCAGCTCACC GTCTTTCATT GCCATACGAA ATTCCGGATG AGCATTCATC AGGCGGGCAA GAATGTGAAT AAAGGCCGGA
TAAAACTTGT GCTTATTTTT GGTCGAGTGG CAGAAAGTAA CGGTATGCTT TAAGGCCTAC TCGTAAGTAG TCCGCCCGTT
CTTACACTTA TTTCCGGCCT ATTTTGAACA CGAATAAAAA CTTTACGGTC TTTAAAAAGG CCGTAATATC CAGCTGAACG
GTCTGGTTAT AGGTACATTG AGCAACTGAC TGAAATGCCT CAAAATGTTC TTTACGATGC GAAATGCCAG AAATTTTTCC
GGCATTATAG GTCGACTTGC CAGACCAATA TCCATGTAAC TCGTTGACTG ACTTTACGGA GTTTTACAAG AAATGCTACG
CATTGGGATA TATCAACGGT GGTATATCCA GTGATTTTTT TCTCCATATG GTTAACCTTA ATTAAGGGGT CGACGGGCCC
GGGATCCGAT GCTCTTCCGC GTAACCCTAT ATAGTTGCCA CCATATAGGT CACTAAAAAA AGAGGTATAC CAATTGGAAT
TAATTCCCCA GCTGCCCGGG CCCTAGGCTA CGAGAAGGCG TAAGATCTTT TACTAGTTCA GTCCATCTCG CCGTGTATGC
GGGCCTGACG GATCAACGTT CCCACCGAGC CAGTCGAGAT GTTCATCTGG TCGGCGATCT ATTCTAGAAA ATGATCAAGT
CAGGTAGAGC GGCACATACG CCCGGACTGC CTAGTTGCAA GGGTGGCTCG GTCAGCTCTA CAAGTAGACC AGCCGCTAGA
GCCGGTACTT CAAACCTTGT TTGCGCAGTT CCACAGCCTT CTTGCGGCGT TCCTGCGCAC GAGCGATGTA GTCGCCTCGG
TCTTCGGCGA CGAGCCGTTT CGGCCATGAA GTTTGGAACA AACGCGTCAA GGTGTCGGAA GAACGCCGCA AGGACGCGTG
CTCGCTACAT CAGCGGAGCC AGAAGCCGCT GCTCGGCAAA GATGGTGCTT TTCGAGACGC CGAACTTGTC AGCCAACTCC
TGCGCGGTCT GCGTGCGACG CATCACGCGT TCTGCAGCAC CCATCAGTCC GTCCCCTCTG CTACCACGAA AAGCTCTGCG
GCTTGAACAG TCGGTTGAGG ACGCGCCAGA CGCACGCTGC GTAGTGCGCA AGACGTCGTG GGTAGTCAGG CAGGGGAGAC
CTGCTGCGAA CAGTGCCGAT CGATCGACCT TCTTGAGCTT CGGCCGCGGC GCGGTGGCGT TCTTCCGTAC CGCTTCCGTT
TTTGCGCTGC TGCTCACTTT GACGACGCTT GTCACGGCTA GCTAGCTGGA AGAACTCGAA GCCGGCGCCG CGCCACCGCA
AGAAGGCATG GCGAAGGCAA AAACGCGACG ACGAGTGAAA GCCGCGGCGT GCCTGGATTT TCGAGAACTC GGCGGCGGTG
AAGGTGCGGT GGGTCCAGTG GGCGACTGAT TTGCCGATCT GCTCGGCCTC GGCCCGACTC CGGCGCCGCA CGGACCTAAA
AGCTCTTGAG CCGCCGCCAC TTCCACGCCA CCCAGGTCAC CCGCTGACTA AACGGCTAGA CGAGCCGGAG CCGGGCTGAG
ATGGGGCCGA TCCCGTCGTT GGCGTCGAGG GTGAAGTTGG TCAGGGCGGT GAAGTCGGTG ACCATCTGCC GCCACACAGT
GATCGACGGG TAGTTCTGTT TACCCCGGCT AGGGCAGCAA CCGCAGCTCC CACTTCAACC AGTCCCGCCA CTTCAGCCAC
TGGTAGACGG CGGTGTGTCA CTAGCTGCCC ATCAAGACAA TCCGGATCTC GCGGTAGGCC CATTCCCGGG TGCGGTCGAA
CAGTTCGACG TTCCGGCCCG TTTCGGTCCT GACCTGTGTC TTGCGGCCGT AGTCCGGTGG AGGCCTAGAG CGCCATCCGG
GTAAGGGCCC ACGCCAGCTT GTCAAGCTGC AAGGCCGGGC AAAGCCAGGA CTGGACACAG AACGCCGGCA TCAGGCCACC
GGCGGGGAAA CGGTCACCGA GCGCTTTTGC GAGGCCTTTG AGCGAGTACG GATCCGAGGG ACCCCAGACC GTCGTCCAGT
GCGGGTGGAT CGGGTTCTGG CCGCCCCTTT GCCAGTGGCT CGCGAAAACG CTCCGGAAAC TCGCTCATGC CTAGGCTCCC
TGGGGTCTGG CAGCAGGTCA CGCCCACCTA GCCCAAGACC GTGAGCTGCT GCGCGTAGCC CTGATCGGCG CCGACCACCG
AGGCGATCAG CCCCTGGTTC ACCCGGTCGT AGAGCCGCAG CGGGCCCTGT CGGGCTGCCT CACTCGACGA CGCGCATCGG
GACTAGCCGC GGCTGGTGGC TCCGCTAGTC GGGGACCAAG TGGGCCAGCA TCTCGGCGTC GCCCGGGACA GCCCGACGGA
GGAGGGTGTA GACCGGGCTT TCGAGCAGCC ACCACAGGTG CGCGTGCTCG GTCGCGGGAT TGATCGTCAT CACGGTCGGA
TCGGGCAGAT CCGCGTTACG CCTCCCACAT CTGGCCCGAA AGCTCGTCGG TGGTGTCCAC GCGCACGAGC CAGCGCCCTA
ACTAGCAGTA GTGCCAGCCT AGCCCGTCTA GGCGCAATGC TGCGGCCCAC TGCGCCTGGT CGTCGTCCAC GTCGAGCACC
AAGCCCAACC TGATCGACGG GGTGCGGGCC GCAATGTAGC GGCGGGTGAG CGCCTCCGCG ACGCCGGGTG ACGCGGACCA
GCAGCAGGTG CAGCTCGTGG TTCGGGTTGG ACTAGCTGCC CCACGCCCGG CGTTACATCG CCGCCCACTC GCGGAGGCGC
CGCGGCTGCG GCCACTGCCC GTCCCGGACG TAGTCATCCG TCGCGTGCGG GTATTTGAAC CGCCAGCGGT CCAACCAGGC
GTCAACAGCA GCGGTCATGA GCGCCGACGC CGGTGACGGG CAGGGCCTGC ATCAGTAGGC AGCGCACGCC CATAAACTTG
GCGGTCGCCA GGTTGGTCCG CAGTTGTCGT CGCCAGTACT CCGCCAAGCT AGGGCCGGAT CTGTACCGAT CGGGGGAGGC
GCGCCGCAAA TTATTTAAGA GTCTCGCTAG CAAACCATGT CAGGTGTTGC GGTGGGTTCC GGCGGTTCGA TCCCGGCCTA
GACATGGCTA GCCCCCTCCG CGCGGCGTTT AATAAATTCT CAGAGCGATC GTTTGGTACA GTCCACAACG CCACCCAAGG
GGGTAAACCT CCACCCGAAT TATTTAAGAG TCTCGCTAGC TAAGCCCTAT CTGATGCTGC GCGGGGGGTC CTTCGCACTG
AATCTCAAAG GTGGCCGGCT CCCATTTGGA GGTGGGCTTA ATAAATTCTC AGAGCGATCG ATTCGGGATA GACTACGACG
CGCCCCCCAG GAAGCGTGAC TTAGAGTTTC CACCGGCCGA GAATTTCGTC GCGCGAAAAC CTCCCTGGAC AGTTCTGGAA
TTCAGCAAGA GGTGTGTCTG AACTTCGGTG TTTTTTTGGG GGGTGACTCC AGCGGGGTGG CTTAAAGCAG CGCGCTTTTG
GAGGGACCTG TCAAGACCTT AAGTCGTTCT CCACACAGAC TTGAAGCCAC AAAAAAACCC CCCACTGAGG TCGCCCCACC
GCACAACGCG AACAGAGACC TTGTGTGTAC GACGGCGGGA GGTAAGTCGG GTACGGCTCG GACTGCGGTA GAGCAACCGT
CGAATCGATT TCGAGCAGAG CGTGTTGCGC TTGTCTCTGG AACACACATG CTGCCGCCCT CCATTCAGCC CATGCCGAGC
CTGACGCCAT CTCGTTGGCA GCTTAGCTAA AGCTCGTCTC CGAGCAGAGC AAGATATTCC AAAACTCCGG GGTTCCTCGG
CGGCCTCCCC CGTCTGTTTG CTCAACCGAG GGAGACCTGG CGGTCCCGCG TTTCCGGACG GCTCGTCTCG TTCTATAAGG
TTTTGAGGCC CCAAGGAGCC GCCGGAGGGG GCAGACAAAC GAGTTGGCTC CCTCTGGACC GCCAGGGCGC AAAGGCCTGC
CGCGGGACCG CCTACCGCTC GAGAGCGGAA GAGCATCTAG ATGCATTCGC GAGGTACCCA GCTTTTGTTC CCTTTAGTGA
GGGTTAATTG CGCGCTTGGC GCGCCCTGGC GGATGGCGAG CTCTCGCCTT CTCGTAGATC TACGTAAGCG CTCCATGGGT
CGAAAACAAG GGAAATCACT CCCAATTAAC GCGCGAACCG GTAATCATGG TCATAGCTGT TTCCTGTGTG AAATTGTTAT
CCGCTCACAA TTCCACACAA CATACGAGCC GGAAGCATAA AGTGTAAAGC CTGGGGTGCC CATTAGTACC AGTATCGACA
AAGGACACAC TTTAACAATA GGCGAGTGTT AAGGTGTGTT GTATGCTCGG CCTTCGTATT TCACATTTCG GACCCCACGG
TAATGAGTGA GCTAACTCAC ATTAATTGCG TTGCGCTCAC TGCCCGCTTT CCAGTCGGGA AACCTGTCGT GCCAGCTGCA
TTAATGAATC GGCCAACGCG ATTACTCACT CGATTGAGTG TAATTAACGC AACGCGAGTG ACGGGCGAAA GGTCAGCCCT
TTGGACAGCA CGGTCGACGT AATTACTTAG CCGGTTGCGC CGGGGAGAGG CGGTTTGCGT ATTGGGCGCA TGCATAAAAA
CTGTTGTAAT TCATTAAGCA TTCTGCCGAC ATGGAAGCCA TCACAAACGG CATGATGAAC GCCCCTCTCC GCCAAACGCA
TAACCCGCGT ACGTATTTTT GACAACATTA AGTAATTCGT AAGACGGCTG TACCTTCGGT AGTGTTTGCC GTACTACTTG
CTGAATCGCC AGCGGCATCA GCACCTTGTC GCCTTGCGTA TAATATTTGC CCATGGGGGT GGGCGAAGAA CTCCAGCATG
AGATCCCCGC GCTGGAGGAT GACTTAGCGG TCGCCGTAGT CGTGGAACAG CGGAACGCAT ATTATAAACG GGTACCCCCA
CCCGCTTCTT GAGGTCGTAC TCTAGGGGCG CGACCTCCTA CATCCAGCCG GCGTCCCGGA AAACGATTCC GAAGCCCAAC
CTTTCATAGA AGGCGGCGGT GGAATCGAAA TCTCGTGATG GCAGGTTGGG CGTCGCTTGG GTAGGTCGGC CGCAGGGCCT
TTTGCTAAGG CTTCGGGTTG GAAAGTATCT TCCGCCGCCA CCTTAGCTTT AGAGCACTAC CGTCCAACCC GCAGCGAACC
TCGGTCATTT CGAACCCCAG AGTCCCGCTC AGAAGAACTC GTCAAGAAGG CGATAGAAGG CGATGCGCTG CGAATCGGGA
GCGGCGATAC CGTAAAGCAC AGCCAGTAAA GCTTGGGGTC TCAGGGCGAG TCTTCTTGAG CAGTTCTTCC GCTATCTTCC
GCTACGCGAC GCTTAGCCCT CGCCGCTATG GCATTTCGTG GAGGAAGCGG TCAGCCCATT CGCCGCCAAG CTCTTCAGCA
ATATCACGGG TAGCCAACGC TATGTCCTGA TAGCGGTCCG CCACACCCAG CCGGCCACAG CTCCTTCGCC AGTCGGGTAA
GCGGCGGTTC GAGAAGTCGT TATAGTGCCC ATCGGTTGCG ATACAGGACT ATCGCCAGGC GGTGTGGGTC GGCCGGTGTC
TCGATGAATC CAGAAAAGCG GCCATTTTCC ACCATGATAT TCGGCAAGCA GGCATCGCCA TGGGTCACGA CGAGATCCTC
GCCGTCGGGC ATGCGCGCCT AGCTACTTAG GTCTTTTCGC CGGTAAAAGG TGGTACTATA AGCCGTTCGT CCGTAGCGGT
ACCCAGTGCT GCTCTAGGAG CGGCAGCCCG TACGCGCGGA TGAGCCTGGC GAACAGTTCG GCTGGCGCGA GCCCCTGATG
CTCTTCGTCC AGATCATCCT GATCGACAAG ACCGGCTTCC ATCCGAGTAC GTGCTCGCTC ACTCGGACCG CTTGTCAAGC
CGACCGCGCT CGGGGACTAC GAGAAGCAGG TCTAGTAGGA CTAGCTGTTC TGGCCGAAGG TAGGCTCATG CACGAGCGAG
GATGCGATGT TTCGCTTGGT GGTCGAATGG GCAGGTAGCC GGATCAAGCG TATGCAGCCG CCGCATTGCA TCAGCCATGA
TGGATACTTT CTCGGCAGGA CTACGCTACA AAGCGAACCA CCAGCTTACC CGTCCATCGG CCTAGTTCGC ATACGTCGGC
GGCGTAACGT AGTCGGTACT ACCTATGAAA GAGCCGTCCT GCAAGGTGAG ATGACAGGAG ATCCTGCCCC GGCACTTCGC
CCAATAGCAG CCAGTCCCTT CCCGCTTCAG TGACAACGTC GAGCACAGCT GCGCAAGGAA CGTTCCACTC TACTGTCCTC
TAGGACGGGG CCGTGAAGCG GGTTATCGTC GGTCAGGGAA GGGCGAAGTC ACTGTTGCAG CTCGTGTCGA CGCGTTCCTT
CGCCCGTCGT GGCCAGCCAC GATAGCCGCG CTGCCTCGTC CTGCAGTTCA TTCAGGGCAC CGGACAGGTC GGTCTTGACA
AAAAGAACCG GGCGCCCCTG GCGGGCAGCA CCGGTCGGTG CTATCGGCGC GACGGAGCAG GACGTCAAGT AAGTCCCGTG
GCCTGTCCAG CCAGAACTGT TTTTCTTGGC CCGCGGGGAC CGCTGACAGC CGGAACACGG CGGCATCAGA GCAGCCGATT
GTCTGTTGTG CCCAGTCATA GCCGAATAGC CTCTCCACCC AAGCGGCCGG AGAACCTGCG GCGACTGTCG GCCTTGTGCC
GCCGTAGTCT CGTCGGCTAA CAGACAACAC GGGTCAGTAT CGGCTTATCG GAGAGGTGGG TTCGCCGGCC TCTTGGACGC
TGCAATCCAT CTTGTTCAAT CATGCGAAAC GATCCTCATC CTGTCTCTTG ATCAGATCTT GATCCCCTGC GCCATCAGAT
CCTTGGCGGC AAGAAAGCCA ACGTTAGGTA GAACAAGTTA GTACGCTTTG CTAGGAGTAG GACAGAGAAC TAGTCTAGAA
CTAGGGGACG CGGTAGTCTA GGAACCGCCG TTCTTTCGGT TCCAGTTTAC TTTGCAGGGC TTCCCAACCT TACCAGAGGG
CGCCCCAGCT GGCAATTCCG GTTCGCTTGC TGTCCATAAA ACCGCCCAGT CTAGCTATCG AGGTCAAATG AAACGTCCCG
AAGGGTTGGA ATGGTCTCCC GCGGGGTCGA CCGTTAAGGC CAAGCGAACG ACAGGTATTT TGGCGGGTCA GATCGATAGC
CCATGTAAGC CCACTGCAAG CTACCTGCTT TCTCTTTGCG CTTGCGTTTT CCCTTGTCCA GATAGCCCAG TAGCTGACAT
TCATCCCAGG TGGCACTTTT GGTACATTCG GGTGACGTTC GATGGACGAA AGAGAAACGC GAACGCAAAA GGGAACAGGT
CTATCGGGTC ATCGACTGTA AGTAGGGTCC ACCGTGAAAA CGGGGAAATG TGCGCGCCCG CGTTCCTGCT GGCGCTGGGC
CTGTTTCTGG CGCTGGACTT CCCGCTGTTC CGTCAGCAGC TTTTCGCCCA CGGCCTTGAT GCCCCTTTAC ACGCGCGGGC
GCAAGGACGA CCGCGACCCG GACAAAGACC GCGACCTGAA GGGCGACAAG GCAGTCGTCG AAAAGCGGGT GCCGGAACTA
GATCGCGGCG GCCTTGGCCT GCATATCCCG ATTCAACGGC CCCAGGGCGT CCAGAACGGG CTTCAGGCGC TCCCGAAGGT
CTCGGGCCGT CTCTTGGGCT CTAGCGCCGC CGGAACCGGA CGTATAGGGC TAAGTTGCCG GGGTCCCGCA GGTCTTGCCC
GAAGTCCGCG AGGGCTTCCA GAGCCCGGCA GAGAACCCGA TGATCGGCCT TCTTGCGCAT CTCACGCGCT CCTGCGGCGG
CCTGTAGGGC AGGCTCATAC CCCTGCCGAA CCGCTTTTGT CAGCCGGTCG GCCACGGCTT ACTAGCCGGA AGAACGCGTA
GAGTGCGCGA GGACGCCGCC GGACATCCCG TCCGAGTATG GGGACGGCTT GGCGAAAACA GTCGGCCAGC CGGTGCCGAA
CCGGCGTCTC AACGCGCTTT GAGATTCCCA GCTTTTCGGC CAATCCCTGC GGTGCATAGG CGCGTGGCTC GACCGCTTGC
GGGCTGATGG TGACGTGGCC GGCCGCAGAG TTGCGCGAAA CTCTAAGGGT CGAAAAGCCG GTTAGGGACG CCACGTATCC
GCGCACCGAG CTGGCGAACG CCCGACTACC ACTGCACCGG CACTGGTGGC CGCTCCAGGG CCTCGTAGAA CGCCTGAATG
CGCGTGTGAC GTGCCTTGCT GCCCTCGATG CCCCGTTGCA GCCCTAGATC GGCCACAGCG GTGACCACCG GCGAGGTCCC
GGAGCATCTT GCGGACTTAC GCGCACACTG CACGGAACGA CGGGAGCTAC GGGGCAACGT CGGGATCTAG CCGGTGTCGC
GCCGCAAACG TGGTCTGGTC GCGGGTCATC TGCGCTTTGT TGCCGATGAA CTCCTTGGCC GACAGCCTGC CGTCCTGCGT
CAGCGGCACC ACGAACGCGG CGGCGTTTGC ACCAGACCAG CGCCCAGTAG ACGCGAAACA ACGGCTACTT GAGGAACCGG
CTGTCGGACG GCAGGACGCA GTCGCCGTGG TGCTTGCGCC TCATGTGCGG GCTGGTTTCG TCACGGTGGA TGCTGGCCGT
CACGATGCGA TCCGCCCCGT ACTTGTCCGC CAGCCACTTG TGCGCCTTCT CGAAGAACGC AGTACACGCC CGACCAAAGC
AGTGCCACCT ACGACCGGCA GTGCTACGCT AGGCGGGGCA TGAACAGGCG GTCGGTGAAC ACGCGGAAGA GCTTCTTGCG
CGCCTGCTGT TCTTGGCTGG CCGACTTCCA CCATTCCGGG CTGGCCGTCA TGACGTACTC GACCGCCAAC ACAGCGTCCT
TGCGCCGCTT CTCTGGCAGC GCGGACGACA AGAACCGACC GGCTGAAGGT GGTAAGGCCC GACCGGCAGT ACTGCATGAG
CTGGCGGTTG TGTCGCAGGA ACGCGGCGAA GAGACCGTCG AACTCGCGCA GTCGGCCCAT CGCTTCATCG GTGCTGCTGG
CCGCCCAGTG CTCGTTCTCT GGCGTCCTGC TGGCGTCAGC GTTGGGCGTC TCGCGCTCGC TTGAGCGCGT CAGCCGGGTA
GCGAAGTAGC CACGACGACC GGCGGGTCAC GAGCAAGAGA CCGCAGGACG ACCGCAGTCG CAACCCGCAG AGCGCGAGCG
GGTAGGCGTG CTTGAGACTG GCCGCCACGT TGCCCATTTT CGCCAGCTTC TTGCATCGCA TGATCGCGTA TGCCGCCATG
CCTGCCCCTC CCTTTTGGTG CCATCCGCAC GAACTCTGAC CGGCGGTGCA ACGGGTAAAA GCGGTCGAAG AACGTAGCGT
ACTAGCGCAT ACGGCGGTAC GGACGGGGAG GGAAAACCAC TCCAACCGGC TCGACGGGGG CAGCGCAAGG CGGTGCCTCC
GGCGGGCCAC TCAATGCTTG AGTATACTCA CTAGACTTTG CTTCGCAAAG TCGTGACCGC AGGTTGGCCG AGCTGCCCCC
GTCGCGTTCC GCCACGGAGG CCGCCCGGTG AGTTACGAAC TCATATGAGT GATCTGAAAC GAAGCGTTTC AGCACTGGCG
CTACGGCGGC TGCGGCGCCC TACGGGCTTG CTCTCCGGGC TTCGCCCTGC GCGGTCGCTG CGCTCCCTTG CCAGCCCGTG
GATATGTGGA CGATGGCCGC GATGCCGCCG ACGCCGCGGG ATGCCCGAAC GAGAGGCCCG AAGCGGGACG CGCCAGCGAC
GCGAGGGAAC GGTCGGGCAC CTATACACCT GCTACCGGCG GAGCGGCCAC CGGCTGGCTC GCTTCGCTCG GCCCGTGGAC
AACCCTGCTG GACAAGCTGA TGGACAGGCT GCGCCTGCCC ACGAGCTTGA CCACAGGGAT CTCGCCGGTG GCCGACCGAG
CGAAGCGAGC CGGGCACCTG TTGGGACGAC CTGTTCGACT ACCTGTCCGA CGCGGACGGG TGCTCGAACT GGTGTCCCTA
TGCCCACCGG CTACCCAGCC TTCGACCACA TACCCACCGG CTCCAACTGC GCGGCCTGCG GCCTTGCCCC ATCAATTTTT
TTAATTTTCT CTGGGGAAAA ACGGGTGGCC GATGGGTCGG AAGCTGGTGT ATGGGTGGCC GAGGTTGACG CGCCGGACGC
CGGAACGGGG TAGTTAAAAA AATTAAAAGA GACCCCTTTT GCCTCCGGCC TGCGGCCTGC GCGCTTCGCT TGCCGGTTGG
ACACCAAGTG GAAGGCGGGT CAAGGCTCGC GCAGCGACCG CGCAGCGGCT TGGCCTTGAC CGGAGGCCGG ACGCCGGACG
CGCGAAGCGA ACGGCCAACC TGTGGTTCAC CTTCCGCCCA GTTCCGAGCG CGTCGCTGGC GCGTCGCCGA ACCGGAACTG
GCGCCTGGAA CGACCCAAGC CTATGCGAGT GGGGGCAGTC GAAGGCGAAG CCCGCCCGCC TGCCCCCCGA GCCTCACGGC
GGCGAGTGCG GGGGTTCCAA CGCGGACCTT GCTGGGTTCG GATACGCTCA CCCCCGTCAG CTTCCGCTTC GGGCGGGCGG
ACGGGGGGCT CGGAGTGCCG CCGCTCACGC CCCCAAGGTT GGGGGCAGCG CCACCTTGGG CAAGGCCGAA GGCCGCGCAG
TCGATCAACA AGCCCCGGAG GGGCCACTTT TTGCCGGAG CCCCCGTCGC GGTGGAACCC GTTCCGGCTT CCGGCGCGTC
AGCTAGTTGT TCGGGGCCTC CCCGGTGAAA AACGGCCTC
SEQ ID NO: 5
MEALFLSSSS SSIVASNKLT RLHNHCVWST VIRDKKRFGP TWCRVGGGGD GGRNSNAERP IRVSSLLKDR
GQVLIREQSS PAMDAETLVL SPNGNGRTIE INGVKTLMPF SGASMVGMKE GLGIISFLQG KKFLITGSTG FLAKVLIEKV
LRMAPDVSKI YLLIKAKSKE AAIERLKNEV LDAELFNTLK ETHGASYMSF MLTKLIPVTG NICDSNIGLQ ADSAEEIAKE
VDVIINSAAN TTFNERYDVA LDINTRGPGN LMGFAKKCKK LKLFLQVSTA YVNGQRQGRI MEKPFSMGDC IATENFLEGN
RKALDVDREM KLALEAARKG TQNQDEAQKM KDLGLERARS YGWQDTYVFT KAMGEMMINS TRGDVPVVII RPSVIESTYK
DPFPGWMEGN RMMDPIVLCY GKGQLTGFLV DPKGVLDVVP ADMVVNATLA AIAKHGMAMS DPEPEINVYQ IASSAINPLV
FEDLAELLYN HYKTSPCMDS KGDPIMVRLM KLFNSVDDFS DHLWRDAQER SGLMSGMSSV DSKMMQKLKF ICKKSVEQAK
HLATIYEPYT FYGGRFDNSN TQRLMENMSE DEKREFGFDV GSINWTDYIT NVHIPGLRRH VLKGRA
SEQ ID NO: 6
MATTNVLATS HAFKLNGVSY FSSFPRKPNH YMPRRRLSHT TRRVQTSCFY GETSFEAVTS LVTPKTETSR
NSDGIGIVRF LEGKSYLVTG ATGFLAKVLI EKLLRESLEI GKIFLLMRSK DQESANKRLY DEIISSDLFK LLKQMHGSSY
EAFMKRKLIP VIGDIEEDNL GIKSEIANMI SEEIDVIISC GGRTTFDDRY DSALSVNALG PGRLLSFGKG CRKLKLFLHF
STAYVTGKRE GTVLETPLCI GENITSDLNI KSELKLASEA VRKFRGREEI KKLKELGFER AQHYGWENSY TFTKAIGEAV
IHSKRGNLPV VIIRPSIIES SYNEPFPGWI QGTRMADPII LAYAKGQISD FWADPQSLMD IIPVDMVANA AIAAMAKHGC
GVPEFKVYNL TSSSHVNPMR AGKLIDLSHQ HLCDFPLEET VIDLEHMKIH SSLEGFTSAL SNTIIKQERV IDNEGGGLST
KGKRKLNYFV SLAKTYEPYT FFQARFDNTN TTSLIQEMSM EEKKTFGFDI KGIDWEHYIV NVHLPGLKKE FLSKKKTE
SEQ ID NO: 7
MESNCVQFLG NKTILITGAP GFLAKVLVEK ILRLQPNVKK IYLLLRAPDE KSAMQRLRSE VMEIDLFKVL RNNLGEDNLN
ALMREKIVPV PGDISIDNLG LKDTDLIQRM WSEIDIIINI AATTNFDERY DIGLGINTFG ALNVLNFAKK CVKGQLLLHV
STAYISGEQP GLLLEKPFKM GETLSGDREL DINIEHDLMK QKLKELQDCS DEEISQTMKD FGMARAKLHG WPNTYVFTKA
MGEMLMGKYR ENLPLVIIRP TMITSTIAEP FPGWIEGLKT LDSVIVAYGK GRLKCFLADS NSVFDLIPAD MVVNAMVAAA
TAHSGDTGIQ AIYHVGSSCK NPVTFGQLHD FTARYFAKRP LIGRNGSPII VVKGTILSTM AQFSLYMTLR YKLPLQILRL
INIVYPWSHG DNYSDLSRKI KLAMRLVELY QPYLLFKGIF DDLNTERLRM KRKENIKELD GSFEFDPKSI DWDNYITNTH
IPGLITHVLK Q
SEQ ID NO: 8
MPELAVRTEF DYSSEIYKDA YSRINAIVIE GEQEAYSNYL QMAELLPEDK EELTRLAKME NRHKKGFQAC GNNLQVNPDM
PYAQEFFAGL HGNFQHAFSE GKVVTCLLIQ ALIIEAFAIA AYNIYIPVAD DFARKITEGV VKDEYTHLNY GEEWLKANFA
TAKEELEQAN KENLPLVWKM LNQVQGDAKV LGMEKEALVE DFMISYGEAL SNIGFSTREI MRMSSYGLAG V
SEQ ID NO: 9
MFGLIGHLTS LEHAQAVAED LGYPEYANQG LDFWCSAPPQ VVDNFQVKSV TGQVIEGKYV ESCFLPEMLT
QRRIKAAIRK ILNAMALAQK VGLDITALGG FSSIVFEEFN LKQNNQVRNV ELDFQRFTTG NTHTAYVICR QVESGAKQLG
IDLSQATVAV CGATGDIGSA VCRWLDSKHQ VKELLLIARN RQRLENLQEE LGRGKIMDLE TALPQADIIV WVASMPKGVE
IAGEMLKKPC LIVDGGYPKN LDTRVKADGV HILKGGIVEH SLDITWEIMK IVEMDIPSRQ MFACFAEAIL LEFEGWRTNF
SWGRNQISVN KMEAIGEASV KHGFCPLVAL
SEQ ID NO: 10
CAGTCAATGG AGAGCATTGC CATAAGTAAA GGCATCCCCT GCGTGATAAG ATTACCTTCA GAAAACAGAT AGTTGCTGGG
TTATCGCAGA TTTTTCTCGC GTCAGTTACC TCTCGTAACG GTATTCATTT CCGTAGGGGA CGCACTATTC TAATGGAAGT
CTTTTGTCTA TCAACGACCC AATAGCGTCT AAAAAGAGCG AACCAAATAA CTGTAAATAA TAACTGTCTC TGGGGCGACG
GTAGGCTTTA TATTGCCAAA TTTCGCCCGT GGGAGAAAGC TAGGCTATTC AATGTTTATG TTGGTTTATT GACATTTATT
ATTGACAGAG ACCCCGCTGC CATCCGAAAT ATAACGGTTT AAAGCGGGCA CCCTCTTTCG ATCCGATAAG TTACAAATAC
GAGGACTCCT
SEQ ID NO: 11
CCTGGCTCAG GACGAACGCT GGCGGCGTGC TTAACACATG CAAGTCGAGC GGTAAGGCCC TTCGGGGTAC ACGAGCGGCG
AACGGGTGAG TAACACGTGG GGACCGAGTC CTGCTTGCGA CCGCCGCACG AATTGTGTAC GTTCAGCTCG CCATTCCGGG
AAGCCCCATG TGCTCGCCGC TTGCCCACTC ATTGTGCACC GTGATCTGCC CTGCACTTCG GGATAAGCCT GGGAAACTGG
GTCTAATACC GGATATGACC TTCGGCTGCA TGGCTGAGGG TGGAAAGGTT TACTGGTGCA CACTAGACGG GACGTGAAGC
CCTATTCGGA CCCTTTGACC CAGATTATGG CCTATACTGG AAGCCGACGT ACCGACTCCC ACCTTTCCAA ATGACCACGT
GGATGGGCCC GCGGCCTATC AGCTTGTTGG TGGGGTAATG GCCTACCAAG GCGACGACGG GTAGCCGACC TGAGAGGGTG
ACCGGCCACA CTGGGACTGA CCTACCCGGG CGCCGGATAG TCGAACAACC ACCCCATTAC CGGATGGTTC CGCTGCTGCC
CATCGGCTGG ACTCTCCCAC TGGCCGGTGT GACCCTGACT GACACGGCCC AGACTCCTAC GGGAGGCAGC AGTGGGGAAT
ATTGCACAAT GGGCGAAAGC CTGATGCAGC GACGCCGCGT GAGGGATGAC GGCCTTCGGG CTGTGCCGGG TCTGAGGATG
CCCTCCGTCG TCACCCCTTA TAACGTGTTA CCCGCTTTCG GACTACGTCG CTGCGGCGCA CTCCCTACTG CCGGAAGCCC
TTGTAAACCT CTTTCAGCAG GGACGAAGCG AAAGTGACGG TACCTGCAGA AGAAGCACCG GCCAACTACG TGCCAGCAGC
CGCGGTAATA CGTAGGGTGC AACATTTGGA GAAAGTCGTC CCTGCTTCGC TTTCACTGCC ATGGACGTCT TCTTCGTGGC
CGGTTGATGC ACGGTCGTCG GCGCCATTAT GCATCCCACG AAGCGTTGTC CGGAATTACT GGGCGTAAAG AGCTCGTAGG
CGGTTTGTCG CGTCGTCTGT GAAAACTCAN AGCTCAACCT CGAGCTTGCA GGCGATACGG TTCGCAACAG GCCTTAATGA
CCCGCATTTC TCGAGCATCC GCCAAACAGC GCAGCAGACA CTTTTGAGTN TCGAGTTGGA GCTCGAACGT CCGCTATGCC
GCAGACTTGA GTACTGCAGG GGAGACTGGA ATTCCTGGTG TAGCGGTGAA ATGCGCAGAT ATCAGGAGGA ACACCGGTGG
CGAAGGCGGG TCTCTGGGCA CGTCTGAACT CATGACGTCC CCTCTGACCT TAAGGACCAC ATCGCCACTT TACGCGTCTA
TAGTCCTCCT TGTGGCCACC GCTTCCGCCC AGAGACCCGT GTAACTGACG CTGAGGAGCG AAAGCGTGGG TAGCAAACAG
GATTAGATAC CCTGGTAGTC CACGCCGTAA ACGGTGGGCG CTAGGTGTGG GTTTCCTTCC CATTGACTGC GACTCCTCGC
TTTCGCACCC ATCGTTTGTC CTAATCTATG GGACCATCAG GTGCGGCATT TGCCACCCGC GATCCACACC CAAAGGAAGG
ACGGGATCCG TGCCGTAGTT AACGCATTAA GCGCCCCGCC TGGGGAGTAC GGCCGCAAGG TTAAAACTCA AAGGAATTGA
CGGGGGCCCG CACAAGCGGC TGCCCTAGGC ACGGCATCAA TTGCGTAATT CGCGGGGCGG ACCCCTCATG CCGGCGTTCC
AATTTTGAGT TTCCTTAACT GCCCCCGGGC GTGTTCGCCG GGAGCATGTG GATTAATTCG ATGCAACGCG AAGAACCTTA
CCTGGGTTTG ACATATACCG GAAAGCCGTA GAGATACCGC CCCCCTTGTG GTCGGTATAC CCTCGTACAC CTAATTAAGC
TACGTTGCGC TTCTTGGAAT GGACCCAAAC TGTATATGGC CTTTCGGCAT CTCTATGGCG GGGGGAACAC CAGCCATATG
AGGTGGTGCA TGGCTGTCGT CAGCTCGTGT CGTGAGATGT TGGGTTAAGT CCCGCAACGA GCGCAACCCT TGTCTTATGT
TGCCAGCACG TAATGGTGGG TCCACCACGT ACCGACAGCA GTCGAGCACA GCACTCTACA ACCCAATTCA GGGCGTTGCT
CGCGTTGGGA ACAGAATACA ACGGTCGTGC ATTACCACCC GACTCGTAAG AGACTGCCGG GGTCAACTCG GAGGAAGGTG
GGGACGACGT CAAGTCATCA TGCCCCTTAT GTCCAGGGCT TCACACATGC TACAATGGCC CTGAGCATTC TCTGACGGCC
CCAGTTGAGC CTCCTTCCAC CCCTGCTGCA GTTCAGTAGT ACGGGGAATA CAGGTCCCGA AGTGTGTACG ATGTTACCGG
GGTACAGAGG GCTGCGATAC CGTGAGGTGG AGCGAATCCC TTAAAGCCGG TCTCAGTTCG GATCGGGGTC TGCAACTCGA
CCCCGTGAAG TCGGAGTCGC CCATGTCTCC CGACGCTATG GCACTCCACC TCGCTTAGGG AATTTCGGCC AGAGTCAAGC
CTAGCCCCAG ACGTTGAGCT GGGGCACTTC AGCCTCAGCG TAGTAATCGC AGATCAGCAA CGCTGCGGTG AATACGTTCC
CGGGCCTTGT ACACACCGCC CGTCACGTCA TGAAAGTCGG TAACACCCGA AGCCGGTGGC ATCATTAGCG TCTAGTCGTT
GCGACGCCAC TTATGCAAGG GCCCGGAACA TGTGTGGCGG GCAGTGCAGT ACTTTCAGCC ATTGTGGGCT TCGGCCACCG
CTAACCCCTT GTGGGAGGGA GCCGTCGAAG GTGGGATCGG CGATTGGGAC GAAGTCGTAA CAAGGTAGCC GTACCGGAAG
GGATTGGGGA ACACCCTCCC TCGGCAGCTT CCACCCTAGC CGCTAACCCT GCTTCAGCAT TGTTCCATCG GCATGGCCTT
CC
SEQ ID NO: 12
TCAACGGAGA GTTTGATCCT GGCTCAGGAC GAACGCTGGC GGCGTGCTTA ACACATGCAA GTCGAGCGGT AAGGCCCTTC
GGGGTACACG AGCGGCGAAC AGTTGCCTCT CAAACTAGGA CCGAGTCCTG CTTGCGACCG CCGCACGAAT TGTGTACGTT
CAGCTCGCCA TTCCGGGAAG CCCCATGTGC TCGCCGCTTG GGGTGAGTAA CACGTGGGTG ATCTGCCCTG CACTTCGGGA
TAAGCCTGGG AAACTGGGTC TAATACCGGA TATGACCTTC GGCTGCATGG CCGTTGGTGG CCCACTCATT GTGCACCCAC
TAGACGGGAC GTGAAGCCCT ATTCGGACCC TTTGACCCAG ATTATGGCCT ATACTGGAAG CCGACGTACC GGCAACCACC
AAAGGTTTAC TGGTGCAGGA TGGGCCCGCG GCCTATCAGC TTGTTGGTGG GGTAATGGCC TACCAAGGCG ACGACGGGTA
GCCGACCTGA GAGGGTGACC TTTCCAAATG ACCACGTCCT ACCCGGGCGC CGGATAGTCG AACAACCACC CCATTACCGG
ATGGTTCCGC TGCTGCCCAT CGGCTGGACT CTCCCACTGG GGCCACACTG GGACTGAGAC ACGGCCCAGA CTCCTACGGG
AGGCAGCAGT GGGGAATATT GCACAATGGG CGAAAGCCTG ATGCAGCGAC GCCGCGTGAG CCGGTGTGAC CCTGACTCTG
TGCCGGGTCT GAGGATGCCC TCCGTCGTCA CCCCTTATAA CGTGTTACCC GCTTTCGGAC TACGTCGCTG CGGCGCACTC
GGATGACGGC CTTCGGGTTG TAAACCTCTT TCAGCAGGGA CGAAGCGAAA GTGACGGTAC CTGCAGAAGA AGCACCGGCC
AACTACGTGC CAGCAGCCGC CCTACTGCCG GAAGCCCAAC ATTTGGAGAA AGTCGTCCCT GCTTCGCTTT CACTGCCATG
GACGTCTTCT TCGTGGCCGG TTGATGCACG GTCGTCGGCG GGTAATACGT AGGGTGCAAG CGTTGTCCGG AATTACTGGG
CGTAAAGAGC TCGTAGGCGG TTTGTCGCGT CGTCTGTGAA AACTCGAGGC TCAACCTCGA CCATTATGCA TCCCACGTTC
GCAACAGGCC TTAATGACCC GCATTTCTCG AGCATCCGCC AAACAGCGCA GCAGACACTT TTGAGCTCCG AGTTGGAGCT
GCTTGCAGGC GATACGGGCA GACTTGAGTA CTGCAGGGGA GACTGGAATT CCTGGTGTAG CGGTGAAATG CGCAGATATC
AGGAGGAACA CCGGTGGCGA CGAACGTCCG CTATGCCCGT CTGAACTCAT GACGTCCCCT CTGACCTTAA GGACCACATC
GCCACTTTAC GCGTCTATAG TCCTCCTTGT GGCCACCGCT AGGCGGGTCT CTGGGCAGTA ACTGACGCTG AGGAGCGAAA
GCGTGGGTAG CGAACAGGAT TAGATACCCT GGTAGTCCAC GCCGTAAACG GTGGGCGCTA TCCGCCCAGA GACCCGTCAT
TGACTGCGAC TCCTCGCTTT CGCACCCATC GCTTGTCCTA ATCTATGGGA CCATCAGGTG CGGCATTTGC CACCCGCGAT
GGTGTGGGTT TCCTTCCACG GGATCCGTGC CGTAGCTAAC GCATTAAGCG CCCCGCCTGG GGAGTACGGC CGCAAGGCTA
AAACTCAAAG GAATTGACGG CCACACCCAA AGGAAGGTGC CCTAGGCACG GCATCGATTG CGTAATTCGC GGGGCGGACC
CCTCATGCCG GCGTTCCGAT TTTGAGTTTC CTTAACTGCC GGGCCCGCAC AAGCGGCGGA GCATGTGGAT TAATTCGATG
CAACGCGAAG AACCTTACCT GGGTTTGACA TATACCGGAA AGCTGCAGAG ATGTGGCCCC CCCGGGCGTG TTCGCCGCCT
CGTACACCTA ATTAAGCTAC GTTGCGCTTC TTGGAATGGA CCCAAACTGT ATATGGCCTT TCGACGTCTC TACACCGGGG
CCTTGTGGTC GGTATACAGG TGGTGCATGG CTGTCGTCAG CTCGTGTCGT GAGATGTTGG GTTAAGTCCC GCAACGAGCG
CAACCCTTGT CTTATGTTGC GGAACACCAG CCATATGTCC ACCACGTACC GACAGCAGTC GAGCACAGCA CTCTACAACC
CAATTCAGGG CGTTGCTCGC GTTGGGAACA GAATACAACG CAGCACGTAA TGGTGGGGAC TCGTAAGAGA CTGCCGGGGT
CAACTCGGAG GAAGGTGGGG ACGACGTCAA GTCATCATGC CCCTTATGTC CAGGGCTTCA GTCGTGCATT ACCACCCCTG
AGCATTCTCT GACGGCCCCA GTTGAGCCTC CTTCCACCCC TGCTGCAGTT CAGTAGTACG GGGAATACAG GTCCCGAAGT
CACATGCTAC AATGGCCGGT ACAGAGGGCT GCGATACCGT GAGGTGGAGC GAATCCCTTA AAGCCGGTCT CAGTTCGGAT
CGGGGTCTGC AACTCGACCC GTGTACGATG TTACCGGCCA TGTCTCCCGA CGCTATGGCA CTCCACCTCG CTTAGGGAAT
TTCGGCCAGA GTCAAGCCTA GCCCCAGACG TTGAGCTGGG CGTGAAGTCG GAGTCGCTAG TAATCGCAGA TCAGCAACGC
TGCGGTGAAT ACGTTCCCGG GCCTTGTACA CACCGCCCGT CACGTCATGA AAGTCGGTAA GCACTTCAGC CTCAGCGATC
ATTAGCGTCT AGTCGTTGCG ACGCCACTTA TGCAAGGGCC CGGAACATGT GTGGCGGGCA GTGCAGTACT TTCAGCCATT
CACCCGAAGC CGGTGGCCTA ACCCCTCGTG GGAGGGAGCC GTCGAAGGTG GGATCGGCGA TTGGGACGAA GTCGTAACAA
GGTAGCCGTA CCGGAAGGTG GTGGGCTTCG GCCACCGGAT TGGGGAGCAC CCTCCCTCGG CAGCTTCCAC CCTAGCCGCT
AACCCTGCTT CAGCATTGTT CCATCGGCAT GGCCTTCCAC CGGCTGGATC ACCTCCTTTC TGCCGACCTA GTGGAGGAAA
GA
SEQ ID NO: 13
ACGTGGCGGC ATGCCTTACA CATGCAAGTC GAACGGCAGC GCGGACTTCG GTCTGGCGGC GAGTGGCGAA CGGGTGAGTA
ATACATCGGA ACGTACCCTG TGCACCGCCG TACGGAATGT GTACGTTCAG CTTGCCGTCG CGCCTGAAGC CAGACCGCCG
CTCACCGCTT GCCCACTCAT TATGTAGCCT TGCATGGGAC TTGTGGGGGA TAACTAGTCG AAAGATTAGC TAATACCGCA
TACGACCTGA GGGTGAAAGT GGGGGACCGC AAGGCCTCAC GCAGCAGGAG CGGCCGATGT AACACCCCCT ATTGATCAGC
TTTCTAATCG ATTATGGCGT ATGCTGGACT CCCACTTTCA CCCCCTGGCG TTCCGGAGTG CGTCGTCCTC GCCGGCTACA
CTGATTAGCT AGTTGGTGGG GTAAAGGCCC ACCAAGGCGA CGATCAGTAG CTGGTCTGAG AGGACGATCA GCCACACTGG
GACTGAGACA CGGCCCAGAC GACTAATCGA TCAACCACCC CATTTCCGGG TGGTTCCGCT GCTAGTCATC GACCAGACTC
TCCTGCTAGT CGGTGTGACC CTGACTCTGT GCCGGGTCTG TCCTACGGGA GGCAGCAGTG GGGAATTTTG GACAATGGGG
GCAACCCTGA TCCAGCAATG CCGCGTGTGT GAAGAAGGCC TTCGGGTTGT AAAGCACTTT AGGATGCCCT CCGTCGTCAC
CCCTTAAAAC CTGTTACCCC CGTTGGGACT AGGTCGTTAC GGCGCACACA CTTCTTCCGG AAGCCCAACA TTTCGTGAAA
TGTCCGGAAA GAAATCGCGC TGGTTAATAC CTGCGTGATG ACGGTACCGG AAGAATAAGC ACCGGCTAAC TACGTGCCAG
CAGCCGCGGT AATACGTAGG ACAGGCCTTT CTTTAGCGCG ACCAATTATG GACGCACTAC TGCCATGGCC TTCTTATTCG
TGGCCGATTG ATGCACGGTC GTCGGCGCCA TTATGCATCC GTGCGAGCGT TAATCGGAAT TACTGGGCGT AAAGCGTGCG
CAGGCGGTTT TGTAAGACAG GCGTGAAATC CCCGGGCTTA ACCTGGGAAT TGCGCTTGTG CACGCTCGCA ATTAGCCTTA
ATGACCCGCA TTTCGCACGC GTCCGCCAAA ACATTCTGTC CGCACTTTAG GGGCCCGAAT TGGACCCTTA ACGCGAACAC
ACTGCAAGGC TAGAGTGCGT CAGAGGGGGG TAGAATTCCA CGTGTAGCAG TGAAATGCGT AGAGATGTGG AGGAATACCG
ATGGCGAAGG CGAGCCCCCT TGACGTTCCG ATCTCACGCA GTCTCCCCCC ATCTTAAGGT GCACATCGTC ACTTTACGCA
TCTCTACACC TCCTTATGGC TACCGCTTCC GCTCGGGGGA GGACCTTGAC TGACGCTCAT GCACGAAAGC GTGGGGAGCA
AACAGGATTA GATACCCTGG TAGTCCACGC CCTAAACGAT GTCAACTAGT TGTTGGGATT CCTGGAACTG ACTGCGAGTA
CGTGCTTTCG CACCCCTCGT TTGTCCTAAT CTATGGGACC ATCAGGTGCG GGATTTGCTA CAGTTGATCA ACAACCCTAA
CATTTTCTCA GTAACGTAGC TAACGCGTGA AGTTGACCGC CTGGGGAGTA CGGCTGCAAG ATTAAAACTC AAAGGAATTG
ACGGGGACCC GCACAAGCGG GTAAAAGAGT CATTGCATCG ATTGCGCACT TCAACTGGCG GACCCCTCAT GCCGACGTTC
TAATTTTGAG TTTCCTTAAC TGCCCCTGGG CGTGTTCGCC TGGATGATGT GGATTAATTC GATGCAACGC GAAAAACCTT
ACCTACCCTT GACATGCCCT AACGAAGCAG AGATGCATTA GTGCCCGCAA AGGGAAAGTG ACCTACTACA CCTAATTAAG
CTACGTTGCG CTTTTTGGAA TGGATGGGAA CTGTACGGGA TTGCTTCGTC TCTACGTAAT CACGGGCGTT TCCCTTTCAC
GGACACAGGT GCTGCATGGC TGTCGTCAGC TCGTGTCGTG AGATGTTGGG TTAAGTCCCG CAACGAGCGC AACCCTTGTC
TCTAGTTGCC TACGCAAGAG CCTGTGTCCA CGACGTACCG ACAGCAGTCG AGCACAGCAC TCTACAACCC AATTCAGGGC
GTTGCTCGCG TTGGGAACAG AGATCAACGG ATGCGTTCTC CACTCTAGAG AGACTGCCGG TGACAAACCG GAGGAAGGTG
GGGATGACGT CAAGTCCTCA TGGCCCTTAT GGGTAGGGCT TCACACGTCA TACAATGGTG GTGAGATCTC TCTGACGGCC
ACTGTTTGGC CTCCTTCCAC CCCTACTGCA GTTCAGGAGT ACCGGGAATA CCCATCCCGA AGTGTGCAGT ATGTTACCAC
CGTACAGAGG GTTGCCAACC CGCGAGGGGG AGCTAATCCC AGAAAACGCA TCGTAGTCCG GATCGTAGTC TGCAACTCGA
CTACGTGAAG CTGGAATCGC GCATGTCTCC CAACGGTTGG GCGCTCCCCC TCGATTAGGG TCTTTTGCGT AGCATCAGGC
CTAGCATCAG ACGTTGAGCT GATGCACTTC GACCTTAGCG TAGTAATCGC GGATCAGCAT GCCGCGGTGA ATACGTTCCC
GGGTCTTGTA CACACCGCCC GTCACACCAT GGGAGTGGGT TTTGCCAGAA GTAGTTAGCC ATCATTAGCG CCTAGTCGTA
CGGCGCCACT TATGCAAGGG CCCAGAACAT GTGTGGCGGG CAGTGTGGTA CCCTCACCCA AAACGGTCTT CATCAATCGG
TAACCGCAAG GAGGGCGATT ACCACGGCAG GGTTCATGAC TGGGGTGAAG TCGTAACAAG GTATTGGCGT TCCTCCCGCT
AATGGTGCCG TCCCAAGTAC TGACCCCACT TCAGCATTGT TCCA
SEQ ID NO: 14
MASIEDILEL EALEKDIFRG AVHPSVLKRT FGGQVAGQSL VSAVRTVDER FEVHSLHGYF LRPGNPTEPT VYLVDRIRDG
RSFCTRRVTG IQDGKAIFTM SASFHSQDEG IEHQDTMPSV PEPEELVDAQ TVEEMAATDL YREWKEWDVR IVPAGCTGKT
PGIAAKQRVW MRYRNKLPDD QVFHICTLAY LSDMTLLGAS KVPHPGVVTQ TASLDHAMWF LRPFRADEWL LYDQTSPSAG
FGRALTQGRM FDRKGTMVAA VVQEGLTRIQ RDQDQRDIET GNMA

In some embodiments, the cell comprises a plasmid that contains one or more exogenous nucleic acid sequences encoding enzymes or proteins that include but are not limited to one or more of the following: an acyl carrier protein, a TE, a FAR, a FadR, a FAD, a fatty aldehyde reductase, and an antibiotic resistance enabling protein; wherein the plasmid is at least 20, 30, 40, 50, 60, 70, 80, 90, 95, 96, 97, 98, or 99% homologous to SEQ ID NO:1, SEQ ID NO:2, SEQ ID NO:3, or SEQ ID NO:4. In some embodiments, the cell of composition comprising a cell comprise at least one exogenous nucleic acid that encodes a FAR or a functional fragment of a FAR derived from one of the following organisms: Arabidopsis thaliana, Arabidopsis lyrata, Vitis vinifera, Populus trichocarpa, Artermisia annua, Ricinus communis, Simmondsia chineis, Oryza sativa japonica, Hevea brasiliensis, Hordeum vulgare, Triticum aestivum, Sorghum bicolor, Zea mays, and Selaginella moelllendorff.

In one embodiment, the exogenous gene encodes a FAR. In some cases, the FAR encoded by the exogenous gene catalyzes the reduction of a 20 to 30-carbon fatty acyl-CoA to a corresponding primary alcohol. In some cases, the FAR encoded by the exogenous gene catalyzes the reduction of an 8 to 18-carbon fatty acyl-CoA to a corresponding primary alcohol. In some cases, the FAR encoded by the exogenous gene catalyzes the reduction of a 10 to 14-carbon fatty acyl-CoA to a corresponding primary alcohol. In one embodiment, the FAR encoded by the exogenous gene catalyzes the reduction of a 12-carbon fatty acyl-CoA to dodecanol.

In some embodiments, the invention is related to the method of modifying the population of fatty acids to produce molecules of desired length by incorporation of different thioesterases. In some embodiments this produces shorter chain fatty acids. In some embodiments the population of fatty acids is modified to add an additional carboxylic acid (—COOH) to fatty acid chains using enzymes including but not limited to cytochrome P450 enzyme, and processes. In some embodiments the population of fatty acids is modified to add an hydroxyl group (—OH) to fatty acid chains using enzymes (hydroxylases) and processes. In some embodiments the population of fatty acids can be desaturated with incorporation of one or more double bonds, using enzymes (desaturases) and processes.

### Dicarboxylic Acids

In some embodiments, the cell comprises a plasmid that contains one or more exogenous nucleic acid sequences encoding enzymes or proteins that include but are not limited to one or more of the following: a cytochrome P450 enzyme (e.g., CYP52) and accompanying NADPH cytochrome P450 reductase for generating dicarboxylic acids and an antibiotic resistance enabling protein; wherein the plasmid is at least 20, 30, 40, 50, 60, 70, 80, 90, 95, 96, 97, 98, or 99% homologous to sequences GenBank: AA073953.1, GenBank: AY230500.1 GenBank: AA073958.1, GenBank: AA073959.1, or GenBank: AA073952.1.

In some embodiments, the cell of composition comprising a cell comprise at least one exogenous nucleic acid that encodes a cytochrome P450 enzymes (e.g., CYP52) and accompanying NADPH cytochrome P450 reductase or a functional fragment of a cytochrome P450 enzymes (e.g., CYP52) and accompanying NADPH cytochrome P450 reductase. In some embodiments, the cell of composition comprising a cell comprise at least one exogenous nucleic acid that encodes a cytochrome P450 enzymes (e.g., CYP52) and accompanying NADPH cytochrome P450 reductase or a functional fragment of a cytochrome P450 enzymes (e.g., CYP52) and accompanying NADPH cytochrome P450 reductase derived from one of the following organisms: Candida tropicalis, Pyrococcus furiosus.

In one embodiment, the exogenous gene encodes a cytochrome P450 enzyme (e.g., CYP52) and accompanying NADPH cytochrome P450 reductase. In some cases, the cytochrome P450 enzyme (e.g., CYP52) and accompanying NADPH cytochrome P450 reductase encoded by the exogenous gene catalyzes the addition of a carboxylic acid to an 20 to 30-carbon chain fatty acid, where the fatty acid may be free or in an ester bond or bound to a co-factor including but not limited to ACP or CoA. In some cases, the cytochrome P450 enzyme (e.g., CYP52) and accompanying NADPH cytochrome P450 reductase encoded by the exogenous gene catalyzes the addition of a carboxylic acid to an 8 to 18-carbon chain fatty acid, where the fatty acid may be free or in an ester bond or bound to a co-factor including but not limited to ACP or CoA. In some cases, the cytochrome P450 enzyme (e.g., CYP52) and accompanying NADPH cytochrome P450 reductase encoded by the exogenous gene catalyzes the addition of a carboxylic acid to a 10 to 14-carbon chain fatty acid, where the fatty acid may be free or in an ester bond or bound to a co-factor including but not limited to ACP or CoA. In one embodiment, the cytochrome P450 enzyme (e.g., CYP52) and accompanying NADPH cytochrome P450 reductase encoded by the exogenous gene catalyzes the addition of a carboxylic acid to an 8-carbon chain fatty acid, where the fatty acid may be free or in an ester bond or bound to a co-factor including but not limited to ACP or CoA.

### Desaturation

In some embodiments, the cell comprises a plasmid that contains one or more exogenous nucleic acid sequences encoding enzymes or proteins that include but are not limited to one or more of the following: a desaturase for introducing double bonds and an antibiotic resistance enabling protein.

In some embodiments, the cell of composition comprising a cell comprise at least one exogenous nucleic acid that encodes a desaturase for introducing double bonds or a functional fragment of a desaturase for introducing double bonds. In some embodiments, the cell of composition comprising a cell comprise at least one exogenous nucleic acid that encodes a desaturase for introducing double bonds or a functional fragment of a desaturase for introducing double bonds derived from one of the following organisms: Arabidopsis thaliana, Arabidopsis lyrata, Vitis vinifera, Populus trichocarpa, Artermisia annua, Ricinus communis, Simmondsia chineis, Oryza sativa japonica, Hevea brasiliensis, Hordeum vulgare, Triticum aestivum, Sorghum bicolor, Zea mays, and Selaginella moelllendorff.

In one embodiment, the exogenous gene encodes a desaturase for introducing double bonds. In some cases, the a desaturase for introducing double bonds encoded by the exogenous gene catalyzes the introduction of one or more double bonds of a 20 to 30-carbon chain hydrocarbon or fatty acid, where the fatty acid may be free or in an ester bond or bound to a co-factor including but not limited to ACP or CoA. In some cases, the a desaturase for introducing double bonds encoded by the exogenous gene catalyzes the introduction of one or more double bonds of an 8 to 18-carbon chain hydrocarbon or fatty acid, where the fatty acid may be free or in an ester bond or bound to a co-factor including but not limited to ACP or CoA. In some cases, the a desaturase for introducing double bonds encoded by the exogenous gene catalyzes the introduction of one or more double bonds of a 10 to 14-carbon chain hydrocarbon or fatty acid, where the fatty acid may be free or in an ester bond or bound to a co-factor including but not limited to ACP or CoA. In one embodiment, the a desaturase for introducing double bonds encoded by the exogenous gene catalyzes the introduction of one or more double bonds of a 12-carbon chain hydrocarbon or fatty acid, where the fatty acid may be free or in an ester bond or bound to a co-factor including but not limited to ACP or CoA.

### Hydroxylation

In some embodiments, the cell comprises a plasmid that contains one or more exogenous nucleic acid sequences encoding enzymes or proteins that include but are not limited to one or more of the following: a cytochrome P450-dependent fatty acid hydroxylase for introducing a hydroxyl group and an antibiotic resistance enabling protein; wherein the plasmid is at least 20, 30, 40, 50, 60, 70, 80, 90, 95, 96, 97, 98, or 99% homologous to Genbank Accession ACF37070, ABE12594, AAC49010.1, AAF03100.1, ABQ01458.1, CAK37451.1, Q029828.1, or genes V94A1_VICSA, V94A2_VISCA, CYP94B1, CYP94B2, CYP94B3, BYP94C1, CYP94A1, CYP94A5, CYP78A1, CYP86A1, CYP86A2, CYP86A8, CYP92B1, CYP81B1, or CYP709C1.

In some embodiments, the cell of composition comprising a cell comprise at least one exogenous nucleic acid that encodes a cytochrome P450-dependent fatty acid hydroxylase for introducing a hydroxyl group or a functional fragment of a cytochrome P450-dependent fatty acid hydroxylase for introducing a hydroxyl group. In some embodiments, the cell of composition comprising a cell comprise at least one exogenous nucleic acid that encodes a cytochrome P450-dependent fatty acid hydroxylase for introducing a hydroxyl group or a functional fragment of a cytochrome P450-dependent fatty acid hydroxylase for introducing a hydroxyl group derived from one of the following organisms: Claviceps purpurea (fungus), Ricinus communis, Lactuca sativa, Physaria lindheimeri, Aspergillus niger, Human P450, Vicia sativa, S. cerevisiae, Arabidopsis thaliana, Nicotiana, Pisum sativum, V. sativa, Arabidopsis thaliana, Zea mays, Petunia hybrida, H. tuberosus.

In one embodiment, the exogenous gene encodes a cytochrome P450-dependent fatty acid hydroxylase that introduces a hydroxyl group. In some cases, the cytochrome P450-dependent fatty acid hydroxylase encoded by the exogenous gene catalyzes the addition of the hydroxyl group to a 20 to 30-carbon fatty acid, where the fatty acid may be free or in an ester bond or bound to a co-factor including but not limited to ACP or CoA. In some cases, the cytochrome P450-dependent fatty acid hydroxylase encoded by the exogenous gene catalyzes the addition of the hydroxyl group to an 8 to 18-carbon fatty acid, where the fatty acid may be free or in an ester bond or bound to a co-factor including but not limited to ACP or CoA. In some cases, the cytochrome P450-dependent fatty acid hydroxylase encoded by the exogenous gene catalyzes addition of the hydroxyl group to a 10 to 14-carbon fatty acid, where the fatty acid may be free or in an ester bond or bound to a co-factor including but not limited to ACP or CoA. In one embodiment, the cytochrome P450-dependent fatty acid hydroxylase encoded by the exogenous gene catalyzes addition of the hydroxyl group to a 12-carbon fatty acid, where the fatty acid may be free or in an ester bond or bound to a co-factor including but not limited to ACP or CoA.

In one embodiment, the exogenous gene encodes a FadR. In some cases, the reductase encoded by the exogenous gene catalyzes the reduction of an 8 to 18-carbon fatty acyl-CoA to a corresponding aldehyde. In one embodiment, the reductase encoded by the exogenous gene catalyzes the reduction of a 12-carbon fatty acyl-CoA to dodecanal.

In some embodiments, the invention relates to a bacterial cell or a compositions comprising at least one bacterial cell that comprises at least a first and a second exogenous nucleic acid sequence, wherein the first nucleic acid sequence encodes a FadR or a functional fragment of a FadR and the second exogenous nucleic acid sequence encodes a fatty acyl-CoA ligase or a functional fragment thereof. In some embodiments, the functional fragments of the enzymes encoded by the one or more exogenous nucleic acid sequences are at least 40, 45, 50, 55, 60, 65, 70, 75, 80, 85, 90, 91, 92, 93, 94, 95, 96, 97, 98, or 99% homologous to the nucleic acid sequences that encode the full-length amino acid sequence upon which the functional fragment is based. Any enzyme disclosed in this application and part of the invention may be replaced with a functional fragment. Any composition or cell disclosed in the application may be used in any disclosed method of this application.

In some embodiments, the genetic constructs contain sequences directing transcription and translation of the relevant exogenous (either heterologous or homologous) gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. In some cells the exogenous gene is coding sequence and is in operable linkage with a promoter, and in some embodiments the promoter is derived from a gene endogenous to a species of the genus Rhodococcus. Initiation control regions or promoters, which are useful to drive expression of the instant ORFs in the desired host cell are numerous and familiar to those skilled in the art. Virtually any promoter capable of driving these genes is suitable for the present invention including but not limited to CYC1, HIS3, GAL1, GAL10, ADH1, PGK, PHO5, GAPDH, ADC1, TRP1, URA3, LEU2, ENO; and lac, ara, tet, trp, IP_{L}, IP_{R}, T7, tac, and trc as well as the amy, apr, npr promoters and various phage promoters useful for expression in the lipid-producing bacteria of the present invention. In other embodiments the promoter is upregulated in response to reduction or elimination of a cofactor in the culture media of the cell, such as at least a 3-fold upregulation as determined by transcript abundance in a cell when the cell is exposed to extracellular environment changes from containing at least 10 mM or 5 mM cofactor to containing no cofactor.

Termination control regions may also be derived from various genes native to the preferred hosts. Optionally, the genetic constructs of the present invention do not comprise a termination control region.

In some embodiments, the bacterial cell or the composition comprising the bacterial cell comprises at least one genetic construct, which comprises one or more coding sequences. In some embodiments, the invention relates to the bacterial cell or the composition comprising at least one bacterial cell wherein the at least one cell comprises two or more genetic constructs, each comprising one or more coding sequences. In some embodiments, the coding sequences of the claimed invention encode at least one protein that modifies or accelerates lipid production in the host cell. In some embodiments the coding sequence encodes at least one protein that alters the levels of individual lipids or hydrocarbons produced by the cell as compared to the same cell not modified by an exogenous nucleic acid sequence. In some embodiments, the coding sequence may encode at least one protein that alters the amount of one specific lipid or hydrocarbon molecule of the cell as compared to the same cell not modified by the nucleic acid. For example, in one embodiment, the one or more exogenous nucleic acid sequence encodes at least one lipid pathway enzyme that causes an increase in the ratio of C14:C16:C18 lipids or hydrocarbons produced or secreted by the cell as compared to the C14:C16:C18 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C14:C16:C18 lipids or hydrocarbons produced or secreted by the cell as compared to the C14:C16:C18 lipids or hydrocarbons produced or secreted by the same cell not transformed with the nucleic acid sequence that encodes the lipid pathway enzyme. In some embodiments, the one or more cells comprising one or more exogenous nucleic acid sequences produces at least 5% more C8 hydrocarbon as compared to the same one or more cells not transformed or modified with the one or more exogenous nucleic acid sequences.

In some embodiments, the one or more cells comprising one or more exogenous nucleic acid sequences produces at least 50% more C8 hydrocarbon as compared to the same one or more cells not transformed or modified with the one or more exogenous nucleic acid sequences.

In some embodiments, the one or more cells comprising one or more exogenous nucleic acid sequences produces at least 5% more C9 hydrocarbon as compared to the same one or more cells not transformed or modified with the one or more exogenous nucleic acid sequences. In some embodiments, the one or more cells comprising one or more exogenous nucleic acid sequences produces at least 50% more C9 hydrocarbon as compared to the same one or more cells not transformed or modified with the one or more exogenous nucleic acid sequences.

In some embodiments, the one or more cells comprising one or more exogenous nucleic acid sequences produces at least 5% more C10 hydrocarbon as compared to the same one or more cells not transformed or modified with the one or more exogenous nucleic acid sequences.

In some embodiments, the one or more cells comprising one or more exogenous nucleic acid sequences produces at least 50% more C10 hydrocarbon as compared to the same one or more cells not transformed or modified with the one or more exogenous nucleic acid sequences.

In some embodiments, the one or more cells comprising one or more exogenous nucleic acid sequences produces at least 5% more C11 hydrocarbon as compared to the same one or more cells not transformed or modified with the one or more exogenous nucleic acid sequences. In some embodiments, the one or more cells comprising one or more exogenous nucleic acid sequences produces at least 50% more C11 hydrocarbon as compared to the same one or more cells not transformed or modified with the one or more exogenous nucleic acid sequences.

In some embodiments, the one or more cells comprising one or more exogenous nucleic acid sequences produces at least 5% more C12 hydrocarbon as compared to the same one or more cells not transformed or modified with the one or more exogenous nucleic acid sequences.

In some embodiments, the one or more cells comprising one or more exogenous nucleic acid sequences produces at least 50% more C12 hydrocarbon as compared to the same one or more cells not transformed or modified with the one or more exogenous nucleic acid sequences.

In some embodiments, the one or more cells comprising one or more exogenous nucleic acid sequences produces at least 5% more C13 hydrocarbon as compared to the same one or more cells not transformed or modified with the one or more exogenous nucleic acid sequences.

In some embodiments, the one or more cells comprising one or more exogenous nucleic acid sequences produces at least 50% more C13 hydrocarbon as compared to the same one or more cells not transformed or modified with the one or more exogenous nucleic acid sequences.

In some embodiments, the one or more cells comprising one or more exogenous nucleic acid sequences produces at least 5% more C14 hydrocarbon as compared to the same one or more cells not transformed or modified with the one or more exogenous nucleic acid sequences.

In some embodiments, the one or more cells comprising one or more exogenous nucleic acid sequences produces at least 50% more C14 hydrocarbon as compared to the same one or more cells not transformed or modified with the one or more exogenous nucleic acid sequences.

In some embodiments, the one or more cells comprising one or more exogenous nucleic acid sequences produces at least 5% more C15 hydrocarbon as compared to the same one or more cells not transformed or modified with the one or more exogenous nucleic acid sequences.

In some embodiments, the one or more cells comprising one or more exogenous nucleic acid sequences produces at least 50% more C15 hydrocarbon as compared to the same one or more cells not transformed or modified with the one or more exogenous nucleic acid sequences.

In some embodiments, the one or more cells comprising one or more exogenous nucleic acid sequences produces at least 5% more C16 hydrocarbon as compared to the same one or more cells not transformed or modified with the one or more exogenous nucleic acid sequences.

In some embodiments, the one or more cells comprising one or more exogenous nucleic acid sequences produces at least 50% more C16 hydrocarbon as compared to the same one or more cells not transformed or modified with the one or more exogenous nucleic acid sequences.

In some embodiments, the one or more cells comprising one or more exogenous nucleic acid sequences produces at least 5% more C17 hydrocarbon as compared to the same one or more cells not transformed or modified with the one or more exogenous nucleic acid sequences.

In some embodiments, the one or more cells comprising one or more exogenous nucleic acid sequences produces at least 50% more C17 hydrocarbon as compared to the same one or more cells not transformed or modified with the one or more exogenous nucleic acid sequences.

In some embodiments, the one or more cells comprising one or more exogenous nucleic acid sequences produces at least 5% more C18 hydrocarbon as compared to the same one or more cells not transformed or modified with the one or more exogenous nucleic acid sequences.

In some embodiments, the one or more cells comprising one or more exogenous nucleic acid sequences produces at least 50% more C18 hydrocarbon as compared to the same one or more cells not transformed or modified with the one or more exogenous nucleic acid sequences.

In some embodiments, the one or more exogenous nucleic acid sequence encodes at least one lipid pathway enzyme that causes an increase in the ratio of C12:C14:C16 lipids or hydrocarbons produced or secreted by the cell as compared to the C12:C14:C16 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C12:C14:C16 lipids or hydrocarbons produced or secreted by the cell as compared to the C12:C14:C16 lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes an increase in the ratio of C13:C15:C17 lipids or hydrocarbons produced or secreted by the cell as compared to the C13:C15:C17 lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of C13:C15:C17 lipids or hydrocarbons produced or secreted by the cell as compared to the C13:C15:C17 lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes an increase in the ratio of odd-numbered lipids or hydrocarbons produced or secreted by the cell as compared to the odd-numbered lipids or hydrocarbons produced or secreted by the same cell not transformed with the nucleic acid sequence that encodes the lipid pathway enzyme. In some embodiments, the one or more exogenous nucleic acid sequence encodes at least one lipid pathway enzyme that causes a decrease in the ratio of odd-numbered lipids or hydrocarbons produced or secreted by the cell as compared to the odd-numbered lipids or hydrocarbons produced or secreted by the same cell not transformed with the nucleic acid sequence that encodes the lipid pathway enzyme. In one embodiment, the one or more exogenous nucleic acid sequence encodes at least one lipid pathway enzyme that causes a decrease in the ratio of even:odd lipids or hydrocarbons produced or secreted by the cell as compared to the ratio of even:odd lipids or hydrocarbons produced or secreted by the same cell not transformed with the nucleic acid sequence that encodes the one or more lipid pathway enzymes. In one embodiment, the one or more exogenous nucleic acid sequence encodes at least one lipid pathway enzyme that causes a increase in the ratio of even:odd lipids or hydrocarbons produced or secreted by the cell as compared to the ratio of even:odd lipids or hydrocarbons produced or secreted by the same cell not transformed with the nucleic acid sequence that encodes the one or more lipid pathway enzymes.

In some embodiments, the one or more exogenous nucleic acid sequence encodes at least one lipid pathway enzyme that causes at least a 5% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 85% increase in the ratio of C12:C14:C16 lipids or hydrocarbons produced or secreted by the cell as compared to the C12:C14:C16 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 5% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments, the one or more exogenous nucleic acid sequence encodes at least one lipid pathway enzyme that causes at least a 85% increase in the ratio of C13:C15:C17 lipids or hydrocarbons produced or secreted by the cell as compared to the C13:C15:C17 lipids or hydrocarbons produced or secreted by the same cell not transformed or modified with the nucleic acid sequence that encodes the lipid pathway enzyme.

In some embodiments the exogenous gene or genes codes for enzymes or proteins including but not limited to one or more of the following: an acyl carrier protein, a TE, a FAR, a FadR, a FAD, a fatty aldehyde reductase, and an antibiotic resistance enabling protein. In some embodiments, the coding sequence comprises an exogenous nucleic acid sequence that encodes a TE that catalyzes hydrolysis of one or more fatty acyl-ACP substrates with chain lengths ranging over C8, C9, C10, C11, C12, C13, C14, C15, C16, C17, or C18. In some embodiments, the cell comprises a plasmid that contains one or more exogenous nucleic acid sequences that encode an amino acid sequence for an enzyme or protein such as but not limited to one or more of the following: an acyl carrier protein, a TE, a FAR, a FadR, a FAD, a fatty aldehyde reductase, and an antibiotic resistance enabling protein. In some embodiments, the one or more exogenous nucleic acid sequences comprise SEQ ID NO:5 or a functional fragment thereof that is at least 70%, 75%, 80%, 85%, 90%, 91%, 92%, 93%, 94%, 95%, 96%, 97%, 98%, or 99% homologous to SEQ ID NO:5. In some embodiments, the one or more exogenous nucleic acid sequences comprise SEQ ID NO:6 or a functional fragment thereof that is at least 70%, 75%, 80%, 85%, 90%, 91%, 92%, 93%, 94%, 95%, 96%, 97%, 98%, or 99% homologous to SEQ ID NO:6. In some embodiments, the one or more exogenous nucleic acid sequences comprise SEQ ID NO:7 or a functional fragment thereof that is at least 70%, 75%, 80%, 85%, 90%, 91%, 92%, 93%, 94%, 95%, 96%, 97%, 98%, or 99% homologous to SEQ ID NO:7. In some embodiments, the one or more exogenous nucleic acid sequences comprise SEQ ID NO:8 or a functional fragment thereof that is at least 70%, 75%, 80%, 85%, 90%, 91%, 92%, 93%, 94%, 95%, 96%, 97%, 98%, or 99% homologous to SEQ ID NO:8. In some embodiments, the one or more exogenous nucleic acid sequences comprise SEQ ID NO:9 or a functional fragment thereof that is at least 70%, 75%, 80%, 85%, 90%, 91%, 92%, 93%, 94%, 95%, 96%, 97%, 98%, or 99% homologous to SEQ ID NO:9.

In further embodiments, at least one coding sequence of the at least one exogenous nucleic acid sequence encodes a lipid pathway enzyme. In some embodiments, the at least one coding sequence of the at least one exogenous nucleic acid sequence encodes a lipid modification enzyme. In some embodiments, the composition or cell comprises a nucleic acid that encodes at least one fatty acid decarbonylase, at least one fatty acid reductase, a thioesterase, or any combination of any one more full-length lipid pathway enzymes or functional fragments thereof. In some embodiments the one or more exogenous nucleic acid sequences are integrated into the genome of the cell. In some embodiments, the one or more exogenous nucleic acid sequences are on an episomal plasmid within the transformed host cell.

### Methods of Isolation and Purification

Following the methods of the present invention microorganisms are grown and maintained for the production of lipids in a medium containing crude glycerol and/or glycerol and/or methanol. In some embodiments, the invention relates to methods of cultivating oleaginous cells for the large scale production of oil and/or fuel. In some embodiments, the invention relates to methods of cultivating oleaginous cells for the large scale production of biodiesel. In some embodiments, the invention relates to methods of cultivating oleaginous cells in bioreactors 50,000 liters or greater in volume, which are conventionally constructed out of low cost, sturdy, and opaque materials such as steel or reinforced concrete or earthworks. The size, depth, and construction of such bioreactors dictate that the cells will be grown in near or total darkness. In some embodiments, the oleaginous microorganisms are cultured for the synthesis of lipids in accordance with the methods of the present invention in a medium containing a low cost or waste energy and carbon source, such as but not limited to crude glycerol and/or glycerol and/or methanol, as the primary or sole energy and carbon source.

To give an illustration, a bioreactor containing nutrient medium is inoculated with of oleaginous bacterial cells; generally there will follow a lag phase prior to the cells beginning to double. After the lag phase, the cell doubling time decreases and the culture goes into the logarithmic phase. The logarithmic phase is eventually followed by an increase of the doubling time that, while not intending to be limited by theory, is thought to result from either a depletion of nutrients including nitrogen sources, or a rise in the concentration of inhibitory chemicals, or quorum sensing by the microbes. The growth slows down and then ceases when the culture goes into the stationary phase. In order to harvest cell mass with high lipid content, the culture is generally harvested late in the logarithmic phase or in the stationary phase. In some embodiments, the cells are harvested in logarithmic phase. In some embodiments, the cells are harvested in stationary phase. The accumulation of lipid can generally be triggered by the depletion of the nitrogen source or another key nutrient excepting the carbon or the energy source (e.g. crude glycerol). This signals the cells to store lipids produced from the excess carbon and energy sources. Optimization of lipid production and the targeting of specific lipid distributions can be achieved by control of bioreactor conditions and/or nutrient levels and/or through genetic modifications of the cells. In some embodiments the lipid production and distribution of lipid molecules produced is optimized through one or more of the following: control of bioreactor conditions, control of nutrient levels, genetic modifications of the cells.

The synthesis of lipids by the microbes disclosed in the present invention can happen during the logarithmic phase and afterwards during the stationary phase when cell doubling has stopped provided there is an ample supply of carbon and energy sources,

In some embodiments, microorganisms grown using conditions described herein and known in the art comprise at least 20% lipid content by weight. In some embodiments, for growth on crude glycerol and/or glycerol and/or methanol, the microorganisms of the present invention comprise at least about 10, 15, 20, 25, 30, 35, or 40% by weight of lipids, at least about 50% by weight, or at least about 60% by weight of lipids. Improved lipid yield and/or lower production costs can be achieved by controlling process parameters. In certain embodiments, a bacterium is grown in a nutrient media and/or gas mix having a nitrogen, oxygen, phosphorous, or sulfur limitation, while a carbon and energy source such as crude glycerol and/or glycerol and/or methanol is provided in excess. Lipid yield is generally higher in microbial cultures grown with a nitrogen limitation versus microbial cultures grown without nitrogen limitation. In certain embodiments, lipid yield rises by at least: 10%, 50%, 100%, 200%, 500%, or 1000%. The microbial growth can occur with nutrient limitation for a part or for all of the fermentation run. Feeding an excess of energy and carbon source to a population of oleaginous microbes, but little or no nitrogen, can produce a rise in cellular lipid content. In some embodiments, microbial growth occurs on limited amounts of nitrogen or in the complete absence of nitrogen.

Genes are well known in the art that code for cofactors useful in the present invention, or that are involved in synthesizing such cofactors.

In another embodiment, genes that code for cofactors useful in the present invention, or that are involved in synthesizing such cofactors, are put in oleaginous bacteria, using the constructs and methods such as described above. Lipid yield is improved in another embodiment by growing an oleaginous bacteria with one or more lipid pathway enzyme cofactor(s) added to the culture environment. The lipid yield is generally improved in the presence of a certain concentration of the cofactor(s) compared to lipid yield without supplemental cofactor(s). In some embodiments, the cofactor(s) are delivered to the culture by having a microbe (e.g., bacteria) present in the culture that contains an exogenous gene coding for the cofactor(s) at a concentration sufficient to increase lipid yield as compared to the lipid yield of the microbe in the absence of the cofactor. Cofactor(s) may also be delivered to a culture by having a microbe (e.g., bacteria) present in the culture that contains an exogenous gene that coding for a protein involved in the cofactor synthesis. In some embodiments, any vitamin needed for the proper function of a lipid pathway enzyme including biotin and/or pantothenate is included in the culture environment.

The specific examples of bioreactors, culture conditions, heterotrophic and chemotrophic growth, maintenance, and lipid production methods described herein can be combined in any suitable manner to improve efficiencies of microbial growth and lipid and/or protein production.

In another aspect of the invention, the invention relates to a method of producing a molecule or mixture of molecules in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing a hydrocarbon or mixture of hydrocarbons in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing a lipid or mixture of lipids in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing an alkane or mixture of alkanes in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing an alkene or mixture of alkenes in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing an alkyne or mixture of alkynes in a microorganism population comprising the cell or the composition described herein, wherein the method comprises: culturing a population of microorganisms comprising the cell or the composition described herein in a feedstock comprising crude glycerol and/or glycerol and/or methanol.

In another aspect of the invention, the invention relates to a method of producing an alkyl ester or mixture of alkyl esters in a microorganism population comprising the cell or the composition described herein, wherein the method comprises: culturing a population of microorganisms comprising the cell or the composition described herein in a feedstock comprising crude glycerol and/or glycerol and/or methanol.

In some embodiments, the methods of the claimed invention do not rely on desulfonation to produce and/or secrete one or more hydrocarbons. In some embodiments, an exogenous nucleic acid is introduced into the cells of the claimed invention to silence or disrupt transcription of endogenous genes of the cell that encode enzymes capable of desulfonation of commercial surfactants under conditions and for a time period sufficient for growth of the cell with a feedstock comprising crude glycerol and/or glycerol and/or methanol.

In another aspect of the invention, the invention relates to a method of producing a primary alcohol in a microorganism population comprising the cell or the composition described herein, wherein the method comprises: culturing a population of microorganisms comprising the cell or the composition described herein in a feedstock comprising crude glycerol and/or glycerol and/or methanol. In some embodiments, the bacterial cell comprises a first and second exogenous nucleic acid sequence, wherein the first nucleic acid sequence encodes a FAR or functional fragment thereof and the second exogenous nucleic acid encodes a fatty-acyl-CoA ligase or functional fragment thereof.

In some embodiments, the feedstock does not include linoleic acid.

The following documents are incorporated by reference in their entirety:
Doan TTP, Carlsson A S, Hamberg M, Bulow L, Stymne S, Olsson P, Functional expression of five Arabidopsis fatty acyl-CoA reductase genes in Escherichia coli, J Plant Phys 166(2008):787-96.
Kavanagh K L, Jornvall H, Persson B, Oppermann U, The SDR superfamily: functional and structural diversity within a family of metabolic and regulatory enzymes, Cell Mol Life Sci 65 (2008) 3895-3906.
Labesse G, Vidal-Cros A, Chomilier J, Gaudry M, Mornon J-P, Structural comparisons lead to the definition of a new superfamily of NAD(P)(H)-accepting oxidoreductases: the single-domain reductases/epimerases/dehydrogenases (the ‘RED’ family), Biochem J (1994) 304:95-99.
   PCT Patent Application No. PCT/US2010/001402
   PCT Patent Application No. PCT/US2011/034218

The following examples are provided to describe the invention in greater detail. They are intended to illustrate, not to limit, the invention. Various publications, including patents, published applications, technical articles and scholarly articles are cited throughout the specification. Each of these cited publications is incorporated by reference herein, in its entirety.

### Examples

Bioreactor growth of R. opacus strain (DSM43205).

Initial flask growth of inoculum for bioreactor

First a test tube inoculum R. opacus strain (DSM43205) was grown on Lysogeny broth (LB) for 18-24 hours. The inoculum size introduced to the flask was 5%.

Media used for flask level growth of the microbe was

Media (a):

| | | |
|---|---|---|
| Na2HPO4•12H2O | 9 | g |
| KH2PO4 | 1.5 | g |
| NH4Cl | 1.0 | g |
| MgSO4•7H2O | 0.2 | g |
| Trace Mineral Soln | 1.0 | ml (media d below) |
| Distilled water (DW) | 1000 | ml |

Medium (b)

| | |
|---|---|
| NaHCO3 | 5 g |
| DW | 100 ml |

Medium (c)

| | |
|---|---|
| Ferric Ammonium Citrate | 50 mg |
| CaCl2•2H2O | 100 mg |
| DW | 100 ml |

Media (d)—Trace Mineral Medium

| | | |
|---|---|---|
| ZnSO4•7H2O | 100 | mg |
| MnCl2•4H2O | 30 | mg |
| H3BO3 | 300 | mg |
| CoCl2•6H2O | 200 | mg |
| CuCl2•2H2O | 10 | mg |
| NiCl2•6H2O | 20 | mg |
| Na2MoO4•2H2O | 30 | mg |
| DW | 1000 | ml |

Mix: 1000 ml media (a)+10 ml Medium (b)+10 ml Medium (c)

This medium is taken from Table 4 “Preparation of Basal Mineral Medium for Cultivating Knallgas Bacteria” in the google book http://books.google.com/books?id, X703AVmT8oEC&pg=PA86&lpg=PA86&dq, H2+knallgas &source=bl&ots=2SKop9LPxC&sig, —nM48q1gX5VPiD75QbftRJdCs_w&h1=en&ei=jvpjTb7oNZC4sAPBzPnFCA&sa=X&oi=book_r esult&ct=result&resnum=2&ved=0CBYQ6AEwAQ#v=onepage&q=H2%20knallgas&f=false

Glycerol was added to the media at a concentration of 20 g/liter.

The media and inoculum were combined to give 40 ml of broth in a 250 ml erlenmeyer flask, which was plugged with a foam plug to allow air exchange. The pH was adjusted to 7. The flasks were shaken at 200-250 RPM at 30° C.

Bioreactor Growth Of R. opacus strain (DSM43205) At 1 Liter Scale

Bioreactor Volume: 1 L

Bioreactor Model: Sartorius Biostat A plus

Inoculum OD/Volume: 0.8, 25 ml

Initial media—See Basal Mineral Medium (BMM) for flask growth

Starting Glycerol Concentration: 20 g/1

Media Prepared For Runtime Additions:—The following stock solutions in (mg/ml) were made and added to the medium at the indicated ratios to the 2N NH4OH usage as discussed below in Runtime Actions.

| Mineral addition solution | | mg/ml |
|---|---|---|
| Phosphate solution | Na2HPO4 | 184.55 |
| | KH2PO4•3H2O | 108.39 |
| Mg solution | MgSO4 | 210.65 |
| Ca/Fe solution | CaCl2•2H2O | 220.52 |
| | FeNH4 citrate | 106.5 |

| | |
|---|---|
| Trace Element Solution | Same as Solution D of |
| | BMM given above |

Runtime Actions:
At all times kept the DO above 30% saturation or 2.5 ppm.
Added glycerol incrementally as demand for oxygen dropped, which was indicated by a marked rise in DO at a given air flow rate.
Used 2N ammonium hydroxide as the pH raising fluid (until nitrogen depletion stage of run as discussed below) keeping track of its usage quantitatively.
Added Mineral addition solutions at the indicated ratio to the 2N NH4OH usage as given in this table

| | Microliters/milliliters of |
|---|---|
| Added Solution | added 2N NH4OH |
| Phosphate solution | 250 |
| Mg Solution | 50 |
| Ca/Fe solution | 12.5 |
| Trace elements solution | 100 |

After logarithmic growth ended and the culture entered the plateau phase, switched to 2N

NaOH for pH adjustment and added no further mineral nutrient amendments, but maintained the glycerol feed as before.

Bioreactor Run Results for R. Opacus Strain (DSM43205) Growth at 1 Liter Scale Following Above Protocol

The agitation for this run was started at 500 RPM and the temperature was maintained through the run at 30 C.

Samples were taken over the course of growth for Optical density (OD), pH, DO, cell dry weight (CDW), and nitrogen. pH was adjusted as needed using NH4OH to maintain the pH around 7.

TABLE
Bioreactor run

| | OD | | DO (% | Bio- | | |
|---|---|---|---|---|---|---|
| Time | (650 | | satu- | mass, | NH4OH | |
| (hr) | nm) | pH | ration) | g/l | (mM) | Comments |
| 0:00 | 0.007 | 7.00 | 100 | | | |
| 24:00 | 1.248 | 6.70 | 27 | | | |
| 24:30 | 1.414 | 6.8 | 22 | | | |
| 25:30 | 2.075 | 6.70 | 22 | | | |
| 26:30 | 3.555 | 6.70 | 17 | | | |
| 27:30 | 5.132 | 6.70 | 24 | | | |
| 28:30 | 4.854 | 6.70 | 24 | 18.08 | 32.4 | stated 500 mg/ml gly |
| | | | | | | feed - 18.3/ml/d |
| 29:37 | 5.933 | 6.78 | 26 | | | |
| 42:30 | 4.994 | 6.70 | 20 | 14.77 | 27.7 | added 150 ml makeup |
| | | | | | | water |
| 43:30 | 5.314 | 6.80 | 22 | 14.93 | 29.7 | |
| 44:30 | 6.194 | 6.80 | 23 | 17.36 | 30.9 | |
| 45:30 | 7.715 | 6.70 | 22 | 18.24 | 31.6 | started 500 ml/ml gly |
| | | | | | | 76.9 ml/d |
| 46:30 | 6.935 | 6.70 | 23 | 21.16 | 33.4 | |
| 47:30 | 8.414 | 6.70 | 22 | 25.17 | 36 | added 100 ml makeup |
| | | | | | | water |
| 49:00 | 7.575 | 6.70 | 20 | 21.14 | 28.5 | |
| 50:30 | 9.774 | 6.70 | 19 | 24.32 | 34 | |
| 52:00 | 6.554 | 6.70 | 17 | 19.33 | 23.5 | |
| 54:00 | 6.480 | | | | | added 100 ml water, 25 |
| | | | | | | ml of 500 mg/ml gly |
| 70:00 | 3.455 | 6.70 | 22 | 8.94 | 18.3 | added 300 ml water, |
| | | | | | | started feed for 25 |
| | | | | | | ml gly |
| 71:30 | 6.594 | 6.80 | 21 | 15.91 | 24 | |
| 72:30 | 6.975 | 6.80 | 17 | 21.66 | | 50 ml makeup water |
| 73:30 | 7.354 | 6.70 | 12 | 18.95 | | |
| 74:30 | 7.195 | 5.10 | 11 | 20.50 | 15.4 | 50 ml makeup water |
| 75:30 | 7.435 | 6.70 | NA | 23.95 | 14.5 | |
| 76:30 | 7.155 | 6.00 | NA | 25.01 | 11.3 | |
| NA | 7.975 | 6.00 | NA | 24.40 | 32.4 | |

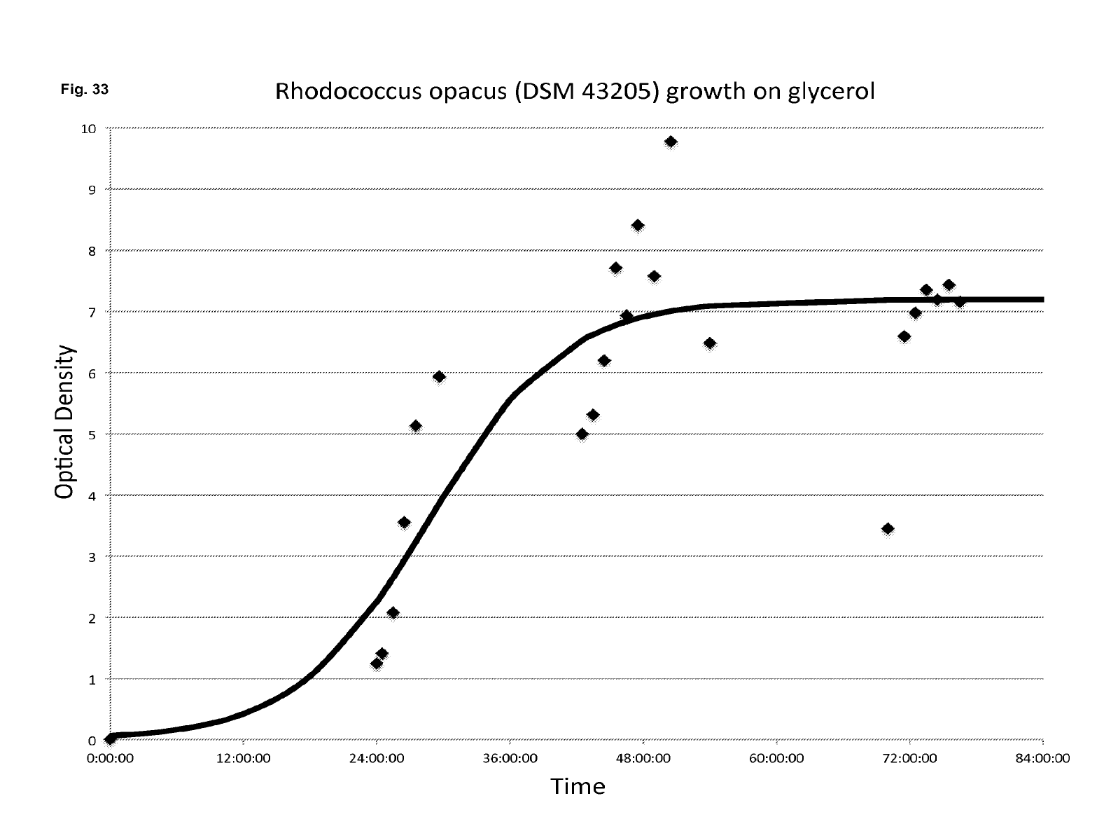

A plot of the growth curve for this 1 liter bioreactor run on glycerol is shown in FIG. 33. The run reached a top dry biomass density of 25.17 g/liter.

The fast growth and high cell yield observed for R. opacus strain (DSM43205) growing on glycerol was an unexpected and nonobvious result because the related strain R. opacus strain (DSM 44193) (also known as R. opacus PD630) has been reported to grow poorly on glycerol [Alvarez, Mayer, Fabritius, Steinbuchel, “Formation of intracytoplasmic lipid inclusions by Rhodococcus opacus strain PD630”, Arch Microbiol (1996) 165:377-386].

Bioreactor Run Results for R. opacus Strain (DSM43205) Growth at 180 Liter Scale Following Above Protocol

| | OD | | DO (% | Agita- | Temper- | |
|---|---|---|---|---|---|---|
| Time | (650 | | satu- | tion | ature | |
| (hr) | nm) | pH | ration) | (RPM) | (C.) | Comments |
| 0:00 | | | | 200 | 37 | Inoc 500 ml from |
| | | | | | | plate |
| 22:15 | | | 100 | 200 | 37 | Inoc 10 L w/500 |
| | | | | | | ml |
| 23:05 | 0.203 | 6.81 | 51.1 | 200 | 37 | Sample |
| 24:15 | 0.421 | 7.06 | 29.7 | 383 | 37 | Sample |
| 25:10 | 0.697 | 7.34 | 28.9 | 381 | 37 | Sample |
| 26:30 | 0.783 | 7.25 | 100 | 250 | 30 | Transfer to 180 L |
| 29:15 | 0.295 | 7.27 | 97.6 | 250 | 30 | Sample |
| 31:30 | 0.306 | 7.2 | 97.5 | 251 | 30 | Sample |
| 33:05 | 0.41 | 7.16 | 93.9 | 251 | 30 | Sample |
| 41:00 | 5.31 | 6.57 | 92.1 | 252 | 30 | Added Minerals |
| 44:00 | 6.13 | 6.72 | 85.4 | 250 | 30 | Added Mineral - |
| | | | | | | sample #1 |
| 46:30 | 7.94 | 6.66 | 104.6 | 250 | 30 | DO Increase/Start |
| | | | | | | Glycerol feed 10 |
| | | | | | | ml/min/Change to |
| | | | | | | NaOH |
| 47:45 | | 7.01 | 82.8 | 255 | 30 | Increase feed to |
| | | | | | | 20 ml/min |
| 48:30 | 8.62 | 7.11 | 106.4 | 250 | 30 | Sample |
| 49:40 | 14.3 | 7.07 | 108.3 | 250 | 30 | Sample |
| 57:30 | 18.1 | 7.02 | 102.4 | 250 | 30 | Sample |
| 65:15 | 21.1 | 6.89 | 100.5 | 250 | 30 | Sample |
| 69:00 | 21.7 | 6.95 | 101.6 | 250 | 30 | Sample |
| 70:15 | 19.2 | 6.98 | 103.7 | 250 | 30 | Sample |
| 74:00 | 21.8 | 6.84 | 106.5 | 250 | 30 | Sample |
| 79:00 | 18.8 | 6.83 | 106 | 250 | 30 | Sample |
| 90:30 | 13.5 | 6.91 | 113.8 | 250 | 30 | Sample |
| 94:00 | | 6.89 | 120.4 | 250 | 30 | Sample/Harvest |

At the end of the run 3.3 kg of wet cell mass was recovered.

Flask Growth of R. opacus Strain (DSM43205) on Methanol

First a serum bottle filled with 20 ml of the Basal Mineral Medium given above for growth on glycerol was used to grow R. opacus strain (DSM43205) on a chemoautotrophic gas mix of 65% H2, 25% air, and 10% CO2. The serum bottle culture of R. opacus strain (DSM43205) was used to provide a 5% sized inoculum for a flask.

The media used for flask level growth of the microbe was the Basal Mineral Medium given above for growth of R. opacus strain (DSM43205) on glycerol.

Methanol was added to the media at a concentration of 25 g/liter.

The media and inoculum were combined to give 40 ml of broth in a 250 ml erlenmeyer flask, which was plugged with a foam plug to allow air exchange. The pH was adjusted to 7. The flasks were shaken at 200-250 RPM at 30° C.

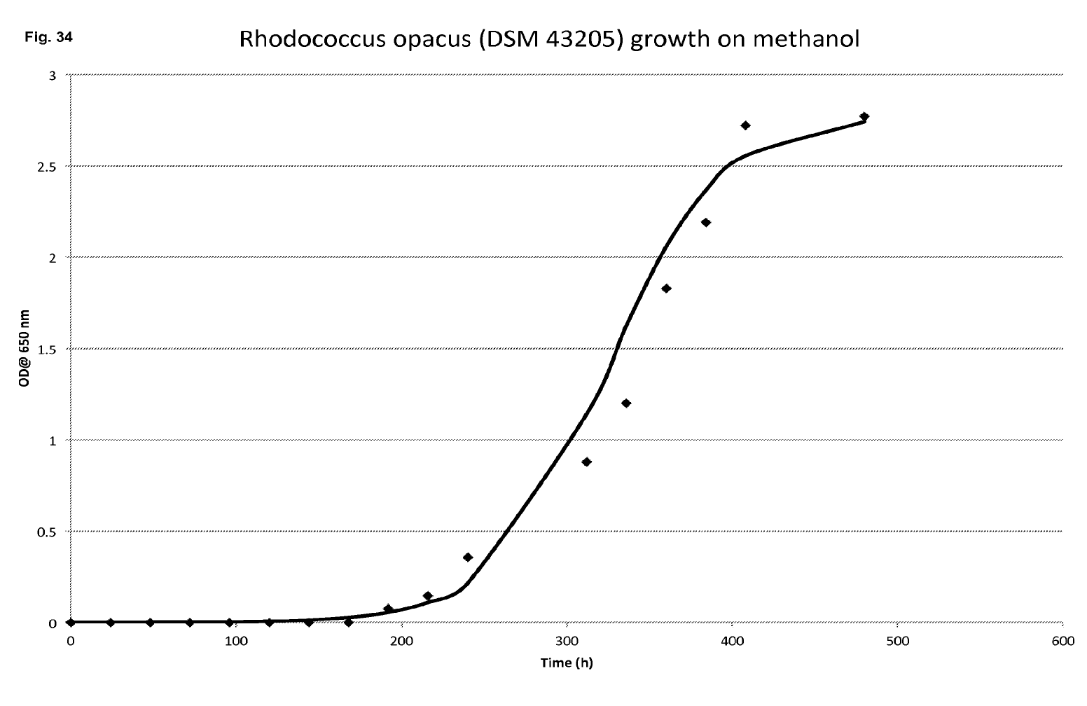

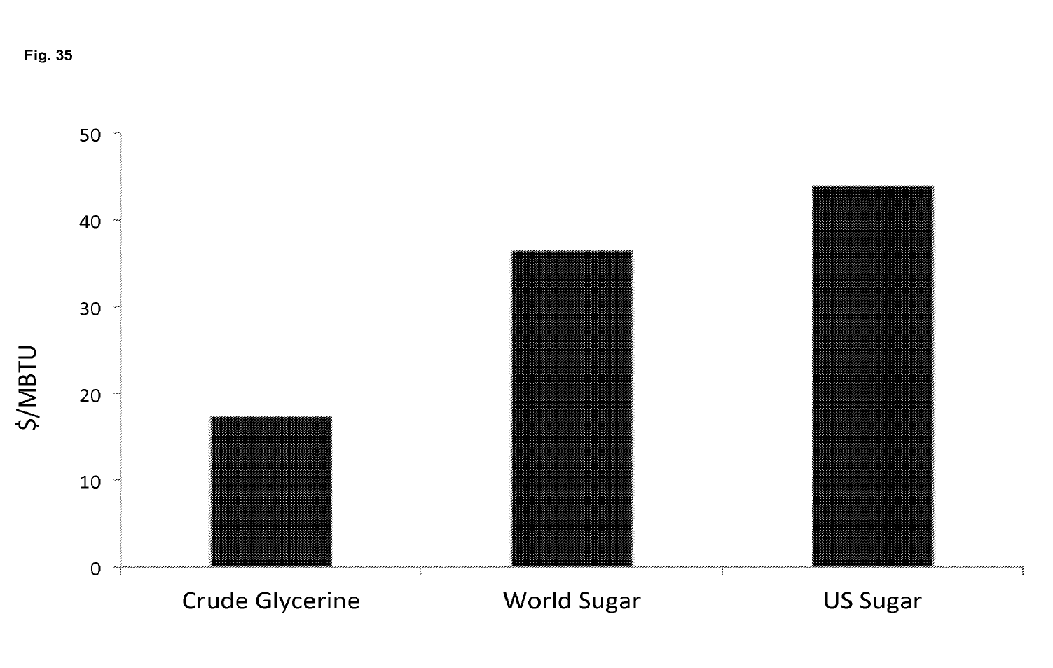

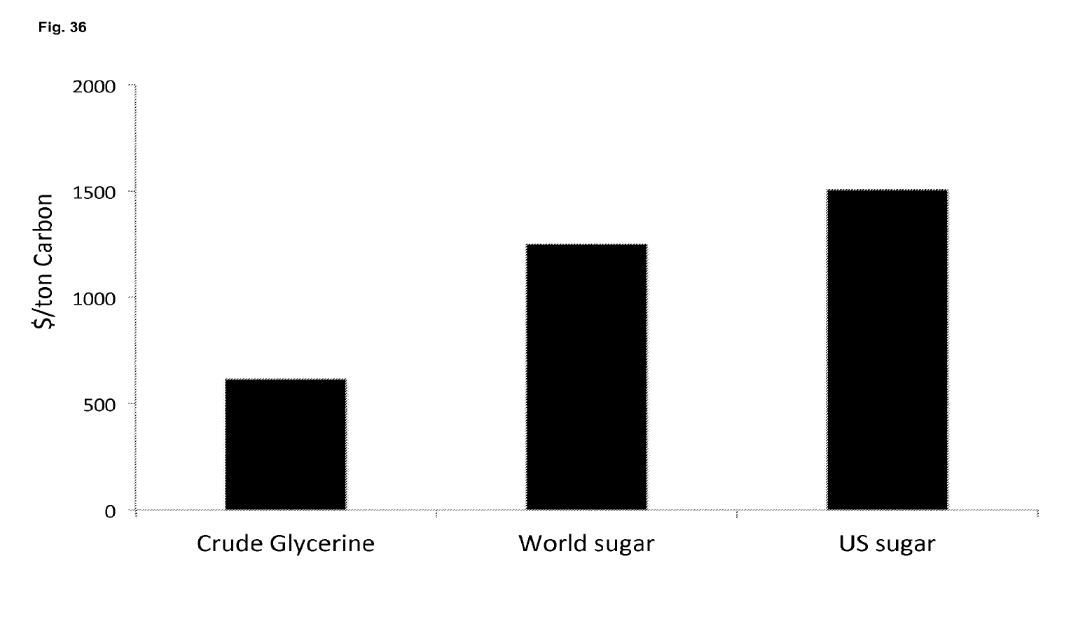

A plot of the growth curve for this flask cultivation of R. opacus strain (DSM43205) on methanol is shown in FIG. 34. Note it has been found that the relatively long lag phase observed at the beginning of cultivation in FIG. 34 can be avoided by inoculating with a culture grown on methanol.

The ability of R. opacus strain (DSM43205) to grow on methanol as the sole carbon and energy source was unexpected and to our knowledge the finding that R. opacus strain (DSM43205) can grow on methanol is a novel result that has never been reported before.

Flask Growth of R. opacus Strain (DSM43205) and R. opacus Strain (DSM43206) on glycerol

Test tube inoculum for R. opacus strain (DSM43205) and R. opacus strain (DSM43206) were grown on Lysogeny broth (LB) for 18-24 hours. The inoculum size introduced to the flask was 5%.

Media used for flask level growth of the microbes were

Media (a):

| | | |
|---|---|---|
| Na2HPO4•12H2O | 9 | g |
| KH2PO4 | 1.5 | g |
| NH4Cl | 1.0 | g |
| MgSO4•7H2O | 0.2 | g |
| Trace Mineral Soln | 1.0 | ml (media d below) |
| Distilled water (DW) | 1000 | ml |

Medium (b)

| | |
|---|---|
| NaHCO3 | 5 g |
| DW | 100 ml |

Medium (c)

| | |
|---|---|
| Ferric Ammonium Citrate | 50 mg |
| CaCl2•2H2O | 100 mg |
| DW | 100 ml |

Media (d)—Trace Mineral Medium

| | | |
|---|---|---|
| ZnSO4•7H2O | 100 | mg |
| MnCl2•4H2O | 30 | mg |
| H3BO3 | 300 | mg |
| CoCl2•6H2O | 200 | mg |
| CuCl2•2H2O | 10 | mg |
| NiCl2•6H2O | 20 | mg |
| Na2MoO4•2H2O | 30 | mg |
| DW | 1000 | ml |

Mix: 1000 ml media (a)+10 ml Medium (b)+10 ml Medium (c)

This medium is taken from Table 4 “Preparation of Basal Mineral Medium for Cultivating Knallgas Bacteria” in the google book http://books.google.com/books?id, X703AVmT8oEC&pg=PA86&lpg=PA86&dq, H2+knallgas &source=bl&ots=2SKop9LPxC&sig, —nM48q1gX5VPiD75QbftRJdCs_w&h1=en&ei=jvpjTb7oNZC4sAPBzPnFCA&sa., X&oi=book_r esult&ct=result&resnum=2&ved=0CBYQ6AEwAQ#v=onepage&q=H2%20knallgas&f=false

Glycerol was added to the media at a concentration of 20 g/liter.

The media and inoculum were combined to give 40 ml of broth in a 250 ml erlenmeyer flask, which was plugged with a foam plug to allow air exchange. The pH was adjusted to 7. The flasks were shaken at 200-250 RPM at 30° C.

The growth for Rhodococcus opacus (DSM 43205) and Rhodococcus opacus (DSM 43206) on glycerol and a basal mineral medium in 250 ml flasks is shown below.

| | | OD | OD |
|---|---|---|---|
| Time | Time | R. opacus | R. opacus |
| (days) | (hours) | (DSM 43205) | (DSM 43206) |
| 0 | 0 | 0.03 | 0.03 |
| 1 | 24 | 0.109 | 4.171 |
| 2 | 48 | 2.847 | 6.982 |
| 5 | 120 | 8.311 | 9.562 |
| 6 | 144 | 9.528 | 10.906 |
| 9 | 216 | 11.221 | 14.442 |

Flask Growth of R. opacus Strain (DSM43205) on Crude Glycerol from a Biodiesel Manufacturer

Using the same basal mineral media given in the previous example R. opacus strain (DSM43205) was grown on a crude glycerol sample received from a biodiesel manufacturer. In one flask a concentration of 10 g/liter of the crude glycerol was used and in another a concentration of 40 g/liter was used. Following growth the cell mass was freeze dried and the polar and neutral lipids extracted. Both flasks grew to an OD of 1. The lipid content by weight was found to be as follows

| Crude | | | |
|---|---|---|---|
| Glycerol | Total Lipids | Neutral Lipids | Polar Lipids |
| g/liter | % | % | % |
| 10 | 21.2 | 10.6 | 10.6 |
| 40 | 34.7 | 15.1 | 19.6 |

The percent neutral and polar lipids by weight for each sample was determined as follows. 0.5 g of lyophilized bacteria was weighed out. A mortar or spatula was used to break down big pieces of material and the powder was added into a 30 ml glass conical centrifugation tube. A digital heat block was heated to 65 degrees celcius. 1.5 ml methanol was added per 100 mg biomass with a glass pipet and the slurry was vortexed briefly. The slurry was incubated for 20 minutes at 65 degrees. The tubes were removed and the sample cooled to room temperature after which methanol was added again to the slurry at twice the volume previously followed by vortexing the slurry again. Then the vial was put in a rack on a shaker and incubated on the shaker for 1 hour at room temperature. Then the vial was incubated on the heat block for 20 minutes at 40 degrees followed by vortexing again. The sample vial was then spun on a tabletop centrifuge at low speed (1000 rpm) for 5 minutes. The chloroform/methanol extract was removed from the vial using a glass pipet to transfer only the supernatant to a fresh vial while leaving the pellet behind. The chloroform/methanol extract was dried down with a flow of Nitrogen. The dried chloroform/methanol extract was then resuspended in hexane (˜⅓ volume of chloroform previously added) and vortexed again. The resuspended extract was centrifuged (1000 rpm) for another three minutes after which the extract was transferred using a glass pipet, taking care to transfer only the supernatant to a preweighed small glass tube (supelco vial). The hexane solvent was dried off with a flow of nitrogen. The tube with the dried hexane extract was then reweighed. The weight of the dried hexane extract divided by the original cell dry weight gave the percent neutral lipid. In the centrifuge vial where the pellet remained after hexane extraction an equal volume of 2:1 chloroform/methanol was added as the volume of hexane previously used. This liquid was then transfered to a pre-weighed glass tube and dried with N2. The tube plus dried extract was then reweighed. The weight of this extract divided by the original cell dry weight gave the percent polar lipid (hexane insoluble-methanol/chloroform soluble).

Demonstrating the Salt Tolerance of R. opacus Strain (DSM43205)

In this experiment R. opacus strain (DSM43205) was grown on the same basal media given above with 80 g/liter glucose added as a carbon and energy source and additional NaCl added to the medium in order to test salt tolerance. Salt is a common constituent in crude glycerol. In three experimental flasks 187.5 mM, 375 mM, and 750 mM NaCl were added respectively. A control flask had 0 mM NaCl added. It was found that growth with 187.5 mM and 375 mM NaCl could occur from an inoculum prepared on Lysogeny broth (LB). In order to grow R. opacus strain (DSM43205) on the media with 750 mM NaCl an inoculum had to be taken from the culture grown with 375 mM NaCl. Trying to use an inoculum prepared with LB did not successfully grow when directed placed in the media with 750 mM NaCl. Thus R. opacus strain (DSM43205) requires conditioning on increasing salt concentrations in order to be able to tolerate relatively high salt concentrations. The final dry cell densities and lipid contents were as follows. Fat contents were determined using Method AOAC 996.06; where AOAC stands for Association of Analytical Communities.

| NaCl | Final Cell Mass Density | Fat content |
|---|---|---|
| (mM) | (g/liter) | (weight %) |
| 0 | 8.65 | 45.02 |
| 187.5 | 7.90 | 48.1 |
| 375 | 7.51 | 38.92 |
| 750 | 4.79 | 33.38 |

R. opacus strain (DSM43205) was found to be able to grow in up to 750 mM NaCl which corresponds to 43.8 g/liter NaCl. This added NaCl is a higher salinity than that found in sea water (35 g/liter). In addition the basal mineral media contributed another 6.6 g/liter of salinity, giving a total salinity under which R. opacus strain (DSM43205) exhibited growth and lipid accumulation that exceeded 50 g/liter.

### Characterization of Organisms Sharing High 16SrRNA Sequence Similarity.

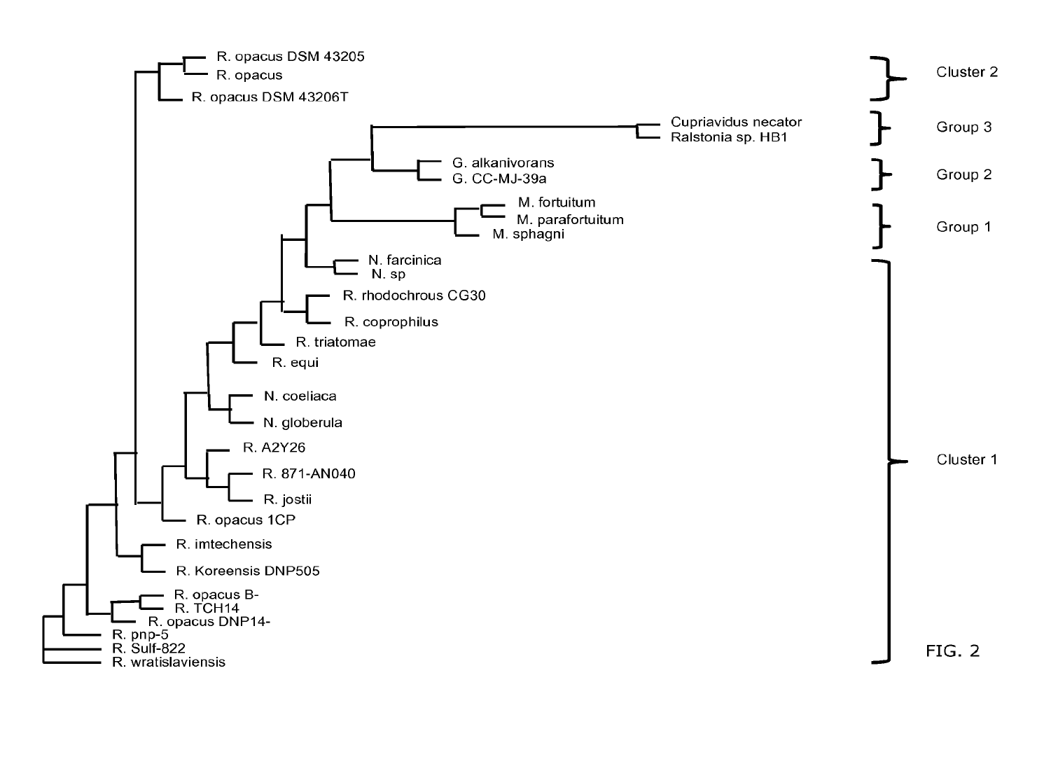

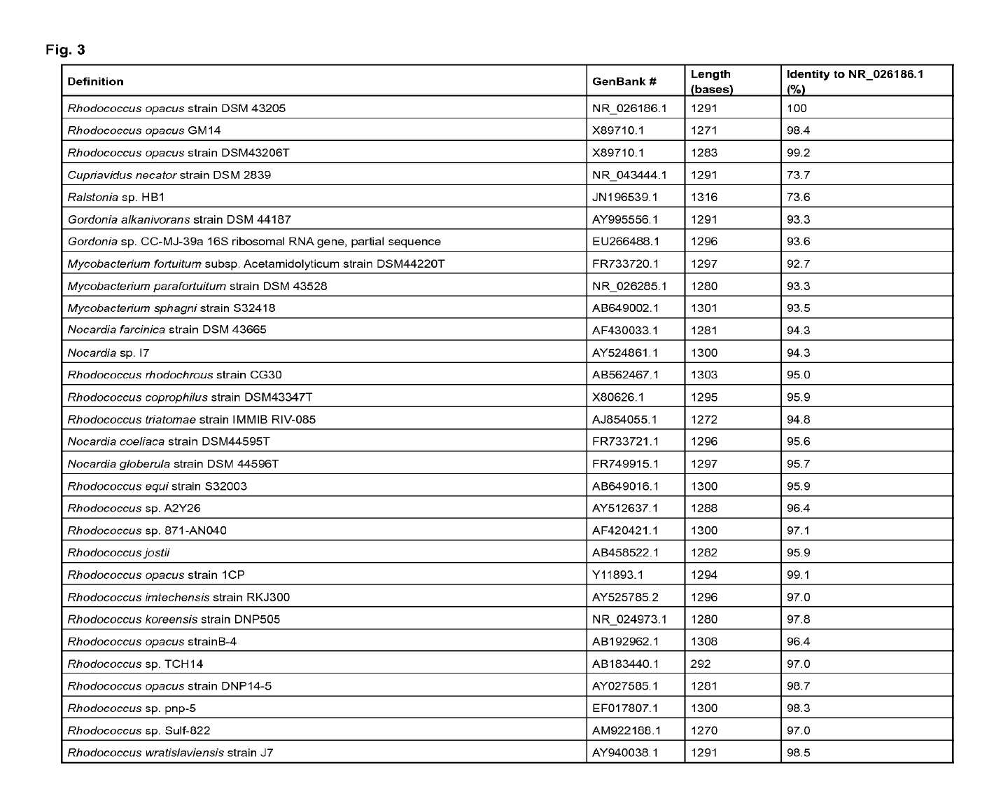

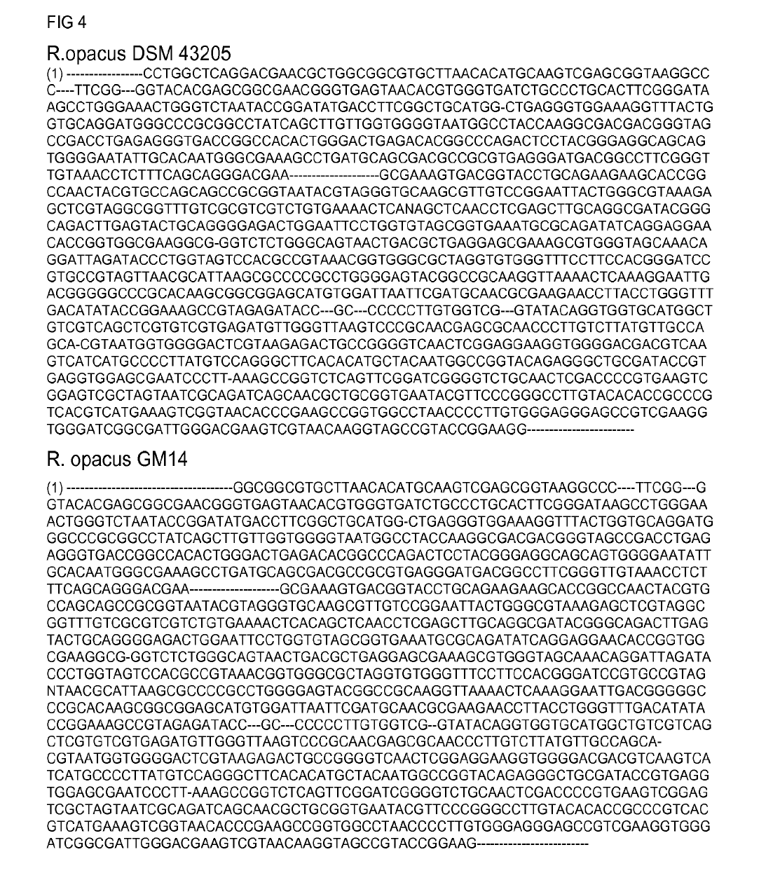

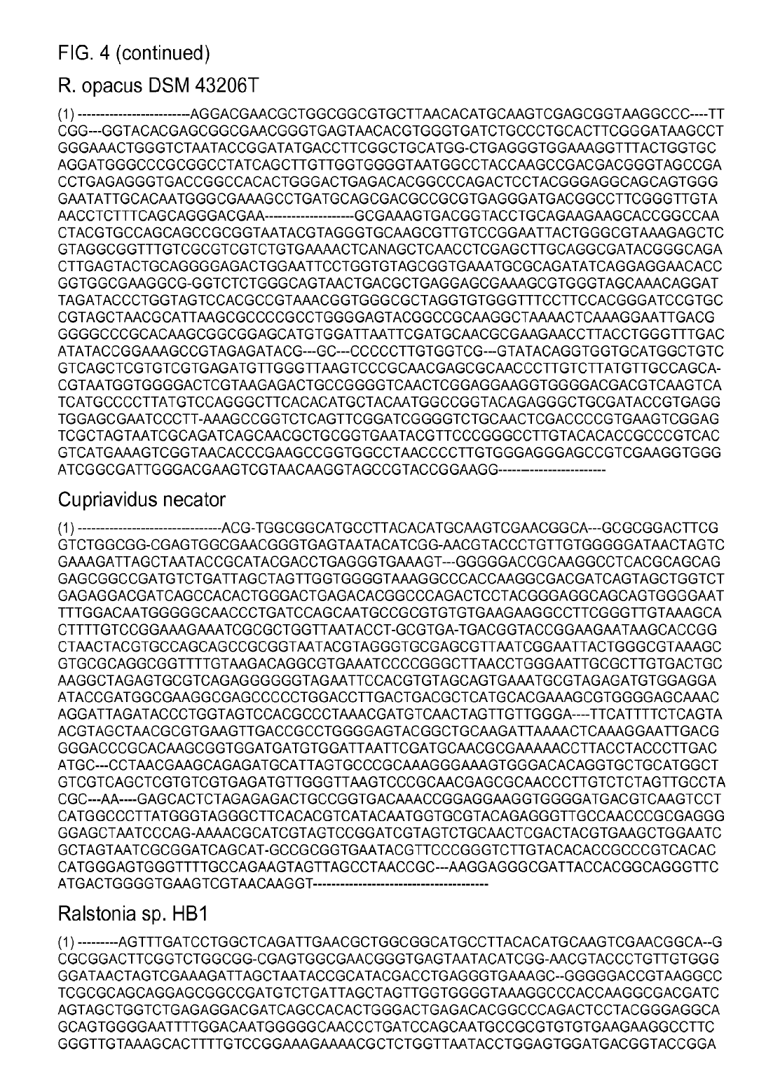

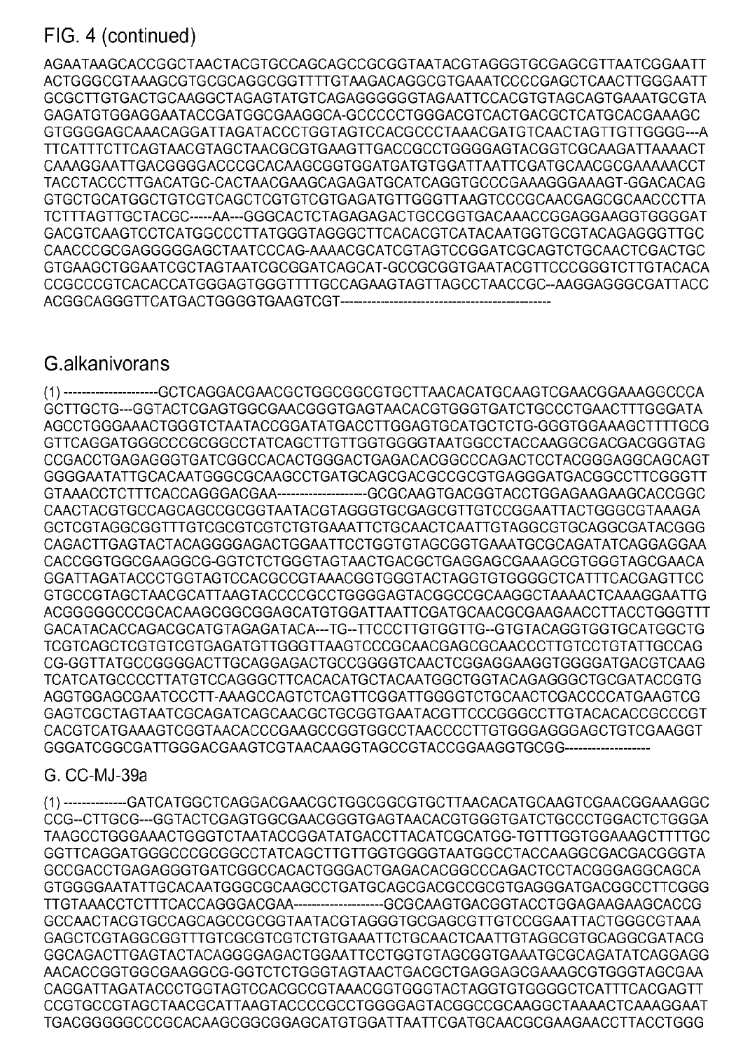

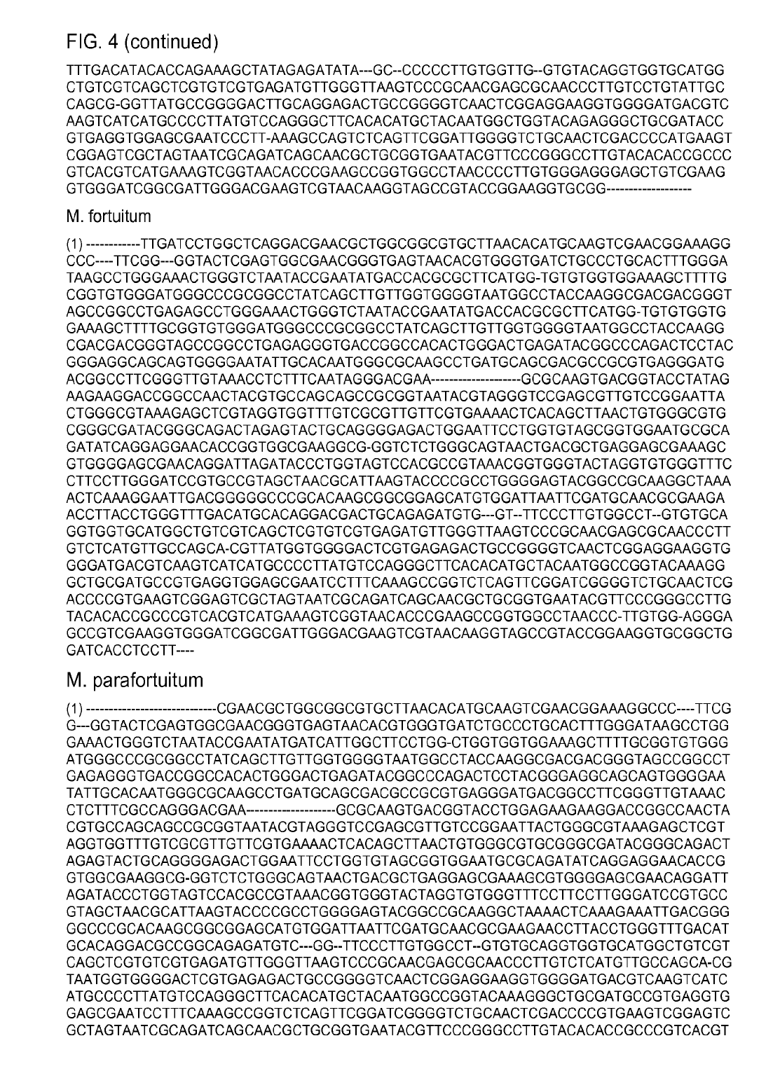

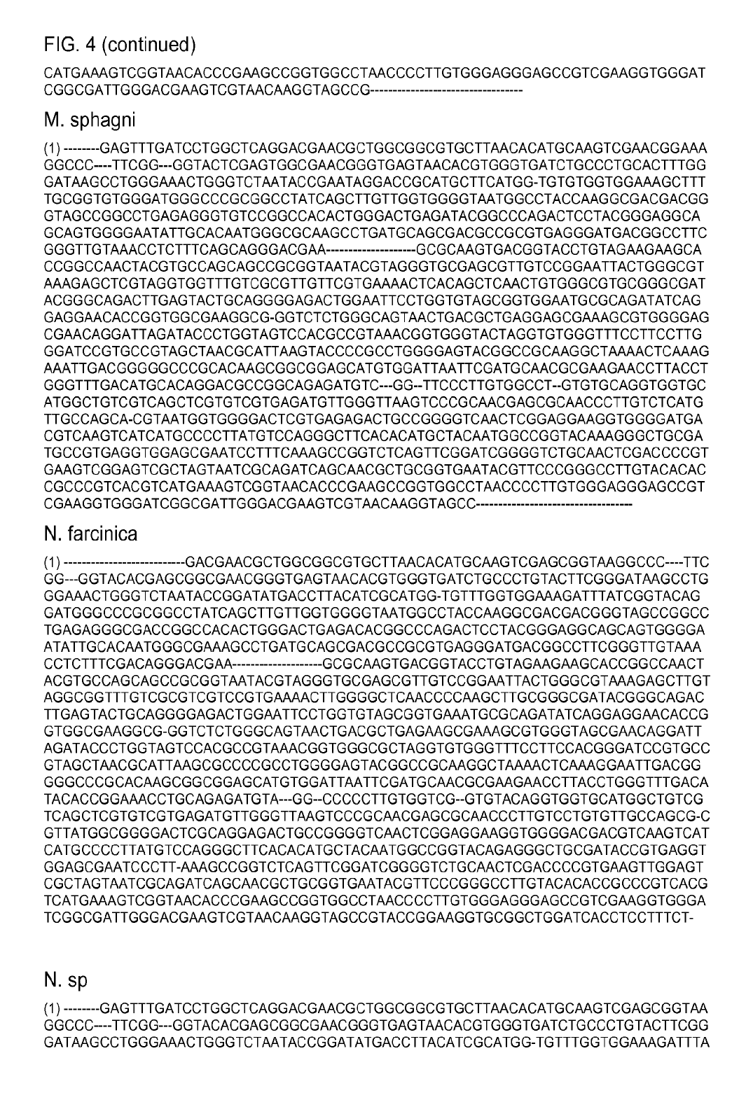

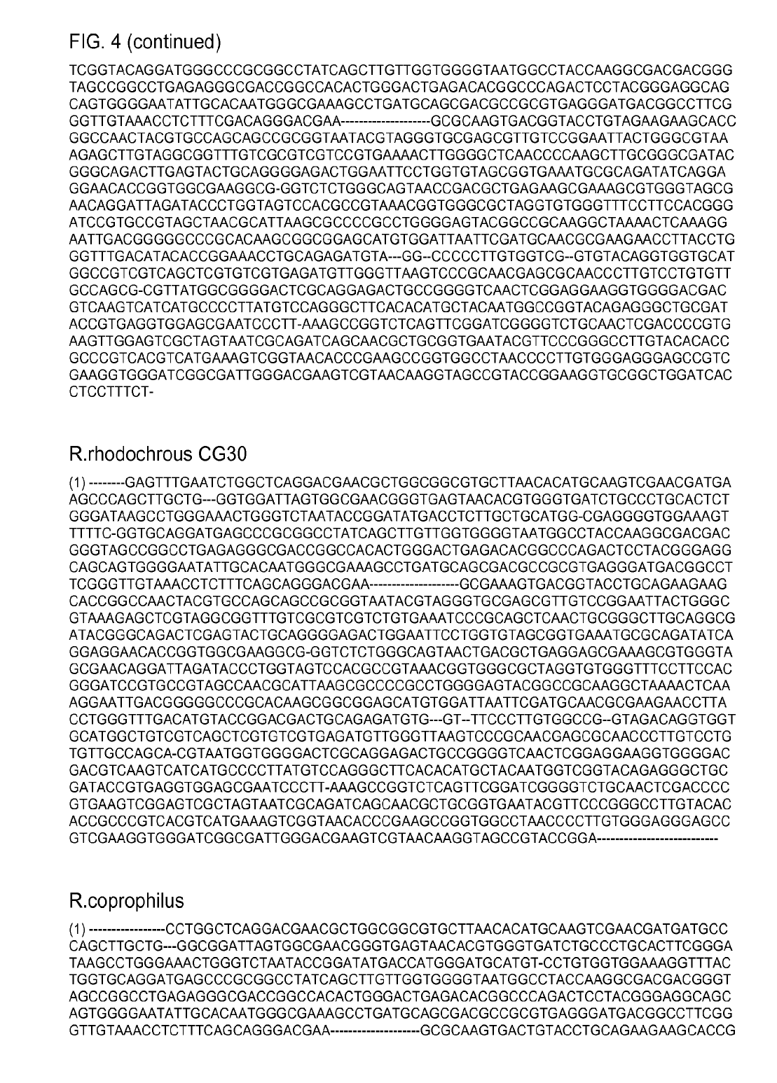

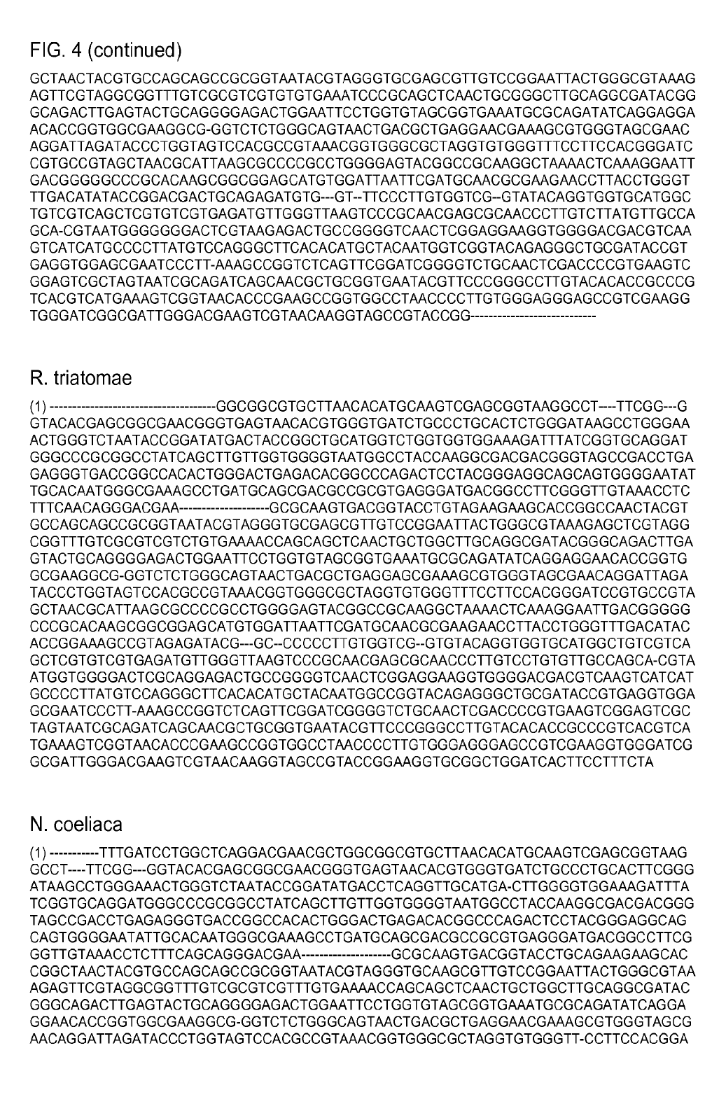

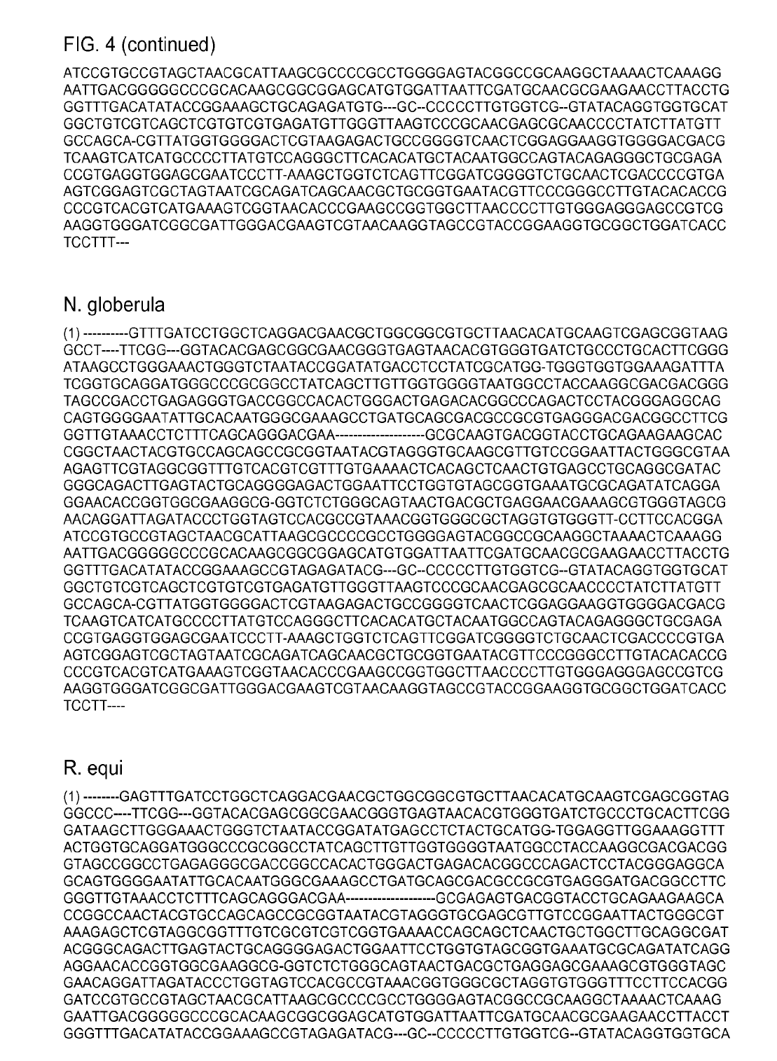

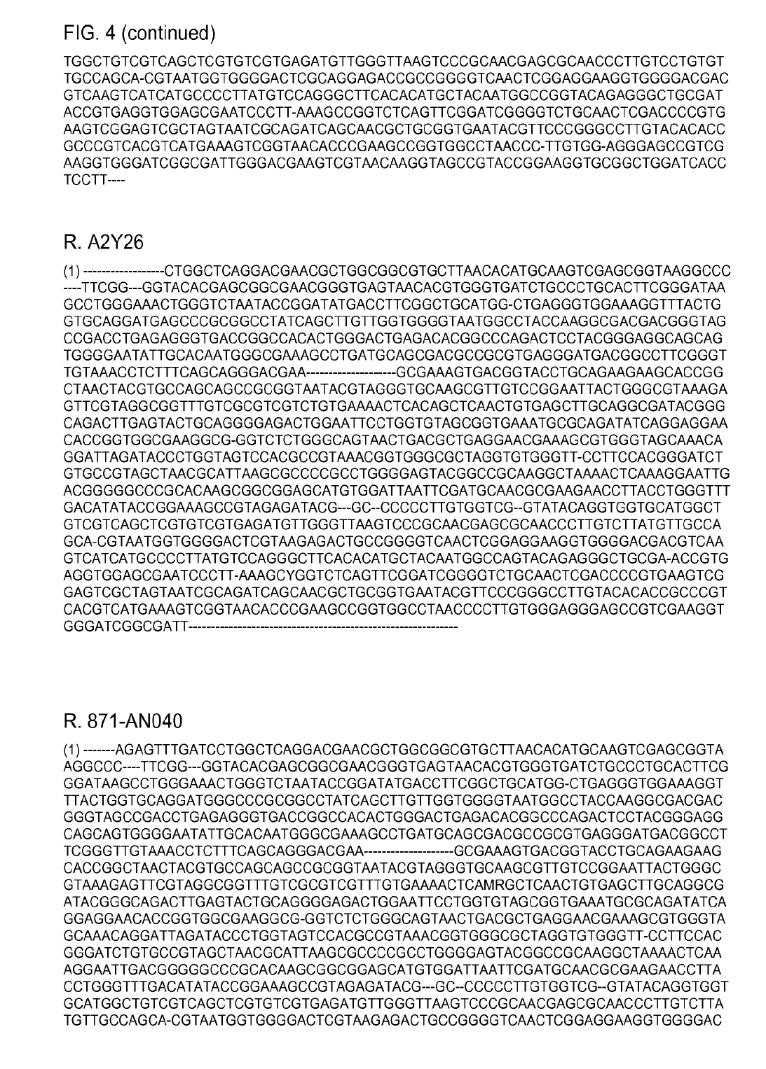

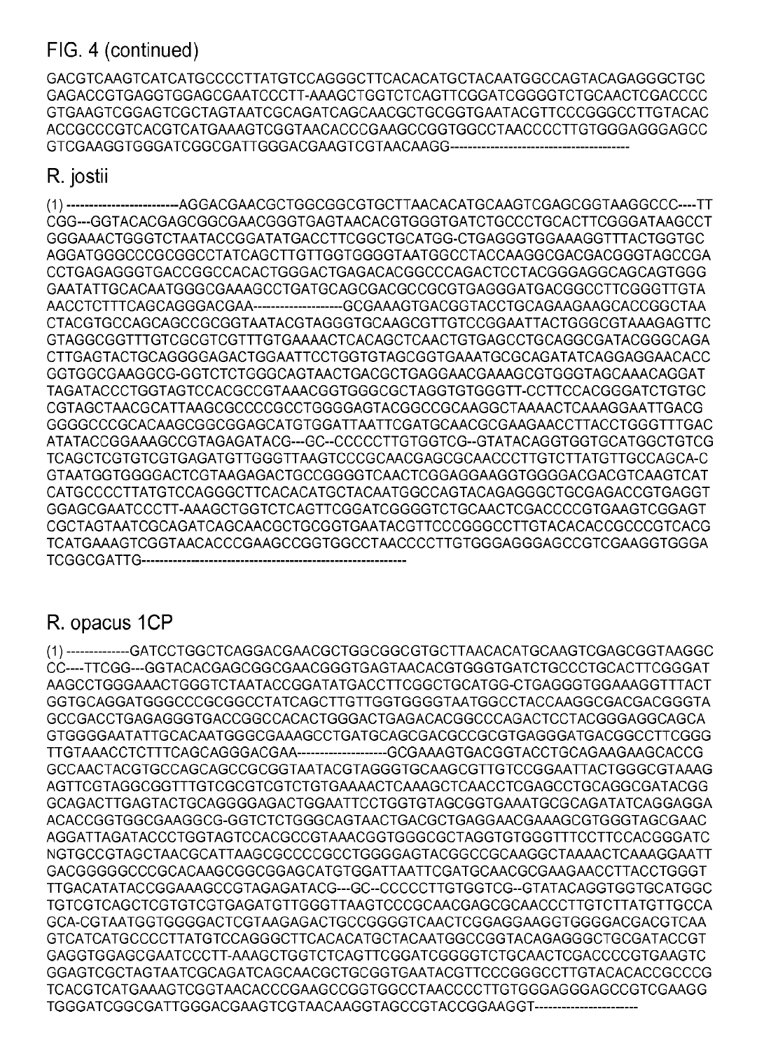

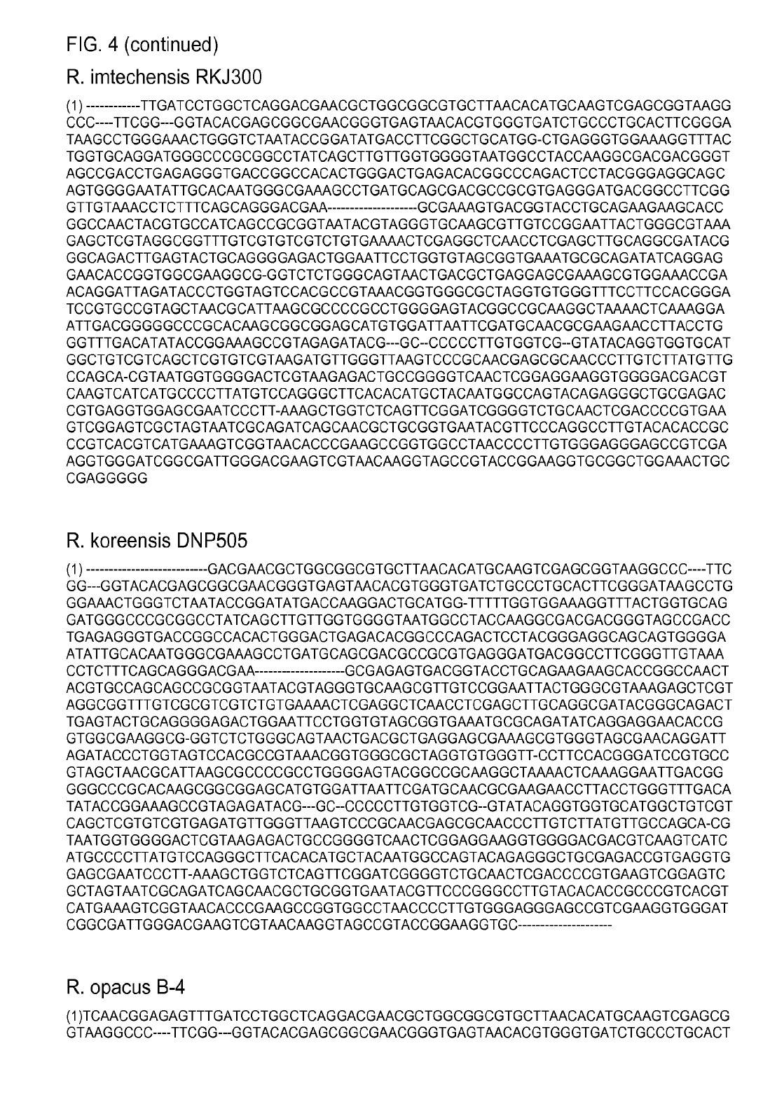

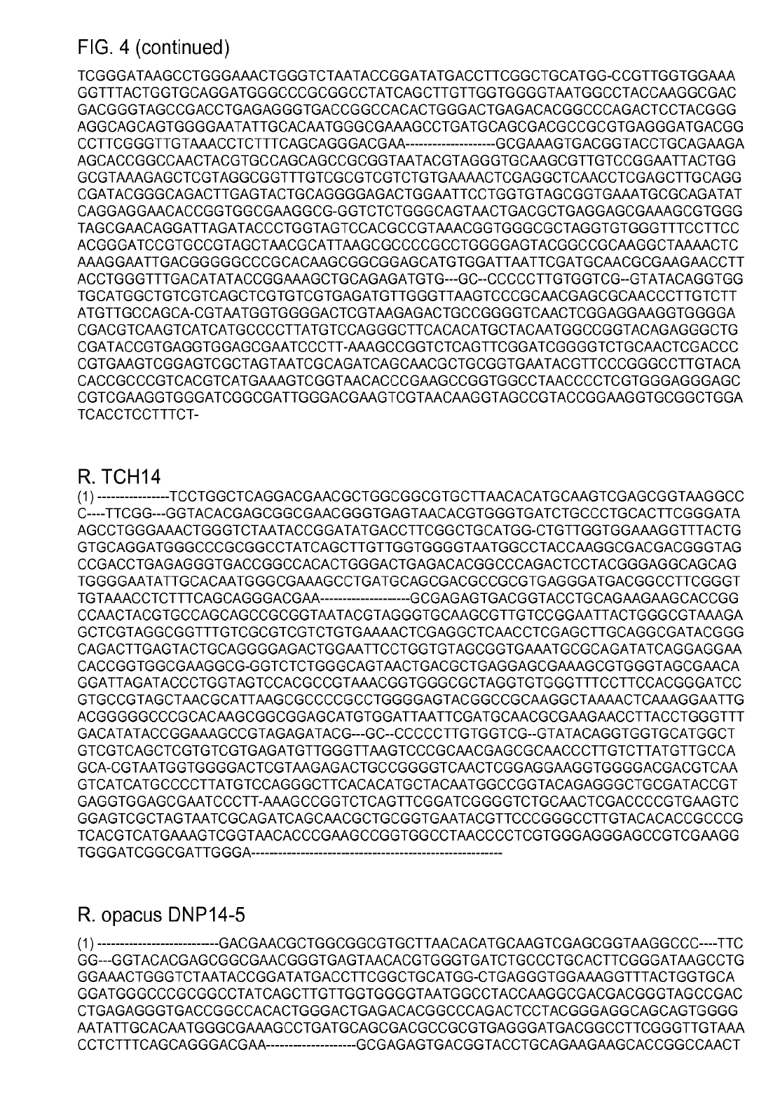

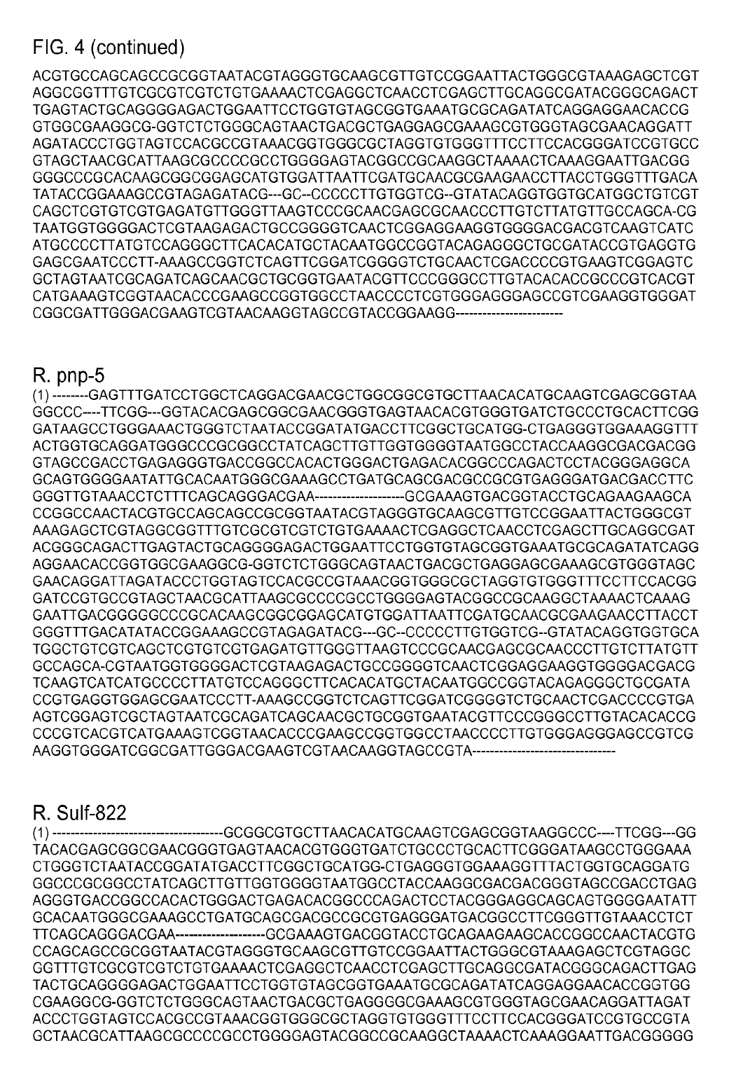

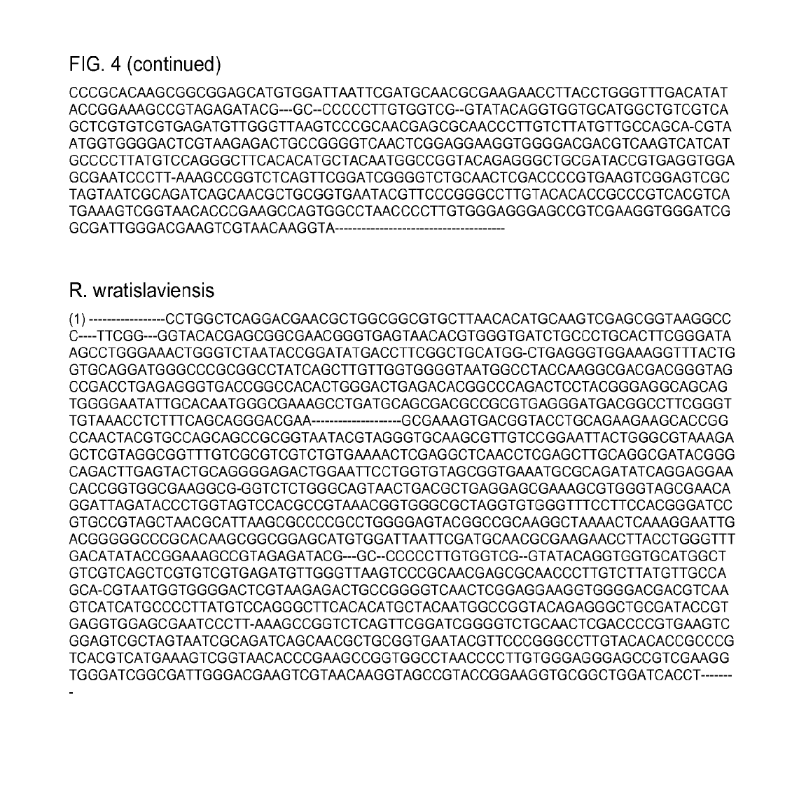

To identify organisms closely related to R. opacus strain (DSM43205), a basic local alignment search (BLAST^{R}) with the BLASTN programs search of nucleotide databases using the 16S rRNA (NR_{—}026186.1) was carried out. The phylogenetic relationships, based on the 16S rRNA gene sequence homology, between the tested strain and the reference strains of the suborder corynebacterineae (corynebacterium, gordoniaceae, mycobacteriaceae and nocardiaceae) and the family burkholderiaceae (genus cupriavidus and ralstonia) are shown in FIG. 2. The nocardiaceae are related and form two clusters of organisms: clusture 1 that contains 20 organisms from the genus nocardia and rhodococcus and cluster 2 that contains 3 R. opacus strains (DSM43205, GM14 and DSM43206). The gordoniaceae, mycobacteriaceae and burkholderiaceae form 3 separated groups (1, 2 and 3). The gram positive chemoautotroph lipid accumulating strain R. opacus (DSM43205; NR_{—}026186.1) exhibits high sequence similarity to cluster 1 (94.3-99.1%) and to the gram positive groups 1 and 2 (92.7-93.5% and 93.3-93.6% respectively) (FIGS. 3 and 4). The sequence similarity to the gram negative chemoautotroph poly(3-hydroxybutyrate) (PHB) accumulating strains in group 3 is 73.7%.

### Plasmid Design and Construction

To generate an E. coli Rhodococci shuttle vector suitable for electroporation, the plasmid pSeqCO1 (SEQ ID: 01) was constructed with the genetic elements described in FIG. 10A. pSeqCO1 consists of the replication gene operon, ampicillin and kanamycin resistance genes, LacZ operon and the multiple cloning site as described in FIG. 10B and FIG. 11A. For replication in Rhodococci, the DNA fragment of the repAB operon (1744 bp downsteam from the XhoI restriction site in the native pKNR01 plasmid of the bacteria Rhodococcus opacus B4; Na et al. 2005,) Biosci Bioeng. 99: 408-414) was synthesized with the restriction sites KpnI and SalI and cloned into PUC18 digested with KpnI and SalI. The resultant vector was digested with SpeI and BglII and ligated with the PCR product of the Kanamycin resistance gene from pBBR1MCS-2 (Kovach et al. 1995 Gene 166: 175-176) digested with the engineered restriction sites SpeI and BglII to give pSeqCO1.

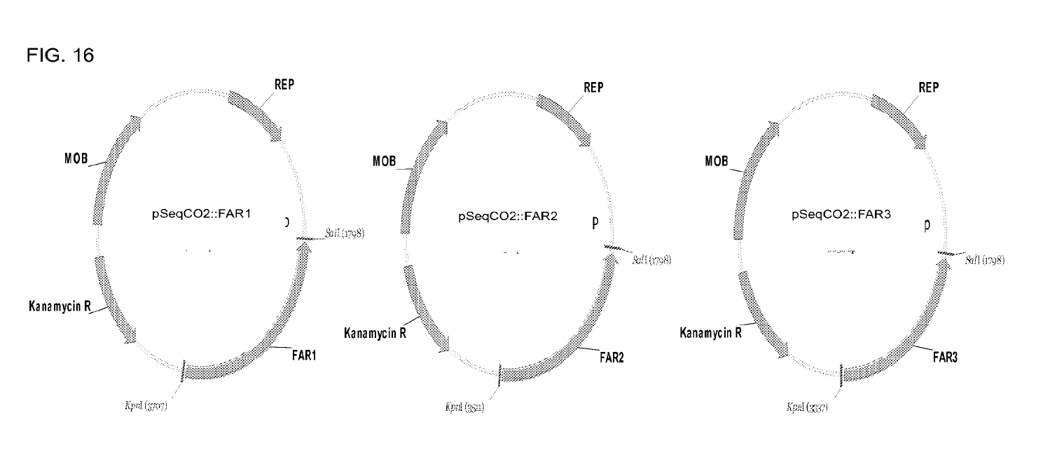

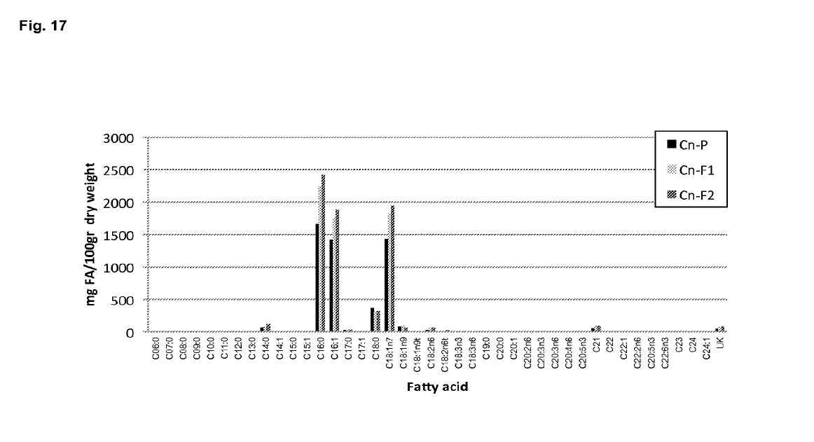

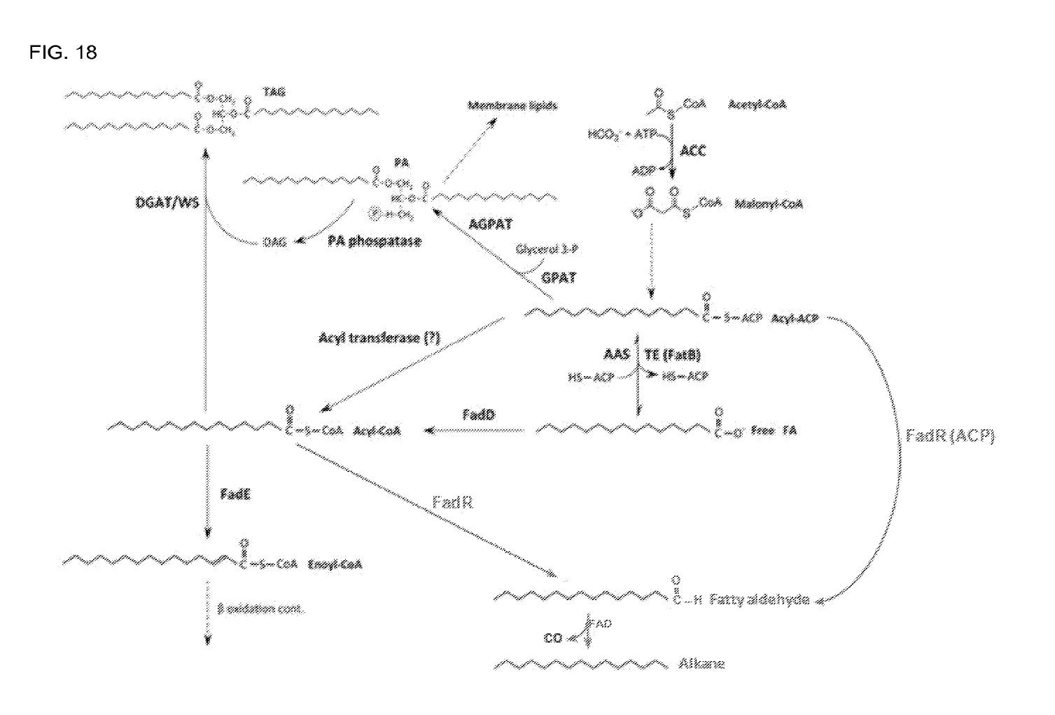

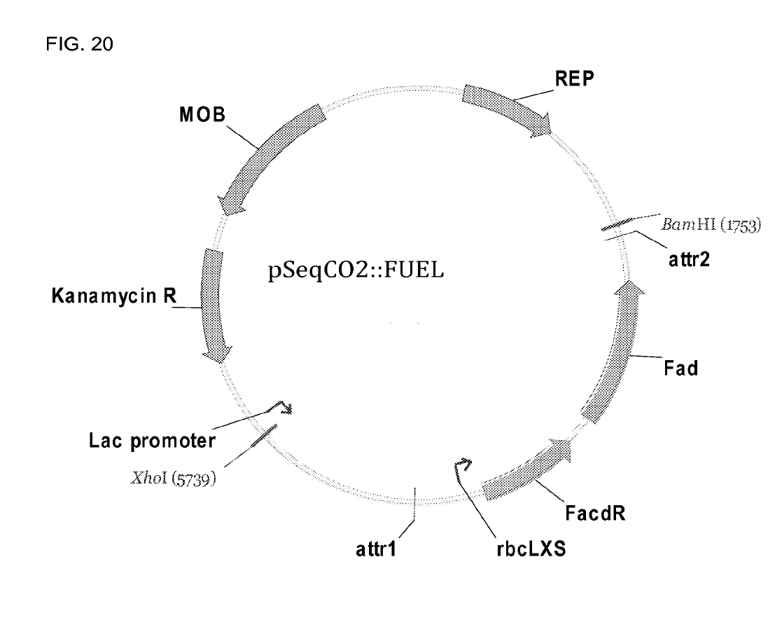

To generate an E. coli-cupriavidus shuttle vector suitable for electroporation and bacterial conjugation, the plasmid pSeqCO2 (SEQ ID: 02) was used with the genetic elements described in FIG. 10A. pSeqCO2 (SEQ ID: 02; FIGS. 10 and 11B) is the plasmid pBBR1MCS-2 described in Kovach et al. (1995 Gene 166: 175-176) that contains the IncQ like replication gene, Mob gene that mobilized when the RK2 transfer functions are provided in trans, kanamycin resistance gene, LacZ operon and the multiple cloning site as described in FIG. 10B and FIG. 11B. Pver1 (SEQ ID: 03; FIGS. 10 and 11C) is an E. coli-cupriavidus-Rhodococci shuttle vector suitable for electroporation and bacterial conjugation. The plasmid was generated by cloning the repAB operon (described in pSeqCO1) into pSeqCO2 using the KpnI and SalI restriction sites. Pver2 (SEQ ID: 04; FIGS. 10 and 11D) is an E. coli-cupriavidus-Rhodococci shuttle vector suitable for electroporation and bacterial conjugation. The plasmid was generated by cloning the synthesized chloramphenicol gene (Alton and Vapnek Nature 1979 282: 864-869) with the engineered restriction sites SalI and HindIII into Pver1. The arabidopsis genes FAR1 (SEQ ID: 05), FAR2 (SEQ ID: 06) and FAR3 (SEQ ID: 07): were synthesized and cloned into the plasmid pUC57. FAR1, FAR2 and FAR3 were rescued from PUC57 using the restriction enzymes KpnI and SalI and cloned into pSeqCO2 digested with KpnI and SalI to give pSeqCO2::FAR1, pSeqCO2::FAR2 and pSeqCO2::FAR3 respectively (FIG. 16). The genes FadDR (SEQ ID: 08) and Fad (SEQ ID: 09) and the rbcLXS promoter (SEQ ID: 10) were PCR amplified from the cyanobacterium Synechocystis sp. PCC 6803 genome and cloned into gateway plasmid to give pFUEL. A 4 kBp XhoI BamHI fragment that contains FadDR, Fad and rbcLXS was rescued from pFUEL and cloned into pSeqCO2 digested XhoI BamHI with to give pSeqCO2::FUEL (FIG. 20).

### Microorganism Transformation

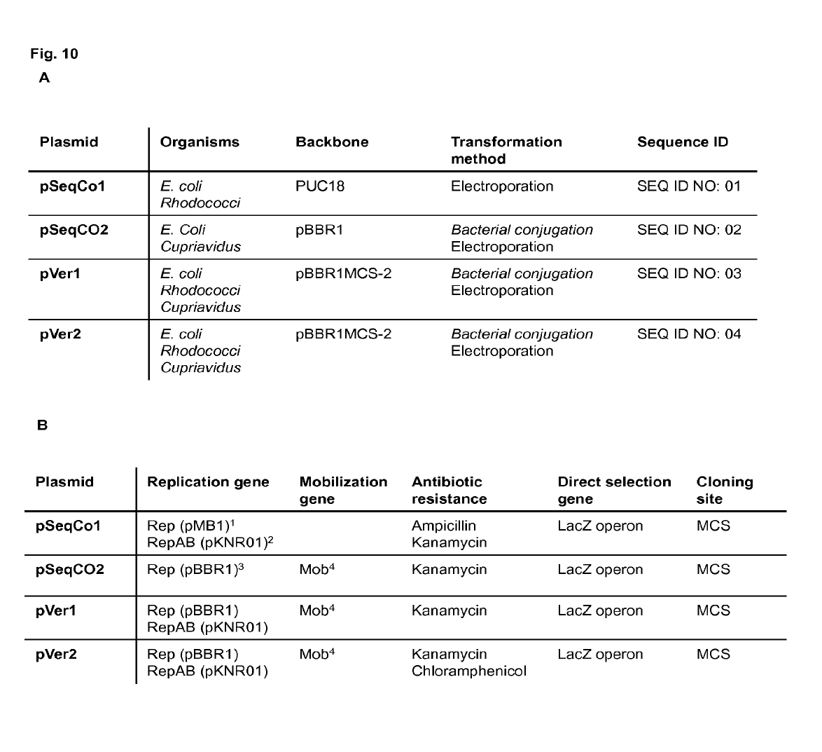

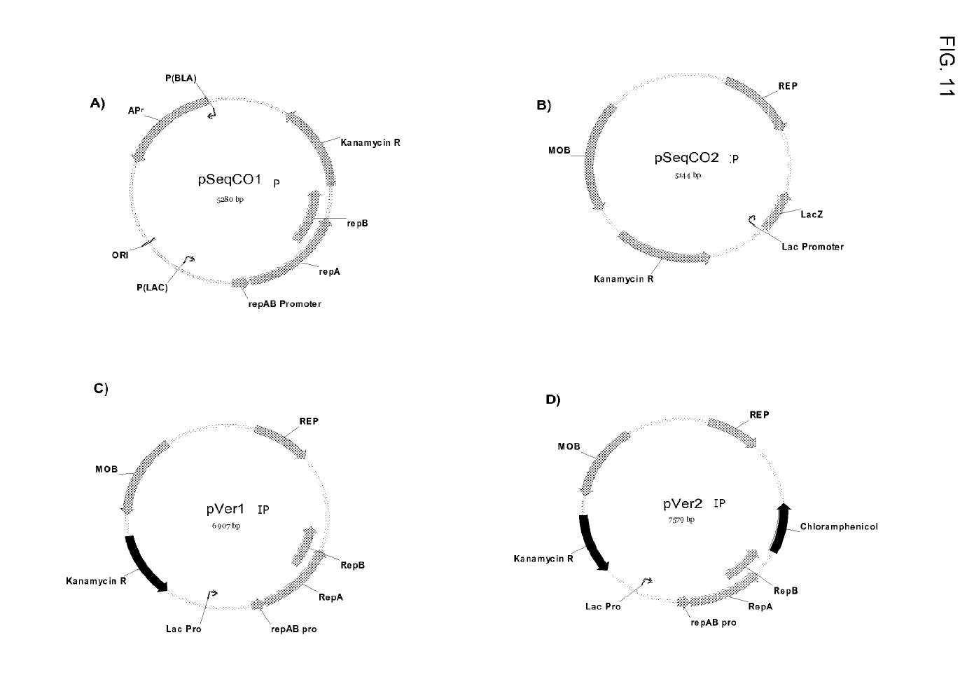

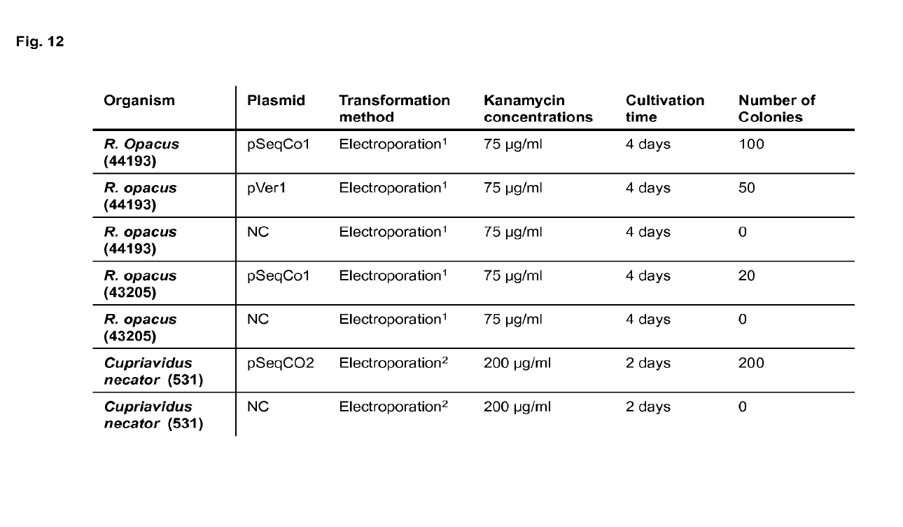

Transformation of Rhodococci was carried out using the plasmids pSeqCO1 and pVer1 (FIG. 12) as described below.

Rhodococci competent cells were prepared by incubating a single colony 2 ml NB medium (5 g/L peptone, 1 g/L meat extract, 2 g/L yeast extract, 5 g/L NaCl; pH=7.0±0.2) at 30° C. overnight. One ml was inoculated to 50 ml NB medium supplemented with 0.85% (w/v) glycine and 1% (w/v) sucrose in a 250 ml baffled Erlenmeyer Flask and incubated to a cell density of O.D₆₀₀=0.5. Cells were collected by centrifugation at 3,000×g for 10 min at 4° C. and washed 3 times with 50 ml (each) of sterile ice-cold double distilled water (ddH₂O). Cells were concentrated 20-fold by re-suspending the collected cells in 2.5 ml of ddH₂O and 400 μl aliquots stored in 1.5 ml tube at −70° C. Electroporation was carried out by thawing the competent cells on ice and mixing with the plasmid DNA (final concentration 0.1-0.25 μg/ml). The competent cells and plasmid DNA mixture was incubated at 40° C. for 5 min, transferred into 0.2 cm width and electroporated using a single-pulse electroporation (10 kV/cm, 600 Ω, 25 μF and 3-5 ms pulse time). The pulsed cells were regenerated at 30° C. for 4 h (DSM 44193) and 6 h (DSM 43205) in the presence of 600 μl NB. Transformants were selected after cultivation for 3-4 days at 30° C. on NB-agar plate containing kanamycin (75 μg/ml). As shown in FIG. 12, the plasmids pSeqCO1 and pVer1confer resistance to kanamycin (75 μg/ml) in transformed R. opacus strains (44193 and 43205). Untransformed R. opacus strains (44193 and 43205) (NC) were sensitive to the concentration described above.

Transformation of genus cupriavidus was carried out using the plasmids pSeqCO2 (FIG. 12) as described below.

Cupriavidus necator (DSM531) competent cells were prepared by incubating a single colony in 5 ml NR medium (10 g/1 polypeptone, 10 g/1 yeast extract, 5 g/1 beef extract and 5 g/1 ammonium sulfate; pH 7.0) at 30° C. overnight. The pre-culture was inoculated into 100 ml of fresh NR medium and incubated to a cell density of O.D₆₀₀=0.8. Cells were collected by centrifugation at 3,000×g for 10 min at 4° C. and washed 3 times with 50 ml (each) of sterile ice-cold ddH₂O. The collected cells were re-suspended in 400 μl of 10% (v/v) sterile glycerol in sterile ice-cold ddH₂O and stored in 50 μl aliquots at −70° C.

For electroporation, the competent cells were thawed on ice, transferred into 0.2 cm width of ice cold cuvette and gently mixed with 1 μg of plasmid DNA. Cells were electroporated using a single-pulse electroporation (11.5 kV/cm, 25 μF and 5 ms pulse time). The pulsed cells were transferred into 1 ml of fresh NR medium and culture for 2 h at 30° C. Transformants were selected after cultivation for 48 h at 30° C. on NR-agar plate containing kanamycin (200 μg/ml). As shown in FIG. 12, the plasmid pSeqCO2 confers resistance to kanamycin (200 μg/ml) in transformed Cupriavidus necator (DSM531). Untransformed Cupriavidus necator (DSM531) cells (NC) were sensitive to the concentration described above.

### Inoculation and Growth Conditions

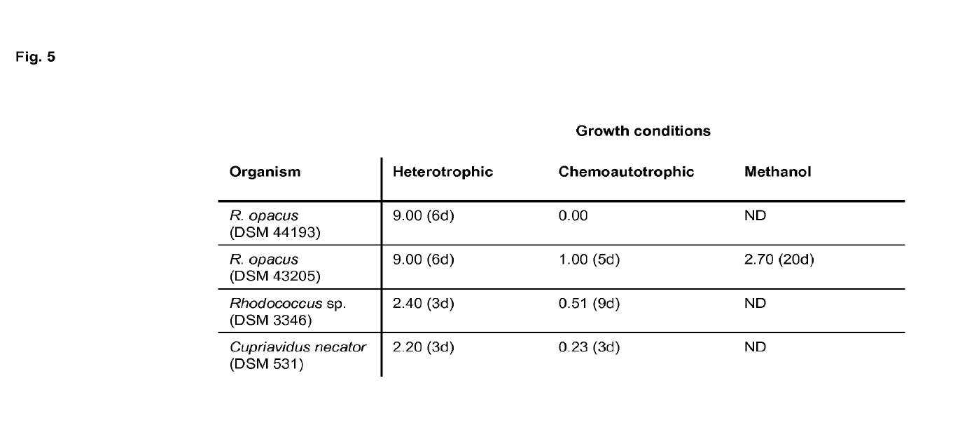

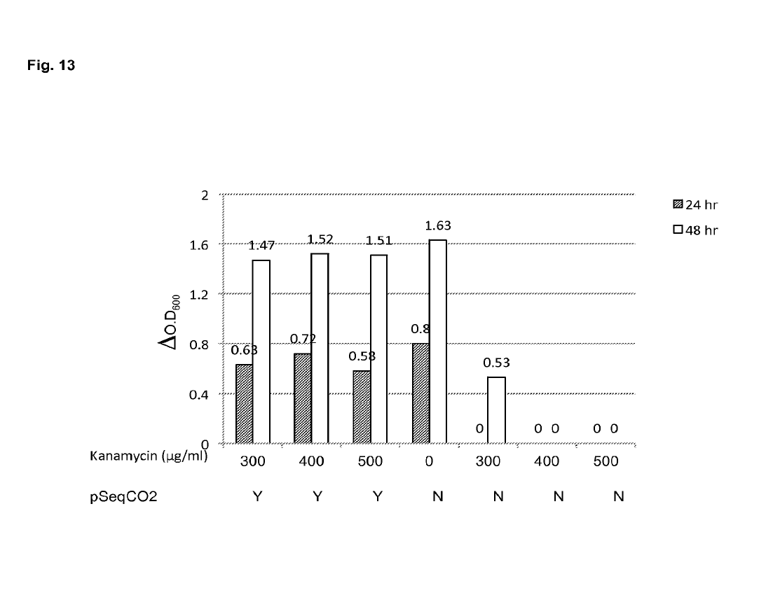

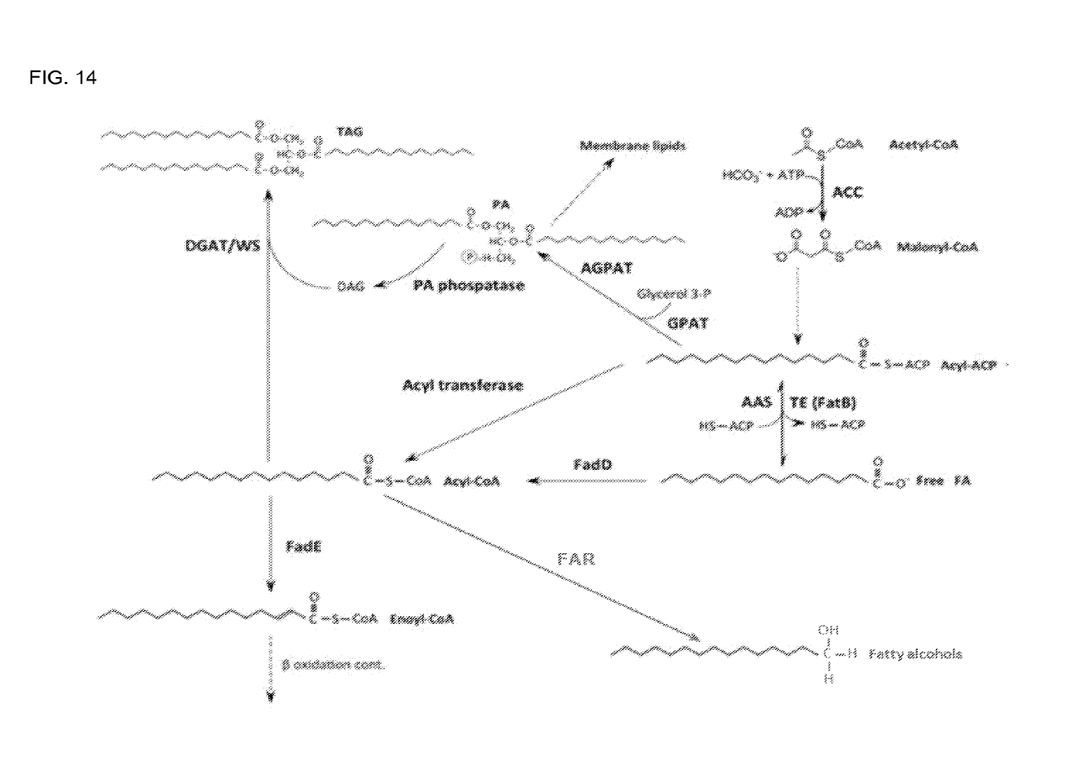

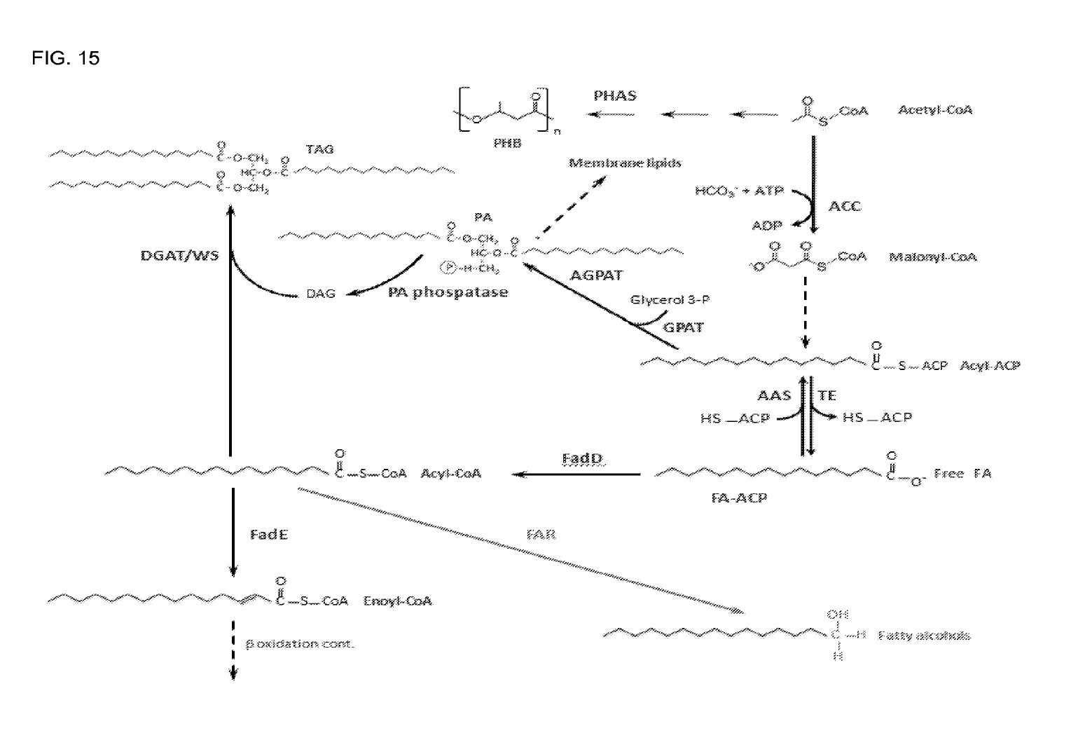

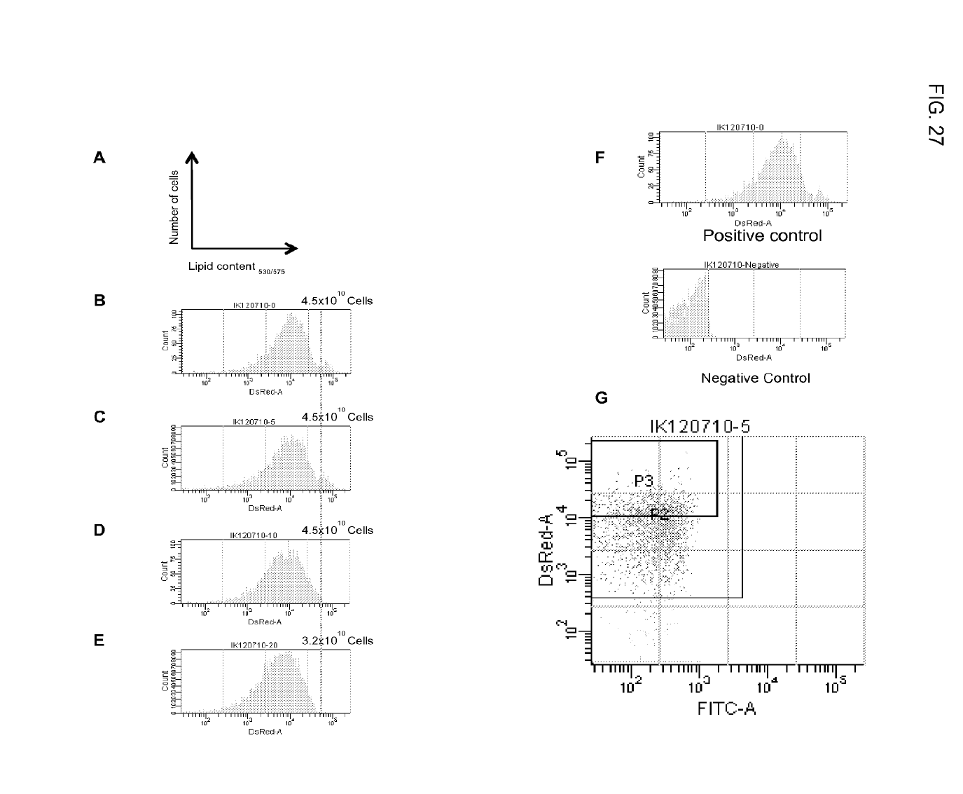

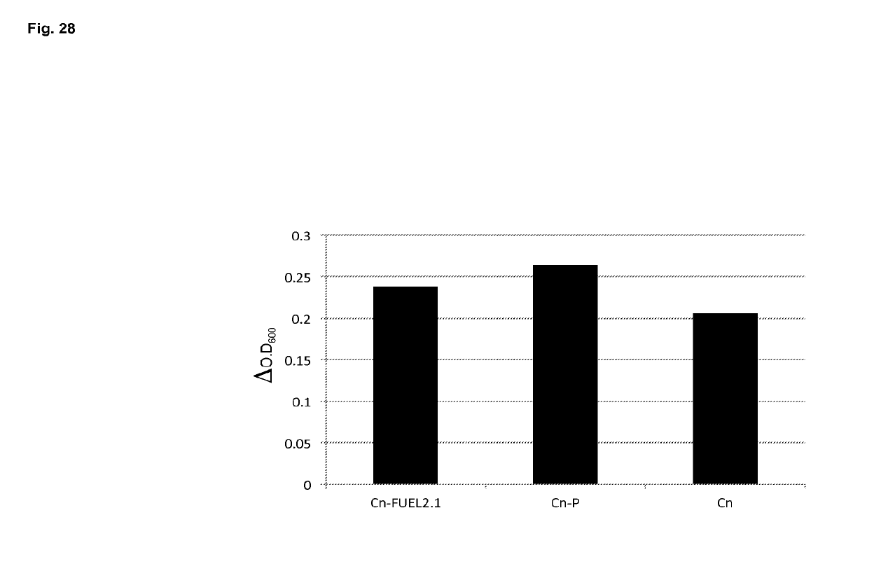

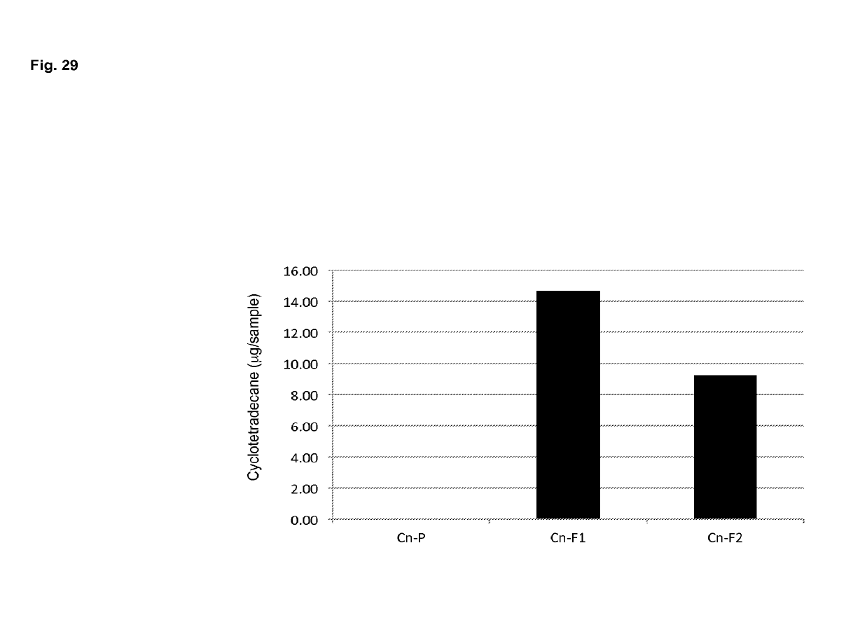

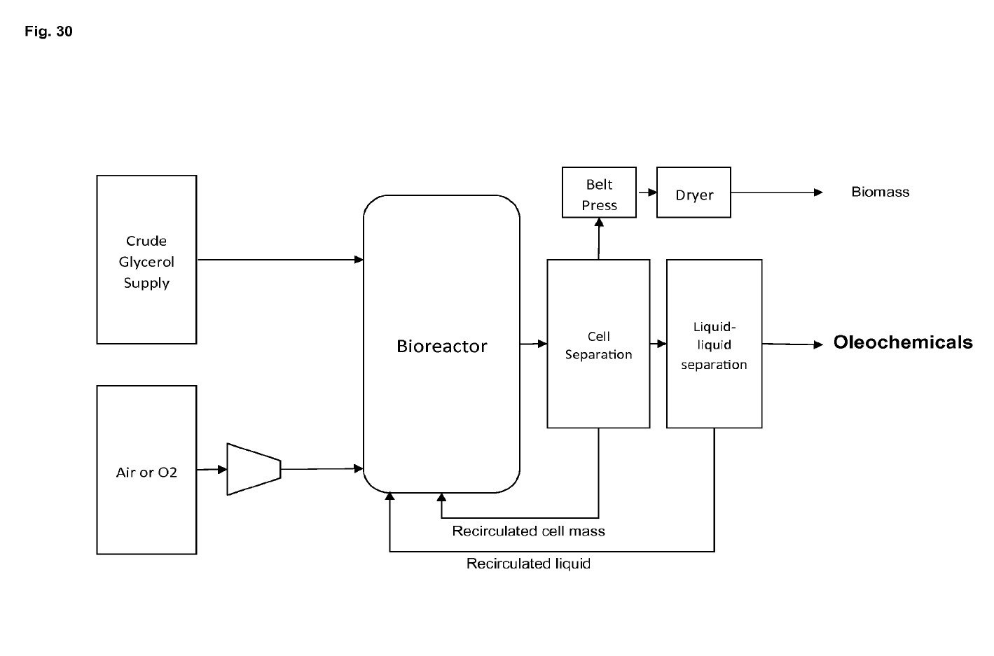

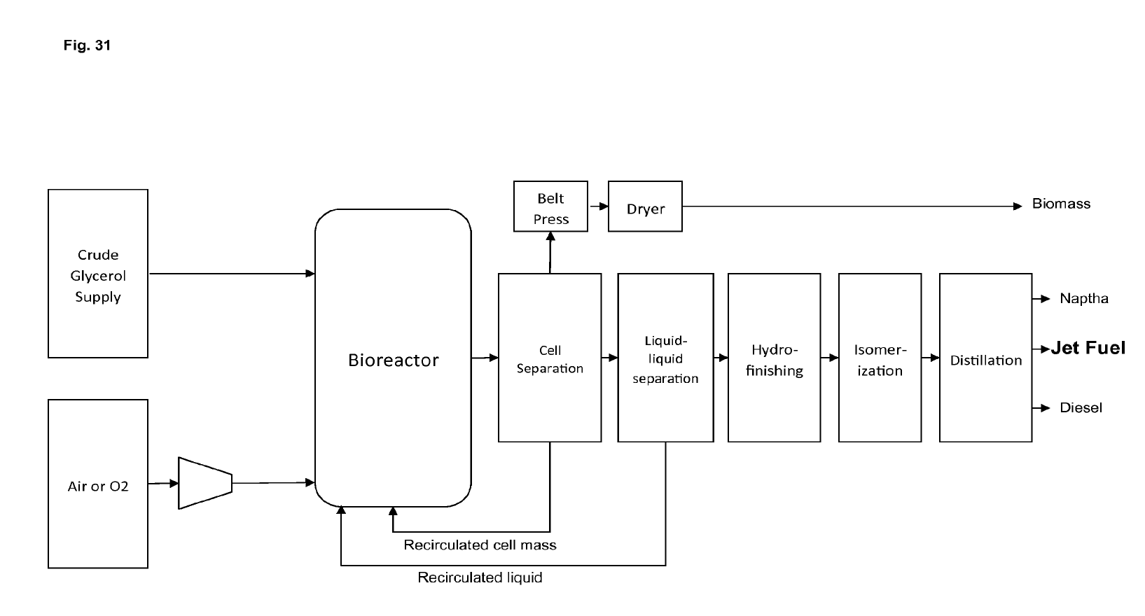

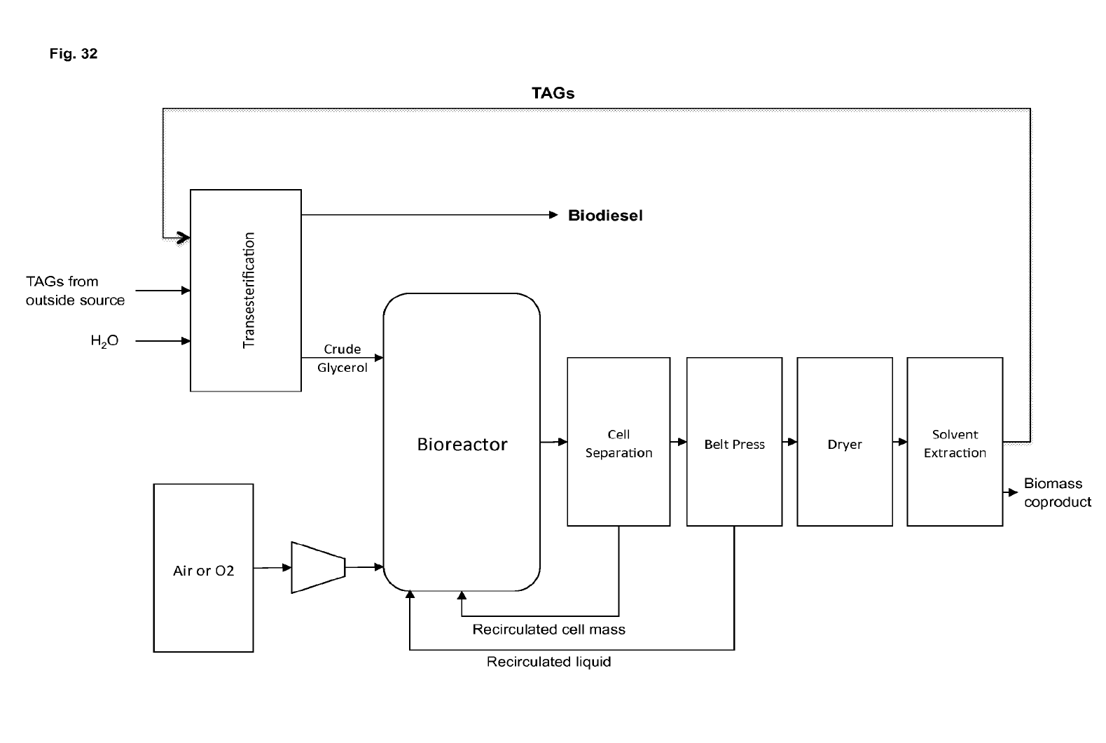

Knallgas microorganisms from the genus rhodococcus and from the genus cupriavidus were tested for their ability to grow on different carbon sources. Colonies from strains grown on LB agar plates at 30° C. were transferred into flasks containing 10% (v/v) of the indicated media for 3-20 days at 30° C. and 250 rpm. R. opacus strain DSM 44193 exhibited growth only under heterotrophic growth conditions as measured by optical density (OD) at 650 nm on MSM medium (1 L Medium A: 9 g Na₂HPO₄12H₂O, 1.5 g H₂PO₄, 1.0 g NH₄Cl and 0.2 g MgSO₄.7H₂O per 1 L; 10 ml Medium B: 50 mg Ferric ammonium citrate and 100 mg CaCl₂ per 100 ml; 10 ml Medium C: 5 g NaHCO₃ per 100 ml; and 1 ml Trace Mineral Solution: 100 mg ZnSO₄.7H₂O, 30 mg MnCl₂. 4H₂O, 300 mg H₃BO₃, 200 mg COCL₂.6H₂O, 10 mg CuCl₂.2H₂O, 20 mg NiCl₂.6H₂O and 30 mg Na₂MoO₄.2H₂O per 1 L) supplemented with 40 g/L glucose. R. opacus strain DSM 43205 showed identical growth rates under heterotrophic conditions reaching O.D=9.0. Strain DSM 43205 was also able to grow on chemoautotrophic conditions (MSM medium supplemented with 66.7% H₂, 9.5% CO₂, 5% O₂ and 18.8% N₂) and heterotrophically on a single carbon compound as the solely carbon source (MSM medium supplemented with 25 g/l methanol). Rhodococcus sp. (DSM 3346) exhibited growth under heterotrophic conditions and chemoautotrophic conditions (DSMZ Medium 81:1 L of Mineral Medium for chemolithotrophic growth: 2.9 g Na₂HPO₄.2H₂O, 2.3 g KH₂PO₄, 1.0 g NH₄Cl, 0.5 g MgSO₄.7H₂O, 0.5 g NaHCO₃, 0.01 g CaCl.2H₂O and 0.05 g Fe(NH₄) citrate per 1 L; and 5 ml Trace Mineral Solution, supplemented with 80% H₂, 10% CO₂ and 10% O₂). Cupriavidus necator (DSM 531) was able to grow under heterotrophic and chemoautotrophic conditions (media described for Strain DSM 43205) (FIG. 5 and FIG. 28). Cupriavidus necator (DSM 531) transformed with pSeqCO2 was able to grow on LB media supplemented with 300 400 and 500 μg/ml kanamycin exhibiting O.D₆₀₀ of 1.47, 1.52 and 1.51 respectively (FIG. 13). Untransformed cells exhibited growth on control (LB only) and some growth on 300 μg/ml kanamycin while no growth was detected on 400 and 500 μg/ml kanamycin.

### Lipid Profiles

### Production of Fatty Acid

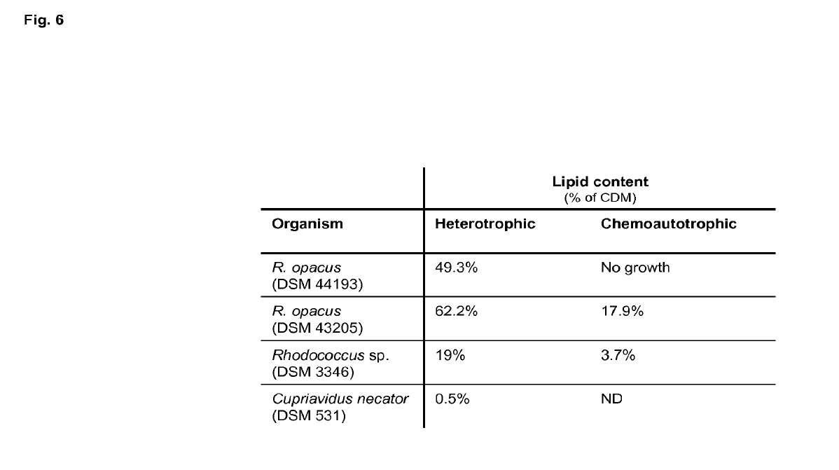

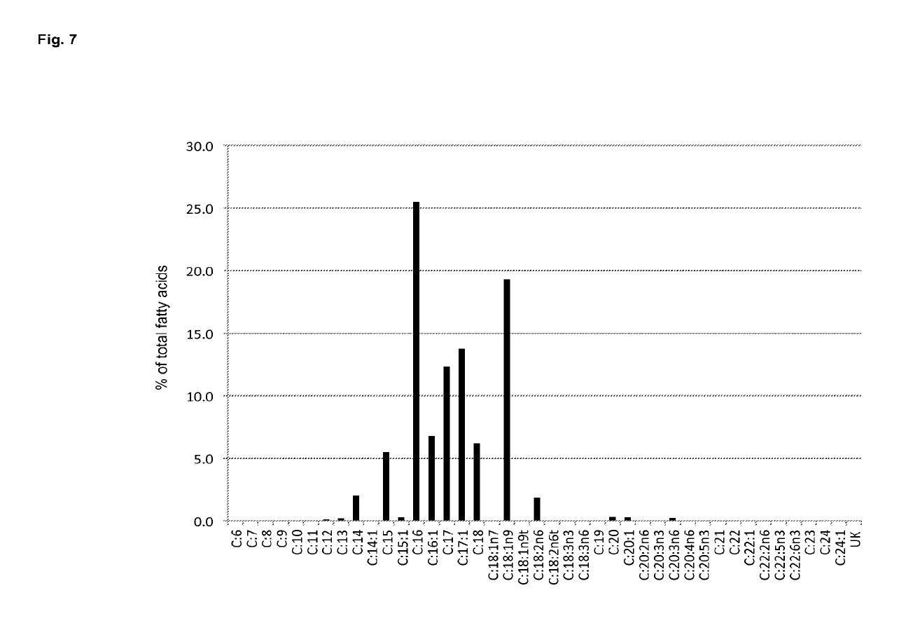

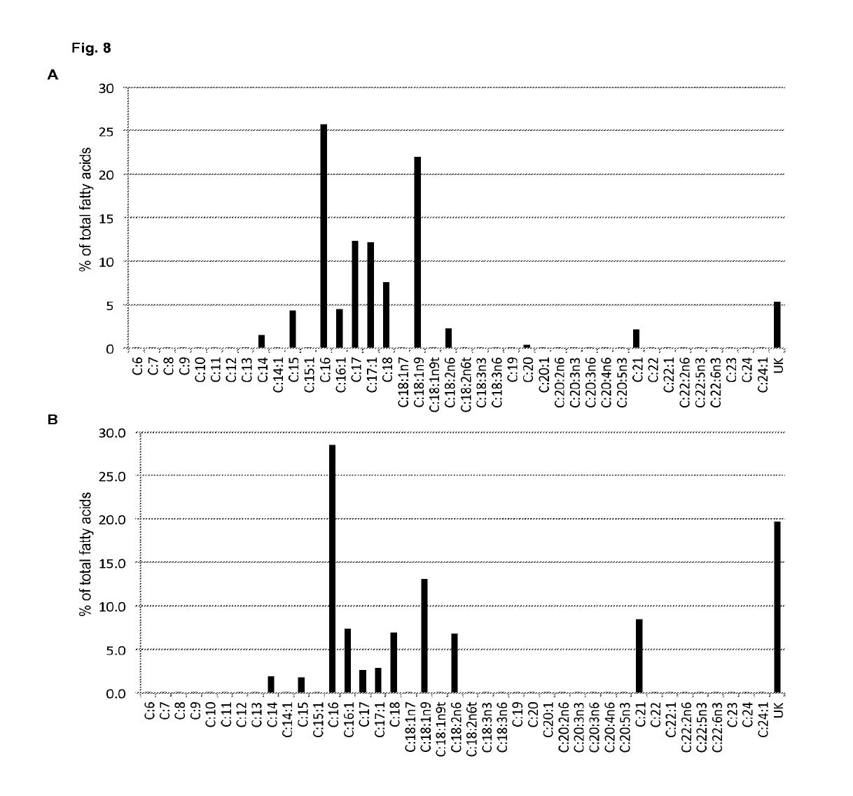

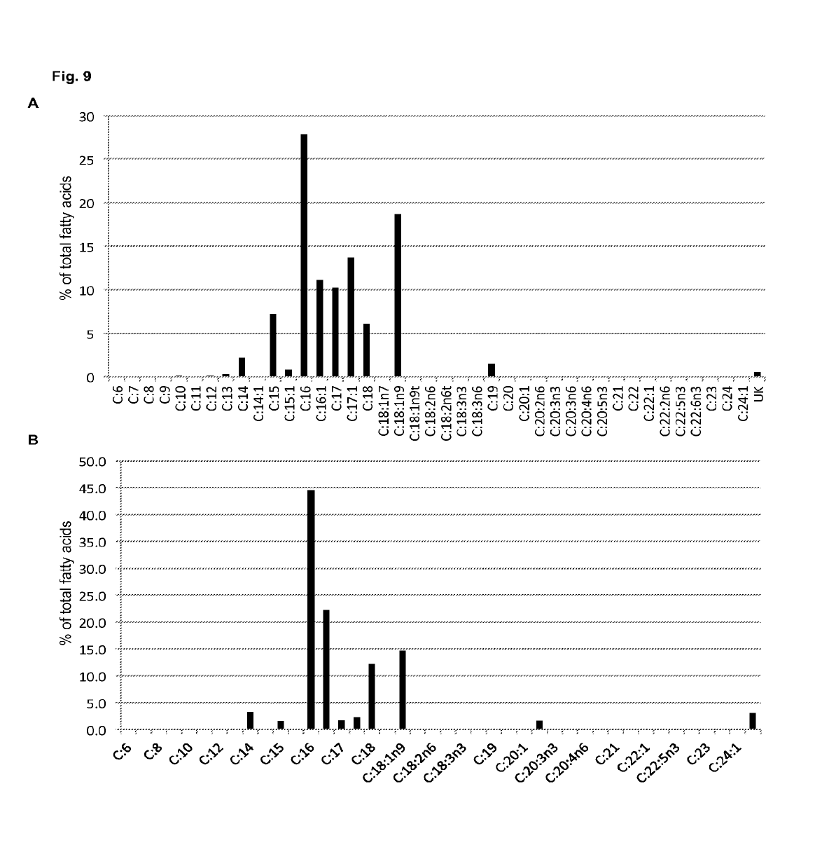

Under heterotrophic growth conditions strains DSM 44193, DSM 43205, DSM 3346 and DSM 531 produce lipid (FIG. 6). Lipid content determined by gas chromatography analysis of cells harvested after 72 hr (unless otherwise indicated) showed over 19% of cellular dry matter (CDM) determined gravimetrically for strains DSM 44193, DSM 43205 and DSM 3346. The lipid content of DSM 43205 reached almost 18% under chemoautotrophic conditions. Under heterotrophic growth conditions DSM 44193 produces 32%, 26% and 21% of 16, 17 and 18-carbon fatty acid respectively (FIG. 7). DSM43205 produces similar amounts of 16, 17 and 18-carbon fatty acid (30%, 24% and 32% respectively) (FIG. 8A). Chemoautotrophic growth condition significantly reduces the 17-carbon fatty acid abundance (6%) and maintains similar levels of 16 and 18-carbon fatty acid (36% and 27% respectively) (FIG. 8B). DSM3346 exhibits similar fatty acid distribution of 16, 17 and 18-carbon fatty acid (39%, 24% and 25% respectively) (FIG. 9A) under heterotrophic growth. Chemoautotrophic growth condition significantly increases the 16-carbon fatty acid levels (66%) and reduces the 17 and 18-carbon fatty acid levels (4%, 14%)(FIG. 9B).

### Production of Alkanes

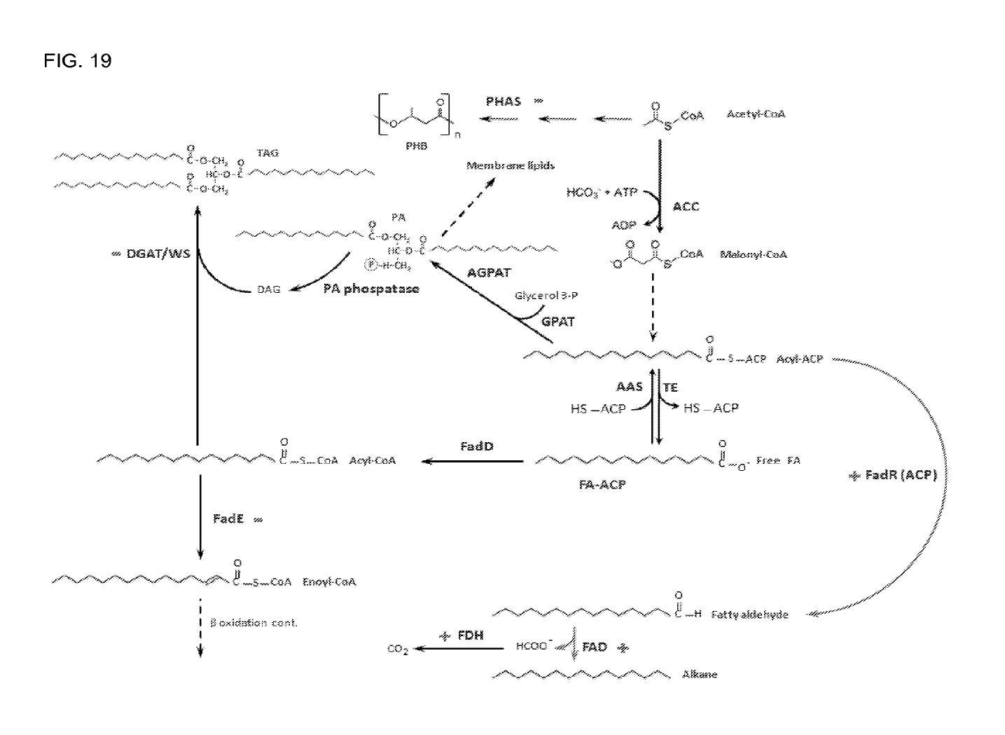

To redirect carbon flux from fatty acid toward alkanes biosynthesis, the genes Fatty acyl-CoA/Fatty acyl-ACP reductase (FadR) and Fatty aldehyde decarbonylase (FAD) from the decarbonylation pathway of cyanobacteria (indicated in red) were expressed in Cupriavidus necator (DSM 531) (FIG. 19).

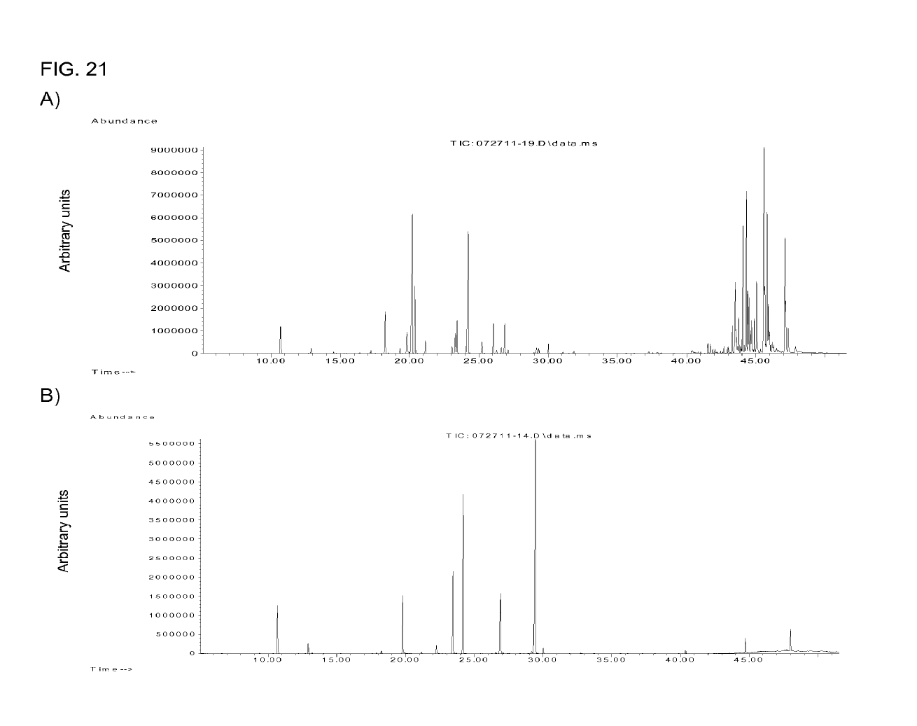

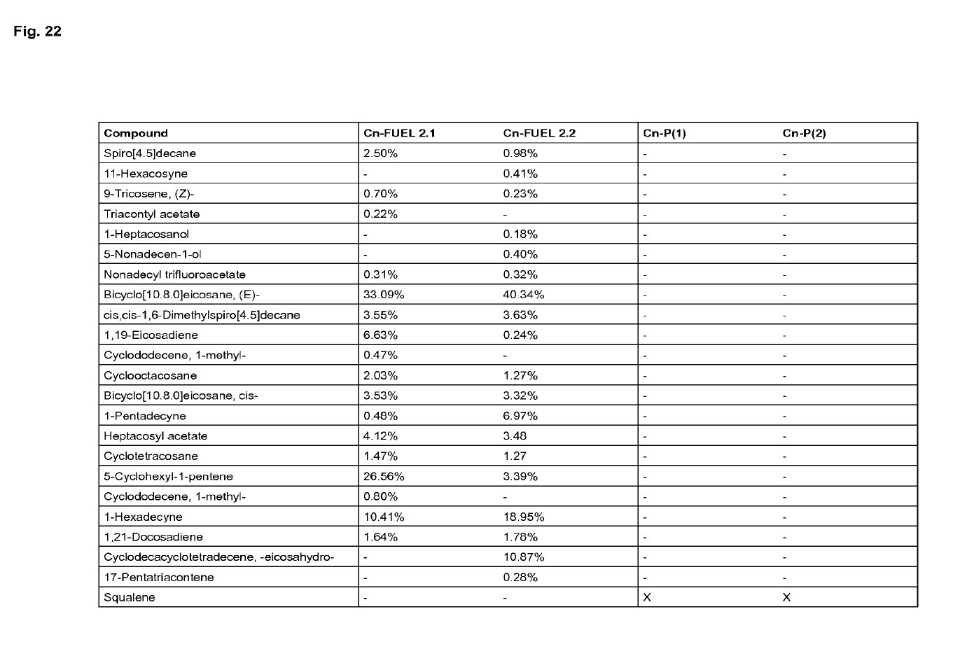

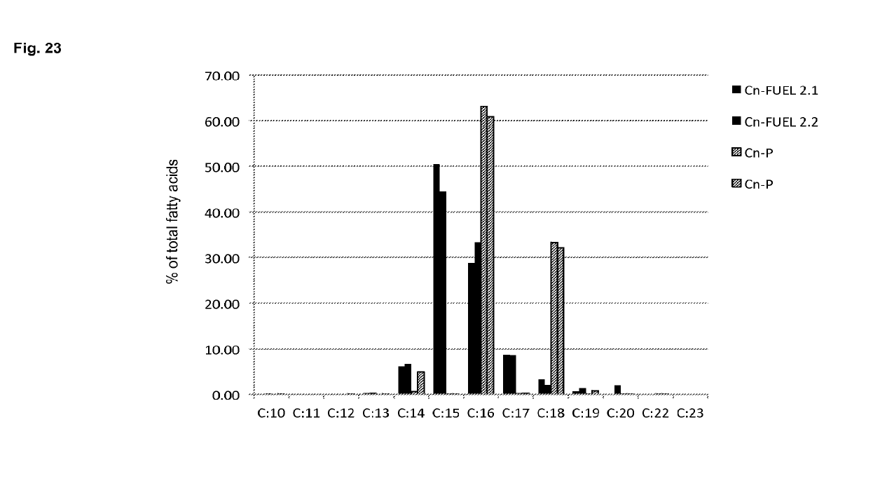

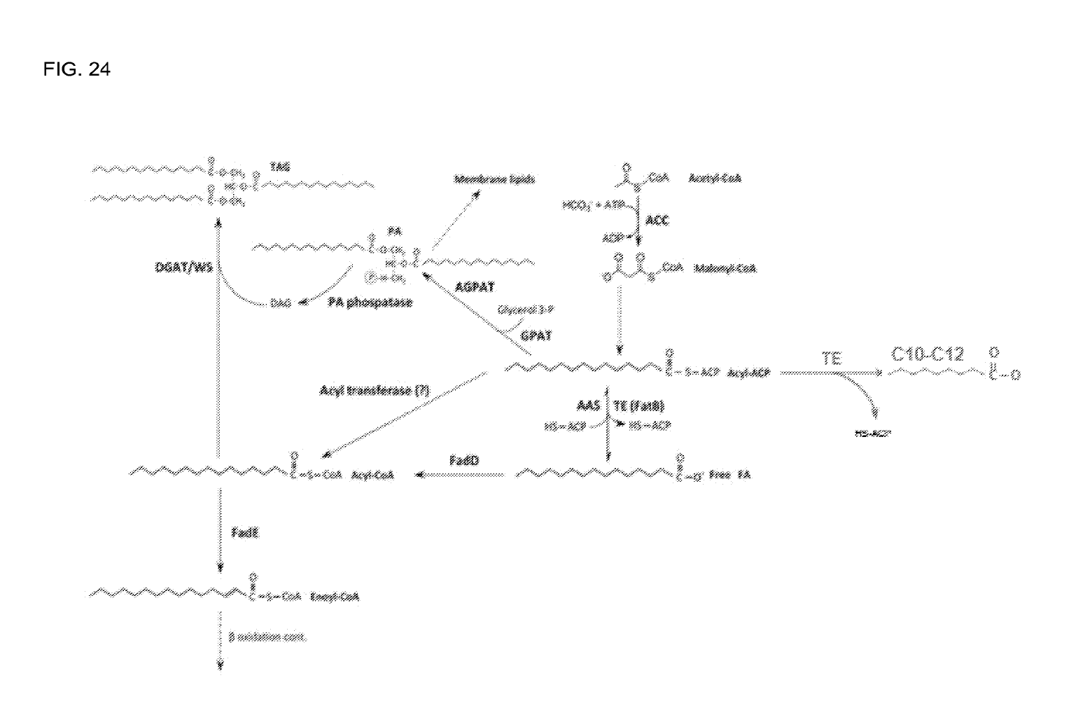

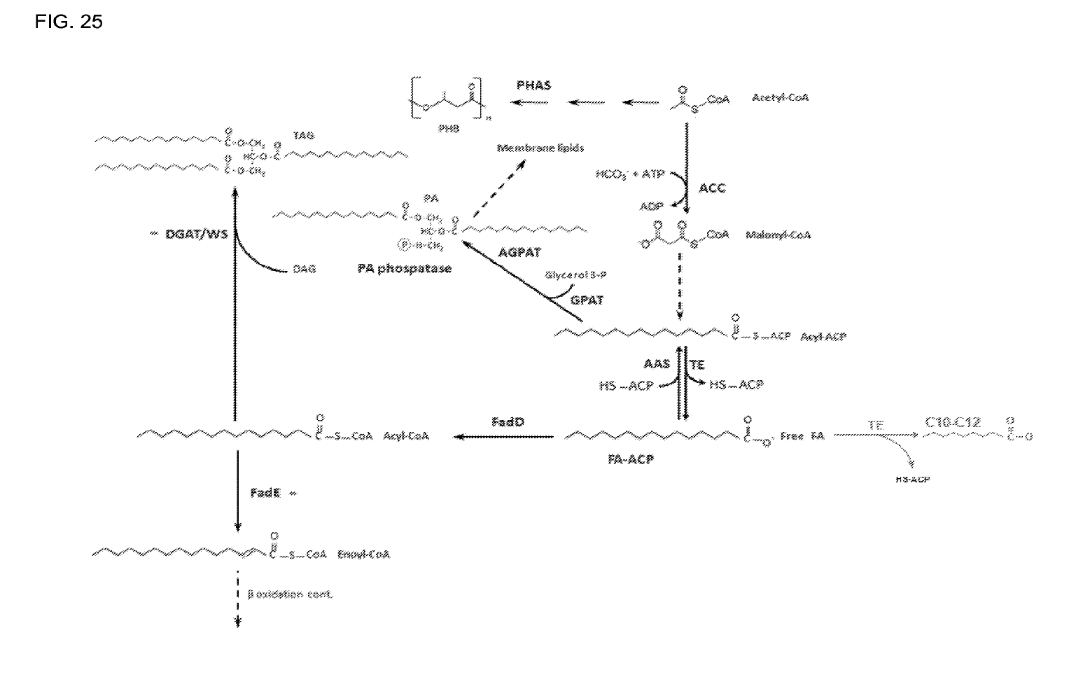

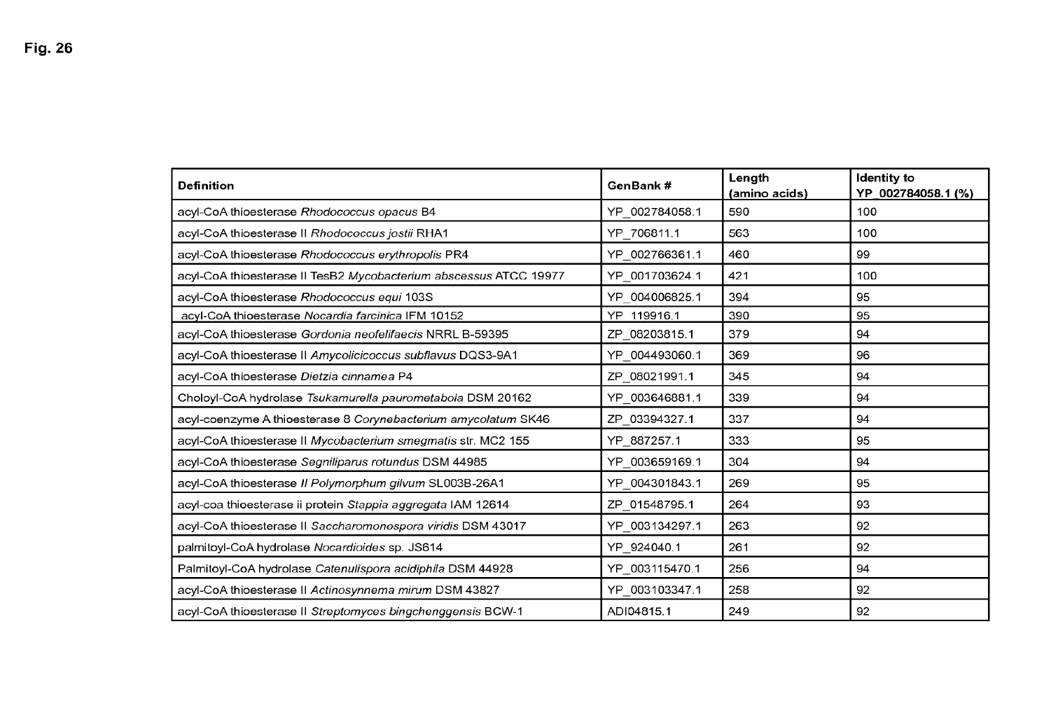

The plasmid pSeqCO2::FUEL (FIG. 20) described in the text was introduced into Cupriavidus necator (DSM 531) as described above and 2 independent transformants (Cn-FUEL2.1 and Cn-FUEL2.2) were selected. One hundred ml of Cn-FUEL2.1, Cn-FUEL2.2 and control cells (empty plasmid: Cn-P) were incubated on LB medium with 400 μg/ml kanamycin for 30 hr. Cells were harvested at 3,000×g for 10 min at 4° C. and pellet was analyzed by GC/MS. Cn-FUEL2.1 (FIG. 21A) and Cn-FUEL2.2 showed a specific peak at 45.00 min compared to control Cn-P (FIG. 21B) indicating the presence of alkanes in the engineered strains. Cn-FUEL2.1, Cn-FUEL2.2 produced high levels (over 2%) of unique molecules such as: Spiro[4.5]decane, Bicyclo[10.8.0]eicosane, cis,cis-1,6-Dimethylspiro[4.5]decane, 1,19-Eicosadiene, Cyclooctacosane, Bicyclo[10.8.0]eicosane, 1-Pentadecyne, Heptacosyl acetate, 5-Cyclohexyl-1-pentene, 1-Hexadecyne and Cyclodecacyclotetradecene, -eicosahydro (FIG. 22).

The effect of the production of alkanes on fatty acid distribution is shown in FIG. 23. The fatty acids profile of 2 independent control experiments (Cn-P) shows predominantly 16-carbon (63% and 61%) and 18-carbon (33% and 32%) fatty acids. In contrast, Cn-FUEL2.1 and Cn-FUEL2.2 exhibit significantly lower levels of 16-carbon (29%, 33% respectively) and 18-carbon (3% and 2% respectively) fatty acids. Cn-FUEL2.1 and Cn-FUEL2.2 show a significant increase in the 15-carbon fatty acid (50% and 45% respectively) compared to 0.08% and 0.09% in the control strains Cn-P.

The formation of alkanes in Cupriavidus necator was demonstrated by the expression of fatty acyl-CoA reductases (FAR) genes. The Arabidopsis genes FAR1 (SEQ ID: 05) and FAR2 (SEQ ID: 06) and FAR3 (SEQ ID: 07) were cloned into pSeqCO2 plasmid using the indicated restriction sites to give pSeqCO2::FAR1 and pSeqCO2::FAR2 respectively (FIG. 16). pSeqCO2::FAR1 and pSeqCO2::FAR2 and control (pSeqCO2, empty plasmid) were introduced into Cupriavidus necator (DSM 531) as described in the text. One hundred ml of transformants of pSeqCO2::FAR1 (Cn-F1), pSeqCO2::FAR2 (Cn-F2) and control cells (empty plasmid: Cn-P) were incubated on LB medium with 400 μg/ml kanamycin for 30 hr. Cells were harvested at 3,000×g for 10 min at 4° C. and pellet was analyzed by GC. Cn-F1 and Cn-F2 produced cyclotetradecane compared to control Cn-P (FIG. 29) indicating the presence of alkanes in the engineered strains. It is believed, without the present invention being limited to any particular theory, that cyclotetradecane is produced within Cupriavidus necator from a C14 fatty alcohol intermediate, that results from the introduction and expression of the FAR gene in Cupriavidus necator. The absence of cyclotetradecane in Cn-P is thought to be due to the lack of FAR gene and hence lack of C14 fatty alcohol intermediate in Cupriavidus necator, without the present invention being limited to any particular theory.

### Purification

### Purification Alkanes

To produce alkanes in bacteria, genes from the decarbonylation pathway of cyanobacteria, including but not limited to, the FadR (SEQ ID: 08) and FAD (SEQ ID: 09) genes will be cloned into pVer2 (SEQ ID: 04) to give pVer2::FUEL. Bacteria, including but not limited to, R. opacus strain (DSM43205) will be transformed with the plasmid pVer2::FUEL by electroporation and grown in 100 ml LB medium supplemented with 75 μg/ml kanamycin for 30 hr. The cells (2×50 ml) will be harvested at 3,000×g for 10 min at 4° C. and the pellet and the supernatant further analyzed. Analysis of alkanes from the cell pellet will be carried out in 25 mm×150 mm glass tube in the presence of 50 μL of Eicosane standard (approx 200 μg/ml) and 50 μl lipid standard (˜200 μg/ml). Pellet will be extracted with 5 mL chloroform, 10 ml methanol, 4 ml phosphate buffer (phosphate buffer reagent: 50 mM, pH 7.4, 8.7 g K₂HPO₄ in 1 L water, and about 2.5 ml 6N HCl to adjust pH=7.4, and 50 ml chloroform per 1 L buffer). The mixture will be vortexed for 30 sec, sonicated for 2 min and incubated in dark for at least 3 hr. Phases will be separated in the presence of 5 mL chloroform and 5 ml ddH₂O, vortexed and spun down 2000 rpm for 1 min. The bottom layer will be transferred with a glass Pasteur pipette to clean 16 mm×125 mm glass tube with Teflon-lined screw top and dried under N2. The dried extract will be re-suspended in hexane and analyzed by Gas Chromatography for the presence of alkanes, including but not limited to 1-Hexadecyne.

### Purification of Fatty Alcohols

To produce fatty alcohols in bacteria, the fatty acyl-CoA reductases (FARs) that catalyze the formation of a fatty alcohol from an acyl-CoA, including but not limited to the FAR1 gene (SEQ ID: 05) will be cloned into pVer2 (SEQ ID: 04) to give pVer2::FAR1. Bacteria including but not limited to R. opacus strain (DSM43205) will be transformed with the plasmid pVer2::FAR1 by electroporation, grown in 100 ml LB medium supplemented with 75 μg/ml kanamycin for 30 hr. The cells (2×50 ml) will be harvested at 3,000×g for 10 min at 4° C. and the pellet and the supernatant further analyzed. Analysis of fatty alcohols from the cell pellet will be carried out in 1.5 ml eppendorf tube in the presence of 50 μl pure HCl and 500 μl ethyl acetate (EtAc). The mixture will be vortexed for 10 sec and spun down at max speed for 1 min. The EtAc (top) layer will be recovered and transferred to a glass GC vial. The sample will be derivatized by adding 100μl of MeOH:HCl (9:1) to the EtAc extract and mixing. About 50-1000 of TMS-diazomethane (2M in hexanes) will be mixed and incubated for 10-15 min. Aliquots of 50μ will be analyzed by Gas Chromatography—Flame Ionization Detector (GC-FID) for the presence of fatty alcohols including but not limited to 1-tetradecanol.

### Purification of Fatty Acids

To modify the fatty acid distribution in bacteria, thioesterases that regulate the fatty acid chain length, including but not limited to the YP_{—}002784058.1 gene will be cloned into pVer2 (SEQ ID: 04) to give pVer2::TE. Bacteria, including but not limited to, R. opacus strain (DSM43205) will be transformed with the plasmid pVer2::TE by electroporation and grown in 100 ml LB medium supplemented with 75 μg/ml kanamycin for 30 hr. The cells (2×50 ml) will be harvested at 3,000×g for 10 min at 4° C. and the pellet and the supernatant further analyzed. Analysis of fatty acids from the cell pellet will be carried out in 25 mm×150 mm glass tube in the presence of 50 μL of Eico sane standard (approx 200 μg/mL) and 50 μL lipid standard (˜200 μg/ml). Pellet will be extracted with 5 ml chloroform, 10 ml methanol, 4 ml phosphate buffer (phosphate buffer reagent: 50 mM, pH 7.4, 8.7 g K₂HPO₄ in 1 L water, and about 2.5 mL 6N HCl to adjust pH=7.4, and 50 ml chloroform per 1 L buffer). The mixture will be vortexed for 30 sec, sonicated for 2 min and incubated in dark for at least 3 hr. Phases will be separated in the presence of 5 ml chloroform and 5 ml ddH₂O, vortexed and spun down 2000 rpm for 1 min. The bottom layer will be transferred with a glass Pasteur pipette to clean 16 mm×125 mm glass tube with Teflon-lined screw top and dried under N2. The dried extract will be re-suspended 1.5 ml of a 10:1:1 mixture of Methanol:CHC13:concentrated HCl, vortexed and incubated in 60° C. for 14-16 hr (overnight). The extracts will be cooled and 2 ml of ddH₂O and 2 ml of hexane will be added, vortexed and centrifuged for 5 min at 2000 rpm for phase separation. The top hexane layer will be transferred to clean 16 mm tube additional two hexane extraction (vortex, centrifugation and phase separation) will be carried out in the extract tube. The hexane extracts will be dried in a GC vial and analyzed by Gas Chromatography for the presence of fatty acids, including but not limited to dodecanoic acid.

### Production of Fatty Acids, Hydroxy-Fatty Acids, Unsaturated Fatty Acids, Fatty Alcohols, Straight Chain Alkanes, Cyclic Alkanes, and Unsaturated Hydrocarbons

The following fatty acids were produced in cultivating of natural microbes and genetically-engineered microbes.

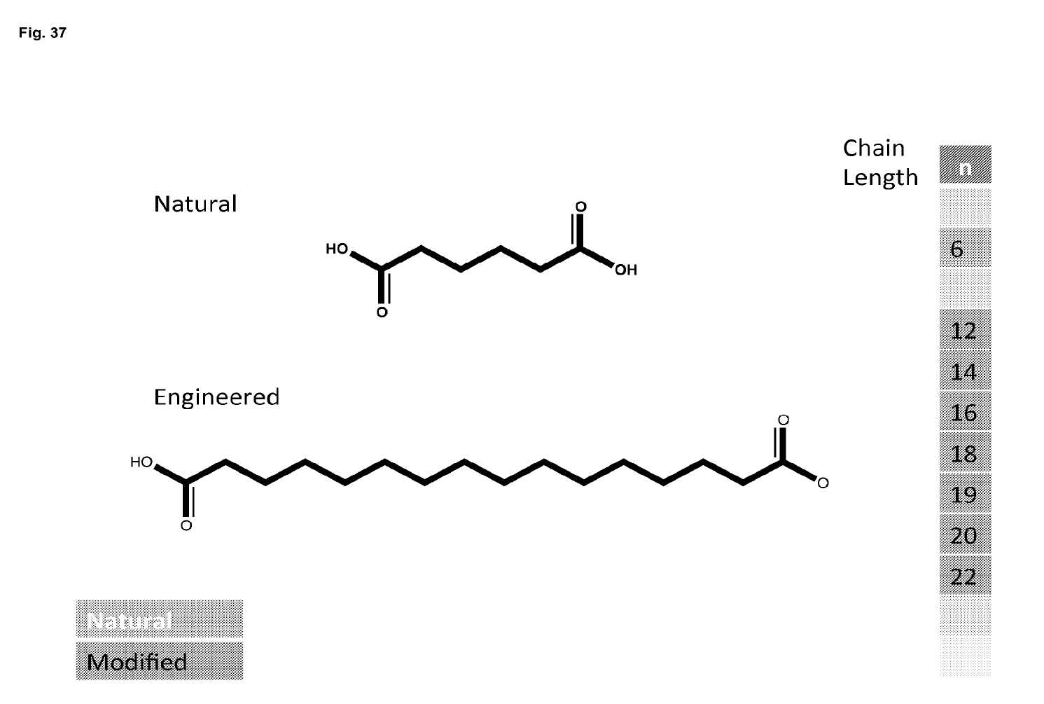

6-Hexanedioic or adipic acid was produced in the natural Cupriavidus necator (DSM 531) strain (See FIG. 37).

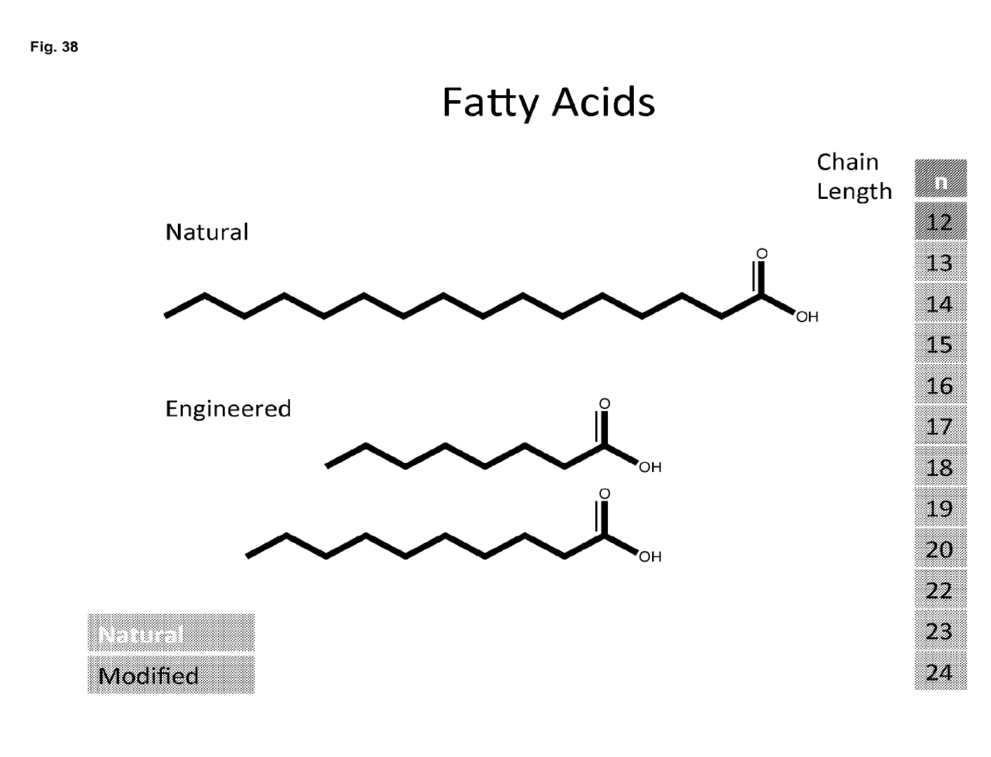

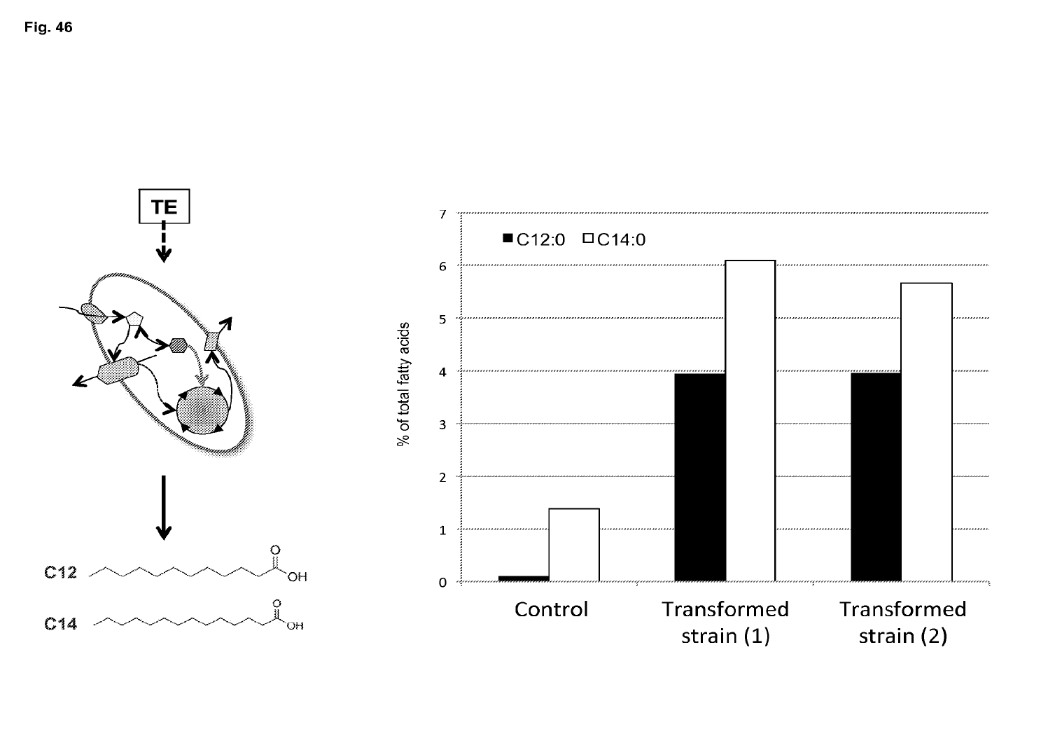

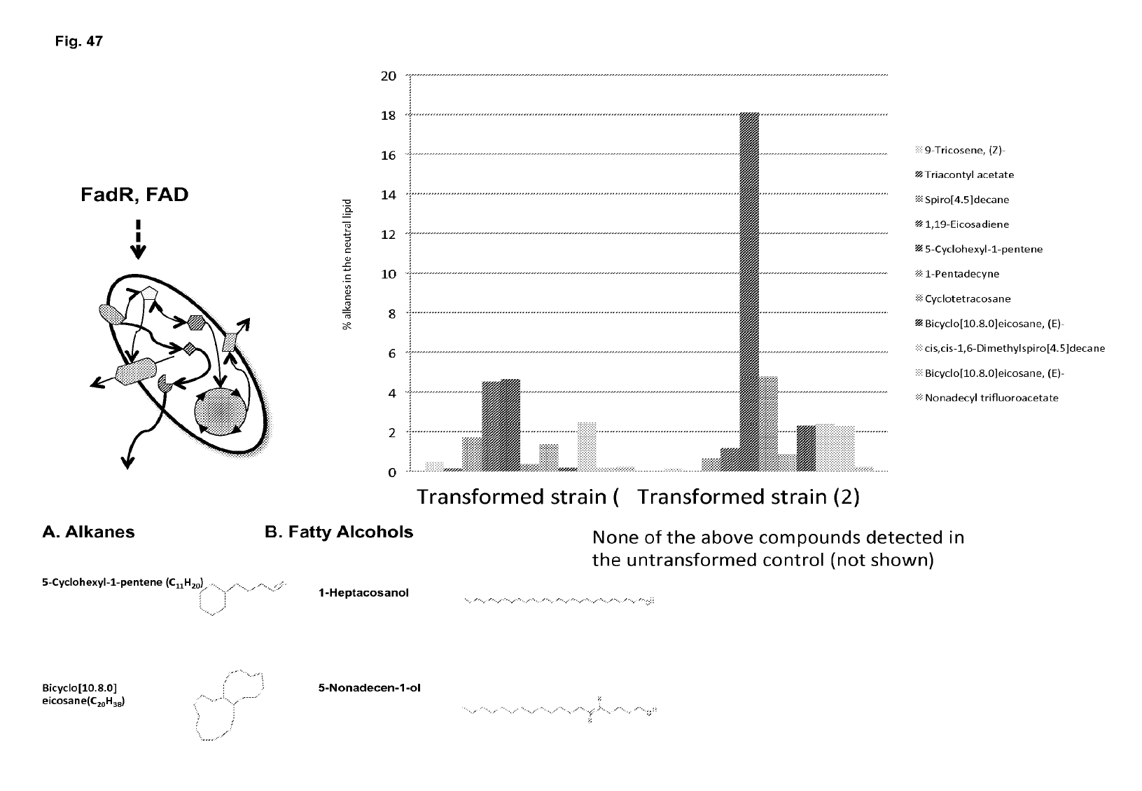

Fatty acids of varying lengths (number of carbons=13, 14, 15, 16, 17, 18, 19, 20, 22, 23, 24) were produced by native Rhodococcus opacus (DSM 43205) and Cupriavidus necator (DSM 531) strains. Introduction of the thioesterase gene, resulted in production of C12:0, not seen in the native strain of Cupriavidus necator (DSM 531). See FIGS. 38 and 46.

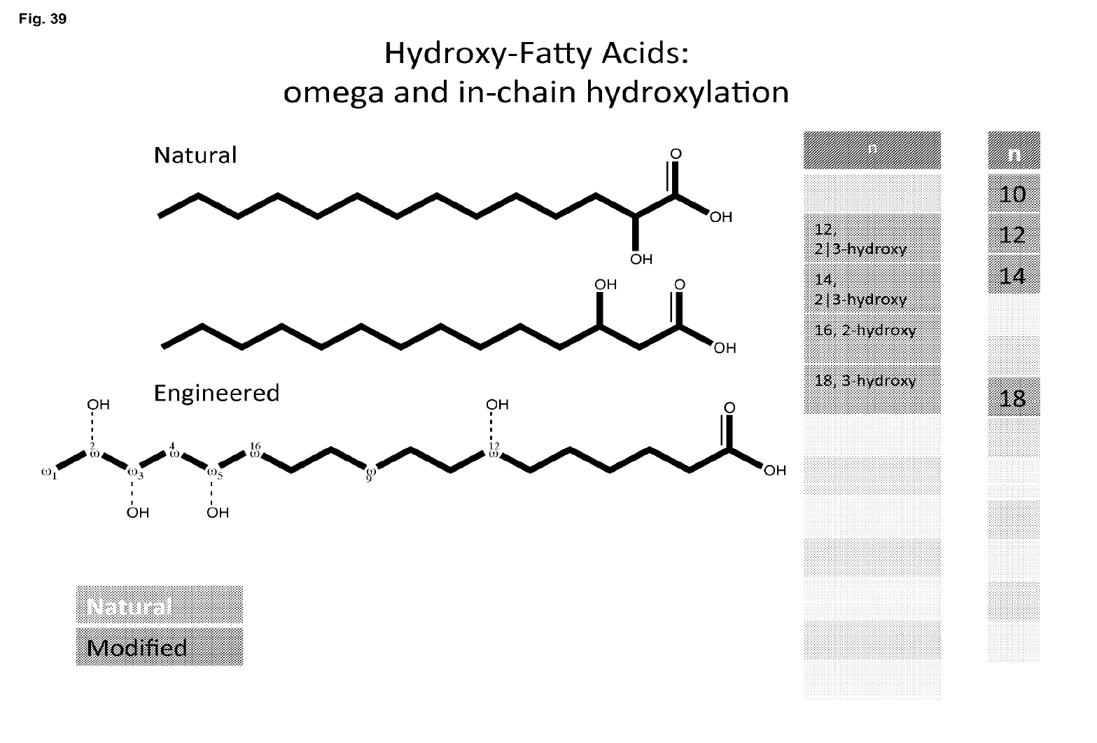

Production of 3-hydroxy-C14 was found in the native Rhodococcus opacus (DSM 43205) and Cupriavidus necator (DSM 531) strains. 3-hydroxy-C18 is produced by native Rhodococcus opacus (DSM 43205) strain. Introduction of the FAR gene into Cupriavidus necator (DSM 531) yielded 3-hydroxy C12 and 2-hydroxy-C14. See FIG. 39.

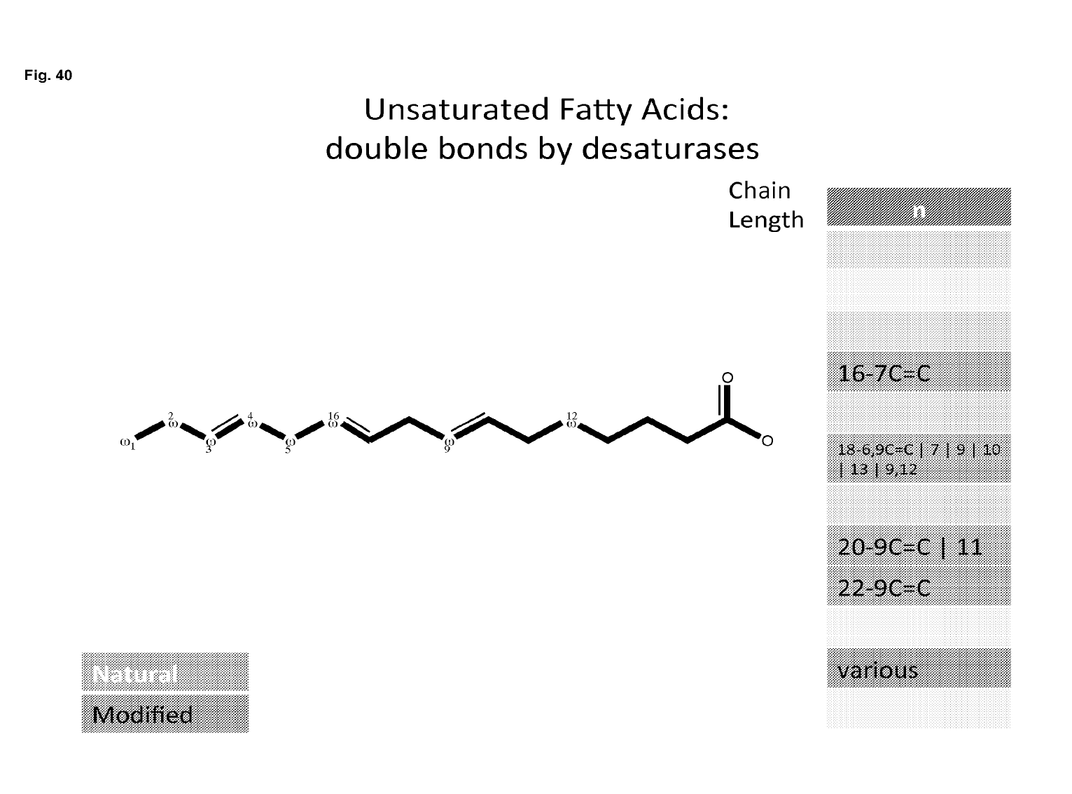

C16, C18, C20, and C22 compound were found to have unsaturated bonds at various positions, including 7, 9, 12 and 13, found in unmodified knallgas strains. See FIG. 40.

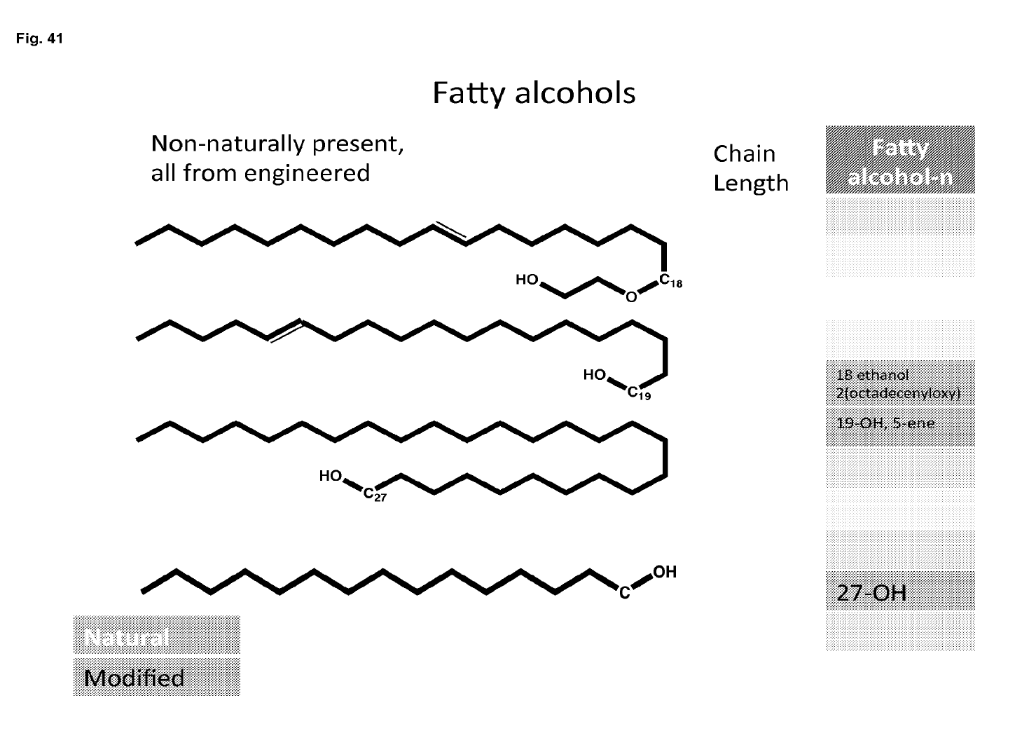

Fatty alcohols were found in FUEL genetically-modified Cupriavidus necator (DSM 531) strains: C18, C19, and C27. See FIG. 41.

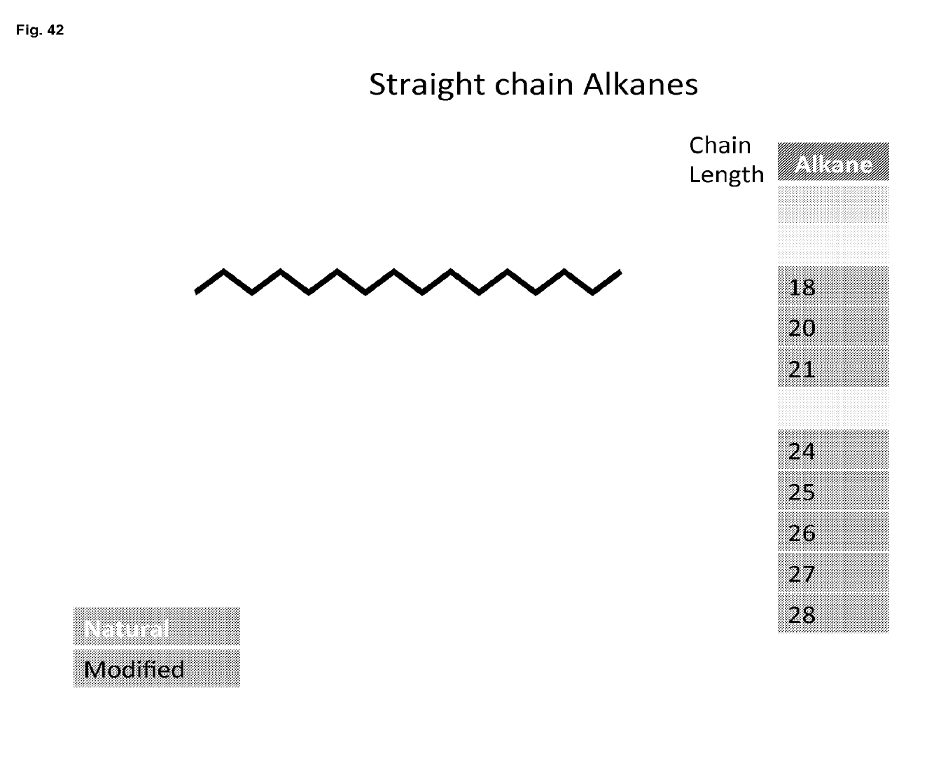

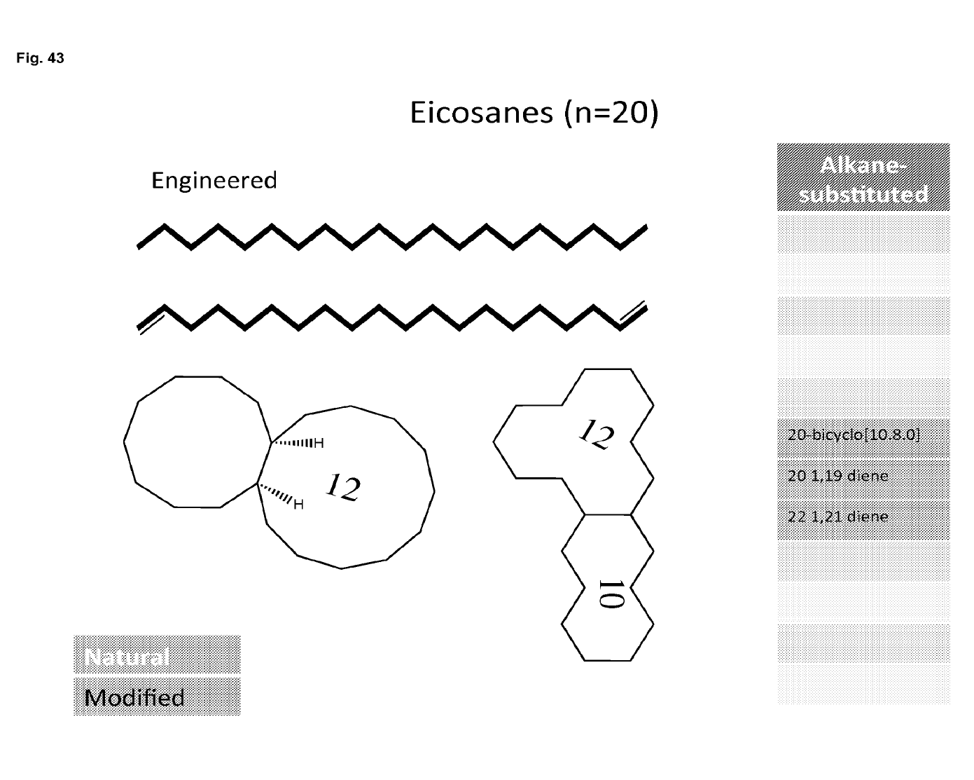

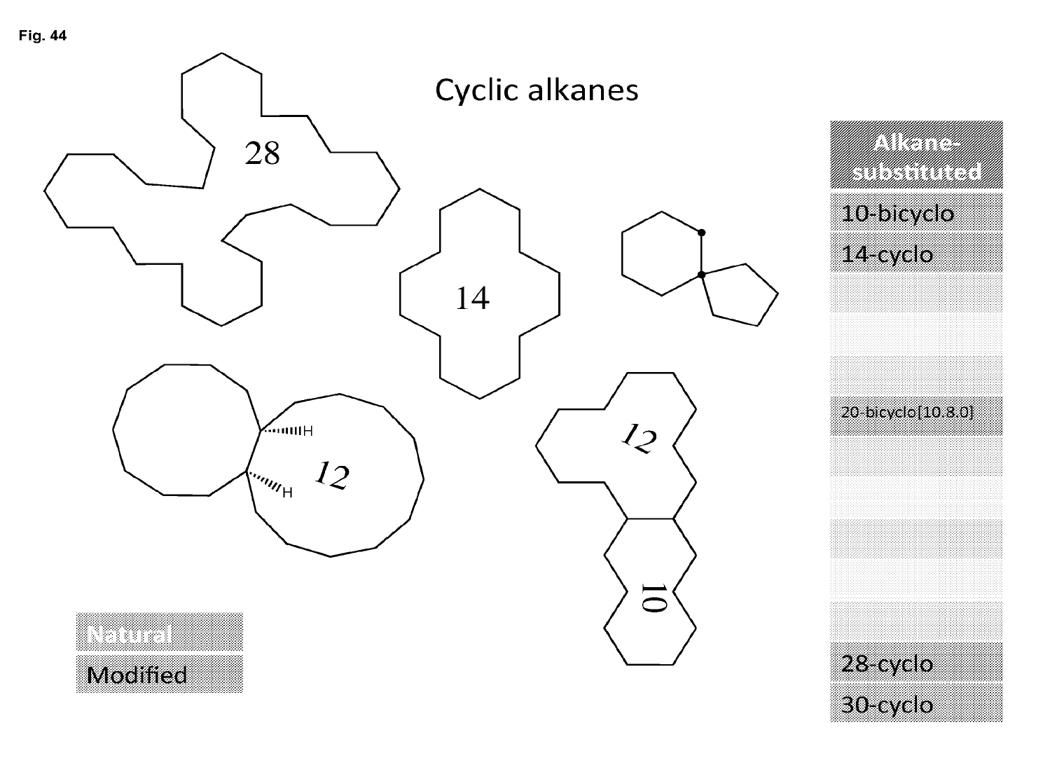

Straight chain alkanes were (carbon number=18, 20, 21, 24, 25, 26, 27, 28) were prevalent in Cupriavidus necator (DSM 531) genetically modified with FUEL genes. These alkanes were not seen in unmodified strains. Most prevalent were four versions of eicosanes (n=20): straight chain eicosane (C20); 1,19-diene-eicosane, and bicyclic Bicyclo[10.8.0]eicosane, (E). Also seen were cyclized alkanes (n=10, 14, 20, 28, and 30). See FIGS. 42, 43, and 44.

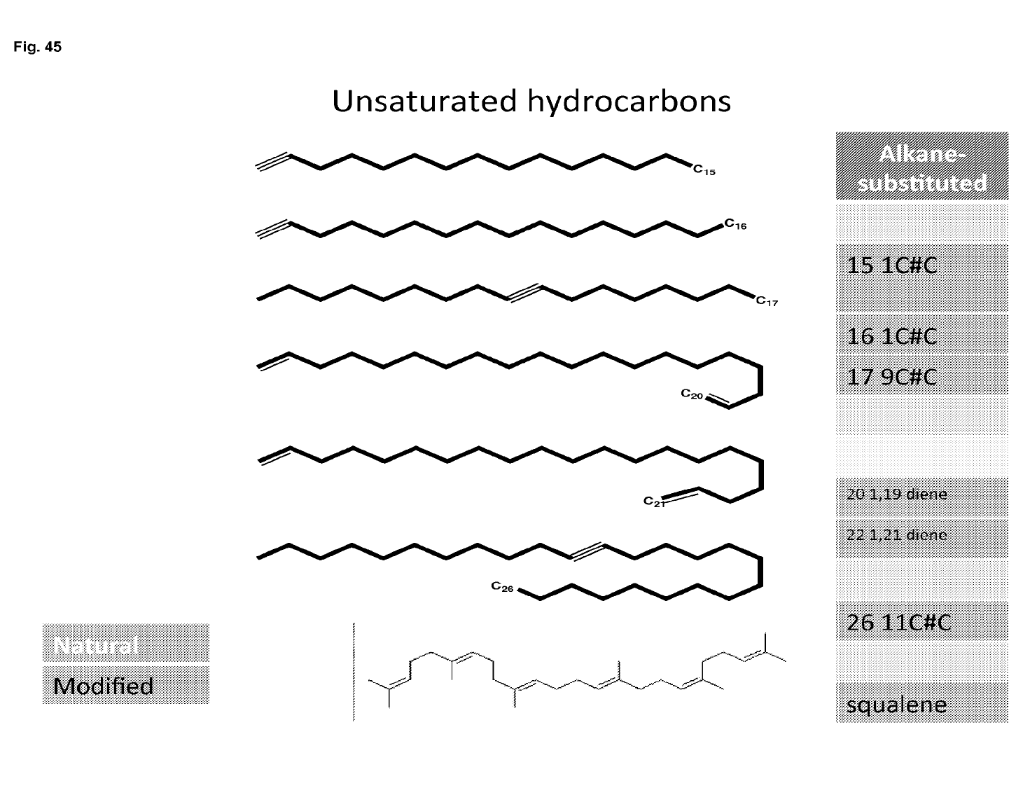

Unsaturated alkanes were prevalent in FUEL genetically-modified Cupriavidus necator (DSM 531) batches. See FIG. 45.

What is claimed is:

1. A composition comprising a bacterial cell that converts crude glycerol or a mixture of glycerol and methanol or ethanol and matter organic non-glycerol (MONG) and salts, into one or more lipids or hydrocarbons.

2. The composition of claim 1, wherein the bacterial cell comprises at least a first exogenous nucleic acid sequence.

3. The composition of claim 1, wherein the microorganism is chosen from the genera Rhodococcus or Gordonia or Ralstonia.

4. The composition of claim 1, wherein the bacterial cell comprises at least a first and a second exogenous nucleic acid sequence but no more than five exogenous nucleic acid sequences.

5. The composition of claim 2, wherein the at least a first exogenous nucleic acid sequence consists of a first exogenous nucleic acid sequence, wherein the first exogenous nucleic acid sequence encodes a thioesterase.

6. The composition of claim 2, wherein the at least a first exogenous nucleic acid sequence consists of first, second, and third exogenous nucleic acid sequences, wherein the first exogenous nucleic acid sequence encodes fatty acid aldehyde acyl-ACP reductase, the second exogenous nucleic acid sequence encodes a fatty acid aldehyde decarbonylase, and the third exogenous nucleic acid sequence encodes a thioesterase.

7. The composition of claim 2, wherein the at least a first exogenous nucleic acid sequence consists of first and second exogenous nucleic acid sequences, wherein the first exogenous nucleic acid sequence encodes fatty acid aldehyde acyl-ACP reductase and the second exogenous nucleic acid sequence encodes a fatty acid aldehyde decarbonylase.

8. The composition of claim 1, wherein the microorganism is Rhodococcus opacus.

9. The composition of claim 1, wherein the one or more lipids or hydrocarbons comprises at least one organic molecule having a carbon chain length of at least 8 carbon atoms and at least one carbon-carbon double bond.

10. The composition of claim 1, wherein the one or more lipids or hydrocarbons comprises one or more lipids comprising at least one hydroxyl acid molecule having a carbon chain length of at least 6 carbon atoms.

11. The composition of claim 1, wherein the one or more lipids or hydrocarbons comprises one or more lipids comprising at least one diacid acid molecule having a carbon chain length of at least 6 carbon atoms.

12. A composition according to claim 1, wherein the bacterial cell is an oxyhydrogen microorganism including oxyhydrogen microorganisms selected from one or more of the following genera: Rhodopseudomonas sp.; Rhodospirillum sp.; Rhodococcus sp.; Nocardia sp.; Mycobacterium sp.; Gordonia sp.; Tsukamurella sp.; Rhodobacter sp.; Rhizobium sp.; Thiocapsa sp.; Pseudomonas sp.; Hydrogenomonas sp.; Hydrogenobacter sp.; Hydrogenovibrio sp.; Helicobacter sp.; Oleomonas sp.; Xanthobacter sp.; Hydrogenophaga sp.; Bradyrhizobium sp.; Ralstonia sp.; Alcaligenes sp.; Variovorax sp.; Acidovorax sp.; Anabaena sp.; Scenedesmus sp.; Chlamydomonas sp.; Ankistrodesmus sp.; and Rhaphidium sp.

13. The composition of claim 1, wherein the one or more lipids or hydrocarbons comprises a mixture of lipids having at least one unsaturated fatty acid molecule having a carbon chain length from 8 carbon atoms to 30 carbon atoms.

14. The composition of claim 1, wherein the one or more lipids or hydrocarbons comprises a mixture of hydrocarbons having at least one desaturated hydrocarbon molecule having a carbon chain length from 8 carbon atoms to 30 carbon atoms.

15. The composition of claim 1, wherein the one or more lipids or hydrocarbons comprise a quantity of at least one alkene, alkyne, hydroxy acid, dicarboxylic acid, and/or unsaturated fatty acid at a level higher than the quantity of the alkene, alkyne, hydroxy acid dicarboxylic acid, and/or unsaturated fatty acid in the same bacterial cell not comprising the exogenous nucleic acid sequence.

16. The composition of claim 1, wherein the bacterial cell is able to grow on methanol as sole carbon source.

17. The composition of claim 1, wherein the bacterial cell is able to tolerate and grow in salinities exceeding 35 grams per liter.

18. A composition according to any of claim 1, wherein said crude glycerol is generated from the manufacture of biodiesel.

19. A composition according to claim 1, wherein said methanol is a component of crude glycerol, or is synthesized via syngas produced from a waste or low values carbon source comprising lignocellulosic energy crops, crop residue, bagasse, saw dust, forestry residue, food waste, municipal solid waste, waste carpet, biogas, landfill gas, stranded natural gas, or pet coke.

20. A composition according to claim 1, wherein said bacterial cell is drawn from suborder corynebacterinaeae or the family burkholderiaceae.


SEQUENCE LISTING (45 sequences)

SEQ ID NO: 1 - DNA, 10560 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgagcgcgcaaagccactactgccacttttggagactgtgtacgtcgagggcctctgccagtgtcgaacagacattcgcctacggccctcgtctgttcgggctcagggcgcgtcagcgggtgttggcgggtgtcggggctggcttaactatgcggcatcagagcagattgtactgagagtgcaccatatgcggtgtgaaataagtcccgcgcagtcgcccacaaccgcccacagccccgaccgaattgatacgccgtagtctcgtctaacatgactctcacgtggtatacgccacactttatccgcacagatgcgtaaggagaaaataccgcatcaggcgccattcgccattcaggctgcgcaactgttgggaagggcgatcggtgcgggcctcttcgctatggcgtgtctacgcattcctcttttatggcgtagtccgcggtaagcggtaagtccgacgcgttgacaacccttcccgctagccacgcccggagaagcgatatacgccagctggcgaaagggggatgtgctgcaaggcgattaagttgggtaacgccagggttttcccagtcacgacgttgtaaaacgacggccagtgccaaatgcggtcgaccgctttccccctacacgacgttccgctaattcaacccattgcggtcccaaaagggtcagtgctgcaacattttgctgccggtcacggttgcttgcatgcctgcaggtcgacgggcccgggatccgatgctcttccgctaagatctgccgcggccgcgtcctcagaagaactcgtcaagaaggcgatagacgaacgtacggacgtccagctgcccgggccctaggctacgagaaggcgattctagacggcgccggcgcaggagtcttcttgagcagttcttccgctatctaggcgatgcgctgcgaatcgggagcggcgataccgtaaagcacgaggaagcggtcagcccattcgccgccaagctcttcagcaatatcacgggtagccaatccgctacgcgacgcttagccctcgccgctatggcatttcgtgctccttcgccagtcgggtaagcggcggttcgagaagtcgttatagtgcccatcggttcgctatgtcctgatagcggtccgccacacccagccggccacagtcgatgaatccagaaaagcggccattttccaccatgatattcggcaagcaggcatcggcgatacaggactatcgccaggcggtgtgggtcggccggtgtcagctacttaggtcttttcgccggtaaaaggtggtactataagccgttcgtccgtagcccatgggtcacgacgagatcctcgccgtcgggcatgcgcgccttgagcctggcgaacagttcggctggcgcgagcccctgatgctcttcgtccagatcatggtacccagtgctgctctaggagcggcagcccgtacgcgcggaactcggaccgcttgtcaagccgaccgcgctcggggactacgagaagcaggtctagtacctgatcgacaagaccggcttccatccgagtacgtgctcgctcgatgcgatgtttcgcttggtggtcgaatgggcaggtagccggatcaagcgtatgcagggactagctgttctggccgaaggtaggctcatgcacgagcgagctacgctacaaagcgaaccaccagcttacccgtccatcggcctagttcgcatacgtcccgccgcattgcatcagccatgatggatactttctcggcaggagcaaggtgggatgacaggagatcctgccccggcacttcgcccaatagcagccagtccggcggcgtaacgtagtcggtactacctatgaaagagccgtcctcgttccaccctactgtcctctaggacggggccgtgaagcgggttatcgtcggtcaggcttcccgcttcagtgacaacgtcgagcacagctgcgcaaggaacgcccgtcgtggccagccacgatagccgcgctgcctcgtcctgcagttcattcaggggaagggcgaagtcactgttgcagctcgtgtcgacgcgttccttgcgggcagcaccggtcggtgctatcggcgcgacggagcaggacgtcaagtaagtccccaccggacaggtcggtcttgacaaaaagaaccgggcgcccctgcgctgacagccggaacacggcggcatcagagcagccgattgtctgttgtgcccagtcgtggcctgtccagccagaactgtttttcttggcccgcggggacgcgactgtcggccttgtgccgccgtagtctcgtcggctaacagacaacacgggtcagatagccgaatagcctctccacccaagcggccggagaacctgcgtgcaatccatcttgttcaatcatgatatcccttaattaaccgttaacactagttcagtatcggcttatcggagaggtgggttcgccggcctcttggacgcacgttaggtagaacaagttagtactatagggaattaattggcaattgtgatcaagtctccatctcgccgtgtatgcgggcctgacggatcaacgttcccaccgagccagtcgagatgttcatctggtcggcgatctgccggtacttcaaaccttgttaggtagagcggcacatacgcccggactgcctagttgcaagggtggctcggtcagctctacaagtagaccagccgctagacggccatgaagtttggaacaatgcgcagttccacagccttcttgcggcgttcctgcgcacgagcgatgtagtcgcctcggtcttcggcgacgagccgtttgatggtgcttttcgagacgccacgcgtcaaggtgtcggaagaacgccgcaaggacgcgtgctcgctacatcagcggagccagaagccgctgctcggcaaactaccacgaaaagctctgcgggaacttgtcagccaactcctgcgcggtctgcgtgcgacgcatcacgcgttctgcagcacccatcagtccgtcccctctgctgctgcgaacagtgccgatccttgaacagtcggttgaggacgcgccagacgcacgctgcgtagtgcgcaagacgtcgtgggtagtcaggcaggggagacgacgacgcttgtcacggctaggatcgaccttcttgagcttcggccgcggcgcggtggcgttcttccgtaccgcttccgtttttgcgctgctgctcactttgccgcggcgtgcctggattttctagctggaagaactcgaagccggcgccgcgccaccgcaagaaggcatggcgaaggcaaaaacgcgacgacgagtgaaacggcgccgcacggacctaaaacgagaactcggcggcggtgaaggtgcggtgggtccagtgggcgactgatttgccgatctgctcggcctcggcccgactcatggggccgatcccgtcgttggctcttgagccgccgccacttccacgccacccaggtcacccgctgactaaacggctagacgagccggagccgggctgagtaccccggctagggcagcaacgcgtcgagggtgaagttggtcagggcggtgaagtcggtgaccatctgccgccacacagtgatcgacgggtagttctgtttccggatctcgcggtaggccccgcagctcccacttcaaccagtcccgccacttcagccactggtagacggcggtgtgtcactagctgcccatcaagacaaaggcctagagcgccatccgggattcccgggtgcggtcgaacagttcgacgttccggcccgtttcggtcctgacctgtgtcttgcggccgtagtccggtggggcggggaaacggtcaccgagtaagggcccacgccagcttgtcaagctgcaaggccgggcaaagccaggactggacacagaacgccggcatcaggccaccccgcccctttgccagtggctccgcttttgcgaggcctttgagcgagtacggatccgagggaccccagaccgtcgtccagtgcgggtggatcgggttctgggtgagctgctgcgcgtagcccgcgaaaacgctccggaaactcgctcatgcctaggctccctggggtctggcagcaggtcacgcccacctagcccaagacccactcgacgacgcgcatcgggtgatcggcgccgaccaccgaggcgatcagcccctggttcacccggtcgtagagccgcagcgggccctgtcgggctgcctggagggtgtagaccgggctttactagccgcggctggtggctccgctagtcggggaccaagtgggccagcatctcggcgtcgcccgggacagcccgacggacctcccacatctggcccgaaacgagcagccaccacaggtgcgcgtgctcggtcgcgggattgatcgtcatcacggtcggatcgggcagatccgcgttacgtgcggcccactgcgcctggtcgctcgtcggtggtgtccacgcgcacgagccagcgccctaactagcagtagtgccagcctagcccgtctaggcgcaatgcacgccgggtgacgcggaccaggtcgtccacgtcgagcaccaagcccaacctgatcgacggggtgcgggccgcaatgtagcggcgggtgagcgcctccgcgcgcggctgcggccactgcccgcagcaggtgcagctcgtggttcgggttggactagctgccccacgcccggcgttacatcgccgcccactcgcggaggcgcgcgccgacgccggtgacgggctcccggacgtagtcatccgtcgcgtgcgggtatttgaaccgccagcggtccaaccaggcgtcaacagcagcggtcatgaccgccaagctagggccggatcagggcctgcatcagtaggcagcgcacgcccataaacttggcggtcgccaggttggtccgcagttgtcgtcgccagtactggcggttcgatcccggcctagtgtaccgatcgggggaggcgcgccgcaaattatttaagagtctcgctagcaaaccatgtcaggtgttgcggtgggttccgggtaaacctccacccgaattacatggctagccccctccgcgcggcgtttaataaattctcagagcgatcgtttggtacagtccacaacgccacccaaggcccatttggaggtgggcttaaatttaagagtctcgctagctaagccctatctgatgctgcgcggggggtccttcgcactgaatctcaaaggtggccggctgaatttcgtcgcgcgaaaacctaaattctcagagcgatcgattcgggatagactacgacgcgccccccaggaagcgtgacttagagtttccaccggccgacttaaagcagcgcgcttttggtccctggacagttctggaattcagcaagaggtgtgtctgaacttcggtgtttttttggggggtgactccagcggggtgggcacaacgcgaacagagacctagggacctgtcaagaccttaagtcgttctccacacagacttgaagccacaaaaaaaccccccactgaggtcgccccacccgtgttgcgcttgtctctggatgtgtgtacgacggcgggaggtaagtcgggtacggctcggactgcggtagagcaaccgtcgaatcgatttcgagcagagcgagcagagcaagatattccaacacacatgctgccgccctccattcagcccatgccgagcctgacgccatctcgttggcagcttagctaaagctcgtctcgctcgtctcgttctataaggtaaactccggggttcctcggcggcctcccccgtctgtttgctcaaccgagggagacctggcggtcccgcgtttccggacgcgcgggaccgcctaccgctcgtttgaggccccaaggagccgccggagggggcagacaaacgagttggctccctctggaccgccagggcgcaaaggcctgcgcgccctggcggatggcgagcagagcggaagagcatctagatgcattcgcgaggtaccgagctcgaattcgtaatcatggtcatagctgtttcctgtgtgaaattgttatccgctcacaattctcgccttctcgtagatctacgtaagcgctccatggctcgagcttaagcattagtaccagtatcgacaaaggacacactttaacaataggcgagtgttatccacacaacatacgagccggaagcataaagtgtaaagcctggggtgcctaatgagtgagctaactcacattaattgcgttgcgctcactgcccgctttcaggtgtgttgtatgctcggccttcgtatttcacatttcggaccccacggattactcactcgattgagtgtaattaacgcaacgcgagtgacgggcgaaagcagtcgggaaacctgtcgtgccagctgcattaatgaatcggccaacgcgcggggagaggcggtttgcgtattgggcgctcttccgcttcctcgctcactggtcagccctttggacagcacggtcgacgtaattacttagccggttgcgcgcccctctccgccaaacgcataacccgcgagaaggcgaaggagcgagtgacactcgctgcgctcggtcgttcggctgcggcgagcggtatcagctcactcaaaggcggtaatacggttatccacagaatcaggggataacgcaggaaagaatgagcgacgcgagccagcaagccgacgccgctcgccatagtcgagtgagtttccgccattatgccaataggtgtcttagtcccctattgcgtcctttcttcatgtgagcaaaaggccagcaaaaggccaggaaccgtaaaaaggccgcgttgctggcgtttttccataggctccgcccccctgacgagcatcacaaaaatgtacactcgttttccggtcgttttccggtccttggcatttttccggcgcaacgaccgcaaaaaggtatccgaggcggggggactgctcgtagtgtttttacgacgctcaagtcagaggtggcgaaacccgacaggactataaagataccaggcgtttccccctggaagctccctcgtgcgctctcctgttccgaccctgcgctgcgagttcagtctccaccgctttgggctgtcctgatatttctatggtccgcaaagggggaccttcgagggagcacgcgagaggacaaggctgggacgcgcttaccggatacctgtccgcctttctcccttcgggaagcgtggcgctttctcatagctcacgctgtaggtatctcagttcggtgtaggtcgttcgctcgcgaatggcctatggacaggcggaaagagggaagcccttcgcaccgcgaaagagtatcgagtgcgacatccatagagtcaagccacatccagcaagcgagcaagctgggctgtgtgcacgaaccccccgttcagcccgaccgctgcgccttatccggtaactatcgtcttgagtccaacccggtaagacacgacttatcggttcgacccgacacacgtgcttggggggcaagtcgggctggcgacgcggaataggccattgatagcagaactcaggttgggccattctgtgctgaatagcccactggcagcagccactggtaacaggattagcagagcgaggtatgtaggcggtgctacagagttcttgaagtggtggcctaactacggctacactagaaggtgaccgtcgtcggtgaccattgtcctaatcgtctcgctccatacatccgccacgatgtctcaagaacttcaccaccggattgatgccgatgtgatcttggacagtatttggtatctgcgctctgctgaagccagttaccttcggaaaaagagttggtagctcttgatccggcaaacaaaccaccgctggtagcggtggcctgtcataaaccatagacgcgagacgacttcggtcaatggaagcctttttctcaaccatcgagaactaggccgtttgtttggtggcgaccatcgccacctttttttgtttgcaagcagcagattacgcgcagaaaaaaaggatctcaagaagatcctttgatcttttctacggggtctgacgctcagtggaacgaaaacaaaaaaacaaacgttcgtcgtctaatgcgcgtctttttttcctagagttcttctaggaaactagaaaagatgccccagactgcgagtcaccttgcttttgtcacgttaagggattttggtcatgagattatcaaaaaggatcttcacctagatccttttaaattaaaaatgaagttttaaatcaatctaaagtatatatgagtgcaattccctaaaaccagtactctaatagtttttcctagaagtggatctaggaaaatttaatttttacttcaaaatttagttagatttcatatatacagtaaacttggtctgacagttaccaatgcttaatcagtgaggcacctatctcagcgatctgtctatttcgttcatccatagttgcctgactccccgtcgttcatttgaaccagactgtcaatggttacgaattagtcactccgtggatagagtcgctagacagataaagcaagtaggtatcaacggactgaggggcagcagtagataactacgatacgggagggcttaccatctggccccagtgctgcaatgataccgcgagacccacgctcaccggctccagatttatcagcaataaaccatctattgatgctatgccctcccgaatggtagaccggggtcacgacgttactatggcgctctgggtgcgagtggccgaggtctaaatagtcgttatttgcagccagccggaagggccgagcgcagaagtggtcctgcaactttatccgcctccatccagtctattaattgttgccgggaagctagagtaagtagttcgcgtcggtcggccttcccggctcgcgtcttcaccaggacgttgaaataggcggaggtaggtcagataattaacaacggcccttcgatctcattcatcaagcgcagttaatagtttgcgcaacgttgttgccattgctacaggcatcgtggtgtcacgctcgtcgtttggtatggcttcattcagctccggttcccaacgatcgtcaattatcaaacgcgttgcaacaacggtaacgatgtccgtagcaccacagtgcgagcagcaaaccataccgaagtaagtcgaggccaagggttgctagaaggcgagttacatgatcccccatgttgtgcaaaaaagcggttagctccttcggtcctccgatcgttgtcagaagtaagttggccgcagtgttatcactcttccgctcaatgtactagggggtacaacacgttttttcgccaatcgaggaagccaggaggctagcaacagtcttcattcaaccggcgtcacaatagtgagatggttatggcagcactgcataattctcttactgtcatgccatccgtaagatgcttttctgtgactggtgagtactcaaccaagtcattctgagaatagttaccaataccgtcgtgacgtattaagagaatgacagtacggtaggcattctacgaaaagacactgaccactcatgagttggttcagtaagactcttatcagtatgcggcgaccgagttgctcttgcccggcgtcaatacgggataataccgcgccacatagcagaactttaaaagtgctcatcattggaaaacgttcttccatacgccgctggctcaacgagaacgggccgcagttatgccctattatggcgcggtgtatcgtcttgaaattttcacgagtagtaaccttttgcaagaagggggcgaaaactctcaaggatcttaccgctgttgagatccagttcgatgtaacccactcgtgcacccaactgatcttcagcatcttttactttcaccagcccccgcttttgagagttcctagaatggcgacaactctaggtcaagctacattgggtgagcacgtgggttgactagaagtcgtagaaaatgaaagtggtcggtttctgggtgagcaaaaacaggaaggcaaaatgccgcaaaaaagggaataagggcgacacggaaatgttgaatactcatactcttcctttttcaatattcaaagacccactcgtttttgtccttccgttttacggcgttttttcccttattcccgctgtgcctttacaacttatgagtatgagaaggaaaaagttataaattgaagcatttatcagggttattgtctcatgagcggatacatatttgaatgtatttagaaaaataaacaaataggggttccgcgcacatttccccgaaataacttcgtaaatagtcccaataacagagtactcgcctatgtataaacttacataaatctttttatttgtttatccccaaggcgcgtgtaaaggggctttagtgccacctgacgtctaagaaaccattattatcatgacattaacctataaaaataggcgtatcacgaggccctttcgtctcacggtggactgcagattctttggtaataatagtactgtaattggatatttttatccgcatagtgctccgggaaagcag

SEQ ID NO: 2 - DNA, 10288 bp, Artificial Sequence
  Description of Artificial Sequence Synthetic polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttatcgataccgtcggatatcccgcttaacctcgaggtggcgccaccgccggcgagatcttgatcacctagggggcccgacgtccttaagctatagttcgaatagctatggcagcacctcgagggggggcccggtacccagcttttgttccctttagtgagggttaattgcgcgcttggcgtaatcatggtcatagctgtttcctgtgtgaaatttggagctcccccccgggccatgggtcgaaaacaagggaaatcactcccaattaacgcgcgaaccgcattagtaccagtatcgacaaaggacacactttaagttatccgctcacaattccacacaacatacgagccggaagcataaagtgtaaagcctggggtgcctaatgagtgagctaactcacattaattgcgttgcgcaataggcgagtgttaaggtgtgttgtatgctcggccttcgtatttcacatttcggaccccacggattactcactcgattgagtgtaattaacgcaacgcctcactgcccgctttccagtcgggaaacctgtcgtgccagctgcattaatgaatcggccaacgcgcggggagaggcggtttgcgtattgggcgcatgcatgagtgacgggcgaaaggtcagccctttggacagcacggtcgacgtaattacttagccggttgcgcgcccctctccgccaaacgcataacccgcgtacgtaaaaaactgttgtaattcattaagcattctgccgacatggaagccatcacaaacggcatgatgaacctgaatcgccagcggcatcagcaccttgtcgcctttttttgacaacattaagtaattcgtaagacggctgtaccttcggtagtgtttgccgtactacttggacttagcggtcgccgtagtcgtggaacagcggaagcgtataatatttgcccatgggggtgggcgaagaactccagcatgagatccccgcgctggaggatcatccagccggcgtcccggaaaacgattccgaagccgcatattataaacgggtacccccacccgcttcttgaggtcgtactctaggggcgcgacctcctagtaggtcggccgcagggccttttgctaaggcttcgccaacctttcatagaaggcggcggtggaatcgaaatctcgtgatggcaggttgggcgtcgcttggtcggtcatttcgaaccccagagtcccgctcagaagggttggaaagtatcttccgccgccaccttagctttagagcactaccgtccaacccgcagcgaaccagccagtaaagcttggggtctcagggcgagtcttcaactcgtcaagaaggcgatagaaggcgatgcgctgcgaatcgggagcggcgataccgtaaagcacgaggaagcggtcagcccattcgccgccaagctcttttgagcagttcttccgctatcttccgctacgcgacgcttagccctcgccgctatggcatttcgtgctccttcgccagtcgggtaagcggcggttcgagaacagcaatatcacgggtagccaacgctatgtcctgatagcggtccgccacacccagccggccacagtcgatgaatccagaaaagcggccattttccaccatgtcgttatagtgcccatcggttgcgatacaggactatcgccaggcggtgtgggtcggccggtgtcagctacttaggtcttttcgccggtaaaaggtggtagatattcggcaagcaggcatcgccatgggtcacgacgagatcctcgccgtcgggcatgcgcgccttgagcctggcgaacagttcggctggcgcgagccccctataagccgttcgtccgtagcggtacccagtgctgctctaggagcggcagcccgtacgcgcggaactcggaccgcttgtcaagccgaccgcgctcggggtgatgctcttcgtccagatcatcctgatcgacaagaccggcttccatccgagtacgtgctcgctcgatgcgatgtttcgcttggtggtcgaatgggcaggactacgagaagcaggtctagtaggactagctgttctggccgaaggtaggctcatgcacgagcgagctacgctacaaagcgaaccaccagcttacccgtcctagccggatcaagcgtatgcagccgccgcattgcatcagccatgatggatactttctcggcaggagcaaggtgagatgacaggagatcctgccccggcacatcggcctagttcgcatacgtcggcggcgtaacgtagtcggtactacctatgaaagagccgtcctcgttccactctactgtcctctaggacggggccgtgttcgcccaatagcagccagtcccttcccgcttcagtgacaacgtcgagcacagctgcgcaaggaacgcccgtcgtggccagccacgatagccgcgctgccaagcgggttatcgtcggtcagggaagggcgaagtcactgttgcagctcgtgtcgacgcgttccttgcgggcagcaccggtcggtgctatcggcgcgacggtcgtcctgcagttcattcagggcaccggacaggtcggtcttgacaaaaagaaccgggcgcccctgcgctgacagccggaacacggcggcatcagagcagcagcaggacgtcaagtaagtcccgtggcctgtccagccagaactgtttttcttggcccgcggggacgcgactgtcggccttgtgccgccgtagtctcgtcgcgattgtctgttgtgcccagtcatagccgaatagcctctccacccaagcggccggagaacctgcgtgcaatccatcttgttcaatcatgcgaaacgatccgctaacagacaacacgggtcagtatcggcttatcggagaggtgggttcgccggcctcttggacgcacgttaggtagaacaagttagtacgctttgctaggtcatcctgtctcttgatcagatcttgatcccctgcgccatcagatccttggcggcaagaaagccatccagtttactttgcagggcttcccaaccttaccaagtaggacagagaactagtctagaactaggggacgcggtagtctaggaaccgccgttctttcggtaggtcaaatgaaacgtcccgaagggttggaatggtgagggcgccccagctggcaattccggttcgcttgctgtccataaaaccgcccagtctagctatcgccatgtaagcccactgcaagctacctgctttctctctcccgcggggtcgaccgttaaggccaagcgaacgacaggtattttggcgggtcagatcgatagcggtacattcgggtgacgttcgatggacgaaagagattgcgcttgcgttttcccttgtccagatagcccagtagctgacattcatcccaggtggcacttttcggggaaatgtgcgcgcccgcgttcctgctggcgcaacgcgaacgcaaaagggaacaggtctatcgggtcatcgactgtaagtagggtccaccgtgaaaagcccctttacacgcgcgggcgcaaggacgaccgcgtgggcctgtttctggcgctggacttcccgctgttccgtcagcagcttttcgcccacggccttgatgatcgcggcggccttggcctgcatatcccgattcaacccggacaaagaccgcgacctgaagggcgacaaggcagtcgtcgaaaagcgggtgccggaactactagcgccgccggaaccggacgtatagggctaagtacggccccagggcgtccagaacgggcttcaggcgctcccgaaggtctcgggccgtctcttgggcttgatcggccttcttgcgcatctcacgcgctcctgctgccggggtcccgcaggtcttgcccgaagtccgcgagggcttccagagcccggcagagaacccgaactagccggaagaacgcgtagagtgcgcgaggacgggcggcctgtagggcaggctcatacccctgccgaaccgcttttgtcagccggtcggccacggcttccggcgtctcaacgcgctttgagattcccagctttccgccggacatcccgtccgagtatggggacggcttggcgaaaacagtcggccagccggtgccgaaggccgcagagttgcgcgaaactctaagggtcgaaatcggccaatccctgcggtgcataggcgcgtggctcgaccgcttgcgggctgatggtgacgtggcccactggtggccgctccagggcctcgtagaacgcctagccggttagggacgccacgtatccgcgcaccgagctggcgaacgcccgactaccactgcaccgggtgaccaccggcgaggtcccggagcatcttgcggagaatgcgcgtgtgacgtgccttgctgccctcgatgccccgttgcagccctagatcggccacagcggccgcaaacgtggtctggtcgcgggtcatctgcgccttacgcgcacactgcacggaacgacgggagctacggggcaacgtcgggatctagccggtgtcgccggcgtttgcaccagaccagcgcccagtagacgcgtttgttgccgatgaactccttggccgacagcctgccgtcctgcgtcagcggcaccacgaacgcggtcatgtgcgggctggtttcgtcacggtggatgctgaaacaacggctacttgaggaaccggctgtcggacggcaggacgcagtcgccgtggtgcttgcgccagtacacgcccgaccaaagcagtgccacctacgacgccgtcacgatgcgatccgccccgtacttgtccgccagccacttgtgcgccttctcgaagaacgccgcctgctgttcttggctggccgacttccaccattcggcagtgctacgctaggcggggcatgaacaggcggtcggtgaacacgcggaagagcttcttgcggcggacgacaagaaccgaccggctgaaggtggtaaccgggctggccgtcatgacgtactcgaccgccaacacagcgtccttgcgccgcttctctggcagcaactcgcgcagtcggcccatcgcttcatcggtgctggcccgaccggcagtactgcatgagctggcggttgtgtcgcaggaacgcggcgaagagaccgtcgttgagcgcgtcagccgggtagcgaagtagccacgagctggccgcccagtgctcgttctctggcgtcctgctggcgtcagcgttgggcgtctcgcgctcgcggtaggcgtgcttgagactggccgccacgttgccccgaccggcgggtcacgagcaagagaccgcaggacgaccgcagtcgcaacccgcagagcgcgagcgccatccgcacgaactctgaccggcggtgcaacgggattttcgccagcttcttgcatcgcatgatcgcgtatgccgccatgcctgcccctcccttttggtgtccaaccggctcgacgggggcagcgcaaggcggtgtaaaagcggtcgaagaacgtagcgtactagcgcatacggcggtacggacggggagggaaaaccacaggttggccgagctgcccccgtcgcgttccgccaccctccggcgggccactcaatgcttgagtatactcactagactttgcttcgcaaagtcgtgaccgcctacggcggctgcggcgccctacgggcttgctctcggaggccgcccggtgagttacgaactcatatgagtgatctgaaacgaagcgtttcagcactggcggatgccgccgacgccgcgggatgcccgaacgagagcgggcttcgccctgcgcggtcgctgcgctcccttgccagcccgtggatatgtggacgatggccgcgagcggccaccggctggctcgcttcgctcggcccggcccgaagcgggacgcgccagcgacgcgagggaacggtcgggcacctatacacctgctaccggcgctcgccggtggccgaccgagcgaagcgagccgggctggacaaccctgctggacaagctgatggacaggctgcgcctgcccacgagcttgaccacagggattgcccaccggctacccagccttcgaccacatacccacctgttgggacgacctgttcgactacctgtccgacgcggacgggtgctcgaactggtgtccctaacgggtggccgatgggtcggaagctggtgtatgggaccggctccaactgcgcggcctgcggccttgccccatcaatttttttaattttctctggggaaaagcctccggcctgcggcctgcgcgcttcgcttgccgtggccgaggttgacgcgccggacgccggaacggggtagttaaaaaaattaaaagagaccccttttcggaggccggacgccggacgcgcgaagcgaacggcgttggacaccaagtggaaggcgggtcaaggctcgcgcagcgaccgcgcagcggcttggccttgacgcgcctggaacgacccaagcctatgcgagtgggggcaacctgtggttcaccttccgcccagttccgagcgcgtcgctggcgcgtcgccgaaccggaactgcgcggaccttgctgggttcggatacgctcaccccccagtcgaaggcgaagcccgcccgcctgccccccgagcctcacggcggcgagtgcgggggttccaagggggcagcgccaccttgggcaaggccgaaggccggtcagcttccgcttcgggcgggcggacggggggctcggagtgccgccgctcacgcccccaaggttcccccgtcgcggtggaacccgttccggcttccggccgcagtcgatcaacaagccccggaggggccactttttgccggaggcgtcagctagttgttcggggcctccccggtgaaaaacggcctc

SEQ ID NO: 3 - DNA, 12758 bp, Artificial Sequence
  Description of Artificial Sequence Synthetic polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttatcgataccgtcggatatcccgcttaacctcgaggtggcgccaccgccggcgagatcttgatcacctagggggcccgacgtccttaagctatagttcgaatagctatggcagcacgggcccgggatccgatgctcttccgctaagatcttttactagttcagtccatctcgccgtgtatgcgggcctgacggatcaacgttcccaccgagccatgcccgggccctaggctacgagaaggcgattctagaaaatgatcaagtcaggtagagcggcacatacgcccggactgcctagttgcaagggtggctcggtgtcgagatgttcatctggtcggcgatctgccggtacttcaaaccttgtttgcgcagttccacagccttcttgcggcgttcctgcgcacgagcgatgtagtcagctctacaagtagaccagccgctagacggccatgaagtttggaacaaacgcgtcaaggtgtcggaagaacgccgcaaggacgcgtgctcgctacatcacgcctcggtcttcggcgacgagccgtttgatggtgcttttcgagacgccgaacttgtcagccaactcctgcgcggtctgcgtgcgacgcatcacgcgttcgcggagccagaagccgctgctcggcaaactaccacgaaaagctctgcggcttgaacagtcggttgaggacgcgccagacgcacgctgcgtagtgcgcaagtgcagcacccatcagtccgtcccctctgctgctgcgaacagtgccgatcgatcgaccttcttgagcttcggccgcggcgcggtggcgttcttccgtaccgacgtcgtgggtagtcaggcaggggagacgacgacgcttgtcacggctagctagctggaagaactcgaagccggcgccgcgccaccgcaagaaggcatggccttccgtttttgcgctgctgctcactttgccgcggcgtgcctggattttcgagaactcggcggcggtgaaggtgcggtgggtccagtgggcgactgatttgaaggcaaaaacgcgacgacgagtgaaacggcgccgcacggacctaaaagctcttgagccgccgccacttccacgccacccaggtcacccgctgactaaagccgatctgctcggcctcggcccgactcatggggccgatcccgtcgttggcgtcgagggtgaagttggtcagggcggtgaagtcggtgaccatctgccgccggctagacgagccggagccgggctgagtaccccggctagggcagcaaccgcagctcccacttcaaccagtcccgccacttcagccactggtagacggcgcacacagtgatcgacgggtagttctgtttccggatctcgcggtaggcccattcccgggtgcggtcgaacagttcgacgttccggcccgtttcggtcctgagtgtgtcactagctgcccatcaagacaaaggcctagagcgccatccgggtaagggcccacgccagcttgtcaagctgcaaggccgggcaaagccaggactcctgtgtcttgcggccgtagtccggtggggcggggaaacggtcaccgagcgcttttgcgaggcctttgagcgagtacggatccgagggaccccagaccgtggacacagaacgccggcatcaggccaccccgcccctttgccagtggctcgcgaaaacgctccggaaactcgctcatgcctaggctccctggggtctggcacgtccagtgcgggtggatcgggttctgggtgagctgctgcgcgtagccctgatcggcgccgaccaccgaggcgatcagcccctggttcacccggtcgtaggcaggtcacgcccacctagcccaagacccactcgacgacgcgcatcgggactagccgcggctggtggctccgctagtcggggaccaagtgggccagcatcagccgcagcgggccctgtcgggctgcctggagggtgtagaccgggctttcgagcagccaccacaggtgcgcgtgctcggtcgcgggattgatcgtcatcatcggcgtcgcccgggacagcccgacggacctcccacatctggcccgaaagctcgtcggtggtgtccacgcgcacgagccagcgccctaactagcagtagtcggtcggatcgggcagatccgcgttacgtgcggcccactgcgcctggtcgtcgtccacgtcgagcaccaagcccaacctgatcgacggggtgcgggccgcgccagcctagcccgtctaggcgcaatgcacgccgggtgacgcggaccagcagcaggtgcagctcgtggttcgggttggactagctgccccacgcccggcgaatgtagcggcgggtgagcgcctccgcgcgcggctgcggccactgcccgtcccggacgtagtcatccgtcgcgtgcgggtatttgaaccgccagcggtccttacatcgccgcccactcgcggaggcgcgcgccgacgccggtgacgggcagggcctgcatcagtaggcagcgcacgcccataaacttggcggtcgccaggaaccaggcgtcaacagcagcggtcatgaccgccaagctagggccggatctgtaccgatcgggggaggcgcgccgcaaattatttaagagtctcgctagcattggtccgcagttgtcgtcgccagtactggcggttcgatcccggcctagacatggctagccccctccgcgcggcgtttaataaattctcagagcgatcgtaaccatgtcaggtgttgcggtgggttccgggtaaacctccacccgaattatttaagagtctcgctagctaagccctatctgatgctgcgcggggggtcctttggtacagtccacaacgccacccaaggcccatttggaggtgggcttaataaattctcagagcgatcgattcgggatagactacgacgcgccccccaggatcgcactgaatctcaaaggtggccggctgaatttcgtcgcgcgaaaacctccctggacagttctggaattcagcaagaggtgtgtctgaacttcggtgttagcgtgacttagagtttccaccggccgacttaaagcagcgcgcttttggagggacctgtcaagaccttaagtcgttctccacacagacttgaagccacaatttttggggggtgactccagcggggtgggcacaacgcgaacagagaccttgtgtgtacgacggcgggaggtaagtcgggtacggctcggactgcggtagaaaaaaccccccactgaggtcgccccacccgtgttgcgcttgtctctggaacacacatgctgccgccctccattcagcccatgccgagcctgacgccatctgcaaccgtcgaatcgatttcgagcagagcgagcagagcaagatattccaaaactccggggttcctcggcggcctcccccgtctgtttgctcaaccgagggcgttggcagcttagctaaagctcgtctcgctcgtctcgttctataaggttttgaggccccaaggagccgccggagggggcagacaaacgagttggctcccagacctggcggtcccgcgtttccggacgcgcgggaccgcctaccgctcgagagcggaagagcatctagatgcattcgcgaggtacccagcttttgttccctctggaccgccagggcgcaaaggcctgcgcgccctggcggatggcgagctctcgccttctcgtagatctacgtaagcgctccatgggtcgaaaacaagggtttagtgagggttaattgcgcgcttggcgtaatcatggtcatagctgtttcctgtgtgaaattgttatccgctcacaattccacacaacatacgagccggaaatcactcccaattaacgcgcgaaccgcattagtaccagtatcgacaaaggacacactttaacaataggcgagtgttaaggtgtgttgtatgctcggccaagcataaagtgtaaagcctggggtgcctaatgagtgagctaactcacattaattgcgttgcgctcactgcccgctttccagtcgggaaacctgtcgtgcttcgtatttcacatttcggaccccacggattactcactcgattgagtgtaattaacgcaacgcgagtgacgggcgaaaggtcagccctttggacagcacgcagctgcattaatgaatcggccaacgcgcggggagaggcggtttgcgtattgggcgcatgcataaaaactgttgtaattcattaagcattctgccgacatgtcgacgtaattacttagccggttgcgcgcccctctccgccaaacgcataacccgcgtacggaagccatcacaaacggcatgatgaacctgaatcgccagcggcatcagcaccttgtcgccttgcgtataatatttgcccatgggggtgggcgaagaactccttcggtagtgtttgccgtactacttggacttagcggtcgccgtagtcgtggaacagcggaacgcatattataaacgggtacccccacccgcttcttgaccagcatgagatccccgcgctggaggatcatccagccggcgtcccggaaaacgattccgaagcccaacctttcatagaaggcggcggtggaatcgaaatcggtcgtactctaggggcgcgacctcctagtaggtcggccgcagggccttttgctaaggcttcgggttggaaagtatcttccgccgccaccttagctttagtcgtgatggcaggttgggcgtcgcttggtcggtcatttcgaaccccagagtcccgctcagaagaactcgtcaagaaggcgatagaaggcgatgcgctgcgagcactaccgtccaacccgcagcgaaccagccagtaaagcttggggtctcagggcgagtcttcttgagcagttcttccgctatcttccgctacgcgacgcaatcgggagcggcgataccgtaaagcacgaggaagcggtcagcccattcgccgccaagctcttcagcaatatcacgggtagccaacgctatgtcctgatattagccctcgccgctatggcatttcgtgctccttcgccagtcgggtaagcggcggttcgagaagtcgttatagtgcccatcggttgcgatacaggactatgcggtccgccacacccagccggccacagtcgatgaatccagaaaagcggccattttccaccatgatattcggcaagcaggcatcgccatgggtcacgacgcgccaggcggtgtgggtcggccggtgtcagctacttaggtcttttcgccggtaaaaggtggtactataagccgttcgtccgtagcggtacccagtgctgcagatcctcgccgtcgggcatgcgcgccttgagcctggcgaacagttcggctggcgcgagcccctgatgctcttcgtccagatcatcctgatcgacaagactctaggagcggcagcccgtacgcgcggaactcggaccgcttgtcaagccgaccgcgctcggggactacgagaagcaggtctagtaggactagctgttctgcggcttccatccgagtacgtgctcgctcgatgcgatgtttcgcttggtggtcgaatgggcaggtagccggatcaagcgtatgcagccgccgcattgcatcgccgaaggtaggctcatgcacgagcgagctacgctacaaagcgaaccaccagcttacccgtccatcggcctagttcgcatacgtcggcggcgtaacgtagagccatgatggatactttctcggcaggagcaaggtgagatgacaggagatcctgccccggcacttcgcccaatagcagccagtcccttcccgcttcagttcggtactacctatgaaagagccgtcctcgttccactctactgtcctctaggacggggccgtgaagcgggttatcgtcggtcagggaagggcgaagtcacacaacgtcgagcacagctgcgcaaggaacgcccgtcgtggccagccacgatagccgcgctgcctcgtcctgcagttcattcagggcaccggacaggtcggtgttgcagctcgtgtcgacgcgttccttgcgggcagcaccggtcggtgctatcggcgcgacggagcaggacgtcaagtaagtcccgtggcctgtccagcctcttgacaaaaagaaccgggcgcccctgcgctgacagccggaacacggcggcatcagagcagccgattgtctgttgtgcccagtcatagccgaatagcctagaactgtttttcttggcccgcggggacgcgactgtcggccttgtgccgccgtagtctcgtcggctaacagacaacacgggtcagtatcggcttatcggactccacccaagcggccggagaacctgcgtgcaatccatcttgttcaatcatgcgaaacgatcctcatcctgtctcttgatcagatcttgatcccctgcgcgaggtgggttcgccggcctcttggacgcacgttaggtagaacaagttagtacgctttgctaggagtaggacagagaactagtctagaactaggggacgcgcatcagatccttggcggcaagaaagccatccagtttactttgcagggcttcccaaccttaccagagggcgccccagctggcaattccggttcgcttgctggtagtctaggaaccgccgttctttcggtaggtcaaatgaaacgtcccgaagggttggaatggtctcccgcggggtcgaccgttaaggccaagcgaacgactccataaaaccgcccagtctagctatcgccatgtaagcccactgcaagctacctgctttctctttgcgcttgcgttttcccttgtccagatagcccagtaaggtattttggcgggtcagatcgatagcggtacattcgggtgacgttcgatggacgaaagagaaacgcgaacgcaaaagggaacaggtctatcgggtcatgctgacattcatcccaggtggcacttttcggggaaatgtgcgcgcccgcgttcctgctggcgctgggcctgtttctggcgctggacttcccgctgttccgcgactgtaagtagggtccaccgtgaaaagcccctttacacgcgcgggcgcaaggacgaccgcgacccggacaaagaccgcgacctgaagggcgacaaggctcagcagcttttcgcccacggccttgatgatcgcggcggccttggcctgcatatcccgattcaacggccccagggcgtccagaacgggcttcaggcgctcagtcgtcgaaaagcgggtgccggaactactagcgccgccggaaccggacgtatagggctaagttgccggggtcccgcaggtcttgcccgaagtccgcgaccgaaggtctcgggccgtctcttgggcttgatcggccttcttgcgcatctcacgcgctcctgcggcggcctgtagggcaggctcatacccctgccgaaccggcttccagagcccggcagagaacccgaactagccggaagaacgcgtagagtgcgcgaggacgccgccggacatcccgtccgagtatggggacggcttgggcttttgtcagccggtcggccacggcttccggcgtctcaacgcgctttgagattcccagcttttcggccaatccctgcggtgcataggcgcgtggctcgacgaaaacagtcggccagccggtgccgaaggccgcagagttgcgcgaaactctaagggtcgaaaagccggttagggacgccacgtatccgcgcaccgagctccgcttgcgggctgatggtgacgtggcccactggtggccgctccagggcctcgtagaacgcctgaatgcgcgtgtgacgtgccttgctgccctcgatgccggcgaacgcccgactaccactgcaccgggtgaccaccggcgaggtcccggagcatcttgcggacttacgcgcacactgcacggaacgacgggagctacggccgttgcagccctagatcggccacagcggccgcaaacgtggtctggtcgcgggtcatctgcgctttgttgccgatgaactccttggccgacagcctgccgggcaacgtcgggatctagccggtgtcgccggcgtttgcaccagaccagcgcccagtagacgcgaaacaacggctacttgaggaaccggctgtcggacggctcctgcgtcagcggcaccacgaacgcggtcatgtgcgggctggtttcgtcacggtggatgctggccgtcacgatgcgatccgccccgtacttgtccgccaaggacgcagtcgccgtggtgcttgcgccagtacacgcccgaccaaagcagtgccacctacgaccggcagtgctacgctaggcggggcatgaacaggcggtgccacttgtgcgccttctcgaagaacgccgcctgctgttcttggctggccgacttccaccattccgggctggccgtcatgacgtactcgaccgccaacaccggtgaacacgcggaagagcttcttgcggcggacgacaagaaccgaccggctgaaggtggtaaggcccgaccggcagtactgcatgagctggcggttgtgagcgtccttgcgccgcttctctggcagcaactcgcgcagtcggcccatcgcttcatcggtgctgctggccgcccagtgctcgttctctggcgtcctgctgtcgcaggaacgcggcgaagagaccgtcgttgagcgcgtcagccgggtagcgaagtagccacgacgaccggcgggtcacgagcaagagaccgcaggacgacgcgtcagcgttgggcgtctcgcgctcgcggtaggcgtgcttgagactggccgccacgttgcccattttcgccagcttcttgcatcgcatgatcgcgtatgcgcagtcgcaacccgcagagcgcgagcgccatccgcacgaactctgaccggcggtgcaacgggtaaaagcggtcgaagaacgtagcgtactagcgcatacccgccatgcctgcccctcccttttggtgtccaaccggctcgacgggggcagcgcaaggcggtgcctccggcgggccactcaatgcttgagtatactcactggcggtacggacggggagggaaaaccacaggttggccgagctgcccccgtcgcgttccgccacggaggccgcccggtgagttacgaactcatatgagtgaagactttgcttcgcaaagtcgtgaccgcctacggcggctgcggcgccctacgggcttgctctccgggcttcgccctgcgcggtcgctgcgctcccttgcctctgaaacgaagcgtttcagcactggcggatgccgccgacgccgcgggatgcccgaacgagaggcccgaagcgggacgcgccagcgacgcgagggaacgg

SEQ ID NO: 4 - DNA, 15158 bp, Artificial Sequence
  Description of Artificial Sequence Synthetic polynucleotide
ggggagccgcgccgaaggcgtgggggaaccccgcaggggtgcccttctttgggcaccaaagaactagatatagggcgaaatgcgaaagacttaaaaatcacccctcggcgcggcttccgcacccccttggggcgtccccacgggaagaaacccgtggtttcttgatctatatcccgctttacgctttctgaatttttagtacaacttaaaaaaggggggtacgcaacagctcattgcggcaccccccgcaatagctcattgcgtaggttaaagaaaatctgtaattgactgccacttttatgttgaattttttccccccatgcgttgtcgagtaacgccgtggggggcgttatcgagtaacgcatccaatttcttttagacattaactgacggtgaaaatcgcaacgcataattgttgtcgcgctgccgaaaagttgcagctgattgcgcatggtgccgcaaccgtgcggcaccctaccgcatggagataagcatggccagcgttgcgtattaacaacagcgcgacggcttttcaacgtcgactaacgcgtaccacggcgttggcacgccgtgggatggcgtacctctattcgtaccggtcgcagtccagagaaatcggcattcaagccaagaacaagcccggtcactgggtgcaaacggaacgcaaagcgcatgaggcgtgggccgggcttattgcgaggcgtcaggtctctttagccgtaagttcggttcttgttcgggccagtgacccacgtttgccttgcgtttcgcgtactccgcacccggcccgaataacgctcgaaacccacggcggcaatgctgctgcatcacctcgtggcgcagatgggccaccagaacgccgtggtggtcagccagaagacactttccaagctcatcggactttgggtgccgccgttacgacgacgtagtggagcaccgcgtctacccggtggtcttgcggcaccaccagtcggtcttctgtgaaaggttcgagtagcctcgttctttgcggacggtccaatacgcagtcaaggacttggtggccgagcgctggatctccgtcgtgaagctcaacggccccggcaccgtgtcggcctacggcaagaaacgcctgccaggttatgcgtcagttcctgaaccaccggctcgcgacctagaggcagcacttcgagttgccggggccgtggcacagccggatgctggtcaatgaccgcgtggcgtggggccagccccgcgaccagttgcgcctgtcggtgttcagtgccgccgtggtggttgatcacgacgaccaggacgaatcaccagttactggcgcaccgcaccccggtcggggcgctggtcaacgcggacagccacaagtcacggcggcaccaccaactagtgctgctggtcctgcttaggctgttggggcatggcgacctgcgccgcatcccgaccctgtatccgggcgagcagcaactaccgaccggccccggcgaggagccgcccagccagcccggccgacaaccccgtaccgctggacgcggcgtagggctgggacataggcccgctcgtcgttgatggctggccggggccgctcctcggcgggtcggtcgggccgattccgggcatggaaccagacctgccagccttgaccgaaacggaggaatgggaacggcgcgggcagcagcgcctgccgatgcccgatgagccgtgttttctaaggcccgtaccttggtctggacggtcggaactggctttgcctccttacccttgccgcgcccgtcgtcgcggacggctacgggctactcggcacaaaagtggacgatggcgagccgttggagccgccgacacgggtcacgctgccgcgccggtagcacttgggttgcgcagcaacccgtaagtgcgctgttccagactaacctgctaccgctcggcaacctcggcggctgtgcccagtgcgacggcgcggccatcgtgaacccaacgcgtcgttgggcattcacgcgacaaggtctgattcggctgtagccgcctcgccgccctataccttgtctgcctccccgcgttgcgtcgcggtgcatggagccgggccacctcgacctgaatggaagccggcggagccgacatcggcggagcggcgggatatggaacagacggaggggcgcaacgcagcgccacgtacctcggcccggtggagctggacttaccttcggccgcccacctcgctaacggattcaccgtttttatcaggctctgggaggcagaataaatgatcatatcgtcaattattacctccacggggagagcctgagcaaactgtggagcgattgcctaagtggcaaaaatagtccgagaccctccgtcttatttactagtatagcagttaataatggaggtgcccctctcggactcgtttgaggcctcaggcatttgagaagcacacggtcacactgcttccggtagtcaataaaccggtaaaccagcaatagacataagcggctatttaacgaccctgcccccggagtccgtaaactcttcgtgtgccagtgtgacgaaggccatcagttatttggccatttggtcgttatctgtattcgccgataaattgctgggacgggtgaaccgacgaccgggtcgaatttgctttcgaatttctgccattcatccgcttattatcacttattcaggcgtagcaccaggcgtttaagggcaccaataacttggctgctggcccagcttaaacgaaagcttaaagacggtaagtaggcgaataatagtgaataagtccgcatcgtggtccgcaaattcccgtggttatactgccttaaaaaaattacgccccgccctgccactcatcgcagtcggcctattggttaaaaaatgagctgatttaacaaaaatttaacgcgaattttaactgacggaatttttttaatgcggggcgggacggtgagtagcgtcagccggataaccaattttttactcgactaaattgtttttaaattgcgcttaaaattgaaaatattaacgcttacaatttccattcgccattcaggctgcgcaactgttgggaagggcgatcggtgcgggcctcttcgctattacgccagctggcgaattttataattgcgaatgttaaaggtaagcggtaagtccgacgcgttgacaacccttcccgctagccacgcccggagaagcgataatgcggtcgaccgcttagggggatgtgctgcaaggcgattaagttgggtaacgccagggttttcccagtcacgacgttgtaaaacgacggccagtgagcgcgcgtaatacgactcatccccctacacgacgttccgctaattcaacccattgcggtcccaaaagggtcagtgctgcaacattttgctgccggtcactcgcgcgcattatgctgagtctatagggcgaattggagctccaccgcggtggcggccgctctagaactagtggatcccccgggctgcaggaattcgatatcaagcttttacgccccgcccgatatcccgcttaacctcgaggtggcgccaccgccggcgagatcttgatcacctagggggcccgacgtccttaagctatagttcgaaaatgcggggcgggtgccactcatcgcagtactgttgtaattcattaagcattctgccgacatggaagccatcacaaacggcatgatgaacctgaatcgccagcggcatcagcaacggtgagtagcgtcatgacaacattaagtaattcgtaagacggctgtaccttcggtagtgtttgccgtactacttggacttagcggtcgccgtagtcgtccttgtcgccttgcgtataatatttgcccatggtgaaaacgggggcgaagaagttgtccatattggccacgtttaaatcaaaactggtgaaactcacccaggaacagcggaacgcatattataaacgggtaccacttttgcccccgcttcttcaacaggtataaccggtgcaaatttagttttgaccactttgagtgggtgggattggctgagacgaaaaacatattctcaataaaccctttagggaaataggccaggttttcaccgtaacacgccacatcttgcgaatatatgtgtagaccctaaccgactctgctttttgtataagagttatttgggaaatccctttatccggtccaaaagtggcattgtgcggtgtagaacgcttatatacacatctaactgccggaaatcgtcgtggtattcactccagagcgatgaaaacgtttcagtttgctcatggaaaacggtgtaacaagggtgaacactatcccatatcattgacggcctttagcagcaccataagtgaggtctcgctacttttgcaaagtcaaacgagtaccttttgccacattgttcccacttgtgatagggtatagtccagctcaccgtctttcattgccatacgaaattccggatgagcattcatcaggcgggcaagaatgtgaataaaggccggataaaacttgtgcttatttttggtcgagtggcagaaagtaacggtatgctttaaggcctactcgtaagtagtccgcccgttcttacacttatttccggcctattttgaacacgaataaaaactttacggtctttaaaaaggccgtaatatccagctgaacggtctggttataggtacattgagcaactgactgaaatgcctcaaaatgttctttacgatgcgaaatgccagaaatttttccggcattataggtcgacttgccagaccaatatccatgtaactcgttgactgactttacggagttttacaagaaatgctacgcattgggatatatcaacggtggtatatccagtgatttttttctccatatggttaaccttaattaaggggtcgacgggcccgggatccgatgctcttccgcgtaaccctatatagttgccaccatataggtcactaaaaaaagaggtataccaattggaattaattccccagctgcccgggccctaggctacgagaaggcgtaagatcttttactagttcagtccatctcgccgtgtatgcgggcctgacggatcaacgttcccaccgagccagtcgagatgttcatctggtcggcgatctattctagaaaatgatcaagtcaggtagagcggcacatacgcccggactgcctagttgcaagggtggctcggtcagctctacaagtagaccagccgctagagccggtacttcaaaccttgtttgcgcagttccacagccttcttgcggcgttcctgcgcacgagcgatgtagtcgcctcggtcttcggcgacgagccgtttcggccatgaagtttggaacaaacgcgtcaaggtgtcggaagaacgccgcaaggacgcgtgctcgctacatcagcggagccagaagccgctgctcggcaaagatggtgcttttcgagacgccgaacttgtcagccaactcctgcgcggtctgcgtgcgacgcatcacgcgttctgcagcacccatcagtccgtcccctctgctaccacgaaaagctctgcggcttgaacagtcggttgaggacgcgccagacgcacgctgcgtagtgcgcaagacgtcgtgggtagtcaggcaggggagacctgctgcgaacagtgccgatcgatcgaccttcttgagcttcggccgcggcgcggtggcgttcttccgtaccgcttccgtttttgcgctgctgctcactttgacgacgcttgtcacggctagctagctggaagaactcgaagccggcgccgcgccaccgcaagaaggcatggcgaaggcaaaaacgcgacgacgagtgaaagccgcggcgtgcctggattttcgagaactcggcggcggtgaaggtgcggtgggtccagtgggcgactgatttgccgatctgctcggcctcggcccgactccggcgccgcacggacctaaaagctcttgagccgccgccacttccacgccacccaggtcacccgctgactaaacggctagacgagccggagccgggctgagatggggccgatcccgtcgttggcgtcgagggtgaagttggtcagggcggtgaagtcggtgaccatctgccgccacacagtgatcgacgggtagttctgtttaccccggctagggcagcaaccgcagctcccacttcaaccagtcccgccacttcagccactggtagacggcggtgtgtcactagctgcccatcaagacaatccggatctcgcggtaggcccattcccgggtgcggtcgaacagttcgacgttccggcccgtttcggtcctgacctgtgtcttgcggccgtagtccggtggaggcctagagcgccatccgggtaagggcccacgccagcttgtcaagctgcaaggccgggcaaagccaggactggacacagaacgccggcatcaggccaccggcggggaaacggtcaccgagcgcttttgcgaggcctttgagcgagtacggatccgagggaccccagaccgtcgtccagtgcgggtggatcgggttctggccgcccctttgccagtggctcgcgaaaacgctccggaaactcgctcatgcctaggctccctggggtctggcagcaggtcacgcccacctagcccaagaccgtgagctgctgcgcgtagccctgatcggcgccgaccaccgaggcgatcagcccctggttcacccggtcgtagagccgcagcgggccctgtcgggctgcctcactcgacgacgcgcatcgggactagccgcggctggtggctccgctagtcggggaccaagtgggccagcatctcggcgtcgcccgggacagcccgacggaggagggtgtagaccgggctttcgagcagccaccacaggtgcgcgtgctcggtcgcgggattgatcgtcatcacggtcggatcgggcagatccgcgttacgcctcccacatctggcccgaaagctcgtcggtggtgtccacgcgcacgagccagcgccctaactagcagtagtgccagcctagcccgtctaggcgcaatgctgcggcccactgcgcctggtcgtcgtccacgtcgagcaccaagcccaacctgatcgacggggtgcgggccgcaatgtagcggcgggtgagcgcctccgcgacgccgggtgacgcggaccagcagcaggtgcagctcgtggttcgggttggactagctgccccacgcccggcgttacatcgccgcccactcgcggaggcgccgcggctgcggccactgcccgtcccggacgtagtcatccgtcgcgtgcgggtatttgaaccgccagcggtccaaccaggcgtcaacagcagcggtcatgagcgccgacgccggtgacgggcagggcctgcatcagtaggcagcgcacgcccataaacttggcggtcgccaggttggtccgcagttgtcgtcgccagtactccgccaagctagggccggatctgtaccgatcgggggaggcgcgccgcaaattatttaagagtctcgctagcaaaccatgtcaggtgttgcggtgggttccggcggttcgatcccggcctagacatggctagccccctccgcgcggcgtttaataaattctcagagcgatcgtttggtacagtccacaacgccacccaagggggtaaacctccacccgaattatttaagagtctcgctagctaagccctatctgatgctgcgcggggggtccttcgcactgaatctcaaaggtggccggctcccatttggaggtgggcttaataaattctcagagcgatcgattcgggatagactacgacgcgccccccaggaagcgtgacttagagtttccaccggccgagaatttcgtcgcgcgaaaacctccctggacagttctggaattcagcaagaggtgtgtctgaacttcggtgtttttttggggggtgactccagcggggtggcttaaagcagcgcgcttttggagggacctgtcaagaccttaagtcgttctccacacagacttgaagccacaaaaaaaccccccactgaggtcgccccaccgcacaacgcgaacagagaccttgtgtgtacgacggcgggaggtaagtcgggtacggctcggactgcggtagagcaaccgtcgaatcgatttcgagcagagcgtgttgcgcttgtctctggaacacacatgctgccgccctccattcagcccatgccgagcctgacgccatctcgttggcagcttagctaaagctcgtctccgagcagagcaagatattccaaaactccggggttcctcggcggcctcccccgtctgtttgctcaaccgagggagacctggcggtcccgcgtttccggacggctcgtctcgttctataaggttttgaggccccaaggagccgccggagggggcagacaaacgagttggctccctctggaccgccagggcgcaaaggcctgccgcgggaccgcctaccgctcgagagcggaagagcatctagatgcattcgcgaggtacccagcttttgttccctttagtgagggttaattgcgcgcttggcgcgccctggcggatggcgagctctcgccttctcgtagatctacgtaagcgctccatgggtcgaaaacaagggaaatcactcccaattaacgcgcgaaccggtaatcatggtcatagctgtttcctgtgtgaaattgttatccgctcacaattccacacaacatacgagccggaagcataaagtgtaaagcctggggtgcccattagtaccagtatcgacaaaggacacactttaacaataggcgagtgttaaggtgtgttgtatgctcggccttcgtatttcacatttcggaccccacggtaatgagtgagctaactcacattaattgcgttgcgctcactgcccgctttccagtcgggaaacctgtcgtgccagctgcattaatgaatcggccaacgcgattactcactcgattgagtgtaattaacgcaacgcgagtgacgggcgaaaggtcagccctttggacagcacggtcgacgtaattacttagccggttgcgccggggagaggcggtttgcgtattgggcgcatgcataaaaactgttgtaattcattaagcattctgccgacatggaagccatcacaaacggcatgatgaacgcccctctccgccaaacgcataacccgcgtacgtatttttgacaacattaagtaattcgtaagacggctgtaccttcggtagtgtttgccgtactacttgctgaatcgccagcggcatcagcaccttgtcgccttgcgtataatatttgcccatgggggtgggcgaagaactccagcatgagatccccgcgctggaggatgacttagcggtcgccgtagtcgtggaacagcggaacgcatattataaacgggtacccccacccgcttcttgaggtcgtactctaggggcgcgacctcctacatccagccggcgtcccggaaaacgattccgaagcccaacctttcatagaaggcggcggtggaatcgaaatctcgtgatggcaggttgggcgtcgcttgggtaggtcggccgcagggccttttgctaaggcttcgggttggaaagtatcttccgccgccaccttagctttagagcactaccgtccaacccgcagcgaacctcggtcatttcgaaccccagagtcccgctcagaagaactcgtcaagaaggcgatagaaggcgatgcgctgcgaatcgggagcggcgataccgtaaagcacagccagtaaagcttggggtctcagggcgagtcttcttgagcagttcttccgctatcttccgctacgcgacgcttagccctcgccgctatggcatttcgtggaggaagcggtcagcccattcgccgccaagctcttcagcaatatcacgggtagccaacgctatgtcctgatagcggtccgccacacccagccggccacagctccttcgccagtcgggtaagcggcggttcgagaagtcgttatagtgcccatcggttgcgatacaggactatcgccaggcggtgtgggtcggccggtgtctcgatgaatccagaaaagcggccattttccaccatgatattcggcaagcaggcatcgccatgggtcacgacgagatcctcgccgtcgggcatgcgcgcctagctacttaggtcttttcgccggtaaaaggtggtactataagccgttcgtccgtagcggtacccagtgctgctctaggagcggcagcccgtacgcgcggatgagcctggcgaacagttcggctggcgcgagcccctgatgctcttcgtccagatcatcctgatcgacaagaccggcttccatccgagtacgtgctcgctcactcggaccgcttgtcaagccgaccgcgctcggggactacgagaagcaggtctagtaggactagctgttctggccgaaggtaggctcatgcacgagcgaggatgcgatgtttcgcttggtggtcgaatgggcaggtagccggatcaagcgtatgcagccgccgcattgcatcagccatgatggatactttctcggcaggactacgctacaaagcgaaccaccagcttacccgtccatcggcctagttcgcatacgtcggcggcgtaacgtagtcggtactacctatgaaagagccgtcctgcaaggtgagatgacaggagatcctgccccggcacttcgcccaatagcagccagtcccttcccgcttcagtgacaacgtcgagcacagctgcgcaaggaacgttccactctactgtcctctaggacggggccgtgaagcgggttatcgtcggtcagggaagggcgaagtcactgttgcagctcgtgtcgacgcgttccttcgcccgtcgtggccagccacgatagccgcgctgcctcgtcctgcagttcattcagggcaccggacaggtcggtcttgacaaaaagaaccgggcgcccctggcgggcagcaccggtcggtgctatcggcgcgacggagcaggacgtcaagtaagtcccgtggcctgtccagccagaactgtttttcttggcccgcggggaccgctgacagccggaacacggcggcatcagagcagccgattgtctgttgtgcccagtcatagccgaatagcctctccacccaagcggccggagaacctgcggcgactgtcggccttgtgccgccgtagtctcgtcggctaacagacaacacgggtcagtatcggcttatcggagaggtgggttcgccggcctcttggacgctgcaatccatcttgttcaatcatgcgaaacgatcctcatcctgtctcttgatcagatcttgatcccctgcgccatcagatccttggcggcaagaaagccaacgttaggtagaacaagttagtacgctttgctaggagtaggacagagaactagtctagaactaggggacgcggtagtctaggaaccgccgttctttcggttccagtttactttgcagggcttcccaaccttaccagagggcgccccagctggcaattccggttcgcttgctgtccataaaaccgcccagtctagctatcgaggtcaaatgaaacgtcccgaagggttggaatggtctcccgcggggtcgaccgttaaggccaagcgaacgacaggtattttggcgggtcagatcgatagcccatgtaagcccactgcaagctacctgctttctctttgcgcttgcgttttcccttgtccagatagcccagtagctgacattcatcccaggtggcacttttggtacattcgggtgacgttcgatggacgaaagagaaacgcgaacgcaaaagggaacaggtctatcgggtcatcgactgtaagtagggtccaccgtgaaaacggggaaatgtgcgcgcccgcgttcctgctggcgctgggcctgtttctggcgctggacttcccgctgttccgtcagcagcttttcgcccacggccttgatgcccctttacacgcgcgggcgcaaggacgaccgcgacccggacaaagaccgcgacctgaagggcgacaaggcagtcgtcgaaaagcgggtgccggaactagatcgcggcggccttggcctgcatatcccgattcaacggccccagggcgtccagaacgggcttcaggcgctcccgaaggtctcgggccgtctcttgggctctagcgccgccggaaccggacgtatagggctaagttgccggggtcccgcaggtcttgcccgaagtccgcgagggcttccagagcccggcagagaacccgatgatcggccttcttgcgcatctcacgcgctcctgcggcggcctgtagggcaggctcatacccctgccgaaccgcttttgtcagccggtcggccacggcttactagccggaagaacgcgtagagtgcgcgaggacgccgccggacatcccgtccgagtatggggacggcttggcgaaaacagtcggccagccggtgccgaaccggcgtctcaacgcgctttgagattcccagcttttcggccaatccctgcggtgcataggcgcgtggctcgaccgcttgcgggctgatggtgacgtggccggccgcagagttgcgcgaaactctaagggtcgaaaagccggttagggacgccacgtatccgcgcaccgagctggcgaacgcccgactaccactgcaccggcactggtggccgctccagggcctcgtagaacgcctgaatgcgcgtgtgacgtgccttgctgccctcgatgccccgttgcagccctagatcggccacagcggtgaccaccggcgaggtcccggagcatcttgcggacttacgcgcacactgcacggaacgacgggagctacggggcaacgtcgggatctagccggtgtcgcgccgcaaacgtggtctggtcgcgggtcatctgcgctttgttgccgatgaactccttggccgacagcctgccgtcctgcgtcagcggcaccacgaacgcggcggcgtttgcaccagaccagcgcccagtagacgcgaaacaacggctacttgaggaaccggctgtcggacggcaggacgcagtcgccgtggtgcttgcgcctcatgtgcgggctggtttcgtcacggtggatgctggccgtcacgatgcgatccgccccgtacttgtccgccagccacttgtgcgccttctcgaagaacgcagtacacgcccgaccaaagcagtgccacctacgaccggcagtgctacgctaggcggggcatgaacaggcggtcggtgaacacgcggaagagcttcttgcgcgcctgctgttcttggctggccgacttccaccattccgggctggccgtcatgacgtactcgaccgccaacacagcgtccttgcgccgcttctctggcagcgcggacgacaagaaccgaccggctgaaggtggtaaggcccgaccggcagtactgcatgagctggcggttgtgtcgcaggaacgcggcgaagagaccgtcgaactcgcgcagtcggcccatcgcttcatcggtgctgctggccgcccagtgctcgttctctggcgtcctgctggcgtcagcgttgggcgtctcgcgctcgcttgagcgcgtcagccgggtagcgaagtagccacgacgaccggcgggtcacgagcaagagaccgcaggacgaccgcagtcgcaacccgcagagcgcgagcgggtaggcgtgcttgagactggccgccacgttgcccattttcgccagcttcttgcatcgcatgatcgcgtatgccgccatgcctgcccctcccttttggtgccatccgcacgaactctgaccggcggtgcaacgggtaaaagcggtcgaagaacgtagcgtactagcgcatacggcggtacggacggggagggaaaaccactccaaccggctcgacgggggcagcgcaaggcggtgcctccggcgggccactcaatgcttgagtatactcactagactttgcttcgcaaagtcgtgaccgcaggttggccgagctgcccccgtcgcgttccgccacggaggccgcccggtgagttacgaactcatatgagtgatctgaaacgaagcgtttcagcactggcgctacggcggctgcggcgccctacgggcttgctctccgggcttcgccctgcgcggtcgctgcgctcccttgccagcccgtggatatgtggacgatggccgcgatgccgccgacgccgcgggatgcccgaacgagaggcccgaagcgggacgcgccagcgacgcgagggaacggtcgggcacctatacacctgctaccggcggagcggccaccggctggctcgcttcgctcggcccgtggacaaccctgctggacaagctgatggacaggctgcgcctgcccacgagcttgaccacagggatctcgccggtggccgaccgagcgaagcgagccgggcacctgttgggacgacctgttcgactacctgtccgacgcggacgggtgctcgaactggtgtccctatgcccaccggctacccagccttcgaccacatacccaccggctccaactgcgcggcctgcggccttgccccatcaatttttttaattttctctggggaaaaacgggtggccgatgggtcggaagctggtgtatgggtggccgaggttgacgcgccggacgccggaacggggtagttaaaaaaattaaaagagaccccttttgcctccggcctgcggcctgcgcgcttcgcttgccggttggacaccaagtggaaggcgggtcaaggctcgcgcagcgaccgcgcagcggcttggccttgaccggaggccggacgccggacgcgcgaagcgaacggccaacctgtggttcaccttccgcccagttccgagcgcgtcgctggcgcgtcgccgaaccggaactggcgcctggaacgacccaagcctatgcgagtgggggcagtcgaaggcgaagcccgcccgcctgccccccgagcctcacggcggcgagtgcgggggttccaacgcggaccttgctgggttcggatacgctcacccccgtcagcttccgcttcgggcgggcggacggggggctcggagtgccgccgctcacgcccccaaggttgggggcagcgccaccttgggcaaggccgaaggccgcgcagtcgatcaacaagccccggaggggccactttttgccggagcccccgtcgcggtggaacccgttccggcttccggcgcgtcagctagttgttcggggcctccccggtgaaaaacggcctc

SEQ ID NO: 5 - protein, 616 aa, Arabidopsis sp.
MEALFLSSSSSSIVASNKLTRLHNHCVWSTVIRDKKRFGPTWCRVGGGGDGGRNSNAERPIRVSSLLKDRGQVLIREQSSPAMDAETLVLSPNGNGRTIEINGVKTLMPFSGASMVGMKEGLGIISFLQGKKFLITGSTGFLAKVLIEKVLRMAPDVSKIYLLIKAKSKEAAIERLKNEVLDAELFNTLKETHGASYMSFMLTKLIPVTGNICDSNIGLQADSAEEIAKEVDVIINSAANTTFNERYDVALDINTRGPGNLMGFAKKCKKLKLFLQVSTAYVNGQRQGRIMEKPFSMGDCIATENFLEGNRKALDVDREMKLALEAARKGTQNQDEAQKMKDLGLERARSYGWQDTYVFTKAMGEMMINSTRGDVPVVIIRPSVIESTYKDPFPGWMEGNRMMDPIVLCYGKGQLTGFLVDPKGVLDVVPADMVVNATLAAIAKHGMAMSDPEPEINVYQIASSAINPLVFEDLAELLYNHYKTSPCMDSKGDPIMVRLMKLFNSVDDFSDHLWRDAQERSGLMSGMSSVDSKMMQKLKFICKKSVEQAKHLATIYEPYTFYGGRFDNSNTQRLMENMSEDEKREFGFDVGSINWTDYITNVHIPGLRRHVLKGRA

SEQ ID NO: 6 - protein, 548 aa, Arabidopsis sp.
MATTNVLATSHAFKLNGVSYFSSFPRKPNHYMPRRRLSHTTRRVQTSCFYGETSFEAVTSLVTPKTETSRNSDGIGIVRFLEGKSYLVTGATGFLAKVLIEKLLRESLEIGKIFLLMRSKDQESANKRLYDEIISSDLFKLLKQMHGSSYEAFMKRKLIPVIGDIEEDNLGIKSEIANMISEEIDVIISCGGRTTFDDRYDSALSVNALGPGRLLSFGKGCRKLKLFLHFSTAYVTGKREGTVLETPLCIGENITSDLNIKSELKLASEAVRKFRGREEIKKLKELGFERAQHYGWENSYTFTKAIGEAVIHSKRGNLPVVIIRPSIIESSYNEPFPGWIQGTRMADPIILAYAKGQISDFWADPQSLMDIIPVDMVANAAIAAMAKHGCGVPEFKVYNLTSSSHVNPMRAGKLIDLSHQHLCDFPLEETVIDLEHMKIHSSLEGFTSALSNTIIKQERVIDNEGGGLSTKGKRKLNYFVSLAKTYEPYTFFQARFDNTNTTSLIQEMSMEEKKTFGFDIKGIDWEHYIVNVHLPGLKKEFLSKKKTE

SEQ ID NO: 7 - protein, 491 aa, Arabidopsis sp.
MESNCVQFLGNKTILITGAPGFLAKVLVEKILRLQPNVKKIYLLLRAPDEKSAMQRLRSEVMEIDLFKVLRNNLGEDNLNALMREKIVPVPGDISIDNLGLKDTDLIQRMWSEIDIIINIAATTNFDERYDIGLGINTFGALNVLNFAKKCVKGQLLLHVSTAYISGEQPGLLLEKPFKMGETLSGDRELDINIEHDLMKQKLKELQDCSDEEISQTMKDFGMARAKLHGWPNTYVFTKAMGEMLMGKYRENLPLVIIRPTMITSTIAEPFPGWIEGLKTLDSVIVAYGKGRLKCFLADSNSVFDLIPADMVVNAMVAAATAHSGDTGIQAIYHVGSSCKNPVTFGQLHDFTARYFAKRPLIGRNGSPIIVVKGTILSTMAQFSLYMTLRYKLPLQILRLINIVYPWSHGDNYSDLSRKIKLAMRLVELYQPYLLFKGIFDDLNTERLRMKRKENIKELDGSFEFDPKSIDWDNYITNTHIPGLITHVLKQ

SEQ ID NO: 8 - protein, 231 aa, Unknown
  Description of Unknown Cyanobacterium polypeptide
MPELAVRTEFDYSSEIYKDAYSRINAIVIEGEQEAYSNYLQMAELLPEDKEELTRLAKMENRHKKGFQACGNNLQVNPDMPYAQEFFAGLHGNFQHAFSEGKVVTCLLIQALIIEAFAIAAYNIYIPVADDFARKITEGVVKDEYTHLNYGEEWLKANFATAKEELEQANKENLPLVWKMLNQVQGDAKVLGMEKEALVEDFMISYGEALSNIGFSTREIMRMSSYGLAGV

SEQ ID NO: 9 - protein, 340 aa, Unknown
  Description of Unknown Cyanobacterium polypeptide
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 10 - DNA, 410 bp, Unknown
  Description of Unknown Cyanobacterium rubisco polypeptide
cagtcaatggagagcattgccataagtaaaggcatcccctgcgtgataagattaccttcagaaaacagatagttgctgggttatcgcagatttttctcgcgtcagttacctctcgtaacggtattcatttccgtaggggacgcactattctaatggaagtcttttgtctatcaacgacccaatagcgtctaaaaagagcgaaccaaataactgtaaataataactgtctctggggcgacggtaggctttatattgccaaatttcgcccgtgggagaaagctaggctattcaatgtttatgttggtttattgacatttattattgacagagaccccgctgccatccgaaatataacggtttaaagcgggcaccctctttcgatccgataagttacaaatacgaggactcct

SEQ ID NO: 11 - DNA, 2962 bp, Rhodococcus opacus
  modified_base (1070)..(1070): a, c, t, g, unknown or other
cctggctcaggacgaacgctggcggcgtgcttaacacatgcaagtcgagcggtaaggcccttcggggtacacgagcggcgaacgggtgagtaacacgtggggaccgagtcctgcttgcgaccgccgcacgaattgtgtacgttcagctcgccattccgggaagccccatgtgctcgccgcttgcccactcattgtgcaccgtgatctgccctgcacttcgggataagcctgggaaactgggtctaataccggatatgaccttcggctgcatggctgagggtggaaaggtttactggtgcacactagacgggacgtgaagccctattcggaccctttgacccagattatggcctatactggaagccgacgtaccgactcccacctttccaaatgaccacgtggatgggcccgcggcctatcagcttgttggtggggtaatggcctaccaaggcgacgacgggtagccgacctgagagggtgaccggccacactgggactgacctacccgggcgccggatagtcgaacaaccaccccattaccggatggttccgctgctgcccatcggctggactctcccactggccggtgtgaccctgactgacacggcccagactcctacgggaggcagcagtggggaatattgcacaatgggcgaaagcctgatgcagcgacgccgcgtgagggatgacggccttcgggctgtgccgggtctgaggatgccctccgtcgtcaccccttataacgtgttacccgctttcggactacgtcgctgcggcgcactccctactgccggaagcccttgtaaacctctttcagcagggacgaagcgaaagtgacggtacctgcagaagaagcaccggccaactacgtgccagcagccgcggtaatacgtagggtgcaacatttggagaaagtcgtccctgcttcgctttcactgccatggacgtcttcttcgtggccggttgatgcacggtcgtcggcgccattatgcatcccacgaagcgttgtccggaattactgggcgtaaagagctcgtaggcggtttgtcgcgtcgtctgtgaaaactcanagctcaacctcgagcttgcaggcgatacggttcgcaacaggccttaatgacccgcatttctcgagcatccgccaaacagcgcagcagacacttttgagtntcgagttggagctcgaacgtccgctatgccgcagacttgagtactgcaggggagactggaattcctggtgtagcggtgaaatgcgcagatatcaggaggaacaccggtggcgaaggcgggtctctgggcacgtctgaactcatgacgtcccctctgaccttaaggaccacatcgccactttacgcgtctatagtcctccttgtggccaccgcttccgcccagagacccgtgtaactgacgctgaggagcgaaagcgtgggtagcaaacaggattagataccctggtagtccacgccgtaaacggtgggcgctaggtgtgggtttccttcccattgactgcgactcctcgctttcgcacccatcgtttgtcctaatctatgggaccatcaggtgcggcatttgccacccgcgatccacacccaaaggaaggacgggatccgtgccgtagttaacgcattaagcgccccgcctggggagtacggccgcaaggttaaaactcaaaggaattgacgggggcccgcacaagcggctgccctaggcacggcatcaattgcgtaattcgcggggcggacccctcatgccggcgttccaattttgagtttccttaactgcccccgggcgtgttcgccgggagcatgtggattaattcgatgcaacgcgaagaaccttacctgggtttgacatataccggaaagccgtagagataccgccccccttgtggtcggtataccctcgtacacctaattaagctacgttgcgcttcttggaatggacccaaactgtatatggcctttcggcatctctatggcggggggaacaccagccatatgaggtggtgcatggctgtcgtcagctcgtgtcgtgagatgttgggttaagtcccgcaacgagcgcaacccttgtcttatgttgccagcacgtaatggtgggtccaccacgtaccgacagcagtcgagcacagcactctacaacccaattcagggcgttgctcgcgttgggaacagaatacaacggtcgtgcattaccacccgactcgtaagagactgccggggtcaactcggaggaaggtggggacgacgtcaagtcatcatgccccttatgtccagggcttcacacatgctacaatggccctgagcattctctgacggccccagttgagcctccttccacccctgctgcagttcagtagtacggggaatacaggtcccgaagtgtgtacgatgttaccggggtacagagggctgcgataccgtgaggtggagcgaatcccttaaagccggtctcagttcggatcggggtctgcaactcgaccccgtgaagtcggagtcgcccatgtctcccgacgctatggcactccacctcgcttagggaatttcggccagagtcaagcctagccccagacgttgagctggggcacttcagcctcagcgtagtaatcgcagatcagcaacgctgcggtgaatacgttcccgggccttgtacacaccgcccgtcacgtcatgaaagtcggtaacacccgaagccggtggcatcattagcgtctagtcgttgcgacgccacttatgcaagggcccggaacatgtgtggcgggcagtgcagtactttcagccattgtgggcttcggccaccgctaaccccttgtgggagggagccgtcgaaggtgggatcggcgattgggacgaagtcgtaacaaggtagccgtaccggaagggattggggaacaccctccctcggcagcttccaccctagccgctaaccctgcttcagcattgttccatcggcatggccttcc

SEQ ID NO: 12 - DNA, 3042 bp, Rhodococcus opacus
tcaacggagagtttgatcctggctcaggacgaacgctggcggcgtgcttaacacatgcaagtcgagcggtaaggcccttcggggtacacgagcggcgaacagttgcctctcaaactaggaccgagtcctgcttgcgaccgccgcacgaattgtgtacgttcagctcgccattccgggaagccccatgtgctcgccgcttggggtgagtaacacgtgggtgatctgccctgcacttcgggataagcctgggaaactgggtctaataccggatatgaccttcggctgcatggccgttggtggcccactcattgtgcacccactagacgggacgtgaagccctattcggaccctttgacccagattatggcctatactggaagccgacgtaccggcaaccaccaaaggtttactggtgcaggatgggcccgcggcctatcagcttgttggtggggtaatggcctaccaaggcgacgacgggtagccgacctgagagggtgacctttccaaatgaccacgtcctacccgggcgccggatagtcgaacaaccaccccattaccggatggttccgctgctgcccatcggctggactctcccactggggccacactgggactgagacacggcccagactcctacgggaggcagcagtggggaatattgcacaatgggcgaaagcctgatgcagcgacgccgcgtgagccggtgtgaccctgactctgtgccgggtctgaggatgccctccgtcgtcaccccttataacgtgttacccgctttcggactacgtcgctgcggcgcactcggatgacggccttcgggttgtaaacctctttcagcagggacgaagcgaaagtgacggtacctgcagaagaagcaccggccaactacgtgccagcagccgccctactgccggaagcccaacatttggagaaagtcgtccctgcttcgctttcactgccatggacgtcttcttcgtggccggttgatgcacggtcgtcggcgggtaatacgtagggtgcaagcgttgtccggaattactgggcgtaaagagctcgtaggcggtttgtcgcgtcgtctgtgaaaactcgaggctcaacctcgaccattatgcatcccacgttcgcaacaggccttaatgacccgcatttctcgagcatccgccaaacagcgcagcagacacttttgagctccgagttggagctgcttgcaggcgatacgggcagacttgagtactgcaggggagactggaattcctggtgtagcggtgaaatgcgcagatatcaggaggaacaccggtggcgacgaacgtccgctatgcccgtctgaactcatgacgtcccctctgaccttaaggaccacatcgccactttacgcgtctatagtcctccttgtggccaccgctaggcgggtctctgggcagtaactgacgctgaggagcgaaagcgtgggtagcgaacaggattagataccctggtagtccacgccgtaaacggtgggcgctatccgcccagagacccgtcattgactgcgactcctcgctttcgcacccatcgcttgtcctaatctatgggaccatcaggtgcggcatttgccacccgcgatggtgtgggtttccttccacgggatccgtgccgtagctaacgcattaagcgccccgcctggggagtacggccgcaaggctaaaactcaaaggaattgacggccacacccaaaggaaggtgccctaggcacggcatcgattgcgtaattcgcggggcggacccctcatgccggcgttccgattttgagtttccttaactgccgggcccgcacaagcggcggagcatgtggattaattcgatgcaacgcgaagaaccttacctgggtttgacatataccggaaagctgcagagatgtggcccccccgggcgtgttcgccgcctcgtacacctaattaagctacgttgcgcttcttggaatggacccaaactgtatatggcctttcgacgtctctacaccggggccttgtggtcggtatacaggtggtgcatggctgtcgtcagctcgtgtcgtgagatgttgggttaagtcccgcaacgagcgcaacccttgtcttatgttgcggaacaccagccatatgtccaccacgtaccgacagcagtcgagcacagcactctacaacccaattcagggcgttgctcgcgttgggaacagaatacaacgcagcacgtaatggtggggactcgtaagagactgccggggtcaactcggaggaaggtggggacgacgtcaagtcatcatgccccttatgtccagggcttcagtcgtgcattaccacccctgagcattctctgacggccccagttgagcctccttccacccctgctgcagttcagtagtacggggaatacaggtcccgaagtcacatgctacaatggccggtacagagggctgcgataccgtgaggtggagcgaatcccttaaagccggtctcagttcggatcggggtctgcaactcgacccgtgtacgatgttaccggccatgtctcccgacgctatggcactccacctcgcttagggaatttcggccagagtcaagcctagccccagacgttgagctgggcgtgaagtcggagtcgctagtaatcgcagatcagcaacgctgcggtgaatacgttcccgggccttgtacacaccgcccgtcacgtcatgaaagtcggtaagcacttcagcctcagcgatcattagcgtctagtcgttgcgacgccacttatgcaagggcccggaacatgtgtggcgggcagtgcagtactttcagccattcacccgaagccggtggcctaacccctcgtgggagggagccgtcgaaggtgggatcggcgattgggacgaagtcgtaacaaggtagccgtaccggaaggtggtgggcttcggccaccggattggggagcaccctccctcggcagcttccaccctagccgctaaccctgcttcagcattgttccatcggcatggccttccaccggctggatcacctcctttctgccgacctagtggaggaaaga

SEQ ID NO: 13 - DNA, 2924 bp, Ralstonia sp.
acgtggcggcatgccttacacatgcaagtcgaacggcagcgcggacttcggtctggcggcgagtggcgaacgggtgagtaatacatcggaacgtaccctgtgcaccgccgtacggaatgtgtacgttcagcttgccgtcgcgcctgaagccagaccgccgctcaccgcttgcccactcattatgtagccttgcatgggacttgtgggggataactagtcgaaagattagctaataccgcatacgacctgagggtgaaagtgggggaccgcaaggcctcacgcagcaggagcggccgatgtaacaccccctattgatcagctttctaatcgattatggcgtatgctggactcccactttcaccccctggcgttccggagtgcgtcgtcctcgccggctacactgattagctagttggtggggtaaaggcccaccaaggcgacgatcagtagctggtctgagaggacgatcagccacactgggactgagacacggcccagacgactaatcgatcaaccaccccatttccgggtggttccgctgctagtcatcgaccagactctcctgctagtcggtgtgaccctgactctgtgccgggtctgtcctacgggaggcagcagtggggaattttggacaatgggggcaaccctgatccagcaatgccgcgtgtgtgaagaaggccttcgggttgtaaagcactttaggatgccctccgtcgtcaccccttaaaacctgttacccccgttgggactaggtcgttacggcgcacacacttcttccggaagcccaacatttcgtgaaatgtccggaaagaaatcgcgctggttaatacctgcgtgatgacggtaccggaagaataagcaccggctaactacgtgccagcagccgcggtaatacgtaggacaggcctttctttagcgcgaccaattatggacgcactactgccatggccttcttattcgtggccgattgatgcacggtcgtcggcgccattatgcatccgtgcgagcgttaatcggaattactgggcgtaaagcgtgcgcaggcggttttgtaagacaggcgtgaaatccccgggcttaacctgggaattgcgcttgtgcacgctcgcaattagccttaatgacccgcatttcgcacgcgtccgccaaaacattctgtccgcactttaggggcccgaattggacccttaacgcgaacacactgcaaggctagagtgcgtcagaggggggtagaattccacgtgtagcagtgaaatgcgtagagatgtggaggaataccgatggcgaaggcgagcccccttgacgttccgatctcacgcagtctccccccatcttaaggtgcacatcgtcactttacgcatctctacacctccttatggctaccgcttccgctcgggggaggaccttgactgacgctcatgcacgaaagcgtggggagcaaacaggattagataccctggtagtccacgccctaaacgatgtcaactagttgttgggattcctggaactgactgcgagtacgtgctttcgcacccctcgtttgtcctaatctatgggaccatcaggtgcgggatttgctacagttgatcaacaaccctaacattttctcagtaacgtagctaacgcgtgaagttgaccgcctggggagtacggctgcaagattaaaactcaaaggaattgacggggacccgcacaagcgggtaaaagagtcattgcatcgattgcgcacttcaactggcggacccctcatgccgacgttctaattttgagtttccttaactgcccctgggcgtgttcgcctggatgatgtggattaattcgatgcaacgcgaaaaaccttacctacccttgacatgccctaacgaagcagagatgcattagtgcccgcaaagggaaagtgacctactacacctaattaagctacgttgcgctttttggaatggatgggaactgtacgggattgcttcgtctctacgtaatcacgggcgtttccctttcacggacacaggtgctgcatggctgtcgtcagctcgtgtcgtgagatgttgggttaagtcccgcaacgagcgcaacccttgtctctagttgcctacgcaagagcctgtgtccacgacgtaccgacagcagtcgagcacagcactctacaacccaattcagggcgttgctcgcgttgggaacagagatcaacggatgcgttctccactctagagagactgccggtgacaaaccggaggaaggtggggatgacgtcaagtcctcatggcccttatgggtagggcttcacacgtcatacaatggtggtgagatctctctgacggccactgtttggcctccttccacccctactgcagttcaggagtaccgggaatacccatcccgaagtgtgcagtatgttaccaccgtacagagggttgccaacccgcgagggggagctaatcccagaaaacgcatcgtagtccggatcgtagtctgcaactcgactacgtgaagctggaatcgcgcatgtctcccaacggttgggcgctccccctcgattagggtcttttgcgtagcatcaggcctagcatcagacgttgagctgatgcacttcgaccttagcgtagtaatcgcggatcagcatgccgcggtgaatacgttcccgggtcttgtacacaccgcccgtcacaccatgggagtgggttttgccagaagtagttagccatcattagcgcctagtcgtacggcgccacttatgcaagggcccagaacatgtgtggcgggcagtgtggtaccctcacccaaaacggtcttcatcaatcggtaaccgcaaggagggcgattaccacggcagggttcatgactggggtgaagtcgtaacaaggtattggcgttcctcccgctaatggtgccgtcccaagtactgaccccacttcagcattgttcca

SEQ ID NO: 14 - protein, 284 aa, Rhodococcus opacus
MASIEDILELEALEKDIFRGAVHPSVLKRTFGGQVAGQSLVSAVRTVDERFEVHSLHGYFLRPGNPTEPTVYLVDRIRDGRSFCTRRVTGIQDGKAIFTMSASFHSQDEGIEHQDTMPSVPEPEELVDAQTVEEMAATDLYREWKEWDVRIVPAGCTGKTPGIAAKQRVWMRYRNKLPDDQVFHICTLAYLSDMTLLGASKVPHPGVVTQTASLDHAMWFLRPFRADEWLLYDQTSPSAGFGRALTQGRMFDRKGTMVAAVVQEGLTRIQRDQDQRDIETGNMA

SEQ ID NO: 15 - DNA, 1460 bp, Rhodococcus opacus
  modified_base (799)..(799): a, c, t, g, unknown or other
ggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcacagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagntaacgcattaagcgccccgcctggggagtacggccgcaaggttaaaactcaaaggaattgacgggggcccgcacaagcggcggagcatgtggattaattcgatgcaacgcgaagaaccttacctgggtttgacatataccggaaagccgtagagataccgccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaag

SEQ ID NO: 16 - DNA, 1473 bp, Rhodococcus opacus
  modified_base (562)..(562): a, c, t, g, unknown or other
aggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaagccgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcanagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaagg

SEQ ID NO: 17 - DNA, 1462 bp, Cupriavidus necator
acgtggcggcatgccttacacatgcaagtcgaacggcagcgcggacttcggtctggcggcgagtggcgaacgggtgagtaatacatcggaacgtaccctgttgtgggggataactagtcgaaagattagctaataccgcatacgacctgagggtgaaagtgggggaccgcaaggcctcacgcagcaggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaatcgcgctggttaatacctgcgtgatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgggcttaacctgggaattgcgcttgtgactgcaaggctagagtgcgtcagaggggggtagaattccacgtgtagcagtgaaatgcgtagagatgtggaggaataccgatggcgaaggcgagccccctggaccttgactgacgctcatgcacgaaagcgtggggagcaaacaggattagataccctggtagtccacgccctaaacgatgtcaactagttgttgggattcattttctcagtaacgtagctaacgcgtgaagttgaccgcctggggagtacggctgcaagattaaaactcaaaggaattgacggggacccgcacaagcggtggatgatgtggattaattcgatgcaacgcgaaaaaccttacctacccttgacatgccctaacgaagcagagatgcattagtgcccgcaaagggaaagtgggacacaggtgctgcatggctgtcgtcagctcgtgtcgtgagatgttgggttaagtcccgcaacgagcgcaacccttgtctctagttgcctacgcaagagcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgtagtctgcaactcgactacgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgtaacaaggt

SEQ ID NO: 18 - DNA, 1479 bp, Ralstonia sp.
agtttgatcctggctcagattgaacgctggcggcatgccttacacatgcaagtcgaacggcagcgcggacttcggtctggcggcgagtggcgaacgggtgagtaatacatcggaacgtaccctgttgtgggggataactagtcgaaagattagctaataccgcatacgacctgagggtgaaagcgggggaccgtaaggcctcgcgcagcaggagcggccgatgtctgattagctagttggtggggtaaaggcccaccaaggcgacgatcagtagctggtctgagaggacgatcagccacactgggactgagacacggcccagactcctacgggaggcagcagtggggaattttggacaatgggggcaaccctgatccagcaatgccgcgtgtgtgaagaaggccttcgggttgtaaagcacttttgtccggaaagaaaacgctctggttaatacctggagtggatgacggtaccggaagaataagcaccggctaactacgtgccagcagccgcggtaatacgtagggtgcgagcgttaatcggaattactgggcgtaaagcgtgcgcaggcggttttgtaagacaggcgtgaaatccccgagctcaacttgggaattgcgcttgtgactgcaaggctagagtatgtcagaggggggtagaattccacgtgtagcagtgaaatgcgtagagatgtggaggaataccgatggcgaaggcagccccctgggacgtcactgacgctcatgcacgaaagcgtggggagcaaacaggattagataccctggtagtccacgccctaaacgatgtcaactagttgttggggattcatttcttcagtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgccactaacgaagcagagatgcatcaggtgcccgaaagggaaagtggacacaggtgctgcatggctgtcgtcagctcgtgtcgtgagatgttgggttaagtcccgcaacgagcgcaacccttatctttagttgctacgcaagggcactctagagagactgccggtgacaaaccggaggaaggtggggatgacgtcaagtcctcatggcccttatgggtagggcttcacacgtcatacaatggtgcgtacagagggttgccaacccgcgagggggagctaatcccagaaaacgcatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggttttgccagaagtagttagcctaaccgcaaggagggcgattaccacggcagggttcatgactggggtgaagtcgt

SEQ ID NO: 19 - DNA, 1486 bp, Gordonia alkanivorans
gctcaggacgaacgctggcggcgtgcttaacacatgcaagtcgaacggaaaggcccagcttgctgggtactcgagtggcgaacgggtgagtaacacgtgggtgatctgccctgaactttgggataagcctgggaaactgggtctaataccggatatgaccttggagtgcatgctctggggtggaaagcttttgcggttcaggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgcaagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgtaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtggggctcatttcacgagttccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcatgtagagatacatgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcgg

SEQ ID NO: 20 - DNA, 1491 bp, Gordonia sp.
gatcatggctcaggacgaacgctggcggcgtgcttaacacatgcaagtcgaacggaaaggcccgcttgcgggtactcgagtggcgaacgggtgagtaacacgtgggtgatctgccctggactctgggataagcctgggaaactgggtctaataccggatatgaccttacatcgcatggtgtttggtggaaagcttttgcggttcaggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgcaagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgtaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtggggctcatttcacgagttccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagaaagctatagagatatagccccccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagctgtcgaaggtgggatcggcgattgggacgaagtcgtaacaaggtagccgtaccggaaggtgcgg

SEQ ID NO: 21 - DNA, 1505 bp, Mycobacterium fortuitum
ttgatcctggctcaggacgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaatatgaccacgcgcttcatggtgtgtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcaatagggacgaagcgcaagtgacggtacctatagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatgcacaggacgactgcagagatgtggtttcccttgtggcctgtgtgcaggtggtgcatggctgtcgtcagctcgtgtcgtgagatgttgggttaagtcccgcaacgagcgcaacccttgtctcatgttgccagcacgttatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctcctt

SEQ ID NO: 22 - DNA, 1460 bp, Mycobacterium parafortuitum
cgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaatatgatcattggcttcctggctggtggtggaaagcttttgcggtgtgggatgggcccgcggcctatcagcttgttggtggggtaatggcctaccaaggcgacgacgggtagccggcctgagagggtgaccggccacactgggactgagatacggcccagactcctacgggaggcagcagtggggaatattgcacaatgggcgcaagcctgatgcagcgacgccgcgtgagggatgacggccttcgggttgtaaacctctttcgccagggacgaagcgcaagtgacggtacctggagaagaaggaccggccaactacgtgccagcagccgcggtaatacgtagggtccgagcgttgtccggaattactgggcgtaaagagctcgtaggtggtttgtcgcgttgttcgtgaaaactcacagcttaactgtgggcgtgcgggcgatacgggcagactagagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccg

SEQ ID NO: 23 - DNA, 1480 bp, Mycobacterium sphagni
gagtttgatcctggctcaggacgaacgctggcggcgtgcttaacacatgcaagtcgaacggaaaggcccttcggggtactcgagtggcgaacgggtgagtaacacgtgggtgatctgccctgcactttgggataagcctgggaaactgggtctaataccgaataggaccgcatgcttcatggtgtgtggtggaaagcttttgcggtgtgggatgggcccgcggcctatcagcttgttggtggggtaatggcctaccaaggcgacgacgggtagccggcctgagagggtgtccggccacactgggactgagatacggcccagactcctacgggaggcagcagtggggaatattgcacaatgggcgcaagcctgatgcagcgacgccgcgtgagggatgacggccttcgggttgtaaacctctttcagcagggacgaagcgcaagtgacggtacctgtagaagaagcaccggccaactacgtgccagcagccgcggtaatacgtagggtgcgagcgttgtccggaattactgggcgtaaagagctcgtaggtggtttgtcgcgttgttcgtgaaaactcacagctcaactgtgggcgtgcgggcgatacgggcagacttgagtactgcaggggagactggaattcctggtgtagcggtggaatgcgcagatatcaggaggaacaccggtggcgaaggcgggtctctgggcagtaactgacgctgaggagcgaaagcgtggggagcgaacaggattagataccctggtagtccacgccgtaaacggtgggtactaggtgtgggtttccttccttgggatccgtgccgtagctaacgcattaagtaccccgcctggggagtacggccgcaaggctaaaactcaaagaaattgacgggggcccgcacaagcggcggagcatgtggattaattcgatgcaacgcgaagaaccttacctgggtttgacatgcacaggacgccggcagagatgtcggttcccttgtggcctgtgtgcaggtggtgcatggctgtcgtcagctcgtgtcgtgagatgttgggttaagtcccgcaacgagcgcaacccttgtctcatgttgccagcacgtaatggtggggactcgtgagagactgccggggtcaactcggaggaaggtggggatgacgtcaagtcatcatgccccttatgtccagggcttcacacatgctacaatggccggtacaaagggctgcgatgccgtgaggtggagcgaatcctttcaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagcc

SEQ ID NO: 24 - DNA, 1494 bp, Nocardia farcinica
gacgaacgctggcggcgtgcttaacacatgcaagtcgagcggtaaggcccttcggggtacacgagcggcgaacgggtgagtaacacgtgggtgatctgccctgtacttcgggataagcctgggaaactgggtctaataccggatatgaccttacatcgcatggtgtttggtggaaagatttatcggtacaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcgacagggacgaagcgcaagtgacggtacctgtagaagaagcaccggccaactacgtgccagcagccgcggtaatacgtagggtgcgagcgttgtccggaattactgggcgtaaagagcttgtaggcggtttgtcgcgtcgtccgtgaaaacttggggctcaaccccaagcttgcgggcgatacgggcagacttgagtactgcaggggagactggaattcctggtgtagcggtgaaatgcgcagatatcaggaggaacaccggtggcgaaggcgggtctctgggcagtaactgacgctgagaagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaacctgcagagatgtaggcccccttgtggtcggtgtacaggtggtgcatggctgtcgtcagctcgtgtcgtgagatgttgggttaagtcccgcaacgagcgcaacccttgtcctgtgttgccagcgcgttatggcggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagttggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacctcctttct

SEQ ID NO: 25 - DNA, 1513 bp, Nocardia sp.
gagtttgatcctggctcaggacgaacgctggcggcgtgcttaacacatgcaagtcgagcggtaaggcccttcggggtacacgagcggcgaacgggtgagtaacacgtgggtgatctgccctgtacttcgggataagcctgggaaactgggtctaataccggatatgaccttacatcgcatggtgtttggtggaaagatttatcggtacaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcgacagggacgaagcgcaagtgacggtacctgtagaagaagcaccggccaactacgtgccagcagccgcggtaatacgtagggtgcgagcgttgtccggaattactgggcgtaaagagcttgtaggcggtttgtcgcgtcgtccgtgaaaacttggggctcaaccccaagcttgcgggcgatacgggcagacttgagtactgcaggggagactggaattcctggtgtagcggtgaaatgcgcagatatcaggaggaacaccggtggcgaaggcgggtctctgggcagtaaccgacgctgagaagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaacctgcagagatgtaggcccccttgtggtcggtgtacaggtggtgcatggccgtcgtcagctcgtgtcgtgagatgttgggttaagtcccgcaacgagcgcaacccttgtcctgtgttgccagcgcgttatggcggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagttggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacctcctttct

SEQ ID NO: 26 - DNA, 1490 bp, Rhodococcus rhodochrous
gagtttgaatctggctcaggacgaacgctggcggcgtgcttaacacatgcaagtcgaacgatgaagcccagcttgctgggtggattagtggcgaacgggtgagtaacacgtgggtgatctgccctgcactctgggataagcctgggaaactgggtctaataccggatatgacctcttgctgcatggcgaggggtggaaagtttttcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcgagcgttgtccggaattactgggcgtaaagagctcgtaggcggtttgtcgcgtcgtctgtgaaatcccgcagctcaactgcgggcttgcaggcgatacgggcagactcgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagccaacgcattaagcgccccgcctggggagtacggccgcaaggctaaaactcaaaggaattgacgggggcccgcacaagcggcggagcatgtggattaattcgatgcaacgcgaagaaccttacctgggtttgacatgtaccggacgactgcagagatgtggtttcccttgtggccggtagacaggtggtgcatggctgtcgtcagctcgtgtcgtgagatgttgggttaagtcccgcaacgagcgcaacccttgtcctgtgttgccagcacgtaatggtggggactcgcaggagactgccggggtcaactcggaggaaggtggggacgacgtcaagtcatcatgccccttatgtccagggcttcacacatgctacaatggtcggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccgga

SEQ ID NO: 27 - DNA, 1481 bp, Rhodococcus coprophilus
cctggctcaggacgaacgctggcggcgtgcttaacacatgcaagtcgaacgatgatgcccagcttgctgggcggattagtggcgaacgggtgagtaacacgtgggtgatctgccctgcacttcgggataagcctgggaaactgggtctaataccggatatgaccatgggatgcatgtcctgtggtggaaaggtttactggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgactgtacctgcagaagaagcaccggctaactacgtgccagcagccgcggtaatacgtagggtgcgagcgttgtccggaattactgggcgtaaagagttcgtaggcggtttgtcgcgtcgtgtgtgaaatcccgcagctcaactgcgggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggacgactgcagagatgtggtttcccttgtggtcggtatacaggtggtgcatggctgtcgtcagctcgtgtcgtgagatgttgggttaagtcccgcaacgagcgcaacccttgtcttatgttgccagcacgtaatgggggggactcgtaagagactgccggggtcaactcggaggaaggtggggacgacgtcaagtcatcatgccccttatgtccagggcttcacacatgctacaatggtcggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccgg

SEQ ID NO: 28 - DNA, 1486 bp, Rhodococcus triatomae
ggcggcgtgcttaacacatgcaagtcgagcggtaaggcctttcggggtacacgagcggcgaacgggtgagtaacacgtgggtgatctgccctgcactctgggataagcctgggaaactgggtctaataccggatatgactaccggctgcatggtctggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcaacagggacgaagcgcaagtgacggtacctgtagaagaagcaccggccaactacgtgccagcagccgcggtaatacgtagggtgcgagcgttgtccggaattactgggcgtaaagagctcgtaggcggtttgtcgcgtcgtctgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatacaccggaaagccgtagagatacggccccccttgtggtcggtgtacaggtggtgcatggctgtcgtcagctcgtgtcgtgagatgttgggttaagtcccgcaacgagcgcaacccttgtcctgtgttgccagcacgtaatggtggggactcgcaggagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacttcctttcta

SEQ ID NO: 29 - DNA, 1507 bp, Nocardia coeliaca
tttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcaggttgcatgacttggggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctccttt

SEQ ID NO: 30 - DNA, 1507 bp, Nocardia globerula
gtttgatcctggctcaggacgaacgctggcggcgtgcttaacacatgcaagtcgagcggtaaggcctttcggggtacacgagcggcgaacgggtgagtaacacgtgggtgatctgccctgcacttcgggataagcctgggaaactgggtctaataccggatatgacctcctatcgcatggtgggtggtggaaagatttatcggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggacgacggccttcgggttgtaaacctctttcagcagggacgaagcgcaagtgacggtacctgcagaagaagcaccggctaactacgtgccagcagccgcggtaatacgtagggtgcaagcgttgtccggaattactgggcgtaaagagttcgtaggcggtttgtcacgtcgtttgtgaaaactcacagctcaactgtgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggttccttccacggaatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccctatcttatgttgccagcacgttatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcttaaccccttgtgggagggagccgtcgaaggtgggatcggcgattgggacgaagtcgtaacaaggtagccgtaccggaaggtgcggctggatcacctcctt

SEQ ID NO: 31 - DNA, 1508 bp, Rhodococcus equi
gagtttgatcctggctcaggacgaacgctggcggcgtgcttaacacatgcaagtcgagcggtagggcccttcggggtacacgagcggcgaacgggtgagtaacacgtgggtgatctgccctgcacttcgggataagcttgggaaactgggtctaataccggatatgagcctctactgcatggtggaggttggaaaggtttactggtgcaggatgggcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcgagcgttgtccggaattactgggcgtaaagagctcgtaggcggtttgtcgcgtcgtcggtgaaaaccagcagctcaactgctggcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcctgtgttgccagcacgtaatggtggggactcgcaggagaccgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccttgtggagggagccgtcgaaggtgggatcggcgattgggacgaagtcgtaacaaggtagccgtaccggaaggtgcggctggatcacctcctt

SEQ ID NO: 32 - DNA, 1442 bp, Rhodococcus sp.
ctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgagcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtctgtgaaaactcacagctcaactgtgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgaaccgtgaggtggagcgaatcccttaaagcyggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgatt

SEQ ID NO: 33 - DNA, 1474 bp, Rhodococcus sp.
agagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaactcamrgctcaactgtgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaagg

SEQ ID NO: 34 - DNA, 1437 bp, Rhodococcus jostii
aggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggctaactacgtgccagcagccgcggtaatacgtagggtgcaagcgttgtccggaattactgggcgtaaagagttcgtaggcggtttgtcgcgtcgtttgtgaaaactcacagctcaactgtgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcaaacaggattagataccctggtagtccacgccgtaaacggtgggcgctaggtgtgggttccttccacgggatctgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattg

SEQ ID NO: 35 - DNA, 1485 bp, Rhodococcus opacus
  modified_base (812)..(812): a, c, t, g, unknown or other
gatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagttcgtaggcggtttgtcgcgtcgtctgtgaaaactcaaagctcaacctcgagcctgcaggcgatacgggcagacttgagtactgcaggggagactggaattcctggtgtagcggtgaaatgcgcagatatcaggaggaacaccggtggcgaaggcgggtctctgggcagtaactgacgctgaggaacgaaagcgtgggtagcgaacaggattagataccctggtagtccacgccgtaaacggtgggcgctaggtgtgggtttccttccacgggatcngtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggt

SEQ ID NO: 36 - DNA, 1510 bp, Rhodococcus imtechensis
ttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccatcagccgcggtaatacgtagggtgcaagcgttgtccggaattactgggcgtaaagagctcgtaggcggtttgtcgtgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtggaaaccgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtaagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccaggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggaaactgccgaggggg

SEQ ID NO: 37 - DNA, 1473 bp, Rhodococcus koreensis
gacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccaaggactgcatggtttttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccagtacagagggctgcgagaccgtgaggtggagcgaatcccttaaagctggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgc

SEQ ID NO: 38 - DNA, 1510 bp, Rhodococcus opacus
gagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggccgttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattgggacgaagtcgtaacaaggtagccgtaccggaaggtgcggctggatcaccctcct

SEQ ID NO: 39 - DNA, 1450 bp, Rhodococcus sp.
tcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattggga

SEQ ID NO: 40 - DNA, 1471 bp, Rhodococcus opacus
gacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgagagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattgggacgaagtcgtaacaaggtagccgtaccggaagg

SEQ ID NO: 41 - DNA, 1482 bp, Rhodococcus sp.
gagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacgaccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgta

SEQ ID NO: 42 - DNA, 1446 bp, Rhodococcus sp.
gcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggggcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccagtggcctaaccccttgtgggagggagccgtcgaaggtgggatcggcgattgggacgaagtcgtaacaaggta

SEQ ID NO: 43 - DNA, 1497 bp, Rhodococcus wratislaviensis
cctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagccgtagagatacggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaaggtgcggctggatcacct

SEQ ID NO: 44 - DNA, 1481 bp, Rhodococcus opacus
  modified_base (570)..(570): a, c, t, g, unknown or other
cctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggctgagggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcanagctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcaaacaggattagataccctggtagtccacgccgtaaacggtgggcgctaggtgtgggtttccttccacgggatccgtgccgtagttaacgcattaagcgccccgcctggggagtacggccgcaaggttaaaactcaaaggaattgacgggggcccgcacaagcggcggagcatgtggattaattcgatgcaacgcgaagaaccttacctgggtttgacatataccggaaagccgtagagataccgccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaaccccttgtgggagggagccgtcgaaggtgggatcggcgattgggacgaagtcgtaacaaggtagccgtaccggaagg

SEQ ID NO: 45 - DNA, 1521 bp, Rhodococcus opacus
tcaacggagagtttgatcctggctcaggacgaacgctggcggcgtgcttaacacatgcaagtcgagcggtaaggcccttcggggtacacgagcggcgaacgggtgagtaacacgtgggtgatctgccctgcacttcgggataagcctgggaaactgggtctaataccggatatgaccttcggctgcatggccgttggtggaaaggtttactggtgcaggatgggcccgcggcctatcagcttgttggtggggtaatggcctaccaaggcgacgacgggtagccgacctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagcgaaagtgacggtacctgcagaagaagcaccggccaactacgtgccagcagccgcggtaatacgtagggtgcaagcgttgtccggaattactgggcgtaaagagctcgtaggcggtttgtcgcgtcgtctgtgaaaactcgaggctcaacctcgagcttgcaggcgatacgggcagacttgagtactgcaggggagactggaattcctggtgtagcggtgaaatgcgcagatatcaggaggaacaccggtggcgaaggcgggtctctgggcagtaactgacgctgaggagcgaaagcgtgggtagcgaacaggattagataccctggtagtccacgccgtaaacggtgggcgctaggtgtgggtttccttccacgggatccgtgccgtagctaacgcattaagcgccccgcctggggagtacggccgcaaggctaaaactcaaaggaattgacgggggcccgcacaagcggcggagcatgtggattaattcgatgcaacgcgaagaaccttacctgggtttgacatataccggaaagctgcagagatgtggccccccttgtggtcggtatacaggtggtgcatggctgtcgtcagctcgtgtcgtgagatgttgggttaagtcccgcaacgagcgcaacccttgtcttatgttgccagcacgtaatggtggggactcgtaagagactgccggggtcaactcggaggaaggtggggacgacgtcaagtcatcatgccccttatgtccagggcttcacacatgctacaatggccggtacagagggctgcgataccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccgaagccggtggcctaacccctcgtgggagggagccgtcgaaggtgggatcggcgattgggacgaagtcgtaacaaggtagccgtaccggaaggtgcggctggatcacctcctttct